SEQUENCE LISTING (2 sequences)

SEQ ID NO: 1 - protein, 29 aa, Homo sapiens
GVEHENGTDPWDTNECRPTEFAVRSGLLT

SEQ ID NO: 2 - protein, 29 aa, Mus musculus
GVEHENGTDPRDTNECRATEFAVRSGLLT

(19) United States
(12) Patent Application Publication
(10) Pub. No.: US 2024/0391928 A1
(43) Pub. Date: Nov. 28, 2024

(54) COMPOSITIONS FOR THE TREATMENT OF FOOD AND CHEMICAL ADDICTION AND METHODS OF MAKING AND USING SAME

(71) Applicant: Duke University, Durham, NC (US)

(72) Inventors: Dan Jansen, Bethesda, MD (US); Juan Jose Marugan, Bethesda, MD (US); Krisztian Toth, Durham, NC (US); Lawrence Barak, Durham, NC (US); Marc G. Caron, Durham, NC (US); Joshua Gross, Durham, NC (US); David Kim, Bethesda, MD (US); Tae Gyun Yang, Bethesda, MD (US)

(21) Appl. No.: 18/694,167

(22) PCT Filed: Feb. 17, 2022
(86) PCT No.: PCT/US2022/016801

§ 371 Date: Mar. 21, 2024

Related U.S. Application Data
(60) Provisional application No. 63/246,934, filed on Sep. 22, 2021.

(51) Int. Cl. C07D 491/052; A61K 31/352; A61K 31/357; A61K 31/407; A61K 31/4178; A61K 31/4439; A61K 31/501; A61K 31/506; A61K 31/5377; A61P 25/14; C07D 311/24; C07D 405/12; C07D 407/06

(52) U.S. Cl. CPC C07D 491/052; A61K 31/352; A61K 31/357; A61K 31/407; A61K 31/4178; A61K 31/4439; A61K 31/501; A61K 31/506; A61K 31/5377; A61P 25/14; C07D 311/24; C07D 405/12; C07D 407/06

(57) ABSTRACT

Embodiments of the instant disclosure relate to novel compounds, compositions, and methods for treating health conditions. In certain embodiments, methods of treating health conditions can include administering an effective amount of at least one of the compounds or compositions disclosed herein to a subject having or suspected of having an imbalance in brain dopamine homeostasis.

### CROSS REFERENCE TO RELATED APPLICATIONS

This application claims priority to U.S. Provisional Patent Application Ser. No. 63/246,934, filed Sep. 22, 2021, the contents of which is hereby incorporated by reference in its entirety.

### FEDERAL FUNDING LEGEND

This invention was made with Government support under Federal Grant Nos. U18DA052417 and P30DA029925 awarded by the National Institutes of Health. The Federal Government has certain rights to this invention.

### BACKGROUND OF THE INVENTION

G protein-coupled receptors (GPCRs) regulate various physiological functions and are frequently targeted in the treatment of disease. The GPCR ghrelin receptor (GHSR1a) is a critical regulator of food intake, energy homeostasis, and reward-seeking behaviors. Therefore, pharmacological agents targeting GHSR1a may have utility in the treatment of several mental health disorders, including eating disorders (e.g., obesity, anorexia) and drug addiction. Ghrelin, the endogenous ligand of GHSR1a, is a brain-gut peptide composed of 28 amino acids. The ghrelin-GHSR1a system can regulate the release of growth hormone (GH), affect learning and memory, and play an anti-inflammatory and anti-apoptotic role when GHSR1a is activated by ghrelin. Numerous peripheral actions of ghrelin can include, but are not limited to, regulation of glucose metabolism, lipogenesis, suppression of brown fat thermogenesis and improvement of cardiovascular and/or neurologic functions. Accordingly, pharmacological intervention of ghrelin-GHSR1a system could benefit a myriad of disorders and diseases. Despite the tremendous therapeutic potential for targeting GHSR1a, there are no GHSR1a agonists or GHSR1a antagonists currently approved for clinical use. Accordingly, there is a need in the field for the development of safe and effective pharmacologic agents that modulate GHSR1a activity.

### BRIEF SUMMARY OF THE DISCLOSURE

The present disclosure provides, in part, novel compounds, compositions, and methods for treating an imbalance in brain dopamine homeostasis in a subject.

In one aspect, disclosed herein, are compounds of Formula (I), analogs, isomers, pharmaceutically acceptable salts, and prodrugs thereof or a pharmaceutically acceptable salt thereof:

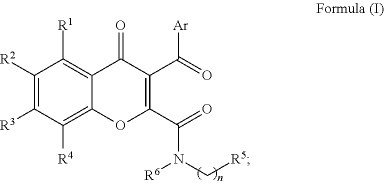

wherein R¹, R², R³, and R⁴ are independently selected from a group consisting of H, NO₂, CN, CHO, F, Cl, Br, I, CF₃, unsubstituted C₁-C₆ alkyl, substituted alkyl, COR¹², CO₂H, CO₂R¹³, CONH₂, CONHR¹⁴, and CONR¹⁵R¹⁶;
R⁵ is selected from a group consisting of OH, OR¹⁷, NH₂, NHR¹⁸, NR¹⁹R²⁰, NHCOR²¹, NHCO₂R²² and heterocyclic;
R⁶ is H or an unsubstituted C₁-C₆ alkyl,
Ar is

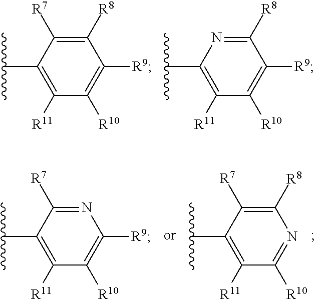

R⁷, R⁸, R⁹, R¹⁰, and R¹¹ are independently H, F, Cl, Br, I, CF₃, CN, OH, OR²³, NH₂, NHR²⁴, NR²⁵R²⁶, NHCOR²⁷, NHCO₂R²⁸, unsubstituted C₁-C₆ alkyl, substituted C₁-C₆ alkyl, unsubstituted C₂-C₆ cycloalkyl, substituted C₂-C₆ cycloalkyl, unsubstituted C₁-C₆ alkenyl, substituted C₁-C₆ alkenyl, unsubstituted C₁-C₆ alkynyl, substituted C₁-C₆ alkynyl, unsubstituted phenyl, substituted phenyl, R¹⁵ and R¹⁶ may be taken together to form an unsubstituted C₁-C₆ alkyl, or a substituted C₁-C₆ alkyl, R⁸ and R⁹ may be taken together to form an unsubstituted C₁-C₆ alkyl, or a substituted C₁-C₆ alkyl, R⁹ and R¹⁰ may be taken together to form an unsubstituted C₁-C₆ alkyl, or a substituted C₁-C₆ alkyl, R¹⁰ and R¹¹ may be taken together to form an unsubstituted C₁-C₆ alkyl, or a substituted C₁-C₆ alkyl; R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷ and R²⁸ are independently selected from a group consisting of H, unsubstituted C₁-C₆ alkyl, substituted C₁-C₆ alkyl, unsubstituted phenyl, substituted phenyl, heterocyclic, R¹⁵ and R¹⁶ may be taken together to form an unsubstituted C₁-C₆ alkyl or a substituted C₁-C₆ alkyl, and R¹⁹ and R²⁰ may be taken together to form an unsubstituted C₁-C₆ alkyl or a substituted C₁-C₆ alkyl; and
n is an integer from 1 to 6;
wherein an unsubstituted alkyl, cycloalkyl, or phenyl group comprises all carbon atoms, and a substituted alkyl, cycloalkyl, or aryl group comprises at least one nitrogen atom, oxygen atom, halogen atom, or a combination thereof for at least one carbon atom.

In another aspect, as disclosed herein, are compounds of Formula (II), analogs, isomers, prodrugs, or a pharmaceutically acceptable salt thereof:

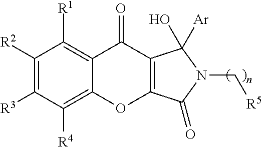

wherein R¹, R², R³, and R⁴ are independently selected from a group consisting of H, NO₂, CN, CHO, F, Cl, Br, I, CF₃, unsubstituted C₁-C₆ alkyl, substituted alkyl, COR¹², CO₂H, CO₂R¹³, CONH₂, CONHR¹⁴, and CONR¹⁵R¹⁶;
R⁵ is selected from a group consisting of OH, OR¹⁷, NH₂, NHR¹⁸, NR¹⁹OR²⁰, NHCOR²¹, NHCO₂R²², and heterocyclic;
Ar is

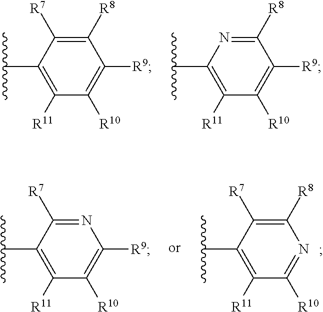

R⁷, R⁸, R⁹, R¹⁰, and R¹¹ are independently H, F, Cl, Br, I, CF₃, CN, OH, OR²³, NH₂, NHR²⁴, NR²⁵R²⁶, NHCOR²⁷, NHCO₂R²⁸, unsubstituted C₁-C₆ alkyl, substituted C₁-C₆ alkyl, unsubstituted C₂-C₆ cycloalkyl, substituted C₂-C₆ cycloalkyl, unsubstituted C₁-C₆ alkenyl, substituted C₁-C₆ alkenyl, unsubstituted C₁-C₆ alkynyl, substituted C₁-C₆ alkynyl, unsubstituted phenyl, substituted phenyl, R⁷ and R⁸ may be taken together to form an unsubstituted C₁-C₆ alkyl, or a substituted C₁-C₆ alkyl, R⁸ and R⁹ may be taken together to form an unsubstituted C₁-C₆ alkyl, or a substituted C₁-C₆ alkyl, R⁹ and R¹⁰ may be taken together to form an unsubstituted C₁-C₆ alkyl, or a substituted C₁-C₆ alkyl, R¹⁰ and R¹¹ may be taken together to form an unsubstituted C₁-C₆ alkyl, or a substituted C₁-C₆ alkyl; R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, and R²⁸ are independently selected from a group consisting of H, unsubstituted C₁-C₆ alkyl, substituted C₁-C₆ alkyl, unsubstituted phenyl, substituted phenyl, heterocyclic, R¹⁵ and R¹⁶ may be taken together to form an unsubstituted C₁-C₆ alkyl or a substituted C₁₋₆ alkyl, and R¹⁹ and R²⁰ may be taken together to form an unsubstituted C₁-C₆ alkyl or a substituted C₁-C₆ alkyl; and
n is an integer from 1 to 6;
wherein an unsubstituted alkyl, cycloalkyl, or phenyl group comprises all carbon atoms, and a substituted alkyl, cycloalkyl, or aryl group comprises at least one nitrogen atom, oxygen atom, halogen atom, or a combination thereof for at least one carbon atom.

In another aspect, as disclosed herein, provides a pharmaceutical composition comprising, consisting of, or consisting essentially of a compound of Formula (I) and/or Formula (II) and a pharmaceutically acceptable carrier and/or excipient.

In yet another aspect, as disclosed herein, provides a method of modulating GHSR1a activity in a cell and/or subject comprising, consisting of, or consisting essentially of administering to the cell and/or subject an effective amount of a compound of Formula (I) and/or Formula (II) such that the GISR1a activity is modulated in the cell and/or subject.

In still another aspect, as disclosed herein, provides a method of modulating β-arrestin (β-arr) activity in a cell and/or subject comprising, consisting of, or consisting essentially of administering to the cell and/or subject an effective amount of a compound of Formula (I) or Formula (II) such that the β-arrestin (β-arr) activity is modulated in the cell and/or subject.

In yet another aspect, as disclosed herein, provides a method of modulating G protein activity in a cell and/or subject comprising, consisting of, or consisting essentially of administering to the cell and/or subject an effective amount of a compound of Formula (I) or Formula (II) such that the G protein activity is modulated in the cell and/or subject.

In some embodiments, the compound activates the G protein, f-arrestin, and/or GHSR1a activity.

In another aspect, as disclosed herein, provides a method of treating and/or preventing a GHSR1a-associated condition in a subject, the method comprising, consisting of, or consisting essentially of administering to the subject a therapeutically effective amount of a compound of Formula (I) and/or Formula (II) such that GISR1a-associated condition is treated and/or prevented in the subject.

In some embodiments, the GHSR1a-associated condition is selected from the group consisting of eating disorders, addiction, and combinations thereof. In one embodiment, the GISR1a-associated condition comprises an eating disorder. In some embodiments, the eating disorder is selected from the group consisting of bulimia nervosa, anorexia nervosa, binge-eating disorder, and combinations thereof. In another embodiment, the GHSR1a-associated condition comprises addiction. In some embodiments, the addition comprises a chemical addiction.

Another aspect of the present disclosure provides all that is described and illustrated herein.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIGS. 1A-1K illustrate the effect of N8279 (NCATS-SM8864) on GHSR1a signaling in accordance with embodiments of the present disclosure. FIG. 1A shows N8279 2D structure and FIG. 1B shows structure determined by single crystal X-ray diffraction. FIG. 1C shows N8279 (1 μM) selectivity for GHSR1a plotted versus one (blue line) and >3-fold (dotted purple line) activity above baseline. FIG. 1D shows linear regression analysis of β-arr-based Tango assay results for GPCRome with each point XY corresponding to a distinct receptor and its co-ordinates defined by X=average of replicates 1 and 2 and Y=average of replicates 3 and 4. FIG. 1E shows [¹²⁵I]ghrelin competition binding in hGHSR1a^{WT}-expressing HEK293/T cells (unlabeled ghrelin curve, black; N8279 curve, red). Data were normalized to vehicle conditions within each experiment and pooled data normalized to the unlabeled (cold) ghrelin Top (100%) and Bottom (0%). FIG. 1F shows iCa²⁺ in hGHSR1a^{WT}- and miAeq-expressing HEK293/N cells after treatment with ghrelin (black), MK-0677 (green), L585 (blue), or N8279 (red). Bottom and Top parameters were constrained to 0% and 100% of ghrelin (% reference); ghrelin and N8279 h>1. FIG. 1G shows Ghrelin-induced iCa²⁺ with concomitant N8279 treatment. Data are normalized to the vehicle Eₘₐₓ (100%) and image displays best-fit three- or four-parameter regressions for each condition. FIG. 1H shows Gα_{q} dissociation (TRUPATH) in hGHSR1a^{WT}-expressing HEK293/T cells. Bottom and Top parameters were constrained to 0% and 100% of ghrelin (% reference) and the h shared (p>0.05). FIGS. 1I-1K show ghrelin, N8279, and L585 heat map (FIG. 1I), yellow=higher potency, blue=lower potency or inactivity; (pEC50) potencies (FIG. 1J), and max efficacies (FIG. 1K) at different G proteins from curves in FIG. 8A-8G. Statistical differences were derived from Dunnett's multiple comparisons relative to each ligand's Gαq response. *, **, ***, **** correspond to p<0.05, 0.01, 0.001, and 0.0001. All data in FIGS. 1A-1K represent the mean±SEM from multiple independent experiments.

FIGS. 2A-2L illustrate the effect of N8279 (NCATS-SM8864) on β-arr2-dependent cellular responses relative to ghrelin in accordance with embodiments of the present disclosure. FIG. 2A shows peak ^{smBiT}β-arr2 recruitment (average, 0-5 minutes) to hGHSR1a^{L}g^{BiT} in HEK293/T cells. Data were baseline normalized within each experiment, then to the ghrelin Eₘₐₓ (% reference). FIG. 2B shows hGHSR1a^{L}g^{BiT}-SmBiTβ-arr2 saturation after treatment with ghrelin (100 nM, black) or N8279 (100 nM, light red; 200 nM, dark red). Hyperbola were fit by one-site regression to derive a Bₘₐₓ (BiTₘₐₓ) and Kd (BiTd), then normalized to the ghrelin BiTₘₐₓ (100%) wherein the inset shows ghrelin and N8279 BiTd values derived from Panel B and analyzed by one-way ANOVA followed by Sidak's multiple comparisons. FIG. 2C shows [¹⁵1]ghrelin competition binding in HEK293/T cells expressing the hGSR1a^{L149G}. Data were normalized as in FIG. 1E. FIG. 2D shows Max ^{Venus}β-arr2 recruitment (over 60 min) to the hGHSR1a^{WT} or hGHSR1a^{L149G} in HEK293/T cells. Data were baseline normalized within each experiment, then the ghrelin-WT Eₘₐₓ (% reference). FIG. 2E shows EC₈₀ ghrelin (40 nM)-induced ^{SmBiT}β-arr2 recruitment to the hGHSR1a^{L}g^{BiT} after pretreatment (5 minutes) with YIL781, JMV2959, or N8279. 100% represents EC₈₀ ghrelin alone and 0% represents baseline. FIG. 2F shows representative images of vehicle-, ghrelin (100 nM)-, or N8279 (100 nM)-induced β-arr2 translocation (45 minutes at 37° C.) in U2OS cells expressing hGHSR1a^{WT} and β-arr2^{GFP}. FIG. 2G shows hGHSR1a^{WT} internalization in HEK293/T cells after 45 minutes at 37° C. Data were expressed as the % GHSR1a expression relative to baseline (100%) and pooled data were normalized to the ghrelin Top (100%) and Bottom (0%). FIG. 2H shows bBRET-based hGHSR1a^{WT-RLucII} internalization in HEK293/T cells with MyrPalm^{Venus}. Data represent the average net BRET (60 minutes) normalized to baseline within each experiment and then to the ghrelin Top (100%) and Bottom (0%). FIG. 2I shows bBRET-based hGHSR1a^{WT}-RLucII trafficking in HEK293/T cells with 2×FYVE^{Venus}. Data represent the average net BRET (60 minutes) normalized to the ghrelin Eₘₐₓ (% reference). FIG. 2J shows GHSR1a trafficking Eₘₐₓ over 120 minutes derived from Panel B. FIG. 2K shows SRF-RE-mediated transcription in HEK293/T cells. Data were normalized to the ghrelin Eₘₐₓ (% reference). FIG. 2L shows N8279 bias factor (RAi model) with 13 (log 10) quantified using ghrelin (black/grey) or L585 (blue) as reference ligands. All data represent the mean±SEM from multiple independent experiments. ***, p<0.001; ** p<0.01; n.s. (non-significant), p>0.05.

FIGS. 3A-3P illustrate distinctive receptor sites and conformational states between N8279 (NCATS-SM8864) and ghrelin interaction with GHSR1a in accordance with embodiments of the present disclosure. FIG. 3A shows an amino acid snake plot of the hGHSR1a^{WT} highlighting Ala204^{ECL2} (red) and the Ala204Glu mutation. FIGS. 3B-3G show ghrelin-induced (FIG. 3B) iCa²⁺, (FIG. 3C) TRUPATH Gq dissociation, and (FIG. 3D) NanoBiT β-arr2 recruitment at the hGHSR1a^{WT} (black) or hGHSR1a^{A204E} (purple). N8279-induced (FIG. 3E) iCa²⁺, (FIG. 3F) TRUPATH Gα_{q} dissociation, and (FIG. 3G) NanoBiT β-arr2 recruitment at the hGHSR1a^{WT} (red) or hGHSR1a^{A204E} (yellow). All data were normalized to the GHSR1a^{WT} Eₘₐₓ. Gα_{q} dissociation and β-arr2 recruitment assays are also baseline normalized. FIG. 3H shows superimposition of the ghrelin-bound model structure (blue) with the antagonist-bound x-ray crystal structure (6KO5, green). FIG. 3A shows proposed GHSR1a^{DTP} (red) and GHSR1a^{ECD} (blue) binding pockets. Gln120, Glu124, Phe279, Arg283 and Phe309 constituted the canonical, orthosteric GHISR1a^{DT} pocket and Asp99, Cys198, and Asn305 constituted predicted interaction sites within the GHSR1aECD binding pocket. FIG. 3J shows N8279 docking pose (green) in the GHSR1a^{DTP}. FIG. 3K shows N8279 docking pose (green) in the GHSR1a^{ECD}. Dash lines indicate hydrogen bonds (yellow), ionic interactions (pink) and π-π stacking interactions (turquoise), or halogen bonds (purple). FIG. 3L shows a snake plot of the hGHSR1^{WT} with experimentally mutated residues: D99A (blue), E197A (green), R199A (orange), P200A (purple), A204E (yellow), N305A (teal), and Glu124 and Cys198 (grey, mutations not made). FIGS. 3M-3P show ghrelin and N8279 Gα_{q} dissociation (TRUPATH) (FIGS. 3M-3N) and iCa²⁺ (FIGS. 3O-3P) pEC50 and Eₘₐₓ at GHSR1a mutants shown in FIG. 3L, derived from FIGS. 10A-10E. All data represent the mean±SEM from multiple independent experiments.

FIGS. 4A-4E illustrate the effects of N8279 (NCATS-SM8864) on aberrant DAergic behavior in accordance with embodiments of the present disclosure. FIG. 4A shows an analysis of brain (red) and plasma [N8279] (orange) over 24 hours in C57BL/6 mice treated with N8279 (5 mg/kg, IP). N8279 Cmax at ˜2 hours=123 ng/mL (259 nM). N8279 (IP) brain half-life (t1/2)=6.6 hours, plasma t1/2=3.8 hours. FIG. 4B shows spontaneous hyperlocomotion in DAT KO mice. 30 minutes (grey box) acclimation prior to injection (black arrow) of N8279 (2.5, 5, or 10 mg/kg, IP) or vehicle (5% DMSO, saline). Horizontal locomotion was monitored for 120 minutes post-injection and beam-breaks were collected in 5 minute bins. Results are presented as mean±SEM. N8279-treated DAT KO mice had reduced locomotion relative to vehicle-treated controls. Post-injection ANOVA, time: [F(7.6, 411.8)=14.7, p<0.0001], dose: [F(3,54)=3.4, p=0.022], time×dose interaction: [F(69,1242) 1.2, p=0.118]. No baseline differences were detected between groups. Baseline ANOVA, time: [F(2.4, 131.0)=3.0, p=0.039], dose: [F(3,54)=0.06, p=0.979], time×dose interaction: [F(15,270)=1.4, p=0.141]. n=12-20 mice per group. FIG. 4C shows one-way ANOVA for total area under the curve (AUC, 35-150 min) derived from FIG. 4B treatment: [F(3,55)=6.50, p=0.0008]. Dunnett's multiple comparisons revealed an effect of 2.5, 5, and 10 mg/kg N8279 relative to vehicle control (0 mg/kg). *, p<0.05, *** p<0.001 vs vehicle. FIGS. 4D-4E show cocaine-induced behavioral sensitization in C57BL6/J mice: experimental design (FIG. 4D) and locomotion (FIG. 4E). Post-injection results are presented as % baseline activities because locomotion was low in the N8279+vehicle group on all days. Within group analyses (significance denoted by +) relative to day 1 showed that the vehicle+cocaine group had increased locomotion on days 2-5 and day 11; whereas, the N8279+cocaine group had increased locomotion only on day 5. Between group comparisons (significance denoted by *) showed that cocaine-induced sensitization was higher on day 4 and on challenge day 11 in the vehicle-cocaine compared to the N8279+cocaine group. RMANOVA: day [F(5,145)=8.797, p<0.001], treatment [F(2,29)=32,523, p<0.001], day×treatment [F(10,145)=3.215, p<0.001]. */+, p<0.05, ***/+++, p<0.001. n=9-12 mice/group.

FIGS. 5A-5C illustrate high-throughput screening (HTS) identification of a novel lead compound in accordance with embodiments of the present disclosure. FIG. 5A shows a study flow chart of HIS compound screening, the discovery of N8279, and the chemical structures of the (FIG. 5B) inactive parental scaffold N1956 and (FIG. 5C) active, lead compound N8279 (NCATS-SM8864).

FIG. 6 illustrates compounds resulting from a high-throughput screen of GHSR1a-β-arr activation in accordance with embodiments of the present disclosure. The initial screening of 47,000 compounds in a β-arr1 DiscoverX Path Hunter assay resulted in 36 hits. The hits were further analyzed by Ca²⁺ mobilization assays using the Ca²⁺ sensor mitochondrial Aequorin (Aeq Max) for Gα_{q/11}-dependent signaling and β-arr2 translocation imaging assay (β-arrestin agonist) for β-arr signaling. The maximum responses (at 30 μM) of the compounds are ordered by Gαq/11 Ca²⁺ response. Data in each assay were normalized to activity of the full receptor agonist L585.

FIGS. 7A-7H illustrate N8279 (NCATS-SM8864) activation of receptor-dependent, Gα_{q/11}-mediated signaling at the apo- and orthosteric agonist-bound GHSR1a in accordance with embodiments of the present disclosure. FIG. 7A shows hGHSR1a^{WT} saturation binding with [¹²⁵I]ghrelin. Hyperbola were fit by one-site, non-linear regression (Bₘₐₓ=755.7 CPM±112.7; Kd=4.2 nM±1.3). FIGS. 7B-3D shows HEK293/N cells stably expressing hGHSR1aWT and miAEQ were stimulated with EC₈₀ (FIG. 7B) N8279, (FIG. 7C) ghrelin, or (FIG. 7D) L585 following 30 minute pretreatment with increasing concentrations of the antagonist YIL781 or JMV2959. The data in each panel were normalized to their respective baseline (100%) and maximal inhibitory effect (0%). Data were fit by three- or four-parameter non-linear regression and represent the mean+/−SEM from 3 independent experiments. FIG. 7E shows 10 μM ghrelin (black)-, N8279 (red)-, and L585 (blue)-induced iCa²⁺ in WT or Gαq/11 KO HEK293/S cells. Data were normalized to ghrelin (% reference; N=2 independent experiments). Two-way ANOVA analysis revealed a significant effect of cell line (F(1,18)=972.8, p<0.0001)) and Sidak's multiple comparisons analyses showed a significant effect between WT and Gαq/11 KO cells for each ligand (****, p<0.0001). FIG. 7F shows N8279-induced iCa²⁺ with (+vehicle, red) or without EC₂₅ MK-0677 (orange) or EC₅₀ ghrelin (purple). Data are normalized to the N8279+vehicle Eₘₐₓ (% N8279 Eₘₐₓ). Curves with h≠1 were fit by four-parameter nonlinear regression (N8279+vehicle: *=p<0.05), whereas curves h=1 were fit by three-parameter regression (N8279+MK0677EC₂₅ and N8279+ghrelinEC₅₀: p>0.05=#), as determined by extra sum-of-squares ANOVA. ANOVA analysis across conditions revealed a significant effect of Hill Slope: F(2,288)=15.23, p<0.0001, Top: F(2,288)=3.2, p=0.041, and Bottom: F(2,288)=75.2, p<0.0001, but not log EC50: F(2,288)=1.2, p=0.290. FIG. 7G shows an in silico model of N8279 (green) docked concomitantly into a NMR-based homology model of the GHSR1a with ghrelin (1-17) bound (purple). N8279 sits atop ghrelin, interacting with Asp191 in ECL2 (docking score=−4.871). FIG. 7H shows hGHSR1a^{WT}-mediated Gα_{q} activation using NanoBiT. Gα_{q} activation was quantified as the average % decrease from baseline (0%) after ligand treatment (over 20 minutes). N8279 and ghrelin were potent (N8279 pEC₅₀=7.60±0.19; ghrelin pEC₅₀=7.99±0.17), full agonists (N8279 Eₘₐₓ=118.3±9.8; ghrelin Eₘₐₓ=100.0±7.35) and potencies did not significantly differ (N8279 vs ghrelin log EC₅₀: F(1,150)=1.51, p=0.22)).

FIGS. 8A-8G illustrate ghrelin-, N8279-, and L585-induced Ga dissociation from the GHSR1aWT using the BRET-based TRUPATH assay in accordance with embodiments of the present disclosure. Ghrelin (black, solid)-, N8279 (red, dotted)-, and L585 (blue, solid)-induced G protein activation at the hGHSR1a^{WT} with (FIG. 8A) Gα_{q} (FIG. 8B) Gαᵢ₁ (FIG. 8C) Gαᵢ₂ (FIG. 8D) Gβ_{oA} (FIG. 8E) Gα₁₂ (FIG. 8F) Gα₁₃ and (FIG. 8G) Gα_{sS}. Data were plotted as the minimum delta net BRET (maximal response) over 30 minutes post-treatment and fit by non-linear regression. All data represent the mean±SEM from >3 independent experiments.

FIG. 9 illustrates bias factor using an assay-standardized calculation in accordance with embodiments of the present disclosure. N8279 (NCATS-SM8864) bias factor (0) relative to ghrelin was calculated using the RAi method as in FIG. 2L. C/R BRET data were derived from FIG. 1H (Gα_{q} dissociation, TRUPATH) and FIG. 2D (GHSR1a^{RLucII_Venus}β-arr2 BRET), whereas C/R NanoBiT data were derived from FIG. S3H (Gα_{q} dissociation, NanoBiT) and FIG. 2A (GHSR1a^{L} g^{BiT_SmBiT}β-arr2 NanoBiT). N8279-BRET=1.16±0.11 (˜14-fold); N8279-NanoBiT β=1.36±0.33 (˜23-fold).

FIGS. 10A-10E illustrate cell surface expression, Gα_{q} dissociation, and iCa²⁺ of GHSR1a mutants in accordance with embodiments of the present disclosure. FIG. 10A shows surface expression of hGHSR1a variants determined by on-cell ELISA. The expression of hGHSR1a^{D99A} (D99A, blue), hGHSR1a^{E197A} (green), hGHSR1a^{R199A} (R199A, orange), hGHSR1a^{Z200A} (P200A, purple), hGHSR1a^{A204E} (A204E, yellow), and hGHSRa^{N305A} (N305A, turquoise) was assessed relative to hGHSR1a^{WT} (100%; WT, black) and data were analyzed by one-way ANOVA. **, p<0.01, ****, p<0.0001. Mean expression±SEM: WT 100±1.2; D99A=37.6±2.5; E197A=67.0±2.6; R199A=114.4±11.9; P200A=128.7±13.0; A204E=55.5±8.9; N305A=89.5±10.1. Data represent the pooled results from >2 independent experiments with at least 2 technical replicates per experiment. FIGS. 10B-10E show C/R curves produced by (FIG. 10D) ghrelin- and (FIG. 10B) N8279-induced Gα_{q} dissociation and (FIG. 10E) ghrelin- and (FIG. 10C) N8279-induced iCa²⁺ assays in HEK293/T cells transiently expressing human GHSR1a WT (red), D99A (blue), E197A (green), R199A (orange), P200A (purple), or N305A (turquoise). Mutants with ˜50% the surface expression of the WT GHSR1a (D99A, E197A, and A204E; FIG. 10A) were transfected at 2× the amount of the GHSR1a^{WT}. For all iCa²⁺ assays with GHSR1a variants, the average N8279- or ghrelin-stimulated response over 10 seconds was calculated and the data were normalized to the GHSR1a^{WT} Eₘₐₓ for each ligand (100%), whereas the max effect (Δ net BRET) over 30 minutes was used for all Gα_{q} dissociation assays. Dashed lines indicate a significant difference in potency or efficacy from the GHSR1a^{WT} control, and dotted lines indicate no measurable agonist-stimulated response. Data represent the pooled results from >3 independent experiments.

FIGS. 11A-11B illustrate docking of N8279 (NCATS-SM8864) into the GHSR1a crystal structure (6KO5) in accordance with embodiments of the present disclosure. FIG. 11A shows differences in the side chain conformations of Asp99, Gln120, Glu124, Phe279, Arg283 and Phe286 between the ghrelin-bound model structure (blue) and the antagonist-bound x-ray crystal structure (6KO5, green). FIG. 11B shows best docking pose of N8279 docked to the antagonist-bound x-ray crystal structure (6KO5), with a docking score of −5.040. Dash lines indicated hydrogen bonds (yellow), π-π stacking interactions (turquoise) and halogen bonds (purple).

FIGS. 12A-12C illustrate N8279 (NCATS-SM8864) in C57BL6 mice after treatment in accordance with embodiments of the present disclosure. Mean concentration (±SD)-time profiles of N8279 in C57BL6 mice after single dose treatment of 1 mg/kg (IV; FIG. 12A), 5 mg/kg (PO; FIG. 12B) and 5 mg/kg (IP; FIG. 12C) are shown.

FIG. 13 illustrates N8279 (NCATS-SM8864)-induced open-field locomotor activity in WT C57BL/6J mice in accordance with embodiments of the present disclosure. Novelty-induced locomotor activity was measured in N8279-treated, male and female WT C57BL/6J mice. Treatment and behaviorally naive mice were administered (IP) vehicle (5% DMSO and saline, black), 2.5 mg/kg N8279 (orange), 5 mg/kg N8279 (red), or 10 mg/kg N8279 (purple) and immediately allowed to explore the open-field chamber. N=14-17 mice per treatment group. No statistically significant differences were identified between any treatment groups (p>0.05).

FIG. 14 illustrates ligand C/R curve parameters and statical comparations from FIGS. 1E-1K in accordance with embodiments of the present disclosure.

FIG. 15 illustrates ligand C/R curve parameters and statical comparations from FIGS. 2A-2K in accordance with embodiments of the present disclosure.

FIG. 16 illustrates ligand C/R curve parameters and statical comparations from FIGS. 3B-3P in accordance with embodiments of the present disclosure.

FIGS. 17A-17C illustrate biochemical evidence of N8279 (NCATS-SM8864) signaling response in a humanized ECL2 in the mouse receptor in accordance with embodiments of the present disclosure. FIG. 17A shows identification of amino acid variations in ECL2. FIGS. 17B-17C shows N8279-stimulated Gαq dissociation (FIG. 17B) and β-arr2 recruitment (FIG. 17C) in hGHSR and mGHSR mutants and WT receptors.

### DETAILED DESCRIPTION

GPCRs, including GHSR1a, are known to signal via distinct pathways: pathways mediated by either G proteins or beta-arrestins (β-arr). GHSR1a activation results in pleiotropic physiological outcomes through distinct and pharmacologically separable C protein- and β-arr-dependent signaling pathways. Thus, as provided in the present disclosure, pathway-selective modulation can enable improved pharmacotherapeutics that may promote therapeutic efficacy while mitigating one or more side effects that have thus far prevented clinical use of pharmacologic agents that modulate GHSR1a activity.

The present disclosure is based, in part, on the discovery of a novel GHSR1a-agonist (NCG00536164-01) and its oxidized version (NCGC00538279-01). NCGC00538279 is also referred to in the present disclosure as “N8279” and “NCATS-SM8864” interchangeably. In certain embodiments, GHSR1a-agonists disclosed herein may target one or more pathophysiological changes in CNS dopamine homeostasis. In certain embodiments, GHSR1a-agonists disclosed herein may normalize dysfunctional dopamine signaling in the brain. In certain embodiments, GHSR1a-agonists disclosed herein may be used to treat and/or prevent one or more brain disorders of mood, cognition, or movement, including addiction, Alzheimer's Disease (AD), Parkinson's Disease (PD), and the like. Accordingly, disclosed herein are GHSR1a-agonists (e.g., NCG00536164-01 and NCGC00538279-01) and methods of making such, pharmaceutical compositions comprising such GHSR1a-agonists, and methods for administering GHSR1a-agonists for treating a target disease, such as a disease originating from the brain.

### I. Definitions

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

As used in the specification, articles “a” and “an” are used herein to refer to one or to more than one (i.e., at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result. The term “about” in association with a numerical value means that the numerical value can vary plus or minus by 5% or less of the numerical value.

Throughout this specification, unless the context requires otherwise, the word “comprise” and “include” and variations (e.g., “comprises,” “comprising,” “includes,” “including”) will be understood to imply the inclusion of a stated component, feature, element, or step or group of components, features, elements or steps but not the exclusion of any other integer or step or group of integers or steps.

As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise-Indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder, or condition.

As used herein, “prevent” or “prevention” refers to eliminating or delaying the onset of a particular disease, disorder, or physiological condition, or to the reduction of the degree of severity of a particular disease, disorder or physiological condition, relative to the time and/or degree of onset or severity in the absence of intervention.

The term “effective amount” or “therapeutically effective amount” refers to an amount sufficient to effect beneficial or desirable biological and/or clinical results.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. In some embodiments, the subject comprises a human. In other embodiments, the subject comprises a human in need of bone repair or bone formation.

Unless otherwise defined, all technical terms used herein have the same meaning as commonly understood by one of ordinary skill in the art to which this disclosure belongs.

### II. GHSR1a-Agonists

The present disclosure is based, in part, on the discovery novel GHSR1a-agonists. As used herein, a GHSR1a-agonist refers to a small molecule (e.g., a chemical) that activates a GHSR1a receptor to produce a biological response. Examples of biological responses stimulated by GHSR1a-agonists disclosed herein may include, but are not limited to, activation and accumulation of phospholipase C γ (PLCγ), activation of β-arrestin, accumulation of inositol phosphatase 3 kinase (IP3K), production of diacylglycerol (DAG), release of Ca+ from the endoplasmic reticulum, facilitating calcium/calmodulin kinase (CaCMK)-catalyzed phosphorylation of adenosine monophosphate-activated protein kinase (pAMPK), increased phosphorylation of ERK via the β-arrestin pathway, increased AKT phosphorylation via increased levels of PI3K, and the like. Other examples of biological responses stimulated by GHSR1a-agonists disclosed herein may include, but are not limited to, food intake stimulation, lower addiction to drugs (e.g., alcohol, cocaine), promote growth hormone secretion, and the like.

In some embodiments, the present disclosure provides a GHSR1a-agonist compound comprising, consisting of, or consisting essentially of the general Formula (I), analogs, isomers, pharmaceutically acceptable salts, and prodrugs thereof or a pharmaceutically acceptable salt thereof.

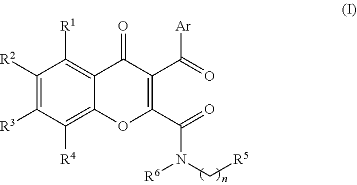

wherein R¹, R², R³, and R⁴ are independently selected from a group consisting of H, NO₂, CN, CHO, F, Cl, Br, I, CF₃, unsubstituted C₁-C₆ alkyl, substituted alkyl, COR¹², CO₂H, CO₂R¹³, CONH₂, CONHR¹⁴, and CONR¹¹R¹⁶;
R⁵ is selected from a group consisting of OH, OR¹⁷, NH₂, NHR⁸, NR¹⁹R²⁰, NHCOR²¹, NHCO₂R²², and heterocyclic;
R⁶ is H or an unsubstituted C₁-C₆ alkyl,
Ar is

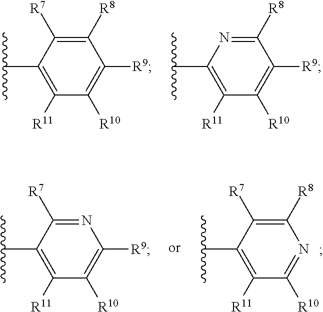

R⁷, R⁸, R⁹, R¹⁰, and R¹¹ are independently H, F, Cl, Br, I, CF₃, CN, OH, OR²³, NH₂, NHR²⁴, NR²⁵R²⁶, NHCOR²⁷, NHCO₂R²⁸, unsubstituted C₁-C₆ alkyl, substituted C₁-C₆ alkyl, unsubstituted C₂-C₆ cycloalkyl, substituted C₂-C₆ cycloalkyl, unsubstituted C₁-C₆ alkenyl, substituted C₁-C₆ alkenyl, unsubstituted C₁-C₆ alkynyl, substituted C₁-C₆ alkynyl, unsubstituted phenyl, substituted phenyl, R⁷ and R⁸ may be taken together to form an unsubstituted C₁-C₆ alkyl, or a substituted C₁-C₆ alkyl, R⁸ and R⁹ may be taken together to form an unsubstituted C₁-C₆ alkyl, or a substituted C₁-C₆ alkyl, R⁹ and R¹⁰ may be taken together to form an unsubstituted C₁-C₆ alkyl, or a substituted C₁-C₆ alkyl, R¹⁰ and R¹¹ may be taken together to form an unsubstituted C₁-C₆ alkyl, or a substituted C₁-C₆ alkyl; R¹², R¹³, R¹⁴, R¹⁵, R¹⁶, R¹⁷, R¹⁸, R¹⁹, R², R²¹, R²², R²³, R²⁴, R²⁵, R²⁶, R²⁷, and R²⁸ are independently selected from a group consisting of H, unsubstituted C₁-C₆ alkyl, substituted C₁-C₆ alkyl, unsubstituted phenyl, substituted phenyl, heterocyclic, R¹⁵ and R¹⁶ may be taken together to form an unsubstituted C₁-C₆ alkyl or a substituted C₁-C₆ alkyl, and R¹⁹ and R²⁰ may be taken together to form an unsubstituted C₁-C₆ alkyl or a substituted C₁-C₆ alkyl; and n is an integer from 1 to 6;
wherein an unsubstituted alkyl, cycloalkyl, or phenyl group comprises all carbon atoms, and a substituted alkyl, cycloalkyl, or aryl group comprises at least one nitrogen atom, oxygen atom, halogen atom, or a combination thereof for at least one carbon atom.

In general, R¹, R², R³, and R⁴ are independently selected from a group consisting of H, NO₂, CN, CHO, F, Cl, Br, I, CF₃, unsubstituted C₁-C₆ alkyl, substituted alkyl, COR¹², CO₂H, CO₂R¹³, CONH₂, CONHR¹⁴, and CONR¹⁵R¹⁶ wherein R¹², R¹³, R¹⁴, R¹⁵, and R¹⁶ are independently selected from a group consisting of H, unsubstituted C₁-C₆ alkyl, substituted C₁-C₆ alkyl, unsubstituted phenyl, substituted phenyl, heterocyclic, R¹⁵ and R¹⁶ may be taken together to form an unsubstituted C₁-C₆ alkyl or a substituted C₁-C₆ alkyl. In some embodiments, R¹, R², R³, and R⁴ are independently selected from a group consisting of H, NO₂, CN, CHO, F, Cl, Br, I, CF₃, unsubstituted C₁-C₆ alkyl, substituted alkyl, COR¹, CO₂H, CO₂R¹³, CONH₂, CONHR¹⁴, and CONR¹⁵R¹⁶ wherein R¹², R¹³, R¹⁴, R¹⁵, and R¹⁶ are independently selected from a group consisting of H, C₁-C₄ alkyl, unsubstituted phenyl, substituted phenyl, heterocycle. In certain embodiments, R¹, R², R³, and R⁴ are independently selected from a group consisting of H, F, Cl, Br, I, CF₃, CH₃, CH₂CH₃, CH₂CH₂CH₃, CH(CH₃)₂, and C(CH₃)₃. In specific embodiments, R¹, R³, and R⁴ are H; R² is Cl.

Generally, R⁵ is selected from a group consisting of OH, OR¹⁷, NH₂, NHR¹⁸, NR¹⁹R²⁰, NHCOR²¹, NHCO₂R²², and heterocyclic wherein R¹⁷, R¹⁸, R¹⁹, R²⁰, R²¹, and R²² are independently selected from a group consisting of H, unsubstituted C₁-C₆ alkyl, substituted C₁-C₆ alkyl, unsubstituted phenyl, substituted phenyl, heterocyclic, R¹⁵ and R¹⁶ may be taken together to form an unsubstituted C₁-C₆ alkyl or a substituted C₁-C₆ alkyl, and R¹⁹ and R²⁰ may be taken together to form an unsubstituted C₁-C₆ alkyl or a substituted C₁-C₆ alkyl. In some embodiments, R⁵ is selected from a group consisting of OH, OR¹⁷, NH₂, NHR¹⁸, NR¹⁹R²⁰, NHCOR²¹, and NHCO₂R²² wherein R¹⁷, R¹⁸, R¹⁹, R²⁰, R²¹, and R²² are independently selected from a group consisting of H, C₁-C₄ alkyl, unsubstituted phenyl, substituted phenyl, heterocycle, and R¹⁹ and R²⁰ may be taken together to form an unsubstituted C₁-C₆ alkyl or a substituted C₁-C₆ alkyl. In certain embodiments, R⁵ is selected from a group consisting of NHCH₃, N(CH₃)₂, NHCH₂CH₃, N(CH₂CH₃)₂, NCH₃CH₂CH₃, NHCH₂CH₂CH₃, N(CH₂CH₂CH₃)₂, NHCH(CH₃)₂, N(CH(CH₃)₂)₂, NCH₃Ph, NCH₂CH₃Ph, NCH₃CH(CH₃)₂, NH(C(CH₃)₃, pyridine, pyrazole, pyridazine, and pyrimidine. In specific embodiments, R⁵ is N(CH₃)₂.

In general, R⁶ is H or an unsubstituted C₁-C₆ alkyl. In some embodiments, R⁶ is H or an unsubstituted C₁-C₆ alkyl. In certain embodiments, R⁶ is H or CH₃. In specific embodiments, R⁶ is H.

In general, Ar is

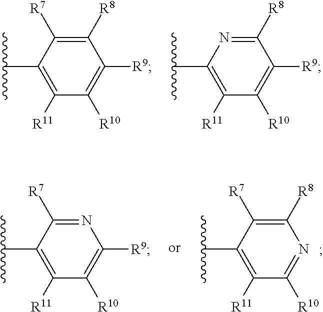

In some embodiments, Ar is

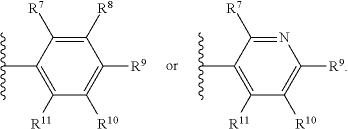

In certain embodiments, Ar is

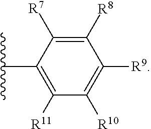

In specific embodiments, Ar is

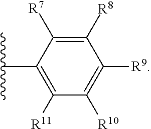

Generally, R⁷, R⁸, R⁹, R¹⁰, and R¹¹ are independently H, F, Cl, Br, I, CF₃, CN, OH, OR²³, NH₂, NHR²⁴, NR²⁵R²⁶, NHCOR²⁷, NHCO₂R²⁸, unsubstituted C₁-C₆ alkyl, substituted C₁-C₆ alkyl, unsubstituted C₂-C₆ cycloalkyl, substituted C₂-C₆ cycloalkyl, unsubstituted C₁-C₆ alkenyl, substituted C₁-C₆ alkenyl, unsubstituted C₁-C₆ alkynyl, substituted C₁-C₆ alkynyl, unsubstituted phenyl, substituted phenyl, R⁷ and R⁸ may be taken together to form an unsubstituted C₁-C₆ alkyl, or a substituted C₁-C₆ alkyl, R⁸ and R⁹ may be taken together to form an unsubstituted C₁-C₆ alkyl, or a substituted C₁-C₆ alkyl, R⁹ and R¹⁰ may be taken together to form an unsubstituted C₁-C₆ alkyl, or a substituted C₁-C₆ alkyl, R¹⁰ and R¹¹ may be taken together to form an unsubstituted C₁-C₆ alkyl, or a substituted C₁-C₆ alkyl wherein R²³, R²⁴, R²⁵, R²⁶, R²⁷, and R²⁸ are independently selected from a group consisting of H, unsubstituted C₁-C₆ alkyl, substituted C₁-C₆ alkyl, unsubstituted phenyl, substituted phenyl, heterocyclic, R¹⁵ and R¹⁶ may be taken together to form an unsubstituted C₁-C₆ alkyl or a substituted C₁-C₆ alkyl, and R¹⁹ and R²⁰ may be taken together to form an unsubstituted C₁-C₆ alkyl or a substituted C₁-C₆ alkyl. In some embodiments, R⁷, R⁸, R⁹, R¹⁰, and R¹¹ are independently H, F, Cl, Br, I, CF₃, CN, OH, OR²³, unsubstituted C₁-C₄ alkyl, substituted C₁-C₄ alkyl, unsubstituted phenyl, or substituted phenyl, R⁷ and R⁸ may be taken together to form an unsubstituted C₁-C₄ alkyl, or a substituted C₁-C₄ alkyl, R⁸ and R⁹ may be taken together to form an unsubstituted C₁-C₄ alkyl, or a substituted C₁-C₄ alkyl, R⁹ and R¹⁰ may be taken together to form an unsubstituted C₁-C₄ alkyl, or a substituted C₁-C₄ alkyl, R¹⁰ and R¹¹ may be taken together to form an unsubstituted C₁-C₄ alkyl, or a substituted C₁-C₄ alkyl wherein R²³ are independently selected from a group consisting of H, C₁-C₄ alkyl, unsubstituted phenyl, substituted phenyl, and heterocycle. In certain embodiments, R⁷, R⁸, R⁹, R¹⁰, and R¹¹ are independently selected from a group consisting of H, F, Cl, Br, I, CF₃, CN, OH, OCH₃, OCH₂CH₃, OPh, CH₃, CHR⁷, R⁸, and R¹¹ are H; R⁸ and R⁹ are OCH₃.

In general, n is an integer from 1 to 6. In some embodiments, n is an integer from 1 to 4. In certain embodiments, n is an integer from 1 to 4. In specific embodiments, n is 3.

In one preferred embodiment, R¹, R³, R⁴, and R⁶ are H; R² is Cl; R⁵ is N(CH₃)₂; Ar is

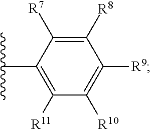

R⁷, R⁸, and R¹¹ are H; R⁸ and R⁹ are OCH₃ and n=3 as shown in the compound comprising Formula (111):

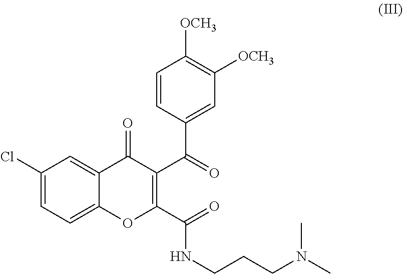 [SMILES: COc1ccc(C(=O)c2c(C(=O)NCCCN(C)C)oc3ccc(Cl)cc3c2=O)cc1OC]

also referred to as NCGC00538279.

In another embodiment, the present disclosure provides a GHSR1a-agonist compound comprising, consisting of, or consisting essentially of the general Formula (II), analogs, isomers, pharmaceutically acceptable salts, and prodrugs thereof or a pharmaceutically acceptable salt thereof:

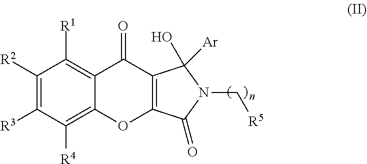

wherein R¹, R², R³, and R⁴ are independently selected from a group consisting of H, NO₂, CN, CHO, F, Cl, Br, I, CF₃, unsubstituted C₁-C₆ alkyl, substituted alkyl, COR¹², CO₂H, CO₂R¹³. CONH₂, CONHR¹⁴, and CONR¹⁵R¹⁶
R⁵ is selected from a group consisting of OH, OR¹⁷, NH₂, NHR¹⁸, NR¹⁹R²⁰, NHCOR²¹, NHCO₂R²² and heterocyclic;
Ar is

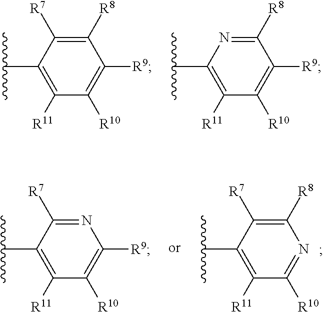

R⁷, R⁸, R⁹, R¹⁰, and R¹¹ are independently H, F, Cl, Br, I, CF₃, CN, OH, OR²³, NH₂, NHR²⁴, NR²⁵R²⁶, NHCOR²⁷, NHCO₂R²⁸, unsubstituted C₁-C₆ alkyl, substituted C₁-C₆ alkyl, unsubstituted C₂-C₆ cycloalkyl, substituted C₂-C₆ cycloalkyl, unsubstituted C₁-C₆ alkenyl, substituted C₁-C₆ alkenyl, unsubstituted C₁-C₆ alkynyl, substituted C₁-C₆ alkynyl, unsubstituted phenyl, substituted phenyl, R⁷ and R⁸ may be taken together to form an unsubstituted C₁-C₆ alkyl, or a substituted C₁-C₆ alkyl, R⁸ and R⁹ may be taken together to form an unsubstituted C₁-C₆ alkyl, or a substituted C₁-C₆ alkyl, R⁹ and R¹⁰ may be taken together to form an unsubstituted C₁-C₆ alkyl, or a substituted C₁-C₆ alkyl, R¹⁰ and R¹¹ may be taken together to form an unsubstituted C₁-C₆ alkyl, or a substituted C₁-C₆ alkyl;
R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷ and R²⁸ are independently selected from a group consisting of H, unsubstituted C₁-C₆ alkyl, substituted C₁-C₆ alkyl, unsubstituted phenyl, substituted phenyl, heterocyclic, R¹⁵ and R¹⁶ may be taken together to form an unsubstituted C₁-C₆ alkyl or a substituted C₁-C₆ alkyl, and R¹⁹ and R²⁰ may be taken together to form an unsubstituted C₁-C₆ alkyl or a substituted C₁-C₆ alkyl; and
n is an integer from 1 to 6;
wherein an unsubstituted alkyl, cycloalkyl, or phenyl group comprises all carbon atoms, and a substituted alkyl, cycloalkyl, or aryl group comprises at least one nitrogen atom, oxygen atom, halogen atom, or a combination thereof for at least one carbon atom.

In general, R¹, R², R³, and R⁴ are independently selected from a group consisting of H, NO₂, CN, CHO, F, Cl, Br, I, CF₃, unsubstituted C₁-C₆ alkyl, substituted alkyl, COR¹², CO₂H, CO₂R¹, CONH₂, CONHR¹⁴, and CONR¹⁵R¹⁶ wherein R¹, R¹³, R¹⁴, R¹⁵, and R¹⁶ are independently selected from a group consisting of H, unsubstituted C₁-C₆ alkyl, substituted C₁-C₆ alkyl, unsubstituted phenyl, substituted phenyl, heterocyclic, R¹⁵ and R¹⁶ may be taken together to form an unsubstituted C₁-C₆ alkyl or a substituted C₁-C₆ alkyl. In some embodiments, R¹, R², R³, and R⁴ are independently selected from a group consisting of H, NO₂, CN, CHO, F, Cl, Br, I, CF₃, unsubstituted C₁-C₆ alkyl, substituted alkyl, COR², CO₂H, CO₂R¹³, CONH₂, CONHR¹⁴, and CONR¹⁵R¹⁶ wherein R¹², R¹³, R¹⁴, R¹⁵, and R¹⁶ are independently selected from a group consisting of H, C₁-C₄ alkyl, unsubstituted phenyl, substituted phenyl, heterocycle. In certain embodiments, R¹, R², R³, and R⁴ are independently selected from a group consisting of H, F, Cl, Br, I, CF₃, CH₃, CH₂CH₃, CH₂CH₂CH₃, CH(CH₃)₂, and C(CH₃)₃. In specific embodiments, R¹, R³, and R⁴ are H; R² is C₁.

Generally, R⁵ is selected from a group consisting of OH, OR¹, NH₂, NHR¹⁸, NR¹⁹R²⁰, NHCOR²¹, NHCO₂R²², and heterocyclic wherein R¹⁷, R¹⁸, R¹⁹, R²⁰, R²¹, and R²² are independently selected from a group consisting of H, unsubstituted C₁-C₆ alkyl, substituted C₁-C₆ alkyl, unsubstituted phenyl, substituted phenyl, heterocyclic, R¹⁵ and R¹⁶ may be taken together to form an unsubstituted C₁-C₆ alkyl or a substituted C₁-C₆ alkyl, and R¹⁹ and R²⁰ may be taken together to form an unsubstituted C₁-C₆ alkyl or a substituted C₁-C₆ alkyl. In some embodiments, R⁵ is selected from a group consisting of OH, OR¹², NH₂, NHR¹⁸, NR¹⁹R²⁰, NHCOR²¹, and NHCO₂R²² wherein R¹⁷, R¹⁸, R¹⁹, R²⁰, R²¹, and R²² are independently selected from a group consisting of H, C₁-C₄ alkyl, unsubstituted phenyl, substituted phenyl, heterocycle, and R¹⁹ and R²⁰ may be taken together to form an unsubstituted C₁-C₆ alkyl or a substituted C₁-C₆ alkyl. In certain embodiments, R⁵ is selected from a group consisting of NHCH₃, N(CH₃)₂, NHCH₂CH₃, N(CH₂CH₃)₂, NCH₃CH₂CH₃, NHCH₂CH₂CH₃, N(CH₂CH₂CH₃)₂, NHCH(CH₃)₂, N(CH(CH₃)₂)₂, NCH₃Ph, NCH₂CH₃Ph, NCH₃CH(CH₃)₂, NH(C(CH₃)₃, pyridine, pyrazole, pyridazine, and pyrimidine. In specific embodiments, R⁵ is N(CH₃)₂.

In general, Ar is

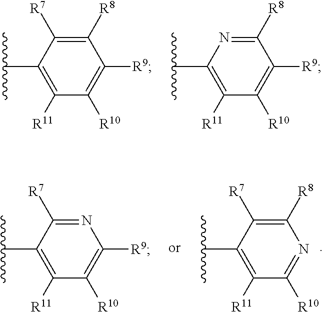

In some embodiments, Ar is

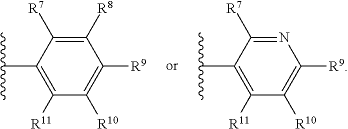

In certain embodiments, Ar is

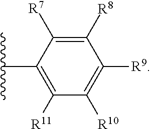

In specific embodiments, Ar is

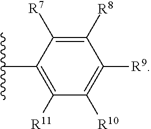

Generally, R⁷, R⁸, R⁹, R¹⁰, and R¹¹ are independently H, F, Cl, Br, I, CF₃, CN, OH, OR³, NH₂, NHR²⁴, NR²⁵R²⁶, NHCOR²⁷, NHCO₂R²⁸, unsubstituted C₁-C₆ alkyl, substituted C₁-C₆ alkyl, unsubstituted C₂-C₆ cycloalkyl, substituted C₂-C₆ cycloalkyl, unsubstituted C₁-C₆ alkenyl, substituted C₁-C₆ alkenyl, unsubstituted C₁-C₆ alkynyl, substituted C₁-C₆ alkynyl, unsubstituted phenyl, substituted phenyl, R⁷ and R⁸ may be taken together to form an unsubstituted C₁-C₆ alkyl, or a substituted C₁-C₆ alkyl, R⁸ and R⁹ may be taken together to form an unsubstituted C₁-C₆ alkyl, or a substituted C₁-C₆ alkyl, R⁹ and R¹⁰ may be taken together to form an unsubstituted C₁-C₆ alkyl, or a substituted C₁-C₆ alkyl, R¹⁰ and R¹¹ may be taken together to form an unsubstituted C₁-C₆ alkyl, or a substituted C₁-C₆ alkyl wherein R², R²⁴, R²⁵, R²⁶, R²⁷, and R²⁸ are independently selected from a group consisting of H, unsubstituted C₁-C₆ alkyl, substituted C₁-C₆ alkyl, unsubstituted phenyl, substituted phenyl, heterocyclic, R¹¹ and R¹⁶ may be taken together to form an unsubstituted C₁-C₆ alkyl or a substituted C₁-C₆ alkyl, and R¹⁹ and R²⁰ may be taken together to form an unsubstituted C₁-C₆ alkyl or a substituted C₁-C₆ alkyl. In some embodiments, R⁷, R⁸, R⁹, R¹⁰ and R¹¹ are independently H, F, Cl, Br, I, CF₃, CN, 01-1, OR²³, unsubstituted C₁-C₄ alkyl, substituted C₁-C₄ alkyl, unsubstituted phenyl, or substituted phenyl, R⁷ and R⁸ may be taken together to form an unsubstituted C₁-C₄ alkyl, or a substituted C₁-C₄ alkyl, R⁸ and R⁹ may be taken together to form an unsubstituted C₁-C₄ alkyl, or a substituted C₁-C₄ alkyl, R⁹ and R¹⁰ may be taken together to form an unsubstituted C₁-C₄ alkyl, or a substituted C₁-C₄ alkyl, R¹⁰ and R¹¹ may be taken together to form an unsubstituted C₁-C₄ alkyl, or a substituted C₁-C₄ alkyl wherein R²³ are independently selected from a group consisting of H, C₁-C₄ alkyl, unsubstituted phenyl, substituted phenyl, and heterocycle. In certain embodiments, R⁷, R⁸, R⁹, R¹⁰, and R¹¹ are independently selected from a group consisting of H, F, Cl, Br, I, CF₃, CN, OH, OCH₃, OCH₂CH₃, OPh, CH₃, CHR⁷, R⁸, and R¹¹ are H; R⁸ and R⁹ are OCH₃.

In general, n is an integer from 1 to 6. In some embodiments, n is an integer from 1 to 4. In certain embodiments, n is an integer from 1 to 4. In specific embodiments, n is 3.

In one preferred embodiment, R¹, R³, and R⁴ are H; R² is Cl; R³ is N(CH₃)₂; Ar is

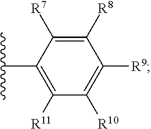

R⁷, R⁸, and R¹¹ are H; R⁸ and R⁹ are OCH₁₃ and n=3 as shown in the compound comprising Formula (IV):

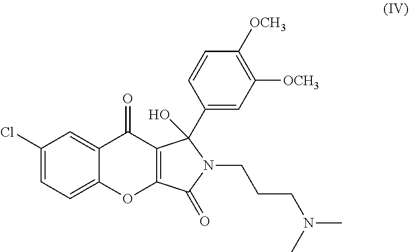 [SMILES: COc1ccc(C2(O)c3c(oc4ccc(Cl)cc4c3=O)C(=O)N2CCCN(C)C)cc1OC]

also referred to as NCG00536164-01.

Definitions of specific functional groups and chemical terms are described in more detail below. For purposes of this disclosure, the chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5th Edition, John Wiley & Sons, Inc., New York, 2001; Larock, Comprehensive Organic Transformations, VCH Publishers, Inc., New York, 1989; Carruthers, Some Modern Methods of Organic Synthesis, 3rd Edition, Cambridge University Press, Cambridge, 1987; the entire contents of each of which are incorporated herein by reference.

The term “alkyl” refers to a straight or branched saturated hydrocarbon chain. Alkyl groups may include a specified number of carbon atoms. For example, C₁-C₁₂ alkyl indicates that the alkyl group may have 1, 2, 3, 4, 5, 6, 7, 8, 9, 10, 11 or 12 carbon atoms. An alkyl group may be, e.g., a C₁ 1-C₁₂ alkyl group, a C₁-C₁₀ alkyl group, a C₁-C₆ alkyl group, a C₁-C₆ alkyl group or a C₁-C₄ alkyl group. For example, exemplary C₁-C₄ alkyl groups include methyl, ethyl, n-propyl, isopropyl, n-butyl, sec-butyl, isobutyl, and tert-butyl groups. An alkyl group may be optionally substituted with one or more substituents.

The term “alkylenyl” refers to a divalent alkyl group, examples of which include but are not limited to —C₂—, —CH₂CH₂—, —CH₂CH₂CH₂— and —CH₂CH(CH₃)CH₂—. An alkylenyl group may be optionally substituted with one or more substituents.

The term “alkenyl” refers to a straight or branched hydrocarbon chain having one or more double bonds. Alkenyl groups may include a specified number of carbon atoms. For example, C₂-C₁₂ alkenyl indicates that the alkenyl group may have 2, 3, 4, 5, 6, 7, 8, 9, 10, 11 or 12 carbon atoms. An alkenyl group may be, e.g., a C₂-C₁₂ alkenyl group, a C₂-C₁₀ alkenyl group, a C₂-C₈ alkenyl group, a C₂-C₆ alkenyl group or a C₂-C₄ alkenyl group. Examples of alkenyl groups include but are not limited to allyl, propenyl, 2-butenyl, 3-hexenyl and 3-octenyl groups. One of the double bond carbons may optionally be the point of attachment of the alkenyl substituent. An alkenyl group may be optionally substituted with one or more substituents.

The term “alkenylenyl” refers to a divalent alkenyl group, examples of which include but are not limited to —CH═CH—, —CH═CH—CH—=2, —CH═CH—CH₂— and —CH₂—CH═CH—CH₂—. An alkenylenyl group may be optionally substituted with one or more substituents.

The term “alkynyl” refers to a straight or branched hydrocarbon chain having one or more triple bonds. Alkynyl groups may include a specified number of carbon atoms. For example, C₂-C₁₂ alkynyl indicates that the alkynyl group may have 2, 3, 4, 5, 6, 7, 8, 9, 10, 11 or 12 carbon atoms. An alkynyl group may be, e.g., a C₂-C₁₂ alkynyl group, a C₂-C₁m alkynyl group, a C₂-C₁₂ alkynyl group, a C₂-C₆ alkynyl group or a C₂-C₄ alkynyl group. Examples of alkynyl groups include but are not limited to ethynyl, propargyl, and 3-hexynyl. One of the triple bond carbons may optionally be the point of attachment of the alkynyl substituent. An alkynyl group may be optionally substituted with one or more substituents.

The term “alkynylenyl” refers to a divalent alkynyl group, examples of which include but are not limited to —CC—, —CC—CH₂—, —C—CH₂—CH₂— and —CH₂—CC—CH₂—. An alkynylenyl group may be optionally substituted with one or more substituents.

The term “amino” as used herein refers to —NR_{N1}R_{N2} wherein R_{N1} and R_{N2} independently may be H, alkyl, aryl, alkenyl, alkynyl, heteroalkyl, cycloalkyl, heteroaryl, or heterocyclyl.

The term “aryl” refers to an aromatic monocyclic, bicyclic, or tricyclic hydrocarbon ring system, wherein any ring atom capable of substitution can be substituted (e.g., with one or more substituents). Examples of aryl moieties include but are not limited to phenyl, naphthyl, and anthracenyl. Aryl groups may be optionally substituted with one or more substituents.

The term “arylalkyl” refers to an alkyl moiety in which at least one alkyl hydrogen atom is replaced with an aryl group. Arylalkyl includes groups in which more than one hydrogen atom has been replaced with an aryl group. Examples of arylalkyl groups include but are not limited to benzyl, 2-phenylethyl, 3-phenylpropyl, 9-fluorenyl, benzhydryl, and trityl groups. Arylalkyl groups may be optionally substituted with one or more substituents, on either the aryl moiety or the alkyl moiety.

The term “carbonyl” as used herein refers to a —C(O)R group, wherein R is alkyl, aryl, alkenyl, alkynyl, alkoxy, heteroalkyl, cycloalkyl, heteroaryl, heterocyclyl, or amino. The term “C₁₋₄ carbonyl” refers to a group that may be preceded by an alkyl group of up to 3 carbon atoms. It may also be called an “alkylcarbonyl”. Examples of C₁₋₄ carbonyl include —C(O)R, —CH₂C(O)R, —CH₂CH₂C(O)R, and —CH₂CH₂CH₂C(O)R.

The term “carboxyl” as used herein refers to a —OC(O)R group, wherein R is alkyl, aryl, alkenyl, alkynyl, alkoxy, heteroalkyl, cycloalkyl, heteroaryl, heterocyclyl, or amino. The term “C₁₋₄ carboxyl” refers to a group that may be preceded by an alkyl group of up to 3 carbon atoms. It may also be called an “alkylcarboxyl”. Examples of C.sub.1-4 carbonyl include —OC(O)R, —CH₂OC(O)R, —CH₂CH₂OC(O)R, and —CH₂CH₂CH₂OC(O)R.

The term “cycloalkyl” as used herein refers to non-aromatic, saturated or partially unsaturated cyclic, bicyclic, tricyclic or polycyclic hydrocarbon groups having 3 to 12 carbons. Any ring atom can be substituted (e.g., with one or more substituents). Cycloalkyl groups can contain fused rings. Fused rings are rings that share one or more common carbon atoms. Examples of cycloalkyl groups include but are not limited to cyclopropyl, cyclobutyl, cyclopentyl, cyclohexyl, cyclohexenyl, cyclohexadienyl, methylcyclohexyl, adamantyl, norbornyl, norbornenyl, tetrahydronaphthalenyl and dihydroindenyl. Cycloalkyl groups may be optionally substituted with one or more substituents.

The term “cycloalkylalkyl”, as used herein, refers to an alkyl group in which at least one hydrogen atom is replaced with a cycloalkyl group. Cycloalkylalkyl groups include those in which more than one hydrogen atom of the alkyl group is replaced with a cycloalkyl group. Examples of cycloalkylalkyl groups include but are not limited to cyclohexylmethyl, cyclopentylmethyl, cyclobutylmethyl and cyclopropylmethyl. Cycloalkylalkyl groups can be optionally substituted with one or more substituents, on either the cycloalkyl moiety or the alkyl moiety.

The term “halo” or “halogen” as used herein refers to any radical of fluorine, chlorine, bromine or iodine.

“Heteroalkyl” refers to an alkyl, alkenyl or alkynyl group as defined herein, wherein at least one carbon atom of the alkyl group is replaced with a heteroatom. Heteroalkyl groups may contain from 1 to 18 non-hydrogen atoms (carbon and heteroatoms) in the chain, or 1 to 12 atoms, or 1 to 6 atoms, or 1 to 4 atoms. Heteroalkyl groups may be straight or branched, and saturated or unsaturated. Unsaturated heteroalkyl groups have one or more double bonds and/or one or more triple bonds. Heteroalkyl groups may be unsubstituted or substituted. Exemplary heteroalkyl groups include but are not limited to alkoxyalkyl (e.g., methoxymethyl), and aminoalkyl (e.g., alkylaminoalkyl and dialkylaminoalkyl). Heteroalkyl groups may be optionally substituted with one or more substituents.

The term “heteralkylenyl” refers to a divalent heteroalkyl group, examples of which include but are not limited to —CH₂OCH₂—, —CH₂NHCH₂—, polyethyleneglycol groups (e.g., —(CH₂CH₂O)ₙ—), polyethyleneimine groups (e.g., —(CH₂CH₂NH)ₙ—), and the like. A heteroalkylenyl group may be optionally substituted with one or more substituents.

The term “heteroaryl” as used herein refers to an aromatic 5-8 membered monocyclic, 8-12 membered bicyclic, or 11-14 membered tricyclic ring system having 13 heteroatoms if monocyclic, 1-6 heteroatoms if bicyclic, or 1-9 heteroatoms if tricyclic, said heteroatoms independently selected from O, N, S, P and Si (e.g., carbon atoms and 13, 1-6, or 1-9 heteroatoms independently selected from O, N, S, P and Si if monocyclic, bicyclic, or tricyclic, respectively). Any ring atom can be substituted (e.g., with one or more substituents). Heteroaryl groups can contain fused rings, which are rings that share one or more common atoms. Examples of heteroaryl groups include but are not limited to radicals of pyridine, pyrimidine, pyrazine, pyridazine, pyrrole, imidazole, pyrazole, oxazole, isoxazole, furan, thiazole, isothiazole, thiophene, quinoline, isoquinoline, quinoxaline, quinazoline, cinnoline, indole, isoindole, indolizine, indazole, benzimidazole, phthalazine, pteridine, carbazole, carboline, phenanthridine, acridine, phenanthroline, phenazine, naphthyridines and purines. Heteroaryl groups may be optionally substituted with one or more substituents.

The term “heteroarylalkyl” refers to an alkyl moiety in which at least one alkyl hydrogen atom is replaced with a heteroaryl group. Heteroarylalkyl includes groups in which more than one hydrogen atom has been replaced with a heteroaryl group. Examples of heteroarylalkyl groups include but are not limited to imidazolylmethyl (e.g., 1H-imidazol-2-ylmethyl and 1H-imidazol-4-ylmethyl), pyridinylmethyl (e.g., pyridin-3-ylmethyl and pyridin-4-ylmethyl), pyrimidinylmethyl (e.g., pyrimidin-5-ylmethyl), furylmethyl (e.g., fur-2-ylmethyl and fur-3-ylmethyl), and thienylmethyl (e.g., thien-2-ylmethyl and thien-3-ylmethyl) groups. Heteroarylalkyl groups may be optionally substituted with one or more substituents, on either the heteroaryl moiety or the alkyl moiety.

The term “heteroatom”, as used herein, refers to a non-carbon or hydrogen atom such as a nitrogen, sulfur, oxygen, silicon, or phosphorus atom. Groups containing more than one heteroatom may contain different heteroatoms.

The term “heterocyclyl” or “heterocyclic”, as used herein, refers to a nonaromatic, saturated or partially unsaturated 3-10 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, S, Si and P (e.g., carbon atoms and 1-3, 1-6, or 1-9 heteroatoms of O, N, S, Si and P if monocyclic, bicyclic, or tricyclic, respectively). Any ring atom can be substituted (e.g., with one or more substituents). Heterocyclyl groups can contain fused rings, which are rings that share one or more common atoms. Examples of heterocyclyl groups include but are not limited to radicals of tetrahydrofuran, tetrahydrothiophene, tetrahydropyran, oxetane, piperidine, piperazine, morpholine, pyrroline, pyrimidine, pyrrolidine, indoline, tetrahydropyridine, dihydropyran, thianthrene, pyran, benzopyran, xanthene, phenoxathiin, phenothiazine, furazan, lactones, lactams such as azetidinones and pyrrolidinones, sultams, sultones, and the like. Heterocyclyl groups may be optionally substituted with one or more substituents.

The term “heterocyclylalkyl” refers to an alkyl moiety in which at least one alkyl hydrogen atom is replaced with a heterocyclyl group. Heterocyclylalkyl includes groups in which more than one hydrogen atom has been replaced with a heterocyclyl group. Examples of heterocyclylalkyl groups include but are not limited to oxetanylmethyl, morpholinomethyl, and pyrrolidinylmethyl groups, and the like. Heterocyclylalkyl groups may be optionally substituted with one or more substituents, on either the heterocyclyl moiety or the alkyl moiety.

The term “hydroxy” refers to an —OH radical. The term “alkoxy” refers to an —O— alkyl radical. The term “aryloxy” refers to an —O-aryl radical.

The term “oxo” refers to an oxygen atom, which forms a carbonyl when attached to carbon, an N-oxide when attached to nitrogen, and a sulfoxide or sulfone when attached to sulfur (i.e. ═O).

The term “substituents” refers to a group “substituted” on an alkyl, alkenyl, alkynyl, cycloalkyl, heterocyclyl, aryl, arylalkyl, heteroaryl or heteroarylalkyl group at any atom of that group. Any atom can be substituted. Suitable substituents include, without limitation: acyl, acylamido, acyloxy, alkoxy, alkyl, alkenyl, alkynyl, amido, amino, carboxy, cyano, ester, halo, hydroxy, imino, nitro, oxo (e.g., C═O), phosphonate, sulfinyl, sulfonyl, sulfonate, sulfonamino, sulfonamido, thioamido, thiol, thioxo (e.g., C═S), and ureido. In embodiments, substituents on a group are independently any one single, or any combination of the aforementioned substituents. In embodiments, a substituent may itself be substituted with any one of the above substituents.

The above substituents may be abbreviated herein. For example, the abbreviations Me, Et, Ph and Bn represent methyl, ethyl, phenyl and benzyl, respectively. A more comprehensive list of standard abbreviations used by organic chemists appears in a table entitled Standard List of Abbreviations of the Journal of Organic Chemistry. The abbreviations contained in said list are hereby incorporated by reference.

For compounds described herein, groups and substituents thereof may be selected in accordance with permitted valence of the atoms and the substituents, and such that the selections and substitutions result in a stable compound, e.g., a compound that does not spontaneously undergo transformation such as by rearrangement, cyclization, elimination, etc.

Where substituent groups are specified by their conventional chemical formulae, written from left to right, they optionally encompass substituents resulting from writing the structure from right to left, e.g., —CH₂O— optionally also recites —OCH₂—.

In some embodiments, GHSR1a-agonist compounds described herein, where applicable, can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. In certain embodiments, GHSR1a-agonist compounds described herein may exist in one or more particular geometric, optical, enantiomeric, diastereomeric, epimeric, atropic, stereoisomer, tautomeric, conformational, or anomeric forms, including but not limited to, cis- and trans-forms; E- and Z-forms; c-, t-, and r-forms; endo- and exo-forms; R-, S-, and meso-forms; D- and L-forms; d- and l-forms; (+) and (−) forms; keto-, enol-, and enolate-forms; syn- and anti-forms; synclinal- and anticlinal-forms; .alpha.- and .beta.-forms; axial and equatorial forms; boat-, chair-, twist-, envelope-, and half chair-forms; and combinations thereof, hereinafter collectively referred to as “isomers” (or “isomeric forms”). Isomers can be isolated from mixtures by methods known to those skilled in the art, including chiral high-pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981). The disclosure additionally encompasses GHSR1a-agonist compounds disclosed herein as individual isomers substantially free of other isomers, and alternatively, as mixtures of various isomers. In some embodiments, GHSR1a-agonist compounds disclosed herein may be (R)-isomers. Alternatively, GHSR1a-agonist compounds disclosed herein may be (S)-isomers. In some embodiments, GHSR1a-agonist compounds disclosed herein may be a mixture of (R) and (S) isomers.

In some embodiments, a GHSR1a-agonist compound disclosed herein may be an enantiomerically enriched isomer of a stereoisomer described herein. For example, a GHSR1a-agonist compound herein may have an enantiomeric excess of at least about 10%, 15%, 20%, 25%, 30%, 35%, 40%, 45%, 50%, 55%, 60%, 65%, 70%, 75%, 80%, 85%, 90%, 95%, 96%, 97%, 98%, or 99%. Enantiomer, when used herein, refers to either of a pair of chemical compounds whose molecular structures have a mirror-image relationship to each other.

In some embodiments, preparation of a GHSR1a-agonist compound disclosed herein may be enriched for an isomer of the compound having a selected stereochemistry, e.g., R or S, corresponding to a selected stereocenter. For example, a GHSR1a-agonist compound herein may have a purity corresponding to a compound having a selected stereochemistry of a selected stereocenter of at least about 60%, 65%, 70%, 75%, 80%, 85%, 90%, 95%, 96%, 97%, 98%, or 99%.

In some embodiments, a GHSR1a-agonist compound disclosed herein may include a preparation of a compound disclosed herein that is enriched for a structure or structures having a selected stereochemistry, e.g., R or S, at a selected stereocenter. Exemplary R/S configurations can be those provided in an example described herein. An “enriched preparation,” as used herein, is enriched for a selected stereoconfiguration of one, two, three or more selected stereocenters within the subject compound. Exemplary selected stereocenters and exemplary stereoconfigurations thereof can be selected from those provided herein, e.g., in an example described herein. By enriched is meant at least 60%, e.g., of the molecules of compound in the preparation have a selected stereochemistry of a selected stereocenter. In an embodiment it is at least 65%, 70%, 75%, 80%, 85%, 90%, 95%, 96%, 97%, 98%, or 99%. Enriched refers to the level of a subject molecule(s) and does not connote a process limitation unless specified.

In some embodiments, GHSR1a-agonist compounds disclosed herein can be in the form of an ester prodrug. The term “ester” herein can refer a compound which is produced by modifying a functional group (e.g. hydroxyl, carboxyl, amino or the like group). Examples of the “ester” include “esters formed with a hydroxyl group” and “esters formed with a carboxyl group.” The term “ester” can mean an ester whose ester residue is a “conventional protecting group” or a “protecting group removable in vivo by a biological method such as hydrolysis”. In some embodiments, the term “conventional protecting group” can mean a protecting group removable by a chemical method such as hydrogenolysis, hydrolysis, electrolysis, or photolysis. In some embodiments, the term “protecting group removable in vivo by a biological method such as hydrolysis” can mean a protecting group removable in vivo by a biological method such as hydrolysis to produce a free acid or its salt.

In some embodiments, GHSR1a-agonist compounds disclosed herein can be in the form of a pharmaceutically acceptable salt. By “salt” or “pharmaceutically acceptable salt”, it is meant those salts which are, within the scope of sound medical judgment, suitable for use in contact with the tissues of humans and lower animals without undue toxicity, irritation, and allergic response, commensurate with a reasonable benefit to risk ratio, and effective for their intended use. A “pharmacologically acceptable salt” can refer to a salt, which can be formed when a GHSR1a-agonist compound has an acidic group such as carboxyl or a basic group such as amino or imino. In some embodiments, GHSR1a-agonist salt formed with an acidic group herein can include alkali metal salts such as a sodium salt, potassium salt or lithium salt, alkaline earth metal salts such as a calcium salt or magnesium salt, metal salts such as an aluminum salt or iron salt; amine salts, e.g., inorganic salts such as an ammonium salt and organic salts such as a t-octylamine salt, dibenzylamine salt, morpholine salt, glucosamine salt, phenylglycine alkyl ester salt, ethylenediamine salt, N-methylglucamine salt, guanidine salt, diethylamine salt, triethylamine salt, dicyclohexylamine salt, N,N′-dibenzylethylenediamine salt, chloroprocaine salt, procaine salt, diethanolamine salt, N-benzylphenethylamine salt, piperazine salt, tetramethylammonium salt or tris(hydroxymethyl)aminomethane salt; and amino acid salts such as a glycine salt, lysine salt, arginine salt, ornithine salt, glutamate or aspartate. In some embodiments, a GHSR1a-agonist salt formed with a basic group herein can include hydro-halides such as a hydrofluoride, hydrochloride, hydrobromide or hydroiodide, inorganic acid salts such as a nitrate, perchlorate, sulfate or phosphate; lower alkanesulfonates such as a methanesulfonate, trifluoromethanesulfonate or ethanesulfonate, arylsulfonates such as a benzenesulfonate or p-toluenesulfonate, organic acid salts such as an acetate, malate, fumarate, succinate, citrate, ascorbate, tartrate, oxalate or maleate; and amino acid salts such as a glycine salt, lysine salt, arginine salt, omithine salt, glutamate or aspartate. In certain embodiments, when a pharmacologically acceptable salt of a GHSR1a-agonist remains in the atmosphere or is recrystallized, it can absorb water to form a hydrate of use in formulations disclosed herein.

In some embodiments, GHSR1a-agonist compounds disclosed herein can be in the form of another aminoglycoside derivative. In some embodiments, the term “other derivative” can mean a derivative of the GHSR1a-agonist compound other than the above-described “ester” or the above-described “pharmacologically acceptable salt” which can be formed, if it has an amino and/or carboxyl group or other conjugate form or other active derivative thereof.

In some embodiments, GHSR1a-agonist compounds disclosed herein may be in a chemically protected form. The term “chemically protected form” is used herein in the conventional chemical sense and pertains to a compound in which one or more reactive functional groups are protected from undesirable chemical reactions under specified conditions (e.g., pH, temperature, radiation, solvent, and the like). In practice, well known chemical methods are employed to reversibly render unreactive a functional group, which otherwise would be reactive, under specified conditions. In a chemically protected form, one or more reactive functional groups are in the form of a protected or protecting group (also known as a masked or masking group or a blocked or blocking group). By protecting a reactive functional group, reactions involving other unprotected reactive functional groups can be performed, without affecting the protected group; the protecting group may be removed, usually in a subsequent step, without substantially affecting the remainder of the molecule. See, for example, Protective Groups in Organic Synthesis (T. Green and P. Wuts; 3rd Edition; John Wiley and Sons, 1999). Unless otherwise specified, a reference to a particular compound also includes chemically protected forms thereof.

A wide variety of such “protecting,” “blocking,” or “masking” methods are widely used and well known in organic synthesis. For example, a compound which has two nonequivalent reactive functional groups, both of which would be reactive under specified conditions, may be derivatized to render one of the functional groups “protected,” and therefore unreactive, under the specified conditions; so protected, the compound may be used as a reactant which has effectively only one reactive functional group. After the desired reaction (involving the other functional group) is complete, the protected group may be “deprotected” to return it to its original functionality. A hydroxy group may be protected as an ether (—OR) or an ester (—OC(O)R), for example, as: a t-butyl ether; a benzyl, benzhydryl (diphenylmethyl), or trityl (triphenylmethyl) ether; a trimethylsilyl or t-butyldimethylsilyl ether; or an acetyl ester (—OC(O)CH₃, —OAc). An aldehyde or ketone group may be protected as an acetal (RCH(OR)₂) or ketal (R₂C(OR)₂), respectively, in which the carbonyl group (R₂C═O) is converted to a diether (R₂C(OR)₂), by reaction with, for example, a primary alcohol. The aldehyde or ketone group is readily regenerated by hydrolysis using a large excess of water in the presence of acid. An amine group may be protected, for example, as an amide (—NRC(O)R) or a urethane (—NRC(O)OR), for example, as: a methyl amide (—NHC(O)CH₃); a benzyloxy amide (—NHC(O)OCH₂C₆H₅, —NH-Cbz); as a t-butoxy amide (—NHC(O)OC(CH)₃, —NH-Boc); a 2-biphenyl-2-propoxy amide (—NHCO(O)C(CH₃)₂C₆H₄C₆H₅, —NH-Bpoc), as a 9-fluorenylmethoxy amide (—N1-Fmoc), as a 6-nitroveratryloxy amide (—NH—Nvoc), as a 2-trimethylsilylethyloxy amide (—NH-Teoc), as a 2,2,2-trichloroethyloxy amide (—NH-Troc), as an allyloxy amide (—NH-Alloc), as a 2(-phenylsulphonyl)ethyloxy amide (—NH—Psec); or, in suitable cases (e.g., cyclic amines), as a nitroxide radical (>N-0<<). A carboxylic acid group may be protected as an ester, for example, as: an alkyl ester (e.g., a methyl ester; a t-butyl ester); a haloalkyl ester (e.g., a haloalkyl ester); a trialkylsilylalkyl ester; or an arylalkyl ester (e.g., a benzyl ester; a nitrobenzyl ester); or as an amide, for example, as a methyl amide. A thiol group may be protected as a thioether (—SR), for example, as: a benzyl thioether; an acetamidomethyl ether (—S—CH₂NHC(O)CH₃).

In addition to salt forms, the present disclosure may also provide compounds that are in a prodrug form. Prodrugs of the compounds described herein are those compounds that readily undergo chemical changes under physiological conditions to provide the compounds described herein. Prodrugs can be converted to the compounds of the present disclosure by chemical or biochemical methods in an ex vivo environment. For example, prodrugs can be slowly converted to the compounds of the present disclosure when placed in a transdermal patch reservoir with a suitable enzyme or chemical reagent.

Note that specifically included in the term “isomer” are compounds with one or more isotopic substitutions. Examples of isotopes suitable for inclusion in the compounds of the invention are hydrogen, carbon, nitrogen, oxygen, phosphorus, sulfur, fluorine, and chlorine, such as, but not limited to ²H, ³H, ¹³C, ¹⁴C, ¹⁵N, ¹⁸O, ¹⁷O, ³¹P, ³²P, ³⁵S, ¹⁸F, and ³⁶Cl, respectively. Substitution with heavier isotopes such as deuterium, i.e. ²H, can afford certain therapeutic advantages resulting from greater metabolic stability, for example, increased in vivo half-life or reduced dosage requirements and, hence, may be preferred in some circumstances. The compound may incorporate positron-emitting isotopes for medical imaging and positron-emitting tomography (PET) studies for determining the distribution of receptors. Suitable positron-emitting isotopes that can be incorporated in compounds of formula (I) are ¹¹C, ¹³N, ¹⁵O, and ¹⁸F. Isotopically-labeled compounds of formula (I) can generally be prepared by conventional techniques known to those skilled in the art or by processes analogous to those described in the accompanying Examples using appropriate isotopically-labeled reagent in place of non-isotopically-labeled reagent. In some embodiments, in compounds of Formula (I) and Formula (III), any hydrogen atom may be deuterium.

In some embodiments, GHSR1a-agonist compounds disclosed herein may be modified to enhance selective biological properties. Such modifications are known in the art and may include those that increase biological penetration into a given biological system (e.g., blood, lymphatic system, central nervous system), increase oral availability, increase solubility to allow administration by injection, alter metabolism, and/or alter rate of excretion. Examples of these modifications include, but are not limited to, esterification with polyethylene glycols, derivatization with pivolates or fatty acid substituents, conversion to carbamates, hydroxylation of aromatic rings, and heteroatom substitution in aromatic rings. In some embodiments, GHSR1a-agonist compounds disclosed herein may be modified to enhance blood brain barrier permeability.

In certain embodiments, GHSR1a-agonist compounds herein (e.g., a compound of Formula I, II, III and/or IV) can have kinetic solubility. Higher kinetic solubility can be important in bioavailability of formulations, such as oral and/or peritoneal formulations. In some embodiments, compounds herein can have a kinetic solubility of at least about 0.1 μM. In some embodiments, compounds herein can have a kinetic solubility ranging from about 0.1 μM to about 110 μM (e.g., about 0.1 μM, about 0.5 μM, about 1 μM, about 5 μM, about 10 μM, about 20 μM, about 30 μM, about 40 μM, about 50 μM, about 60 μM, about 70 μM, about 80 μM, about 90 μM, about 100 μM, about 110 μM). In some embodiments, compounds herein can maintain a kinetic solubility for about 1 hours to about 48 hours (e.g., about 1 hour, about 3 hours, about 6 hours, about 12 hours, about 24 hours, about 36 hours, 48 hours). In some embodiments, compounds herein can maintain a kinetic solubility ranging from about 0.1 μM to about 110 μM (e.g., about 0.1 μM, about 0.5 μM, about 1 μM, about 5 μM, about 10 μM, about 20 μM, about 30 μM, about 40 μM, about 50 μM, about 60 μM, about 70 μM, about 80 μM, about 90 μM, about 100 μM, about 110 μM) for about 1 hours to about 48 hours (e.g., about 1 hour, about 3 hours, about 6 hours, about 12 hours, about 24 hours, about 36 hours, 48 hours). In some embodiments, compounds herein can maintain a kinetic solubility at temperatures ranging from about 4° C. to about 80° C. (e.g., about 4° C., about 6° C., about 8° C., about 10° C., about 20° C., about 30° C., about 40° C., about 50° C., about 60° C., about 70° C., about 80° C.). In some embodiments, compounds herein can maintain a kinetic solubility ranging from about 0.1 μM to about 110 μM (e.g., about 0.1 μM, about 0.5 μM, about 1 μM, about 5 μM, about 10 μM, about 20 μM, about 30 μM, about 40 μM, about 50 μM, about 60 μM, about 70 μM, about 80 μM, about 90 μM, about 100 μM, about 110 μM) at temperatures ranging from about 4° C. to about 80° C. (e.g., about 4° C., about 6° C., about 8° C., about 10° C., about 20° C., about 30° C., about 40° C., about 50° C., about 60° C., about 70° C., about 80° C.). In some embodiments, compounds herein can maintain a kinetic solubility ranging from about 0.1 μM to about 110 μM (e.g., about 0.1 μM, about 0.5 μM, about 1 μM, about 5 μM, about 10 μM, about 20 μM, about 30 μM, about 40 μM, about 50 μM, about 60 μM, about 70 μM, about 80 μM, about 90 μM, about 100 μM, about 110 μM) at room temperature (i.e., 25° C.±3° C.). In some embodiments, compounds herein can maintain a kinetic solubility ranging from about 0.1 μM to about 110 μM (e.g., about 0.1 μM, about 0.5 μM, about 1 μM, about 5 μM, about 10 μM, about 20 μM, about 30 μM, about 40 μM, about 50 μM, about 60 μM, about 70 μM, about 80 μM, about 90 μM, about 100 μM, about 110 μM) at room temperature (i.e., 25° C.±3° C.) for about 1 hours to about 48 hours (e.g., about 1 hour, about 3 hours, about 6 hours, about 12 hours, about 24 hours, about 36 hours, 48 hours or more depending on the compound).

In certain embodiments, GHSR1a-agonist compounds disclosed herein (e.g., a compound of Formula I, II, III and/or IV) can have a central nervous system multiparameter optimization (CNS MPO) score. The CNS MPO score is determined by an algorithm that uses a weighted scoring function to assess six fundamental physicochemical properties [(a) lipophilicity, calculated partition coefficient (C log P); (b) calculated distribution coefficient at pH 7.4 (C log D); (c) molecular weight (MW); (d) topological polar surface area (TPSA); (e) number of hydrogen-bond donors (HBDs); and (f) most basic center (pKₐ)]. The algorithm assigns a compound a collective score ranging from 0 to 6, wherein higher CNS MPO scores are indicative of the compound's capability of crossing the blood-brain-barrier (BBB). In some embodiments, compounds herein can have a CNS MPO score indicative of BBB permeability. In some embodiments, compounds disclosed herein can have a CNS MPO score greater than or equal to 4.0.

In certain embodiments, GHSR1a-agonist compounds disclosed herein (e.g., a compound of Formula I, II, III and/or IV) may maintain sustained concentrations in the brain following administration. In some embodiments, compounds disclosed herein may maintain sustained concentrations in the brain following systemic administration (e.g., by oral administration, intraperitoneal administration, intravenous administration). In some embodiments, compounds disclosed herein may maintain sustained concentrations in the brain for about 5 minutes to about 24 hours following administration. In some embodiments, compounds disclosed herein may maintain sustained concentrations in the brain for about 5 minutes, about 10 minutes, about 15 minutes, about 30 minutes, about 1 hour, about 2 hours, about 4 hours, about 6 hours, about 8 hours, about 10 hours, about 12 hours, about 14 hours, about 16 hours, about 18 hours, about 20 hours, about 22 hours, or about 24 hours following administration. In some embodiments, compounds disclosed herein (e.g., a compound of Formula I, II, III and/or IV) may maintain a brain:plasma ratio of about 0.2:1 to about 1:1 following administration. In some embodiments, compounds disclosed herein may maintain a brain:plasma ratio of about 0.2:1, about 0.3:1, about 0.4:1, about 0.5:1, about 0.6:1, about 0.7:1, about 0.8:1, about 0.9:1, or about 1:1 following administration. In some embodiments, compounds disclosed herein may maintain a brain:plasma ratio of about 0.2:1 to about 1:1 for about 5 minutes to about 24 hours following administration.

GHSR1a-agonist compounds disclosed herein may modulate heterotrimeric G protein activity, β-arrestin activity, or a combination thereof. In certain embodiments, GHSR1a-agonist compounds disclosed herein may be biased agonists. Ligand bias, or “true” biased agonism, refers to differences in signaling due to the molecular variation that governs the interaction between the ligand (e.g., GHSR1a-agonist compounds disclosed herein) and the transduction proteins at the receptor (e.g., GHSR1a). For GPCRs, such as GHSR1a, the easiest bias to observe is that between selective activation of heterotrimeric 0 proteins (G protein-bias) and β-arrestin (β-arrestin-bias) adapter proteins because G proteins and f-arrestins typically activate distinct signaling pathways, with G proteins typically activating second messengers and β-arrestins regulating receptor desensitization, internalization and activation of MAP kinases. In this regard, “agonists,” as used herein, may also be “biased agonists” with the ability to selectively stimulate a subset of GHSR1a's signaling activities, for example, the selective activation of G-protein or β-arrestin function. In some embodiments, GHSR1a-agonist compounds disclosed herein may be biased agonists that select for G-protein coupled signaling over arrestin coupled signaling. In some embodiments, GHSR1a-agonist compounds disclosed herein may be biased agonists that selectively activate one or more heterotrimeric G proteins (i.e., “G protein-bias”). In some embodiments, GHSR1a-agonist compounds disclosed herein may be biased agonists that do not selectively activate β-arrestin (i.e., “β-arrestin-bias”) adapter proteins.

In some embodiments, GHSR1a-agonist compounds disclosed herein may be biased agonists that selectively activate one or more of the G protein subunits. In accordance with these embodiments, biased GHSR1a-agonist disclosed herein may selectively activate one or more of the G protein subunits comprising Gα, Gβ, and/or Gγ. In some embodiments, GHSR1a-agonist compounds disclosed herein may be biased agonists that selectively activate one or more Gα subunits. In some embodiments, GHSR1a-agonist compounds disclosed herein may be biased agonists that selectively activate Gαₛ, Gα_{olf}, Gαᵢ₁, Gαᵢ₂, Gαᵢ₃, Gα_{oA}, Gα_{oB}, Gα_{z}, Gα_{q}, Gα₁₁, Gα₁₄, Gα₁₅, Gα₁₂, Gα₁₃, or any combination thereof. In some embodiments, GHSR1a-agonist compounds disclosed herein may be biased agonists that selectively activate Gα_{q}. In some embodiments, GHSR1a-agonist compounds disclosed herein may be biased agonists that selectively activate Gα_{q/11}.

In some embodiments, GHSR1a-agonist compounds disclosed herein may not compete with ghrelin peptide binding to the GHSR1a. In some embodiments, a GHSR1a-agonist compound disclosed herein and a ghrelin peptide may bind to the GHSR1a simultaneously.

### III. Pharmaceutical Compositions

In certain embodiments, compositions disclosed herein are pharmaceutical compositions. In some embodiments, pharmaceutical compositions can include at least one compound disclosed herein and at least one pharmaceutically acceptable carrier. In some embodiments, pharmaceutical compositions can include pharmaceutically acceptable carriers, excipients, and/or stabilizers are nontoxic to recipients at dosages and/or concentrations used to practice the methods disclosed herein. In some embodiments, pharmaceutically acceptable carriers, excipients, and/or stabilizers can include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

In certain embodiments, pharmaceutical compositions disclosed herein can be in unit dosage forms such as tablets, pills, capsules, powders, granules, solutions or suspensions, or suppositories, for oral, parenteral, or rectal administration, or administration by inhalation or insufflation. In some embodiments, for preparing solid compositions such as tablets, the principal active ingredient (e.g., a compound disclosed herein) can be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid pre-formulation composition containing a homogeneous mixture of a compound of the present invention, or a non-toxic pharmaceutically acceptable salt thereof. When referring to these pre-formulation compositions as homogeneous, it is meant that the active ingredient is dispersed evenly throughout the composition so that the composition can be readily subdivided into equally effective unit dosage forms such as tablets, pills, and capsules. In some embodiments, solid pre-formulation compositions herein can then subdivided into unit dosage forms of the type described above containing from 0.1 mg to about 500 mg (e.g., about 0.1 mg, about 0.5 mg, about 1.0 mg, about 5.0 mg, about 10 mg, about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg) of a compound disclosed herein.

In some embodiments, tablets and/or pills disclosed herein can be coated or otherwise compounded to provide a dosage form affording the advantage of prolonged action. In some embodiments, a tablet and/or pill herein can have an inner dosage and an outer dosage component, the latter being in the form of an envelope over the former. In accordance with embodiments herein, the two components can be separated by an enteric layer that serves to resist disintegration in the stomach and permits the inner component to pass intact into the duodenum or to be delayed in release. In some embodiments, tablets and/or pills disclosed herein can include one or more materials that can be used for such enteric layers or coatings, such materials including a number of polymeric acids and mixtures of polymeric acids with such materials as shellac, cetyl alcohol and cellulose acetate. Non-limiting surface-active agents (surfactants) suitable for use herein can include non-ionic agents, such as polyoxyethylenesorbitans (e.g., Tween 20, 40, 60, 80 or 85) and other sorbitans (e.g., Span 20, 40, 60, 80 or 85). In some embodiments, compositions herein with a surface-active agent can have between about 0.05 and about 5% surface-active agent. In some embodiments, other ingredients can be added to pharmaceutical compositions herein, for example mannitol or other pharmaceutically acceptable vehicles, if necessary.

In some embodiments, pharmaceutical compositions herein can be tablets. In accordance with some embodiments herein, tablets contemplated herein can contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxy-propylcellulose (HPC), sucrose, gelatin and acacia. In accordance with some embodiments herein, tablets contemplated herein can contain lubricating agents such as magnesium stearate, stearic acid, glyceryl behenate and talc. In some embodiments, pharmaceutical compositions herein can be solid compositions employed as fillers in gelatin capsules. In accordance with some embodiments herein, excipients included in gelatin capsules contemplated herein can include lactose, starch, cellulose, milk sugar or high molecular weight polyethylene glycols.

In some embodiments, pharmaceutical compositions herein can be emulsions. In some embodiments, emulsions herein can be prepared using commercially available fat emulsions, such as Intralipid, Liposyn, Infonutrol, Lipofundin, and Lipiphysan. The active ingredient (e.g., the one or more aminopeptidase inhibitors and/or one or more platinum-based chemotherapeutics) can be either dissolved in a pre-mixed emulsion composition or alternatively it can be dissolved in an oil (e.g., soybean oil, safflower oil, cottonseed oil, sesame oil, corn oil or almond oil) and an emulsion formed upon mixing with a phospholipid (e.g., egg phospholipids, soybean phospholipids or soybean lecithin) and water. In some embodiments, other ingredients can be added to the compositions herein, for example glycerol or glucose, to adjust the tonicity of the emulsion. In other embodiments, emulsions can contain up to about 20% oil, for example, between about 5% and about 20%. In some embodiments, emulsions can have fat droplets between about 0.1 μm and about 1.0 μm in diameter and/or have a pH in the range of about 5.5 to about 8.0.

In certain embodiments, pharmaceutical compositions herein can be formulated for parenteral administration, such as intravenous, intracerebroventricular injection, intra-cisterna magna injection, intra-parenchymal injection, or a combination thereof. In some embodiments, pharmaceutical compositions herein formulated for parenteral administration can include one or more sterile liquids as pharmaceutically acceptable carriers. Non-limiting examples of sterile liquids suitable for use as pharmaceutically acceptable carriers herein can be water and oil, including those of petroleum, animal, vegetable, or synthetic origin, such as peanut oil, soybean oil, mineral oil, and the like. Saline solutions and aqueous dextrose, polyethylene glycol (PEG) and glycerol solutions can also be employed as liquid carriers, particularly for injectable solutions. Pharmaceutical compositions disclosed herein may further comprise additional ingredients, for example preservatives, buffers, tonicity agents, antioxidants and stabilizers, nonionic wetting or clarifying agents, viscosity-increasing agents, and the like. In some embodiments, pharmaceutical compositions disclosed herein can be packaged in single unit dosages or in multi-dosage forms.

In some embodiments, pharmaceutical compositions herein suitable for parenteral administration can include aqueous and non-aqueous sterile injection solutions which can further contain anti-oxidants, buffers, bacteriostats and solutes which render the formulation isotonic with the blood of the intended recipient; and aqueous and non-aqueous sterile suspensions which may include suspending agents and thickening agents. Aqueous solutions may be suitably buffered (preferably to a pH of from about 3 to about 9). The preparation of suitable parenteral formulations under sterile conditions is readily accomplished by standard pharmaceutical techniques well known to those skilled in the art.

In some embodiments, pharmaceutical compositions described herein can further include an anti-microbial agent. In accordance with these embodiments, the anti-microbial agent can, in an example, be an anti-viral, bactericidal agent, anti-fungal, or anti-bacterial agent. For example, the anti-microbial agent can be an anti-bacterial agent (antibiotic) such as doxycycline or other antibiotics such as a general antibiotic.

### IV. Methods of Use

Any of the GHSR1a-agonist compounds (e.g., a compound of Formula I, II, III and/or IV) disclosed herein may be used for preventing, alleviating and/or treating pathological disruptions of brain dopamine (DA) homeostasis. DA, or 4-(2-aminoethyl)-1,2-benzenediol, is an organic chemical of the catecholamine and phenethylamine families and constitutes about 80% of the catecholamine content in the brain. The brain includes several distinct dopamine pathways, some of which play a major role in the motivational component of reward-motivated behavior. Both imbalances in brain dopamine homeostasis and alterations of brain circuits where dopamine is a key factor are involved in a variety of neurological and neuropsychiatric diseases. Non-limiting examples of disorders and/or diseases that arise from disruptions of brain dopamine homeostasis can include, but are not limited to, Parkinson's disease, attention deficit hyperactivity disorder, Tourette syndrome, schizophrenia, bipolar disorder, Alzheimer's Disease, eating disorders, and addiction (e.g., food/alcohol/drug).

In certain embodiments, the present disclosure provides methods for modulating dopaminergic neurotransmission in a subject in need thereof. In some embodiments, methods for modulating dopaminergic neurotransmission in a subject in need thereof may include administering a subject in need thereof an effective amount of any of the GHSR1a-agonist compounds (e.g., a compound of Formula I, II, III and/or IV) and/or pharmaceutical compositions disclosed herein. In some embodiments, a subject in need of modulation dopaminergic neurotransmission may have or be suspected of having one or more disruptions of brain dopamine (DA) homeostasis. In some embodiments, a subject in need of modulation dopaminergic neurotransmission may have or be suspected of having one or more of (but not limited to) the following: Parkinson's disease, attention deficit hyperactivity disorder, Tourette syndrome, one or more eating disorders, schizophrenia, bipolar disorder, Alzheimer's Disease, narcolepsy, depression, obesity, addiction (e.g., food/alcohol/drug), or any combination thereof.

To perform the methods disclosed herein, an effective amount of the compounds or compositions disclosed herein may be administered to a subject who needs treatment via a suitable route (e.g., orally, intravenous, intracerebroventricular injection, intra-cisterna magna injection, or intra-parenchymal injection) at a suitable amount as disclosed herein.

As used herein, the term “treating” refers to the application or administration of a composition including one or more active agents to a subject, who is in need of the treatment, for example, having a target disease or disorder, a symptom of the disease/disorder, or a predisposition toward the disease/disorder, with the purpose to cure, heal, alleviate, relieve, alter, remedy, ameliorate, improve, or affect the disorder, the symptom of the disease, or the predisposition toward the disease or disorder.

Alleviating a target disease/disorder includes delaying the development or progression of the disease or reducing disease severity. Alleviating the disease does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a target disease or disorder includes initial onset and/or recurrence.

In certain embodiments, methods disclosed herein may be used for preventing, alleviating and/or treating a neurological disorder associated with involuntary motor movements. In some embodiments, methods disclosed herein may be used for preventing, alleviating and/or treating Parkinson's disease. Thus, in some aspects, the present disclosure provides methods for alleviating one or more symptoms and/or for treating Parkinson's disease in a subject in need thereof by administration of any of the compounds disclosed herein, as well as a pharmaceutical composition comprising such. In some embodiments, methods herein may reduce hyperlocomotion in a subject treated with a compound or composition disclosed herein compared to an untreated subject with identical disease condition and predicted outcome. In some embodiments, methods herein may reduce hyperlocomotion by about 2% to about 99% (e.g., about 5%, about 10%, about 20%, about 30%, about 40%, about 50%, about 60%, about 70%, about 80%, about 90%, about 99%) in a subject treated with a compound or composition disclosed herein compared to an untreated subject with identical disease condition and predicted outcome.

In certain embodiments, methods disclosed herein may be used for preventing, alleviating and/or treating at least one addictive behavior in a subject. In some embodiments, methods disclosed herein may be used for preventing, alleviating and/or treating a drug addition, an alcohol addition, an addiction to food, or a combination thereof. The “drugs” according to the present disclosure can be, but are not limited to, narcotics, medicaments, tobacco, and/or alcohol. In some aspects, the drugs that are the source of the addition to be treated herein may be cocaine, crack, cannabis, morphine, opioids, heroin, ecstasy, LSD, amphetamines, ketamine, tobacco, alcohol, or any combination thereof. As will be clear to those skilled in the art, tobacco addiction is generally a nicotine addiction and alcohol addiction according is generally an ethanol addiction. In some embodiments, methods disclosed herein may be used for preventing relapse into addiction. In some embodiments, methods disclosed herein may be useful in reducing the addictive effect of re-exposure or continuous exposure to at least one agent, behavior, and/or stimulus which induces the addictive behavior in an addicted subject or in a subject having a risk of developing an addiction. In some embodiments, the addiction is not induced by re-exposure or continuous exposure to at least one agent or behavior in a subject after administration of any one of the compounds and/or compositions disclosed herein.

In certain embodiments, methods disclosed herein may be used for preventing, alleviating and/or treating at least one eating disorder in a subject. Eating disorders referred to herein may be characterized by abnormal compulsions to avoid eating or uncontrollable impulses to consume abnormally large amounts of food. In some embodiments, methods disclosed herein may be used for preventing, alleviating and/or treating bulimia nervosa, anorexia nervosa, binge-eating disorder, a food addition, or any combinations thereof. In some embodiments, methods disclosed herein may be used for preventing, alleviating and/or treating binge eating disorders, obesity resulting from binge eating behavior, and/or depression. In some embodiments, methods disclosed herein may treat an underlying mechanism of an eating disorders, including but not limited to hunger and satiety; palatability and aversion; hedonism; reward/addiction behavior; mood; anxiety; depression, taste, smell, and the like. In some embodiments, methods disclosed herein may be used for preventing, alleviating and/or treating an eating disorder that is an undesired side-effect of a therapeutic (e.g., chemotherapy, pharmacological treatments for mood disorders).

Conventional methods, known to those of ordinary skill in the art of medicine, can be used to administer any of the compounds or compositions disclosed herein to a subject in need thereof. In some embodiments, a compound or composition disclosed herein can be administered parenterally, e.g., by intravenous injection, intracerebroventricular injection, intra-cisterna magna injection, intra-parenchymal injection, or a combination thereof. In some embodiments, a compound or composition disclosed herein can be administered orally.

In certain embodiments, a compound or composition disclosed herein can be administered in combination with at least one additional therapeutic agent. In some embodiments, a compound or composition disclosed herein can be administered in combination with at least one additional therapeutic agent for treatment and/or prevention of any one of the diseases and/or disorders disclosed herein. In some embodiments, a compound or composition disclosed herein can be administered in combination with at least one additional therapeutic agent for treatment and/or prevention of one or more GHSR1a-associated conditions. In accordance with these embodiments, therapeutic agents suitable for administration to a subject in combination with any of the compounds or compositions disclosed herein may include, but are not limited to, methadone, naltrexone, acamprosate, disulfiram, bupropion, varenicline, nicotine replacement therapies, and any combination thereof. In some embodiments, a compound or composition disclosed herein can be administered before at least one additional therapeutic agent is administered to a subject. In some embodiments, a compound or composition disclosed herein can be administered after at least one additional therapeutic agent is administered to a subject. In some embodiments, a compound or composition disclosed herein can be administered during the course of treatment with at least one additional therapeutic agent administered to a subject.

In certain embodiments, a compound disclosed herein may be administered to a subject orally at a concentration ranging from about 0.5 mg/kg to about 50 mg/kg. In some embodiments, a compound disclosed herein may be administered to a subject orally at a concentration of about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 15 mg/kg, about 20 mg/kg, about 25 mg/kg, about 30 mg/kg, about 35 mg/kg, about 40 mg/kg, about 45 mg/kg, or about 50 mg/kg. In some embodiments, a compound disclosed herein may be administered to a subject orally at least once a day, at least twice a day, at least three times a day or more.

In certain embodiments, a compound disclosed herein may be administered to a subject intraperitoneally (IP) at a concentration ranging from about 0.5 mg/kg to about 50 mg/kg. In some embodiments, a compound disclosed herein may be administered to a subject intraperitoneally at a concentration of about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 15 mg/kg, about 20 mg/kg, about 25 mg/kg, about 30 mg/kg, about 35 mg/kg, about 40 mg/kg, about 45 mg/kg, or about 50 mg/kg. In some embodiments, a compound disclosed herein may be administered to a subject intraperitoneally at least once a day, at least twice a day, at least three times a day or more.

In certain embodiments, a compound disclosed herein may be administered to a subject intravenously at a concentration ranging from about 0.05 mg/kg to about 20 mg/kg. In some embodiments, a compound disclosed herein may be administered to a subject intravenously at a concentration of about 0.05 mg/kg, about 0.1 mg/kg, about 0.5 mg/kg, about 1 mg/kg, about 2 mg/kg, about 3 mg/kg, about 4 mg/kg, about 5 mg/kg, about 10 mg/kg, about 15 mg/kg, or about 20 mg/kg. In some embodiments, a compound disclosed herein may be administered to a subject intravenously at least once a day, at least twice a day, at least three times a day or more.

In certain embodiments, methods disclosed herein may be used to administer any of the compounds or compositions disclosed herein to a subject with high bioavailability. In some embodiments, a compound disclosed herein may have an oral bioavailability of about 1% to about 15%. As used herein “oral bioavailability” and “bioavailability upon oral administration” refer to the systemic availability (i.e., blood/plasma levels) of a given amount of a compound administered orally to a subject according to the methods herein. In some embodiments, a compound disclosed herein may have an oral bioavailability of about 1%, about 2%, about 3%, about 4%, about 5%, about 6%, about 7%, about 8%, about 9%, about 10%, about 11%, about 12%, about 13%, about 14%, or about 15%.

In some embodiments, a compound disclosed herein may have an IP bioavailability of about 10% to about 35%. As used herein “IP bioavailability” and “bioavailability upon IP administration” refer to the systemic availability (i.e., blood/plasma levels) of a given amount of a compound administered intraperitoneally (IP) to a subject according to the methods herein. In some embodiments, a compound disclosed herein may have an IP bioavailability of about 10%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, or about 35%.

### V. Kits

In certain embodiments, kits are provided herein for use in treating or alleviating a targeted disease (e.g., Parkinson's disease, addiction, eating disorder) or condition treatable by use of a compound disclosed herein. In some embodiments, kits herein can include instructions for use in accordance with any of the methods described herein. In other embodiments, instructions can include a description of administering a compound and/or pharmaceutical composition disclosed herein to a subject at risk of the target disease. In certain embodiments, kits disclosed herein can include instructions for using the components of the kit, for example relating to the use of a compound and/or pharmaceutical composition disclosed herein. In accordance with embodiments herein, kits can include instructions that provide information as to dosage, dosing schedule, and route of administration for the intended treatment.

In some embodiments, kits disclosed herein can include at least one container. In accordance with embodiments herein, containers can be any container such as tubes, vials, bottles, syringe, such as unit doses, bulk packages (e.g., multi-dose packages) or sub-unit doses. Instructions supplied in the kits of the invention can be written instructions on a label or package insert (e.g., a paper sheet included in the kit), but machine-readable instructions (e.g., instructions carried on a magnetic or optical storage disk) are also acceptable. The label or package insert indicates that the composition is used for treating, delaying the onset and/or alleviating the disease (e.g., Parkinson's disease, an addiction). Instructions can be provided for practicing any of the methods described herein.

Kits disclosed herein can include suitable packaging. Suitable packaging includes, but is not limited to, vials, bottles, jars, flexible packaging (e.g., sealed Mylar or plastic bags), and the like. Also contemplated herein are packages for use in combination with a specific device, such as an inhaler, nasal administration device (e.g., an atomizer) or an infusion device such as a minipump. A kit can have a sterile access port (for example the container can be an intravenous solution bag or a vial having a stopper pierceable by a hypodermic injection needle). The container can also have a sterile access port (for example the container can be an intravenous solution bag or a vial having a stopper pierceable by a hypodermic injection needle). At least one active agent in the composition can be a compound disclosed herein.

Kits can optionally provide additional components such as buffers and interpretive information. Normally, the kit includes a container and a label or package insert(s) on or associated with the container. In some embodiments, the invention provides articles of manufacture including contents of the kits described above.

### EXAMPLES

While the present disclosure has been described with reference to the specific embodiments thereof, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit, and scope of the present disclosure. All such modifications are intended to be within the scope of the disclosure.

### Example 1. Discovery of GHSR1a-Selective, Small Molecules by High-Throughput GHSR1a Screening and Structural Characterization

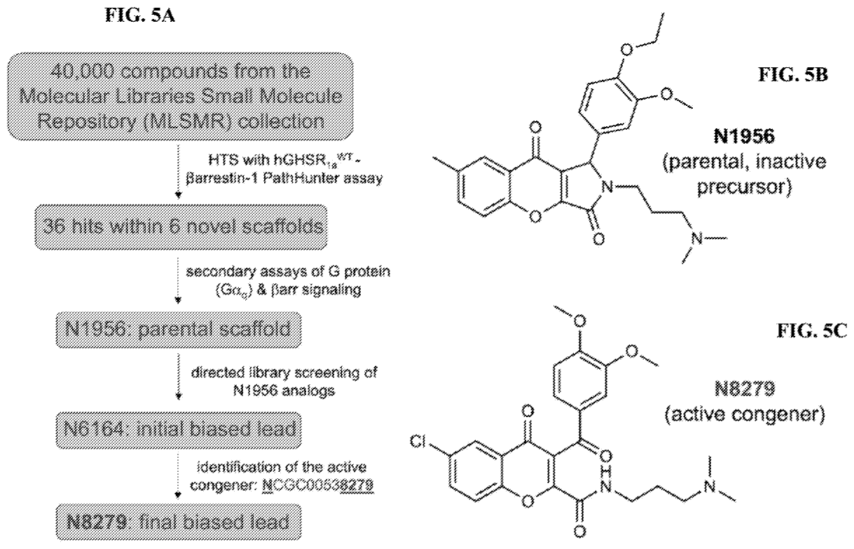

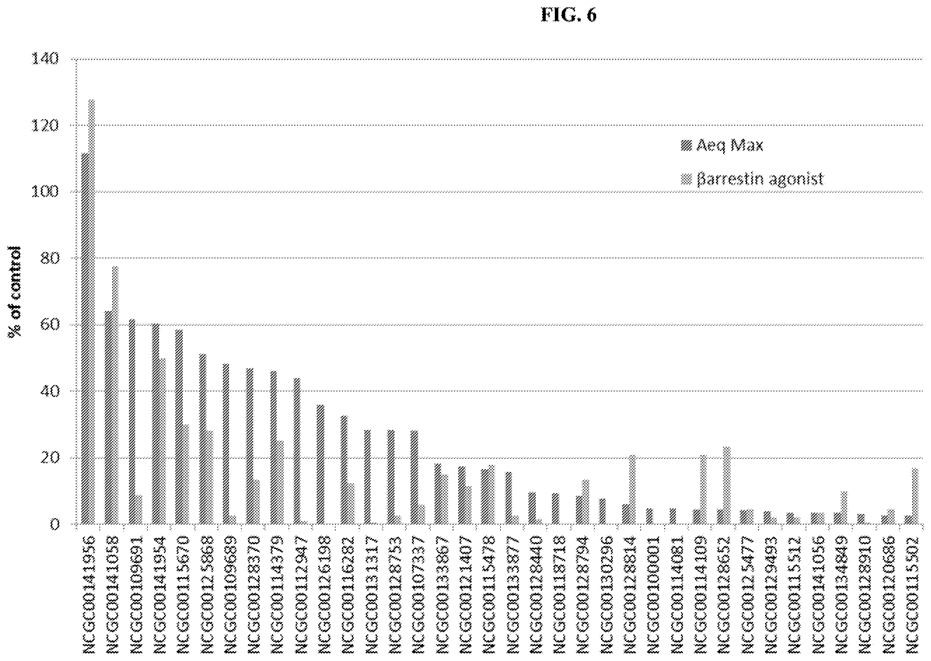

To discover novel, biased GHSR1a ligands, a cell-based, human GHSR1a/beta-arrestin1 (β-arr1) chemiluminescent assay (DiscoverX, PathHunter) was used to screen 47K compounds from the Sytravon library and the NCATS Pharmacological collection (NPC) (FIG. 5A). Thirty-six hits were identified (0.09% hit rate) with activities greater than 50% of the activity shown by the human acyl-ghrelin peptide (FIG. 6). Structure-cluster analysis of the hits revealed 6 novel chemical scaffolds from the 36 compounds. The hit compounds were reassessed in secondary assays for Gα_{q/11}-dependent, intracellular Ca²⁺ mobilization (iCa²⁺) and β-arr2^{GFP} translocation (Table 1).

TABLE 1

| | Calcium signaling | | | Beta-arrestin translocation | | |
|---|---|---|---|---|---|---|
| | EC₅₀ | 95% | ECₘₐₓ | EC₅₀ | 95% | ECₘₐₓ |
| Molecule ID | [M] | CI | (%) | [M] | CI | (%) |
| L626.585 | 1.46E−08 | 1.101e−008 to | 100 | 2.40E−08 | 2.783e−009 to | 100 |
| | | 1.948e−008 | | | 2.064e−007 | |
| NCGC00141956 | 3.32E−07 | 2.175e−007 to | 105 | 2.31E−06 | 5.597e−007 to | 107 |
| | | 5.052e−007 | | | 9.564e−006 | |
| NCGC00136164 | 6.17E−08 | 4.646e−008 to | 97 | 2.53E−07 | 2.511e−008 to | 98 |
| | | 8.190e−008 | | | 2.545e−006 | |
| MLS000103531 | 3.20E−10 | 2.263e−012 to | 8 | 6.30E−06 | 3.690e−007 to | 204 |
| | | 4.536e−008 | | | 0.0001075 | |
| MLS000115599 | 2.04E−05 | 1.228e−009 to | 71 | 2.86E+00 | | N/A |
| | | 0.3396 | | | | |
| MLS000115606 | 2.29E−01 | N/A | N/A | 3.12E−08 | 7.451e−012 to | 24 |
| | | | | | 0.0001306 | |
| MLS002585710 | 1.76E−08 | 1.896e−009 to | 56 | 1.85E+00 | | N/A |
| | | 1.635e−007 | | | | |
| MLS000115604 | 8.04E−09 | 1.598e−010 to | 51 | 2.98E−09 | 4.727e−016 to | 38 |
| | | 4.044e−007 | | | 0.01877 | |
| MLS000115603 | 6.81E−09 | 3.904e−010 to | 48 | ~0.004690 | (Very w ide) | N/A |
| | | 1.186e−007 | | | | |
| MLS000036469 | 3.06E−09 | 3.820e−010 to | 50 | 1.47E−04 | 1.275e−016 to | N/A |
| | | 2.456e−008 | | | 1.704e+008 | |
| MLS000115621 | 2.38E−06 | 1.165e−006 to | 126 | N/A | N/A | 0 |
| | | 4.875e−006 | | | | |
| NCGC00101911 | 2.26E−09 | 1.422e−010 to | 44 | 3.59E−01 | N/A | N/A |
| | | 3.585e−008 | | | | |
| NCGC00101920 | ~0.01848 | (Very wide) | N/A | 1.65E−08 | 6.914e−012 to | 9 |
| | | | | | 3.957e−005 | |
| NCGC00136166 | ~0.0002955 | (Very wide) | N/A | 7.16E−08 | 2.123e−027 to | 11 |
| | | | | | 2.411e+012 | |
| NCGC00136184 | 1.31E−05 | 2.769e−006 to | 129 | 2.33E−06 | 8.463e−011 to | 24 |
| | | 6.231e−005 | | | 0.06430 | |
| NCGC00101634 | 7.71E−06 | 3.903e−007 to | 61 | 4.26E−07 | 1.016e−019 to | 10 |
| | | 0.0001525 | | | 1.787e+006 | |
| NCGC00101917 | 4.30E−09 | 7.476e−011 to | 39 | 4.24E−09 | 1.341e−014 to | 20 |
| | | 2.478e−007 | | | 0.001341 | |
| NCGC00101942 | 8.22E−15 | N/A | 57 | 5.28E−07 | 6.135e−016 to | 12 |
| | | | | | 454.2 | |
| NCGC00101945 | 1.20E−06 | 6.134e−008 to | 63 | 2.00E−06 | 1.223e−012 to | 7 |
| | | 2.342e−005 | | | 3.267 | |
| NCGC00101939 | 4.90E−06 | 2.430e−006 to | 121 | 1.16E−08 | 4.039e−012 to | 20 |
| | | 9.860e−006 | | | 3.315e−005 | |
| NCGC00101941 | ~0.02693 | (Very wide) | N/A | 9.50E−08 | 6.833e−009 to | 83 |
| | | | | | 1.321e−006 | |
| NCGC00141056 | 9.38E−07 | 7.854e−008 to | 68 | 2.19E−09 | 8.034e−016 to | 19 |
| | | 1.119e−005 | | | 0.005953 | |
| NCGC00141954 | 7.14E−07 | 2.775e−007 to | 105 | 5.33E−06 | 2.970e−007 to | 196 |
| | | 1.835e−006 | | | 9.575e−005 | |
| NCGC00119774 | 5.62E−06 | 3.974e−007 to | 75 | 3.05E−06 | 9.342e−008 to | 163 |
| | | 7.948e−005 | | | 9.943e−005 | |
| NCGC00101673 | 3.24E−05 | 8.270e−011 to | 115 | 2.40E−06 | 5.250e−009 to | 49 |
| | | 12.68 | | | 0.001097 | |
| NCGC00119776 | 1.20E−05 | 4.349e−006 to | 125 | 4.99E−08 | 3.464e−010 to | 26 |
| | | 3.302e−005 | | | 7.181e−006 | |
| NCGC00119778 | ~0.06196 | (Very wide) | N/A | 2.97E+00 | N/A | N/A |
| NCGC00116418 | ~0.003007 | (Very wide) | N/A | N/A | N/A | N/A |
| NCGC00101907 | 3.35E−06 | 1.355e−007 to | 40 | 5.58E−07 | 2.145e−011 to | 39 |
| | | 8.282e−005 | | | 0.01451 | |
| NCGC00101948 | ~0.01115 | (Very wide) | N/A | N/A | N/A | N/A |
| NCGC00101937 | 2.78E−06 | 1.819e−006 to | 112 | 8.55E−07 | 4.555e−008 to | 49 |
| | | 4.243e−006 | | | 1.603e−005 | |
| NCGC00101919 | ~0.001517 | (Very wide) | N/A | 1.74E−06 | 1.186e−007 to | 99 |
| | | | | | 2.545e−005 | |
| NCGC00101915 | ~0.01916 | (Very wide) | N/A | 1.36E−05 | 2.339e−007 to | 368 |
| | | | | | 0.0007879 | |
| NCGC00101909 | ~0.003015 | (Very wide) | N/A | 2.54E−07 | 6.660e−010 to | 64 |
| | | | | | 9.708e−005 | |
| NCGC00141058 | 1.29E−06 | 5.769e−007 to | 97 | 2.03E−06 | 2.389e−007 to | 142 |
| | | 2.878e−006 | | | 1.720e−005 | |
| NCGC00101913 | ~0.01803 | (Very wide) | N/A | 1.55E−06 | 1.386e−007 to | 153 |
| | | | | | 1.739e−005 | |

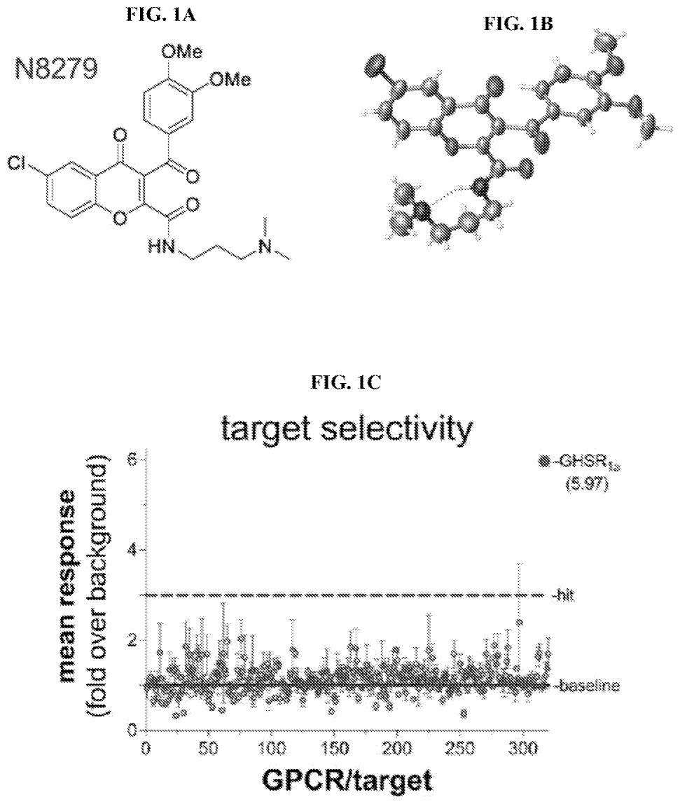

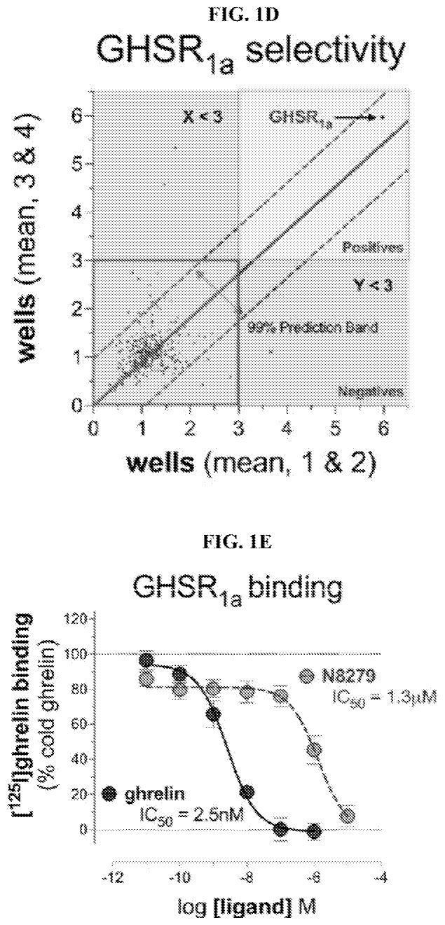

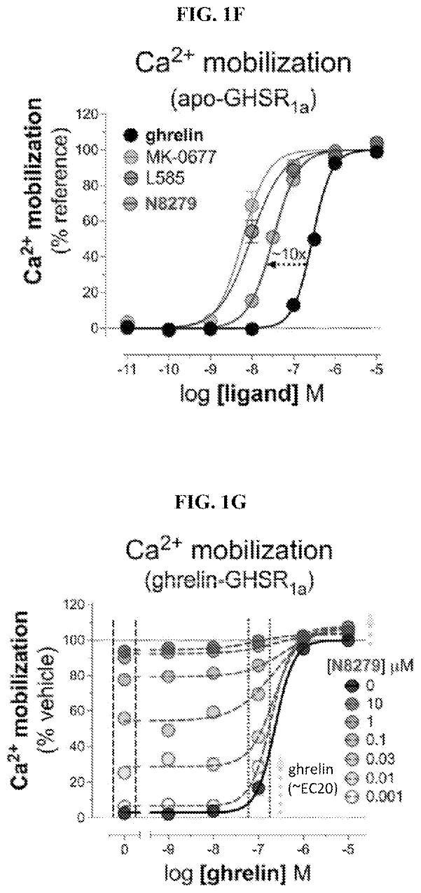

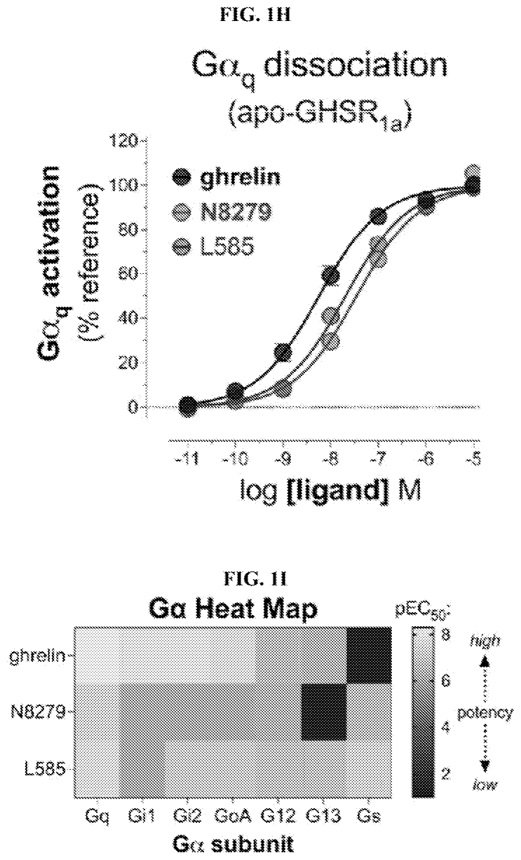

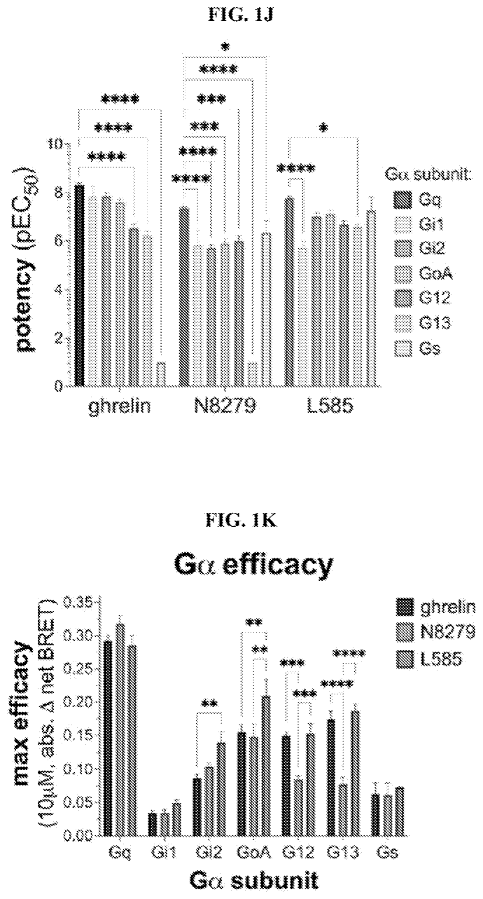

From these experiments, NCGC141956 (N1956) (FIG. 5B, Table 1) was selected for further characterization based on its sub-μM potency and full efficacy relative to the unbiased, small molecule GHSR1a agonist, L692,585 (L585). A further directed library screen of commercial N1956 analogs identified NCGC00136164 (N6164), which unexpectedly, was determined to be a Gα_{q/11}-biased GHSR1a agonist relative to β-arr2 translocation (Table 1). However, the activity of N1956 and N6164 could not be confirmed upon re-synthesizing these molecules. A LC-MS examination of the DMSO aliquots used in the screening campaign disclosed impurities within N1956 and N6164 solutions, corresponding to oxidated derivatives of the I-phenyl-chromeno-pyrrole-dione scaffold. Further characterization, investigation using NMR and MS methods (Table 2), and re-synthesis of pure oxidated products resulted in the determination of the active molecule NCGC00538279 (also referred to herein as “N8279” and “NCATS-SM8864” interchangeably) (FIGS. 1A and 5C), which contained a 2-carboxamide-3-benzoyl-4-chromenone backbone. In solution, N8279 equilibrated between open and closed conformers, which in specific solvents and conditions were observed by ¹H NMR (Table 2). The structure of the active, open form of N8279 was confirmed by single crystal X-ray diffraction (FIG. 1B, Table 2).

TABLE 2

| | |
|---|---|
| Identification code | N8279 (NCATS-SM8864) |
| Empirical formula | C24 H25 Cl N2 O6 |
| Molecular formula | C24 H25 Cl N2 O6 |
| Formula weight | 472.91 |
| Temperature | 285 K |
| Wavelength | 1.54178 Å |
| Crystal system | Monoclinic |
| Space group | P 1 21/c 1 |
| Unit cell dimensions | a = 15.5969(5) Å; α = 90° |
| | b = 14.5091(6) Å; |
| | β = 100.450(2)° |
| | c = 21.7738(9) Å; γ = 90°. |
| Volume | 4845.6(3) Å3 |
| Z | 8 |
| Density (calculated) | 1.296 Mg/m3 |
| Absorption coefficient | 1.747 mm−1 |
| F(000) | 1984 |
| Crystal size | 0.267 × 0.243 × 0.228 mm3 |
| Crystal color, habit | Colorless Block |
| Theta range for data | 2.881 to 59.009°. |
| collection | |
| Index ranges | 17 <= h <= 17, −16 <= k <= 16, −24 <= |
| | l <= 24 |
| Reflections collected | 58959 |
| Independent reflections | 6947 [R(int) = 0.0596, R(sigma) = 0.0335] |
| Completeness to theta = | 99.5% |
| 59.009° | |
| Absorption correction | Semi-empirical from equivalents |
| Max. and min. transmission | 0.3085 and 0.1652 |
| Refinement method | Full-matrix least-squares on F2 |
| Data/restraints/parameters | 6947/2/611 |
| Goodness-of-fit on F2 | 1.049 |
| Final R indices [I > | R1 = 0.0641, wR2 = 0.1578 |
| 2sigma(I)] | |
| R indices (all data) | R1 = 0.0848, wR2 = 0.1705 |
| Extinction coefficient | NA |
| Largest diff. peak and hole | 0.266 and −0.271 e · Å−3 |

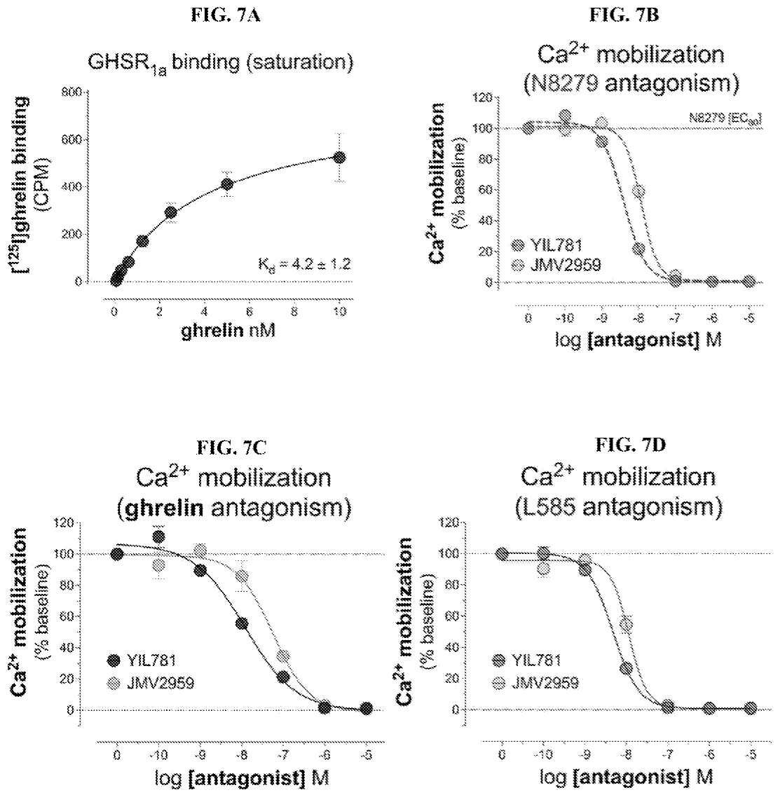

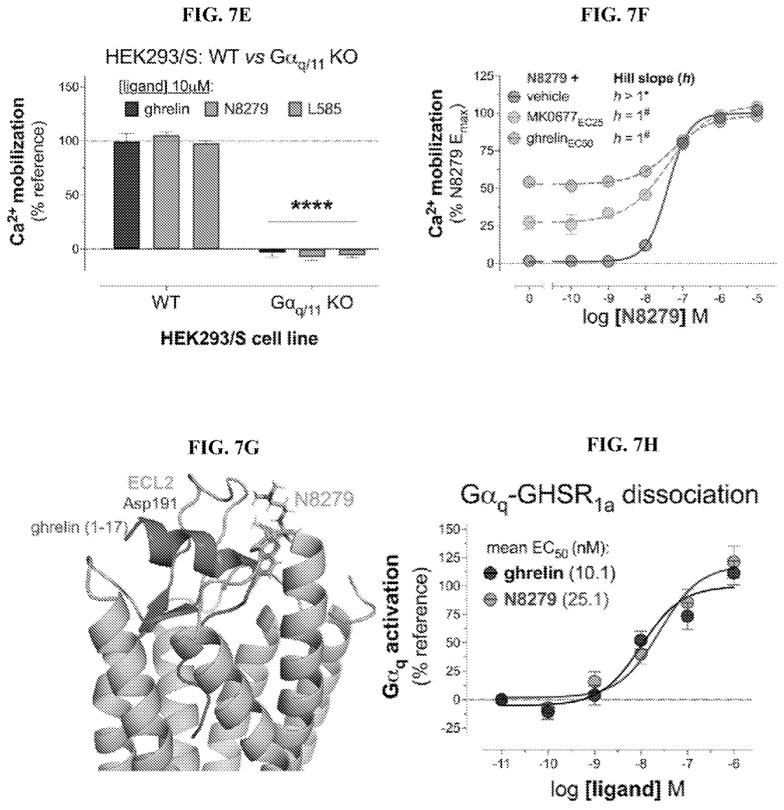

To determine target selectivity, N8279 activity was evaluated across the GPCRome (at 320 receptors) using the PRESTO-Tango platform by high-throughput screening with a β-arr2 recruitment assay similar to the method described in Kroeze et al., Nat Struct Mol Biol. 2015; 22(5):362-9, the disclosure of which is incorporated herein in its entirety. Hits were defined as >3-fold activation above baseline. N8279 stimulated 6-fold activation at the GHSR1a and did not exceed >3-fold activation at any other GPCR (FIG. 1C). The assay quality was assessed by plotting duplicate, independent trial averages (derived from 4 independent wells) for each receptor as a point (X,Y). The corresponding plot of points for an ideal assay were fit by a regression line with the slope=1. A plot of the assay points produced a regression line with slope of 0.98 (FIG. 1D) and was shown with its accompanying 99% prediction band that contained GHSR1a as the only hit. Next, the relative affinity of N8279 for the hGHSR1aWT (GHSR1a) was determined by radioligand binding with [¹²⁵I]ghrelin. Initial saturation studies confirmed that [¹²⁵I]ghrelin bound the GHSR1a asymptotically with nM affinity (FIG. 7A). Subsequent competition binding using [¹²⁵I]ghrelin at its ˜Kd demonstrated that both unlabeled ghrelin and N8279 displaced [¹²⁵I]ghrelin completely from the GHSR1a with high and relatively low affinity (IC₅₀ [nM]=2.5 and 1,300, respectively) (FIG. 1E).

### Example 2. Analytical Methods Used for GHSR1a-Selective Compounds

The following analytical methods were used to evaluate the GHSR1a-selective compounds, in the following examples. Analytical analysis was performed on an Agilent LC/MS (Agilent Technologies, Santa Clara, CA). Method 1: A 7-min gradient of 4% to 100% acetonitrile (containing 0.025% trifluoroacetic acid) in water (containing 0.05% trifluoroacetic acid) was used with an 8-min run time at a flow rate of 1.0 mL/min.

Method 2: A 3-min gradient of 4% to 100% acetonitrile (containing 0.025% trifluoroacetic acid) in water (containing 0.05% trifluoroacetic acid) was used with a 4.5-min run time at a flow rate of 1.0 mL/min. A Phenomenex Luna Cis column (3-micron, 3×75 nm) was used at a temperature of 50° C. Purity determination was performed using an Agilent diode array detector for both Method 1 and Method 2. Mass determination was performed using an Agilent 6130 mass spectrometer with electrospray ionization in the positive mode.

¹H NMR data was collected on a Bruker 400 MHz NMR.

Solvents from Sigma Aldrich were purchased commercially and used without further purification.

### Example 3: Preparation of 6-Chloro-3-(3,4-dimethoxybenzoyl)-N-(3-(dimethylamino)propyl)-4-oxo-4H-chromene-2-carboxamide (5) NCGC00538279 (N8279)

The preparation of 6-chloro-3-(3,4-dimethoxybenzoyl)-N-(3-(dimethylamino)propyl)-4-oxo-4H-chromene-2-carboxamide (5) was conducted according to the Chemical Scheme 1 shown below.

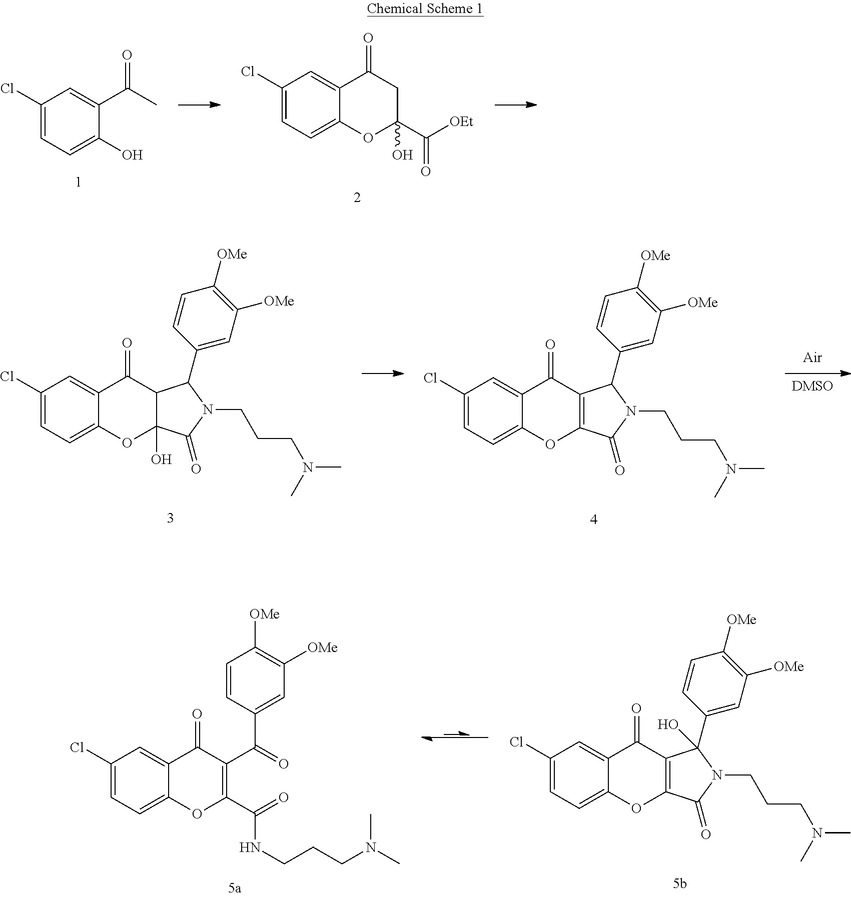

### Preparation of Ethyl 6-chloro-2-hydroxy-4-oxochromane-2-carboxylate

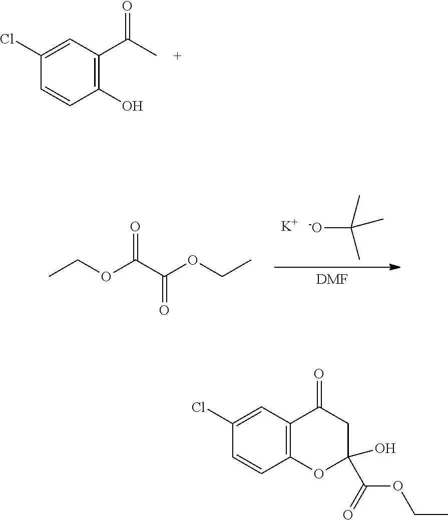

1-(5-Chloro-2-hydroxyphenyl) ethan-1-one (5 g, 29.3 mmol) was dissolved in DMF (100 ml), then the solution was placed under an ice bath. Potassium 2-methylpropane-2-olate (64.5 ml, 64.5 mmol) was slowly added to the solution. After 5 min, diethyl oxalate (5.97 ml, 44.0 mmol) was added to the reaction mixture with one portion. A yellow solid appeared. The reaction mixture was kept at room temperature overnight. The reaction mixture was diluted with ethyl acetate. Then, 1N HCl was added to the reaction mixture to extract the organic layer. The organic layer containing insoluble yellow solid was removed by filtration. The organic layer was dried with sodium sulfate. The crude product was obtained by evaporation of the solvent, which was purified by silica column (10 to 30% EA in Hex). Yield (92%). ¹H NMR (400 MHz, DMSO-d₆) δ 8.33 (d, J=1.9 Hz, 1H), 7.71-7.60 (m, 2H), 7.15 (d, J=8.7 Hz, 1H), 4.22 (qd, J=7.1, 1.0 Hz, 2H), 3.32 (d, J=1.7 Hz, 1H), 2.91 (dd, J=16.5, 1.1 Hz, 1H), 1.22 (td, J=7.1, 1.1 Hz, 3H).

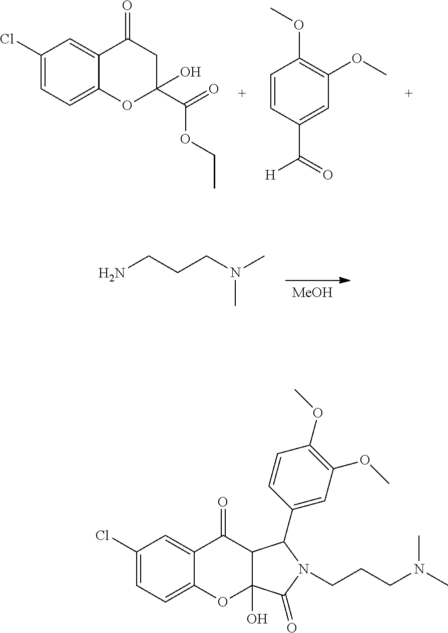

3,4-Dimethoxybenzaldehyde (100 mg, 0.602 mmol) was placed in the microwave reaction vessel and dissolved in MeOH (5 ml). To the solution was added N¹,N¹-dimethylpropane-1,3-diamine (76 pal, 0.602 mmol). The microwave-assisted reaction was run at 110° C. for 30 min. After cooling the reaction vessel to room temperature, ethyl 6-chloro-2-hydroxy-4-oxochromane-2-carboxylate (200 mg, 0.739 mmol) was added to the reaction vessel and the microwave-assisted reaction was run at 70° C. for 30 min. The reaction mixture was rotovaped and the crude product was fractionally filtered through silica gel (Methanol/DCM) to afford the desired crude product. This crude product was taken directly to the next step.

### Example 5: Preparation of 7-Chloro-1-(3,4-dimethoxyphenyl)-2-(3-(dimethylamino)propyl)-1,2-dihydrochromeno[2,3-c]pyrrole-3,9-dione (4)

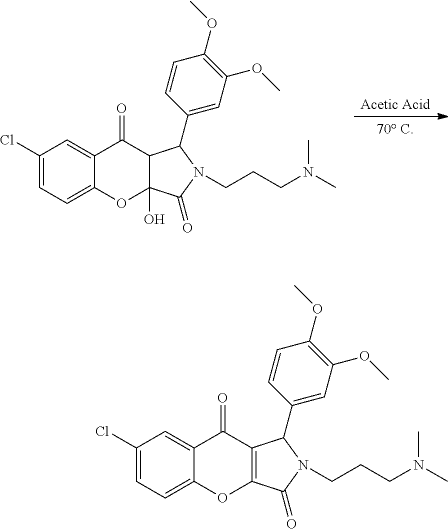 [SMILES: COc1ccc(C2C3C(=O)c4cc(Cl)ccc4OC3(O)C(=O)N2CCCN(C)C)cc1OC ; "70° C." ; "Acetic Acid" ; COc1ccc(C2c3c(oc4ccc(Cl)cc4c3=O)C(=O)N2CCCN(C)C)cc1OC]

7-Chloro-1-(3,4-dimethoxyphenyl)-2-(3-(dimethylamino)propyl)-3a-hydroxy-1,2,3a,9a-tetrahydrochromeno[2,3-c]pyrrole-3,9-dione (100 mg, 0.211 mmol) was placed in the microwave reaction vessel and dissolved in acetic acid (5 ml). The microwave-assisted reaction was run at 70° C. for one hour. The reaction mixture was rotovaped and then dissolved in EtOH. After heating, the product came out upon cooling to room temperature. The product was filtered and dried (86 mg, 90%). ¹H NMR (400 MHz, CDCl₃) δ 8.12 (d, J=2.3 Hz, 1H), 7.71-7.61 (m, 2H), 6.92-6.81 (m, 2H), 6.65 (d, J=1.9 Hz, 1H), 5.48 (s, 1H), 3.81-3.90 (m, 7H), 2.99 (ddd, J=14.0, 8.2, 5.9 Hz, 1H), 2.29-2.19 (m, 2H), 2.15 (s, 6H), 1.85-1.63 (m, 2H); LCMS RT (Method 1)=4.035 min, m/z 457.2 [M+H⁺]; HRMS (ESI) m/z calcd for C₂₄H₂₆ClN₂O₅⁺ [M+H⁺] 457.1530, found 457.1537.

The title compound was prepared according to Vydzhak, R. N.; Panchishin, S. Y. Synthesis of 2-Alkyl-1-aryl-1,2-dihydrochromeno[2,3-c]pyrrole-3,9-dione Derivatives. Russ. J. Gen. Chem. 2008, 78, 2391-2397.

### Example 6: Preparation of 6-Chloro-3-(3,4-dimethoxybenzoyl)-N-(3-(dimethylamino)propyl)-4-oxo-4H-chromene-2-carboxamide (5) NCGC00538279 (N8279)

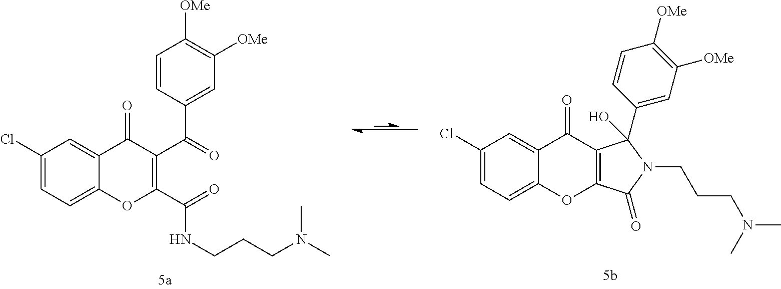 [SMILES: COc1ccc(C(=O)c2c(C(=O)NCCCN(C)C)oc3ccc(Cl)cc3c2=O)cc1OC ; COc1ccc(C2(O)c3c(oc4ccc(Cl)cc4c3=O)C(=O)N2CCCN(C)C)cc1OC]

A solution of 7-chloro-1-(3,4-dimethoxyphenyl)-2-(3-(dimethylamino)propyl)-1,2-dihydrochromeno[2,3-c]pyrrole-3,9-dione (200 mg, 0.438 mmol) was dissolved in DMSO (10 mL). To this solution was bubbled air until complete conversion was observed by LCMS. The reaction mixture was blown dry and purified by column chromatography (Methanol/DCM) to afford the desired product (2:1 ratio 5a:5b) as a light brown solid (86.7 mg, 42%).

5a: ¹H NMR (400 MHz, CD₂Cl₂) δ 9.48 (s, 1H), 8.12 (d, J=2.5 Hz, 1H), 7.73-7.62 (m, 2H), 7.08 (d, J=2.2 Hz, 1H), 6.98 (dd, J=8.4, 2.2 Hz, 1H), 6.84 (d, J=8.4 Hz, 1H), 3.83 (s, 3H), 3.82 (s, 3H), 3.69 (ddd, J=14.7, 5.5, 3.9 Hz, 2H), 2.88 (ddd, J=14.1, 10.4, 3.2 Hz, 2H), 2.33 (m, 2H), 2.22 (s, 6H); 5b: ¹H NMR (400 MHz, CD₂Cl₂) δ 8.15 (d, J=2.6 Hz, 1H), 7.77 (dd, J=8.9, 2.6 Hz, 1H), 7.73-7.64 (m, 1H), 7.58 (d, J=2.0 Hz, 1H), 7.30 (dd, J=8.4, 2.0 Hz, 1H), 6.84 (d, J=8.4 Hz, 1H), 3.92 (s, 3H), 3.89 (s, 3H), 3.51-3.43 (m, 1H), 2.74 (m, 1H), 2.60-2.52 (m, 1H), 2.53 (s, 6H), 2.33 (m, 1H), 1.87 (m, 1H), 1.71 (m, 1H); LCMS RT (Method 1)=3.921 min, m/z 473.2 [M+H⁺]; HRMS (ESI) m/z calcd for C₂₄H₂₆ClN₂O₆⁺ [M+H⁺] 473.1479, found 473.1467.

### Example 7: Preparation of 6-Chloro-9a-(3,4-dimethoxyphenyl)-1-(2-(diethylamino)ethyl)-3a,9a-dihydrochromeno[2,3-b]pyrrole-2,3,4(1H)-trione NCGC00650076-01

The preparation of 6-chloro-9a-(3,4-dimethoxyphenyl)-1-(2-(dimethylamino)ethyl)-3a,9a-dihydrochromeno[2,3-b]pyrrole-2,3,4(1H)-trione was conducted according to the Chemical Scheme 2 shown below.

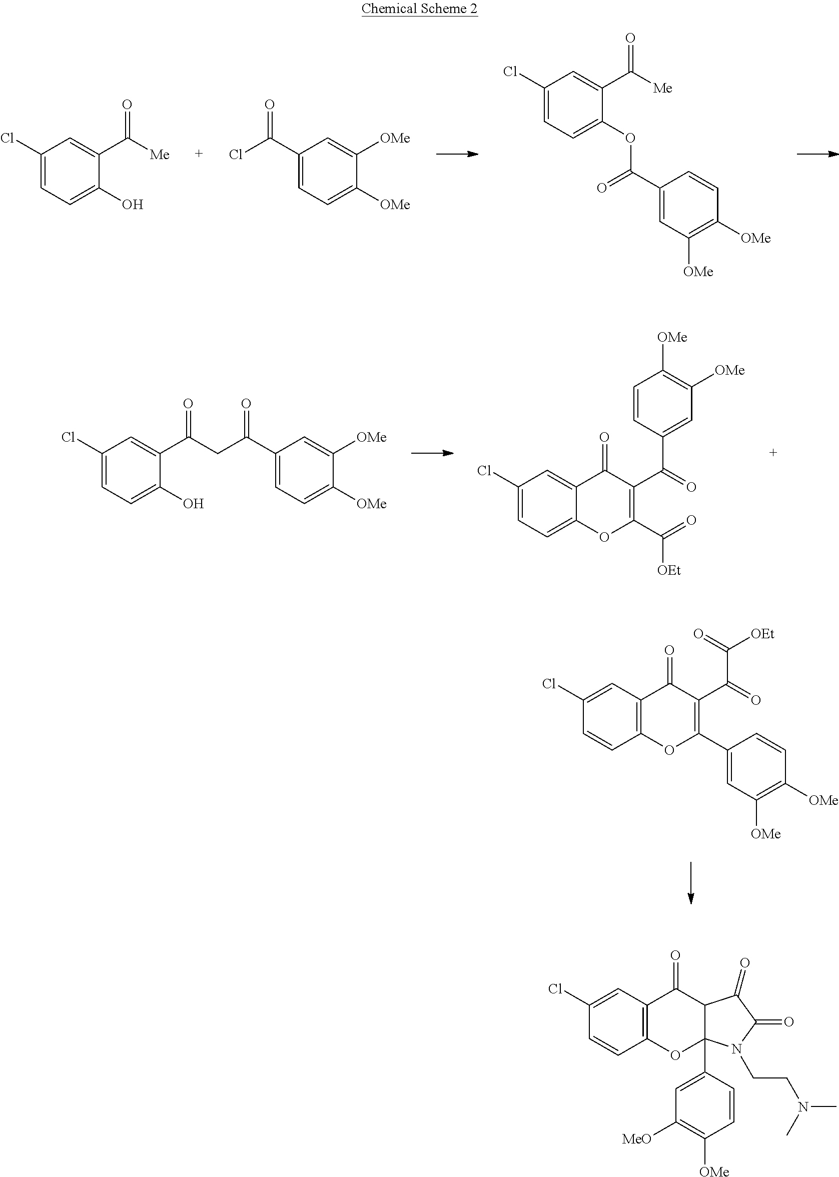

### Example 8: Preparation of 2-Acetyl-4-chlorophenyl 3,4-dimethoxybenzoate

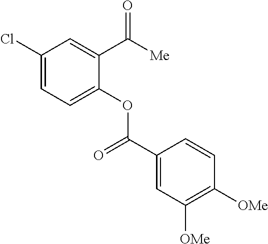 [SMILES: COc1ccc(C(=O)Oc2ccc(Cl)cc2C(C)=O)cc1OC]

To a suspension of 1-(5-chloro-2-hydroxyphenyl)ethan-1-one (2 g, 11.72 mmol) and 3,4-dimethoxybenzoyl chloride (2.58 g, 12.86 mmol) in pyridine (30 mL) was added a catalytic amount of 4-DMAP and heated to 50° C. under N₂. Pyridine was rotovaped off and the resulting crude product was dissolved in DCM and washed with 1N HCl. The organic layer was dried with MgSO₄, filtered, and rotovaped. The crude product was purified by column chromatography (hexanes/EtOAc) to yield the desired product as a white solid (3.6 g, 92%). ¹H NMR (400 MHz, DMSO-d₆) δ 7.96 (d, J=2.6 Hz, 1H), 7.75 (ddd, J=13.4, 8.5, 2.3 Hz, 2H), 7.56 (d, J=2.1 Hz, 1H), 7.42 (d, J=8.7 Hz, 1H), 7.16 (d, J=8.6 Hz, 1H), 3.88 (s, 3H), 3.84 (s, 3H); 2.51 (s, 3H).

### Example 9: Preparation of (Z)-1-(5-chloro-2-hydroxyphenyl)-3-(3,4-dimethoxyphenyl)-3-hydroxyprop-2-en-1-one

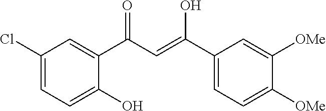 [SMILES: COc1ccc(/C(O)=C/C(=O)c2cc(Cl)ccc2O)cc1OC]

A suspension of 2-acetyl-4-chlorophenyl 3,4-dimethoxybenzoate (2 g, 5.97 mmol) and potassium hydroxide (503 mg, 8.97 mmol) in pyridine (20 ml) was flushed with N₂, sealed, and heated to 50° C. Pyridine was rotovaped off and the resulting crude product was dissolved in DCM and washed with 1N HCL. The organic layer was dried With MgSO₄, filtered and rotovaped. The crude product was purified by column chromatography (hexanes/EtOAc) to yield the desired product (3:1 conformational isomers) as a yellow solid (1.55 g, 78%). ¹H NMR (400 MHz, DMSO-d₆) δ 16.73 (s, 1H), 11.34 (s, 1H), 7.97 (d, J=2.7 Hz, 1H), 7.71 (dd, J=8.5, 2.1 Hz, 1H), 7.54 (d, J=2.1 Hz, H), 7.51-7.45 (m, 1H), 7.37 (s, 1H), 7.14 (d, J=8.5 Hz, 1H), 7.03 (d, J=8.8 Hz, 1H), 3.87 (s, 3H), 3.86 (s, 3H).

### Example 10: Preparation of Ethyl 6-chloro-3-(3,4-dimethoxybenzoyl)-4-oxo-4H-chromene-2-carboxylate (product A) NCGC00688026-01, ethyl2-(6-chloro-2-(3,4-dimethoxyphenyl)-4-oxo-4H-chromen-3-yl)-2-oxoacetate (product B)

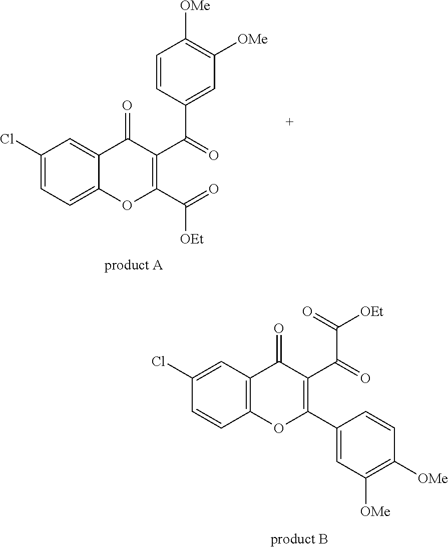 [SMILES: CCOC(=O)c1oc2ccc(Cl)cc2c(=O)c1C(=O)c1ccc(OC)c(OC)c1 ; CCOC(=O)C(=O)c1c(-c2ccc(OC)c(OC)c2)oc2ccc(Cl)cc2c1=O]

A solution of 1-(5-chloro-2-hydroxyphenyl)-3-(3,4-dimethoxyphenyl)propane-1,3-dione (1.55 g, 4.63 mmol) in pyridine (40 mL) was cooled to 0° C. Then, ethyl 2-chloro-2-oxoacetate (1.03 mL, 9.22 mmol) was added dropwise. The reaction was stirred under N₂ and slowly warmed to room temperature. Pyridine was rotovaped off and the resulting crude product was dissolved in DCM and washed with 1N HCl. The organic layer was dried with MgSO₄, filtered, and rotovaped. The crude product was purified by column chromatography (hexanes/EtOAc) to yield the desired products as a yellow solid. product A (520 mg, 26.9%) and product B (750 mg, 38.9%). Product A: ¹H NMR (400 MHz, CD₂Cl₂) a 8.15 (d, J=2.6 Hz, 1H), 7.79 (dd, J=9.0, 2.6 Hz, 1H), 7.66 (d, J=9.0 Hz, 1H), 7.58 (d, J=2.0 Hz, 1H), 7.33 (dd, J=8.4, 2.0 Hz, 1H), 6.87 (d, J=8.4 Hz, 1H), 4.27 (q, J=7.1 Hz, 2H), 3.92 (d, J=4.5 Hz, 6H), 1.17 (t, J=7.1 Hz, 3H). LCMS RT (Method 1)=5.411 min, m/z 417.0 [M+H⁺]; HRMS (EST) in z calcd for C₂₁H₁₈ClO₇⁺ [M+H⁺] 417.0741, found 417.0752. Product B: ¹H NMR (400 MHz, CD₂C₂) δ 8.17 (d, J=2.6 Hz, 1H), 7.74 (dd, J=8.9, 2.6 Hz, 1H), 7.59 (d, J=8.9 Hz, 1H), 7.25 (dd, J=8.4, 2.2 Hz, 1H), 7.19 (d, J=2.1 Hz, 1H), 6.98 (d, J=8.4 Hz, 1H), 4.23 (q, J=7.1 Hz, 2H), 3.93 (s, 3H), 3.90 (s, 3H), 1.27 (t, J=7.1 Hz, 3H). LCMS RT (Method 2)=3.426 min, m/z 417.1 [M+H⁺].

### Example 11: Preparation of 6-Chloro-9a-(3,4-dimethoxyphenyl)-1-(2-(dimethylamino)ethyl)-3a,9a-dihydrochromeno[2,3-b]pyrrole-2,3,4(1H)-trione

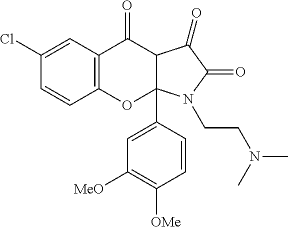 [SMILES: COc1ccc(C23Oc4ccc(Cl)cc4C(=O)C2C(=O)C(=O)N3CCN(C)C)cc1OC]

To a solution of ethyl 2-(6-chloro-2-(3,4-dimethoxyphenyl)-4-oxo-4H-chromen-3-yl)-2-oxoacetate (30 mg, 0.072 mmol) in acetonitrile (3 mL) was added N1,N1-dimethylethane-1,2-diamine (10.5 mL, 0.096 mmol). The reaction was stirred at room temperature under N₂. The reaction mixture was rotovaped off and purified by column chromatography (hexanes/EtOAc) to yield the desired product as a white solid (30 mg, 91%).

¹H NMR (400 MHz, DMSO-d6) δ 9.64 (bs, 1H), 7.48 (d, J=2.7 Hz, 1H), 7.37 (dd, J=8.7, 2.7 Hz, 1H), 7.24 (d, J=8.7 Hz, 1H), 7.12 (s, 1H), 6.99 (dd, J=8.5, 2.1 Hz, 1H), 6.79 (d, J=8.5 Hz, 1H), 3.76-3.62 (m, 7H), 3.25-2.05 (m, 2H), 2.95-2.80 (m, 1H), 2.81 (s, 61H); LCMS RT (Method 1)=4.268 min, m/z 458.8 [M+H⁺]; HRMS (ESI) nm/z calcd for C₂₃H₂₄ClN₂O₆⁺ [M+H⁺] 459.1323, found 459.1218.

### Example 12: Preparation of 6-chloro-9a-(3,4-dimethoxyphenyl)-1-(3-(dimethylamino)propyl)-3a,9a-dihydrochromeno[2,3-b]pyrrole-2,3,4(1H)-trione

### NCGC00649013-01

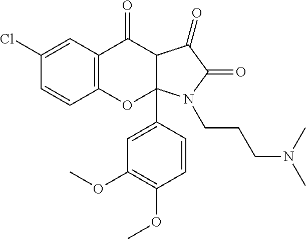 [SMILES: COc1ccc(C23Oc4ccc(Cl)cc4C(=O)C2C(=O)C(=O)N3CCCN(C)C)cc1OC]

The preparation of 6-chloro-9a-(3,4-dimethoxyphenyl)-1-(3-(dimethylamino)propyl)-3a,9a-dihydrochromeno[2,3-b]pyrrole-2,3,4(1H)-trione followed the above Chemical Scheme 2 denoted above. ¹H NMR (400 MHz, DMSO-d₆) δ 9.45 (s, 1H), 7.48 (d, J=2.7 Hz, 1H), 7.36 (dd, J=8.7, 2.8 Hz, 1H), 7.18 (d, J=8.7 Hz, 1H), 6.91 (dd, J 8.4, 2.2 Hz, 1H), 6.85-6.79 (m, 2H), 3.67 (s, 3H), 3.64 (s, 3H), 3.44-3.37 (m, 1H), 3.01-2.85 (m, 3H), 2.74 (s, 6H), 1.85-1.74 (m, 2H); L CMS RT (Method 1)=3.911 min, m/z 473.1 [M+H⁺]; HRMS (EST) m/z calcd for C₂₄H₂₆ClN₂O₆⁺ [M+H⁺] 473.1479, found 473.1246.

### Example 13: Preparation of 3-(3,4-dimethoxybenzoyl)-N-(3-(dimethylamino)propyl)-6-iodo-4-oxo-4H-chromene-2-carboxamide

The preparation of 3-(3,4-dimethoxybenzoyl)-N-(3-(dimethylamino)propyl)-6-iodo-4-oxo-4H-chromene-2-carboxamide was conducted according to Chemical Scheme 3 shown below.

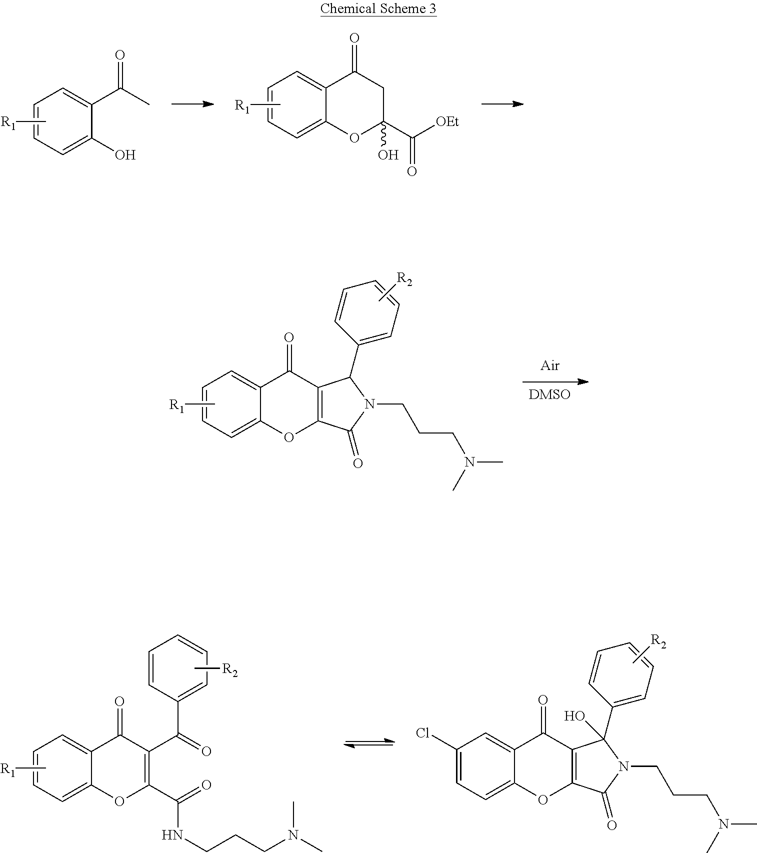

### Example 14: Preparation of ethyl 2-hydroxy-6-iodo-4-oxochromane-2-carboxylate

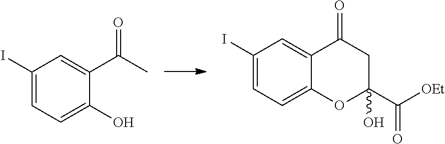 [SMILES: CC(=O)c1cc(I)ccc1O ; CCOC(=O)C1(O)CC(=O)c2cc(I)ccc2O1]

1-(5-Iodo-2-hydroxyphenyl) ethan-1-one (200 mg, 0.763 mmol) was dissolved in THF (10 ml), then the solution was placed under an ice bath. 1M Potassium 2-methylpropane-2-olate in THF (1.7 mL, 2.227 mmol) was slowly added to the solution. After 5 min, diethyl oxalate (103.5 mL, 0.763 mmol) was added to the reaction mixture in one portion. A yellow solid appeared. The reaction mixture was kept at room temperature overnight. The reaction mixture was diluted with ethyl acetate. Then, 1N HCl was added to the reaction mixture to extract the organic layer. The organic layer containing the insoluble yellow solid was removed by filtration. The organic layer was dried over sodium sulfate. The crude product was obtained by evaporation of the solvent, which was purified by silica gel chromatography (10 to 30% EA in Hex). Yield (227 mg, 92%). ¹H NMR (400 MHz, DMSO-d₆) δ 8.31 (d, J=1.9 Hz, 1H), 7.94 (d, J=2.2 Hz, 1H), 7.87 (dd, J=8.7, 2.3 Hz, 1H), 6.93 (d, J=8.6 Hz, 1H), 4.20 (q, J=7.1 Hz, 2H), 3.30 (dd, J=16.4, 1.9 Hz, 1H), 2.88 (d, J=16.5 Hz, 1H), 1.21 (t, J=7.1 Hz, 3H).

### Example 15: Preparation of 1-(3,4-dimethoxyphenyl)-2-(3-(dimethylamino)propyl)-7-iodo-7,2-dihydrochromeno[2,3-c]pyrrole-3,9-dione

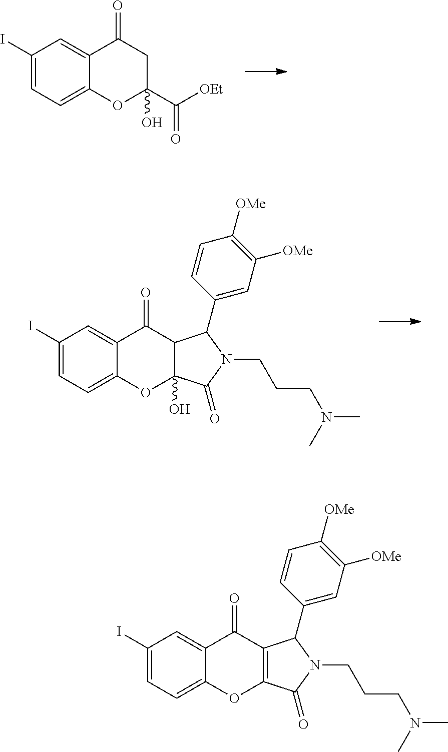 [SMILES: CCOC(=O)C1(O)CC(=O)c2cc(I)ccc2O1 ; COc1ccc(C2C3C(=O)c4cc(I)ccc4OC3(O)C(=O)N2CCCN(C)C)cc1OC ; COc1ccc(C2c3c(oc4ccc(I)cc4c3=O)C(=O)N2CCCN(C)C)cc1OC]

3,4-Dimethoxybenzaldehyde (52.8 mg, 0.318 mmol) was placed in the microwave reaction vessel and dissolved in MeOH (5 ml). To the solution was added N¹,N¹-dimethylpropane-1,3-diamine (40 μl, 0.318 mmol). The microwave-assisted reaction was run at 110° C. for 30 min. After cooling the reaction vessel to room temperature, ethyl 6-chloro-2-hydroxy-4-oxochromane-2-carboxylate (115 mg, 0.318 mmol) was added to the reaction vessel and the microwave-assisted reaction was conducted at 70° C. for 30 min. The reaction mixture was rotovaped and the crude product was fractionally filtered through silica gel (Methanol/DCM) to afford the desired crude product. This was taken directly to the next step. Crude 1-(3,4-dimethoxyphenyl)-2-(3-(dimethylamino)propyl)-3a-hydroxy-7-iodo-1,2,3a,9a-tetrahydrochromeno [2,3-c]pyrrole-3,9-dione was placed in the microwave reaction vessel and dissolved in acetic acid (5 ml). The microwave-assisted reaction was run at 70° C. for one hour. The reaction mixture was rotovaped and then dissolved in EtOH. The EtOH solution was warmed and then cooled to room temperature. Upon cooling to room temperature, the product precipitated. The product was filtered and dried (90 mg, 52%). ¹H NMR (400 MHz, DMSO-d₆) δ 8.26 (d, J=2.2 Hz, 1H), 8.16 (dd, J=8.8, 2.2 Hz, 1H), 7.70 (d, J=8.8 Hz, 1H), 6.97-6.81 (m, 3H), 5.61 (s, 1H), 3.73 (s, 3H), 3.66 (s, 3H), 3.56 (dt, J=14.4, 7.8 Hz, 1H), 2.88 (ddd, J=14.2, 8.6, 6.0 Hz, 1H), 2.21-2.08 (m, 2H), 2.03 (s, 6H), 1.62 (dq, J=13.6, 7.0 Hz, 1H), 1.45 (dq, J=14.1, 7.1 Hz, 1H); LCMS RT (Method 2)=2.864 min, m/z 549.0 [M+H⁺].

The title compound was prepared according to Vydzhak, R. N.; Panchishin, S. Y. Synthesis of 2-Alkyl-1-aryl-1,2-dihydrochromeno[2,3-c]pyrrole-3,9-dione Derivatives. Russ. J. Gen. Chem. 2008, 78, 2391-2397.

### Example 16: Preparation of 3-(3,4-dimethoxybenzoyl)-N-(3-(dimethylamino)propyl)-6-iodo-4-oxo-4H-chromene-2-carboxamide NCGC00842388-01

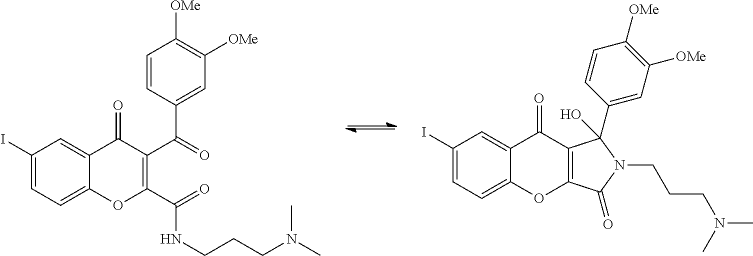 [SMILES: COc1ccc(C(=O)c2c(C(=O)NCCCN(C)C)oc3ccc(I)cc3c2=O)cc1OC ; COc1ccc(C2(O)c3c(oc4ccc(I)cc4c3=O)C(=O)N2CCCN(C)C)cc1OC]

The compound was prepared as described above. ¹H NMR (400 MHz, CD₂Cl₂) δ 8.48 (d, J=2.2 Hz, 1H), 8.01 (dd, J=8.8, 2.2 Hz, 1H), 7.45 (d, J=8.8 Hz, 1H), 7.08 (d, J=2.1 Hz, 1H), 6.98 (dd, J=8.4, 2.2 Hz, 1H), 6.84 (d, J=8.4 Hz, 1H), 3.83 (s, 3H), 3.82 (s, 3H), 3.68 (ddd, J=14.6, 5.3, 3.8 Hz, 1H), 2.87 (ddd, J=14.2, 10.5, 3.2 Hz, 1H), 2.60-2.47 (m, 2H), 2.39-2.26 (m, 1H), 2.20 (s, 6H), 1.75-1.65 (m, 1H). 2:1 ratio. Major to minor. Only major NMR shown; LCMS RT (Method 1)=3.975 min, m/z 565.1 [M+H⁺]; HRMS (ESI) in m/z calcd for C₂₄H₂₆IN₂O₆⁺ [M+H⁺] 565.0836, found 565.0718.

### Example 17: Preparation of 7-chloro-2-(3-(dimethylamino)propyl)-1-(4-hydroxy-3-methoxyphenyl)-1,2-dihydrochromeno[2,3-c]pyrrole-3,9-dione

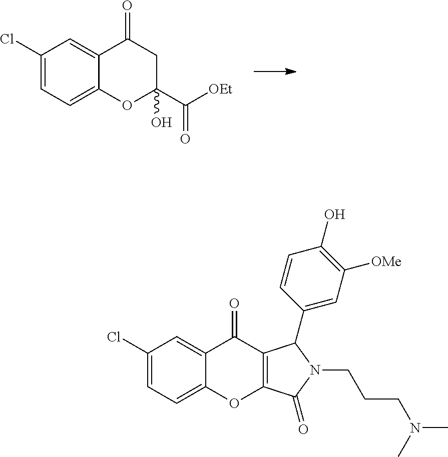 [SMILES: CCOC(=O)C1(O)CC(=O)c2cc(Cl)ccc2O1 ; COc1cc(C2c3c(oc4ccc(Cl)cc4c3=O)C(=O)N2CCCN(C)C)ccc1O]

A solution of 4-hydroxy-3-methoxybenzaldehyde (28.2 mg, 0.185 mmol) and N1,N1-dimethylpropane-1,3-diamine (23.3 mL, 0.185 mmol) in MeOH (5 mL) was heated to 70° C. for 30 minutes, cooled, then ethyl 6-chloro-2-hydroxy-4-oxochromane-2-carboxylate (50 mg, 0.185 mmol) was added. The reaction mixture was heated to 50° C. and stirred for 2 hrs. Reaction mixture was filtered and the resulting solid was collected and taken to the next step. The crude product was dissolved in acetic acid (5 mL) and heated to 50° C. for 3 hrs. The reaction mixture was rotovaped, triturated in EtOH, and then the desired product was filtered to yield a white solid (35 mg, 43%). ¹H NMR (400 MHz, CDCl₃) δ 8.13 (d, J=2.3 Hz, 1H), 7.72-7.62 (m, 2H), 6.91 (d, J=8.1 Hz, 1H), 6.81 (dd, J=8.1, 2.0 Hz, 1H), 6.67 (s, 1H), 5.66 (s, 1H), 5.48 (s, 1H), 3.85 (s, 3H), 3.00 (dt, J=14.0, 6.9 Hz, 1H), 2.30-2.21 (m, 21H), 2.16 (s, 61H), 1.85-1.63 (m, 211), 1.26-1.23 (m, 1H); LCMS RT (Method 2)=2.019 min, m/z 443.1 [M+H⁺].

### Example 18: Preparation of 6-chloro-N-(3-(dimethylamino)propyl)-3-(4-hydroxy-3-methoxybenzoyl)-4-oxo-4H-chromene-2-carboxamide NCGC00842251-0

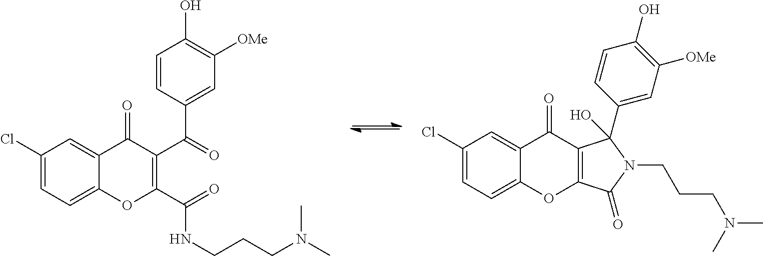 [SMILES: COc1cc(C(=O)c2c(C(=O)NCCCN(C)C)oc3ccc(Cl)cc3c2=O)ccc1O ; COc1cc(C2(O)c3c(oc4ccc(Cl)cc4c3=O)C(=O)N2CCCN(C)C)ccc1O]

¹H NMR (400 MHz, CD₂Cl₂) δ 8.43 (s, 1H), 8.12 (d, J=2.5 Hz, 1H), 7.73-7.63 (m, 2H), 7.28-7.16 (m, 2H), 6.84 (s, 1H), 3.92 (s, 3H), 3.68 (ddd, J=14.6, 5.8, 3.8 Hz, 1H), 2.89 (ddd, J=14.1, 10.1, 3.2 Hz, 1H), 2.61-2.55 (m, 2H), 2.35-2.22 (m, 1H), 2.25 (s, 6H), 1.78-1.72 (m, 1H). 3:2 ratio. Major to minor. Only major NMR shown; LCMS RT (Method 1)=3.639 min, nm/z 459.1 [M+H⁺]; HRMS (ESI) m/z calcd for C₂₃H₂₄ClN₂O₆⁺[M+H⁺] 459.1323, found 459.1105.

### Example 19: Preparation of 8-chloro-1-(3,4-dimethoxyphenyl)-2-(3-(dimethylamino)propyl)-1,2-dihydrochromeno[2,3-c]pyrrole-3,9-dione

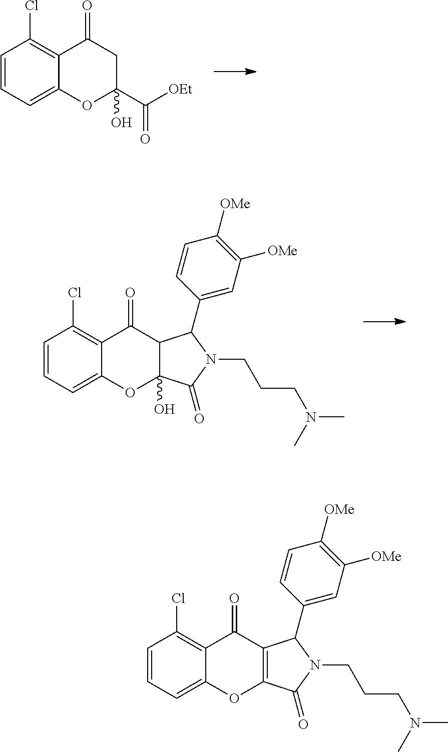 [SMILES: CCOC(=O)C1(O)CC(=O)c2c(Cl)cccc2O1 ; COc1ccc(C2C3C(=O)c4c(Cl)cccc4OC3(O)C(=O)N2CCCN(C)C)cc1OC ; COc1ccc(C2c3c(oc4cccc(Cl)c4c3=O)C(=O)N2CCCN(C)C)cc1OC]

1-(2-Chloro-6-hydroxyphenyl) ethan-1-one (300 mg, 1.759 mmol) was dissolved in 20 mL of THF. The reaction was cooled in an ice-bath and potassium t-butoxide (3.9 mL, 3.9 mmol) was added. After 5 minutes with stirring, to the reaction mixture was added diethyl oxalate (0.240 mL, 1.767 mmol). The reaction mixture was heated to 50° C. and kept for 1 hour. Then the reaction was cooled down to room temperature, followed by quenching it with 0.1N HCl solution. The ethyl acetate was added to the reaction solution. The organic phase was separated and dried with anhydrous Na₂SO₄. The organic phase was evaporation to obtain the crude product. The product was purified by silica column (20-80% ethyl acetate in hexane). Yield=92%. ¹H NMR (400 MHz, CDCl₃) δ 7.42-7.30 (m, 1H), 7.10 (dt, J=7.9, 1.0 Hz, 1H), 6.92 (dq, J=8.4, 1.1 Hz, 1H), 4.58-4.48 (m, 1H), 4.42-4.30 (i, 2H), 3.40 (ddd, J=16.0, 2.6, 1.5 Hz, 1H), 2.96 (dt, J=16.0, 0.9 Hz, 1H), 1.35 (ddd, J 8.1, 6.7, 1.0 Hz, 3H).

### Example 20: Preparation of 8-chloro-1-(3,4-dimethoxyphenyl)-2-(3-(dimethylamino)propyl)-1,2-dihydrochromeno[2,3-c]pyrrole-3,9-dione NCGC00842996-01

3,4-Dimethoxybenzaldehyde (123 mg, 0.739 mmol) was placed in the microwave reaction vessel and dissolved in MeOH (5 ml). To the solution was added N¹,N¹-dimethylpropane-1,3-diamine (93 μl, 0.739 mmol). The microwave-assisted reaction was run at 110° C. for 30 min. After cooling the reaction vessel to room temperature, ethyl 5-chloro-2-hydroxy-4-oxochromane-2-carboxylate (200 mg, 0.739 mmol) was added to the reaction vessel and the microwave-assisted reaction was conducted at 70° C. for 30 min. The yellow solid was precipitated, filtered, and washed with MeOH three times. The product was used for the next step without further purification.

8-Chloro-1-(3,4-dimethoxyphenyl)-2-(3-(dimethylamino)propyl)-3a-hydroxy-1,2,3a,9a-tetrahydrochromeno[2,3-c]pyrrole-3,9-dione (328.9 mg, 0.693 mmol) was dissolved in AcOH (10 ml). The solution was heated up to 70° C. and stirred for 1 hour. Yield 91%. ¹H NMR (400 MHz, CDCl₃) δ 7.64-7.54 (m, 2H), 7.43 (dd, J=6.3, 2.8 Hz, 1H), 6.92-6.81 (m, 2H), 6.66 (d, J=2.0 Hz, 1H), 5.53 (s, 1H), 3.84 (d, J=11.7 Hz, 6H), 3.82-3.77 (m, 1H), 3.00 (dt, J=14.3, 5.9 Hz, 1H), 2.84 (td, J=11.7, 5.2 Hz, 1H), 2.68 (ddd, J=12.0, 10.5, 5.4 Hz, 1H), 2.53 (s, 6H), 1.98-1.86 (m, 2H). LCMS RT (Method 1)=2.598 min, m/z=457.1 [M+H⁺].

### Example 21: Preparation of 7-chloro-2-(3-(dimethylamino)propyl)-1-(3-hydroxy-4-methoxyphenyl)-1,2-dihydrochromeno[2,3-c]pyrrole-3,9-dione

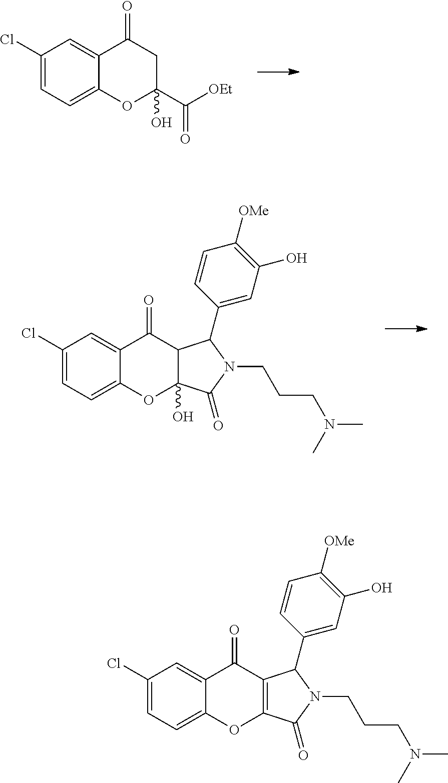 [SMILES: CCOC(=O)C1(O)CC(=O)c2cc(Cl)ccc2O1 ; COc1ccc(C2C3C(=O)c4cc(Cl)ccc4OC3(O)C(=O)N2CCCN(C)C)cc1O ; COc1ccc(C2c3c(oc4ccc(Cl)cc4c3=O)C(=O)N2CCCN(C)C)cc1O]

3-Hydroxy-4-methoxybenzaldehyde (225 mg, 1.478 mmol) and N1,N1-dimethylpropane-1,3-diamine (186 μl, 1.478 mmol) was dissolved in MeOH (5 ml). The reaction mixture was microwave-assisted heated up to 110° C. for 30 min. The ethyl 6-chloro-2-hydroxy-4-oxochromane-2-carboxylate (400 mg, 1.478 mmol) was added to the imine solution of MeOH. The mixture was heated to 70° C. for one hour. Yellow solid was precipitated. The solid was filtered and washed with methanol. The product was used for the next step without further purification. 7-Chloro-2-(3-(dimethylamino)propyl)-3a-hydroxy-1-(3-hydroxy-4-methoxyphenyl)-1,2,3a,9a-tetrahydrochromeno[2,3-c]pyrrole-3,9-dione (550 mg, 1.193 mmol) was placed in the microwave reaction vessel and dissolved in acetic acid (10 ml). The microwave-assisted reaction was run at 70° C. for one hour. The acetic acid in the vessel was dried and dissolved in ethanol. The ethanol was evaporated and dried under vacuum. Overall yield=80%. ¹H NMR (400 MHz, CDCl₃) δ 8.16-8.06 (m, 1H), 7.67 (dd, J=3.9, 1.4 Hz, 2H), 6.85 (d, J=3.4 Hz, 2H), 6.69 (d, J=1.6 Hz, 1H), 5.45 (s, 1H), 3.95-3.72 (m, 4H), 2.99 (ddd, J=14.0, 7.9, 6.3 Hz, 1H), 2.24 (q, J=7.0 Hz, 2H), 2.16 (s, 6H), 1.82-1.65 (m, 2H). LCMS RT (Method 1)=2.715 min, m/z=443.1 [M+H⁺].

### Example 22: Preparation of 5-(7-chloro-2-(3-(dimethylamino)propyl)-3,9-dioxo-1,2,3,9-tetrahydrochromeno[2,3-c]pyrrol-1-yl)-2-methoxybenzonitrile

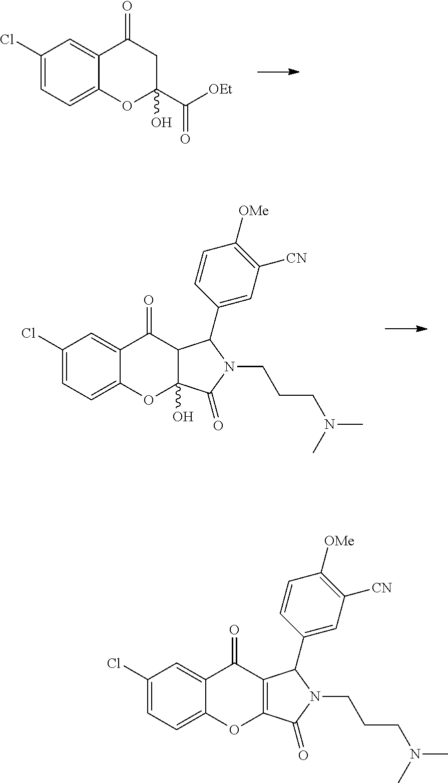 [SMILES: CCOC(=O)C1(O)CC(=O)c2cc(Cl)ccc2O1 ; COc1ccc(C2C3C(=O)c4cc(Cl)ccc4OC3(O)C(=O)N2CCCN(C)C)cc1C#N ; COc1ccc(C2c3c(oc4ccc(Cl)cc4c3=O)C(=O)N2CCCN(C)C)cc1C#N]

5-Formyl-2-methoxybenzonitrile (125 mg, 0.739 mmol) was placed in the microwave reaction vessel and dissolved in MeOH (5 ml). To the solution was added N¹,N¹-dimethylpropane-1,3-diamine (93 μl, 0.739 mmol). The microwave-assisted reaction was run at 110° C. for 30 min. After cooling the reaction vessel to room temperature, ethyl 6-chloro-2-hydroxy-4-oxochromane-2-carboxylate (200 mg, 0.739 mmol) was added to the reaction vessel and the microwave-assisted reaction was run at 70° C. for 30 min. A yellow solid precipitated and removed by filtration. The solid was washed with MeOH three times. The product was used for the next step without further purification.

The preparation of 5-(7-chloro-2-(3-(dimethylamino)propyl)-3,9-dioxo-1,2,3,9-tetrahydrochromeno[2,3-c]pyrrol-1-yl)-2-methoxybenzonitrile was conducted according to the chemical reaction scheme as shown above. ¹H NMR (400 MHz, CDCl₃) δ 8.12 (d, J=2.4 Hz, 1H), 7.71 (dd, J=9.0, 2.5 Hz, 1H), 7.66 (d, J=9.0 Hz, 1H), 7.55 (dd, J=8.8, 2.4 Hz, 1H), 7.43 (d, J=2.4 Hz, 1H), 7.00 (d, J=8.8 Hz, 1H), 5.64 (s, 1H), 3.93 (s, 3H), 3.92-3.84 (m, 1H), 2.96 (ddd, J=14.3, 6.7, 5.1 Hz, 1H), 2.83-2.74 (m, 1H), 2.68-2.59 (m, 1H), 2.49 (s, 6H), 2.00-1.89 (m, 2H). LCMS RT (Method 1) 2.889 min, m/z=468.1 [M+H⁺].

### Example 23: Preparation of 6-Chloro-N-(3-(dimethylamino)propyl)-4-oxo-3-(3,4,5-trimethoxybenzoyl)-4H-chromene-2-carboxamide NCGC0589432-01

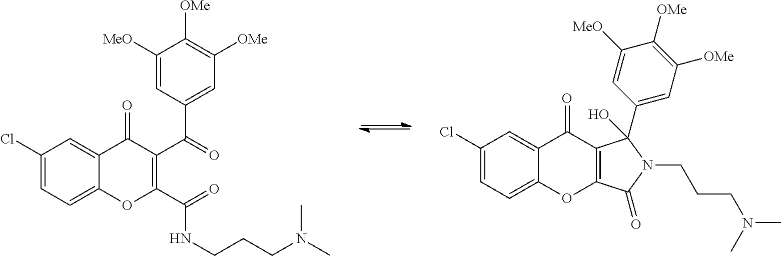 [SMILES: COc1cc(C(=O)c2c(C(=O)NCCCN(C)C)oc3ccc(Cl)cc3c2=O)cc(OC)c1OC ; COc1cc(C2(O)c3c(oc4ccc(Cl)cc4c3=O)C(=O)N2CCCN(C)C)cc(OC)c1OC]

The preparation of 6-chloro-N-(3-(dimethylamino)propyl)-4-oxo-3-(3,4,5-trimethoxybenzoyl)-4H-chromene-2-carboxamide was conducted according to the chemical reaction scheme as shown above. [00174] 4H NMR (400 MHz, CD₂Cl₂) δ 8.14-8.13 (m, 1H), 7.73-7.65 (m, 2H), 6.72 (s, 2H), 3.82 (s, 6H), 3.77 (s, 3H), 3.70 (dt, J=14.6, 4.6 Hz, 1H), 2.90 (td, J=11.2, 5.5 Hz, 1H), 2.57-2.60 (m, 2H), 2.37-2.25 (m, 1H), 2.25 (s, 6H), 1.80-1.72 (m, 1H). 4:1 ratio. Major to minor. Only major NMR shown; LCMS RT (Method 1)=3.991 min, m/z 503.2 [M+H⁺]; HRMS (ESI) m/z calcd for C₂₅H₂₈ClN₂O₇⁺[M+H⁺]503.1585, found 503.1568.

### Example 24: Preparation of 6-chloro-3-(3,4-dimethoxybenzoyl)-N-(3-morpholinopropyl)-4-oxo-4H-chromene-2-carboxamide NCGC0589776-01

The preparation of 6-chloro-3-(3,4-dimethoxybenzoyl)-N-(3-morpholinopropyl)-4-oxo-4H-chromene-2-carboxamide was conducted according to the chemical reaction scheme as shown above.

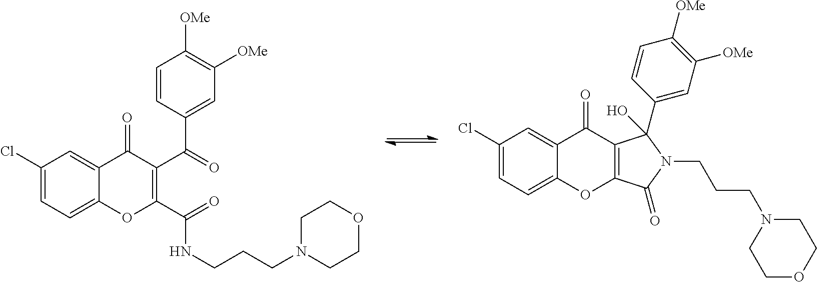 [SMILES: COc1ccc(C(=O)c2c(C(=O)NCCCN3CCOCC3)oc3ccc(Cl)cc3c2=O)cc1OC ; COc1ccc(C2(O)c3c(oc4ccc(Cl)cc4c3=O)C(=O)N2CCCN2CCOCC2)cc1OC]

¹H NMR (400 MHz, CD₂Cl₂) δ 8.13 (d, J=2.5 Hz, 1H), 7.74-7.62 (m, 2H), 7.07-7.02 (m, 2H), 6.86 (d, J=7.9 Hz, 1H), 3.83-3.82 (m, 6H), 3.70-3.58 (m, 5H), 2.90 (ddd, J=14.5, 10.8, 3.4 Hz, 1H), 2.70-2.33 (m, 7H), 1.70-1.78 (m, 1H). 2:1 ratio. Major to minor. Only major NMR shown; LCMS RT (Method 1)=3.902 min, m/z 515.2 [M+H⁺]; HRMS (ESI) m/z calcd for C₂₆H₂₈ClN₂O₇⁺ [M+H⁺] 515.1585, found 515.1451.

### Example 25: Preparation of 6-Chloro-N-(3-(dimethylamino)propyl)-3-(3-methoxybenzoyl)-4-oxo-4H-chromene-2-carboxamide

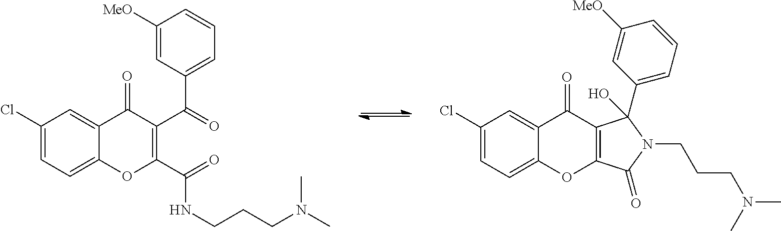 [SMILES: COc1cccc(C(=O)c2c(C(=O)NCCCN(C)C)oc3ccc(Cl)cc3c2=O)c1 ; COc1cccc(C2(O)c3c(oc4ccc(Cl)cc4c3=O)C(=O)N2CCCN(C)C)c1]

The preparation of 6-chloro-N-(3-(dimethylamino)propyl)-3-(3-methoxybenzoyl)-4-oxo-4H-chromene-2-carboxamide was conducted according to the chemical reaction scheme as shown above. ¹H NMR (400 MHz, CD₂Cl₂) δ 8.12 (d, J=2.4 Hz, 1H), 7.71-7.65 (m, 2H), 7.27 (t, J=8.0 Hz, 1H), 7.15 (d, J=2.6 Hz, 1H), 6.99 (d, J=7.7 Hz, 1H), 6.87 (dd, J=8.2, 2.6 Hz, 1H), 3.81 (s, 3H), 3.68 (dt, J=14.7, 4.7 Hz, 1H), 2.87 (ddd, J=14.1, 10.0, 3.2 Hz, 1H), 2.61 (m, 2H), 2.35-2.25 (3, 7H), 1.70-1.78 (m, 1H). 10:1 ratio. Major to minor. Only major NMR shown; LCMS RT (Method 1)=4.031 min, m/z 443.1 [M+H⁺]; HRMS (ESI) m/z calcd for C₂₃H₂₄ClN₂O₅⁺[M+H⁺] 443.1374, found 443.1203.

### Example 26: Preparation of 6-Chloro-3-(4-chlorobenzoyl)-N-(3-(dimethylamino)propyl)-4-oxo-4H-chromene-2-carboxamide. NCGC00589434

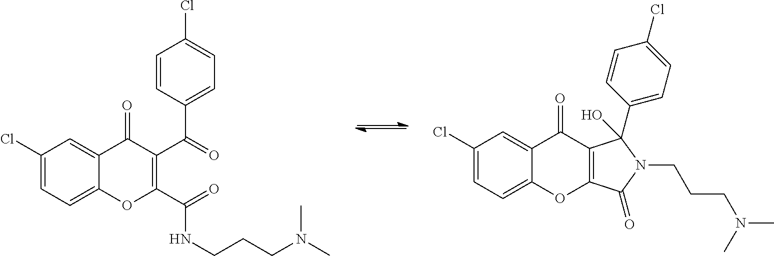 [SMILES: CN(C)CCCNC(=O)c1oc2ccc(Cl)cc2c(=O)c1C(=O)c1ccc(Cl)cc1 ; CN(C)CCCN1C(=O)c2oc3ccc(Cl)cc3c(=O)c2C1(O)c1ccc(Cl)cc1]

The preparation of 6-chloro-3-(4-chlorobenzoyl)-N-(3-(dimethylamino)propyl)-4-oxo-4H-chromene-2-carboxamide was conducted according to the chemical reaction scheme as shown above. ¹H NMR (400 MHz, CD₂Cl₂) δ 8.11 (m, 1H), 7.72-7.64 (m, 2H), 7.49-7.44 (m, 2H), 7.39-7.32 (m, 2H), 3.70 (dt, J=14.7, 4.7 Hz, 1H), 2.85 (ddd, J=14.1, 10.0, 3.1 Hz, 1H), 2.65-2.55 (m, 2H), 2.35-2.22 (m, 7H), 1.72-1.78 (m, 1H). 10:1 ratio. Major to minor. Only major NMR shown; LCMS RT (Method 1)=4.347 min, m/z 447.1 [M+H⁺]; HRMS (ESI) m/z calcd for C₂₂H₂₁Cl₂N₂O₄⁺ [M+H⁺] 447.0878, found 447.0700.

### Example 27: Preparation of 6-Chloro-N-(3-(dimethylamino)propyl)-3-(4-isopropylbenzoyl)-4-oxo-4H-chromene-2-carboxamide NCGC00589437-01

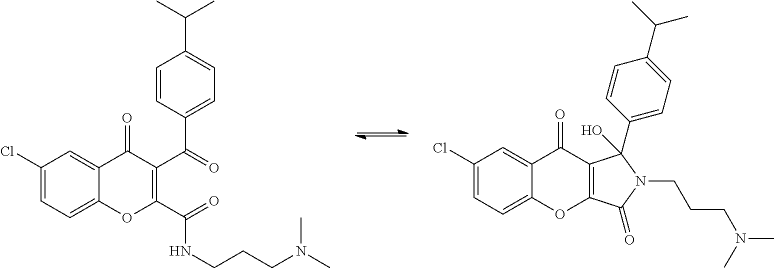 [SMILES: CC(C)c1ccc(C(=O)c2c(C(=O)NCCCN(C)C)oc3ccc(Cl)cc3c2=O)cc1 ; CC(C)c1ccc(C2(O)c3c(oc4ccc(Cl)cc4c3=O)C(=O)N2CCCN(C)C)cc1]

The preparation of 6-chloro-N-(3-(dimethylamino)propyl)-3-(4-isopropylbenzoyl)-4-oxo-4H-chromene-2-carboxamide was conducted according to the chemical reaction scheme as shown above. ¹H NMR (400 MHz, CD₂Cl₂) δ 8.11 (d, J=2.4 Hz, 1H), 7.80-7.62 (m, 21H), 7.41 (d, J=7.9 Hz, 211), 7.23 (d, J=8.0 Hz, 2H), 3.67 (dt, J=14.5, 4.5 Hz, 1H), 2.99-2.79 (m, 2H), 2.60-2.45 (m, 2H), 2.36-2.22 (m, 1H), 2.22 (s, 6H), 1.78-1.65 (0, 1H), 1.24 (d, J=6.9 Hz, 6H); LCMS RT (Method 1)=4.688 min, m/z 455.2 [M+H⁺]; HRMS (ESI) m/z calcd for C₂₅H₂₈ClN₂O₄⁺[M+H⁺] 455.1738, found 455.1575.

### Example 28: Preparation of 6-chloro-N-(3-(dimethylamino)propyl)-3-(2-fluorobenzoyl)-4-oxo-4H-chromene-2-carboxamide NCGC00589468-01

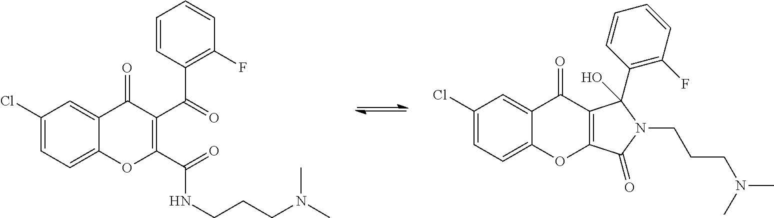 [SMILES: CN(C)CCCNC(=O)c1oc2ccc(Cl)cc2c(=O)c1C(=O)c1ccccc1F ; CN(C)CCCN1C(=O)c2oc3ccc(Cl)cc3c(=O)c2C1(O)c1ccccc1F]

The preparation of 6-chloro-N-(3-(dimethylamino)propyl)-3-(2-fluorobenzoyl)-4-oxo-4H-chromene-2-carboxamide was conducted according to the chemical reaction scheme as shown above. ¹H NMR (400 MHz, CD₂Cl₂) δ 8.19 (td, J=8.1, 1.9 Hz, 1H), 8.10 (d, J=2.4 Hz, 1H), 7.74-7.63 (m, 2H), 7.34 (dtd, J=16.5, 7.5, 1.7 Hz, 2H), 6.97-6.88 (m, 1H), 3.74-3.62 (m, 1H), 2.80 (ddd, J=14.2, 10.3, 3.1 Hz, 1H), 2.63-2.54 (m, 2H), 2.35-2.22 (m, 7H), 1.78-1.65 (m, 1H); LCMS RT (Method 1)=4.108 min, n z 431.1 [M+H⁺]; HRMS (ESI) m/z calcd for C₂₂H₂₁ClFN₂O₄⁺ [M+H⁺] 431.1174, found 431.0986.

### Example 29: Preparation of 6-Chloro-N-(3-(dimethylamino)propyl)-3-(3,4-dimethoxybenzoyl)-4-oxo-4H-chromene-2-carboxamide NCGC00589473-01

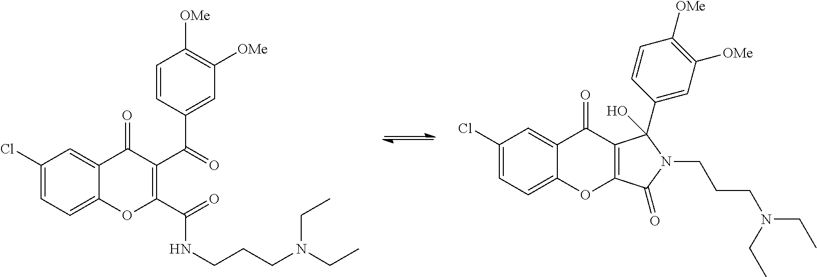 [SMILES: CCN(CC)CCCNC(=O)c1oc2ccc(Cl)cc2c(=O)c1C(=O)c1ccc(OC)c(OC)c1 ; CCN(CC)CCCN1C(=O)c2oc3ccc(Cl)cc3c(=O)c2C1(O)c1ccc(OC)c(OC)c1]

The preparation of 6-chloro-N-(3-(diethylamino)propyl)-3-(3,4-dimethoxybenzoyl)-4-oxo-4H-chromene-2-carboxamide was conducted according to the chemical reaction scheme as shown above. ¹H NMR (400 MHz, CD₂Cl₂) δ 8.12 (d, J=2.5 Hz, 1H), 7.74-7.62 (m, 2H), 7.09 (s, 1H), 6.98 (d, J=8.9 Hz, 1H), 6.84 (d, J=8.4 Hz, 1H), 3.82 (d, J=2.9 Hz, 6H), 3.77-3.65 (m, 1H), 2.89-2.83 (m, 1H), 2.63-2.55 (m, 6H), 2.25 (bs, 1H), 1.81 (bs, 1H), 1.00-0.99 (m, 6H). 1:1 ratio of 2 conformational isomers; LCMS RT (Method 1)=4.092 min, m/z 501.2 [M+H⁺]; HRMS (ESI) m/z calcd for C₂₆H₃₀ClN₂O₆⁺ [M+H⁺] 501.1792, found 501.1613.

### Example 30: Preparation of 6-Cloro-3-(4-chlorobenzoyl)-N-(3-(dimethylamino)propyl)-4-oxo-4H-chromene-2-carboxamide. NCGC00589434-01

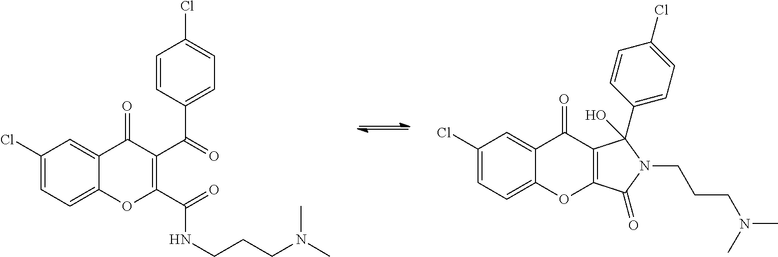 [SMILES: CN(C)CCCNC(=O)c1oc2ccc(Cl)cc2c(=O)c1C(=O)c1ccc(Cl)cc1 ; CN(C)CCCN1C(=O)c2oc3ccc(Cl)cc3c(=O)c2C1(O)c1ccc(Cl)cc1]

The preparation of 6-chloro-3-(4-chlorobenzoyl)-N-(3-(dimethylamino)propyl)-4-oxo-4H-chromene-2-carboxamide was conducted according to the chemical reaction scheme shown above. ¹H NMR (400 MHz, CD₂Cl₂) δ 8.11 (m, 1H), 7.72-7.64 (m, 2H), 7.49-7.44 (m, 2H), 7.39-7.32 (m, 2H), 3.70 (dt, J=14.7, 4.7 Hz, 1H), 2.85 (ddd, J=14.1, 10.0, 3.1 Hz, 1H), 2.65-2.55 (m, 2H), 2.35-2.22 (m, 7H), 1.72-1.78 (m, 1H). 10:1 ratio. Major to minor. Only major NMR shown; LCMS RT (Method 1)=4.347 min, n z 447.1 [M+H⁺]; HRMS (ESI) m/z calcd for C₂₂H₂₁Cl₂N₂O₄⁺ [M+H⁺] 447.0878, found 447.0700.

### Example 31: Preparation of 6-Chloro-N-(3-(dimethylamino)propyl)-3-(4-fluorobenzoyl)-4-oxo-4H-chromene-2-carboxamide NCGC00589431-01

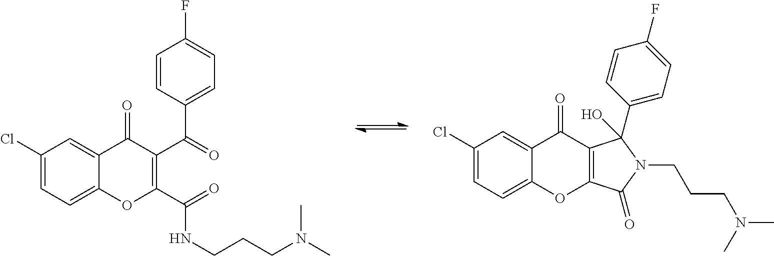 [SMILES: CN(C)CCCNC(=O)c1oc2ccc(Cl)cc2c(=O)c1C(=O)c1ccc(F)cc1 ; CN(C)CCCN1C(=O)c2oc3ccc(Cl)cc3c(=O)c2C1(O)c1ccc(F)cc1]

The preparation of 6-chloro-N-(3-(dimethylamino)propyl)-3-(4-fluorobenzoyl)-4-oxo-4H-chromene-2-carboxamide. was conducted according to the chemical reaction scheme as shown above. ¹H NMR (400 MHz, CD₂Cl₂) δ 8.11 (d, J=2.4 Hz, 1H), 7.73-7.63 (m, 2H), 7.51 (dd, J=8.6, 5.3 Hz, 2H), 7.06 (t, J=8.6 Hz, 2H), 3.70 (dt, J=14.6, 4.6 Hz, 1H), 2.85 (ddd, J=14.2, 10.2, 3.2 Hz, 1H), 2.67-2.46 (m, 2H), 2.38-2.29 (m, 1H), 2.24 (s, 6H), 1.78-1.70 (m, 1H). 8:1 ratio. Major to minor. Only major NMR shown; LCMS RT (Method 1)=4.133 min, m/z 431.1 [M+H⁺]; HRMS (ESI) in z calcd for C₂₂H₂₁ClFN₂O₄⁺[M+H⁺] 431.1174, found 431.1045.

### Example 32: Preparation of 3-(4-(2-Amino-2-oxoethoxy)benzoyl)-6-chloro-N-(3-(dimethylamino)propyl)-4-oxo-4H-chromene-2-carboxamide NCGC00589438-01

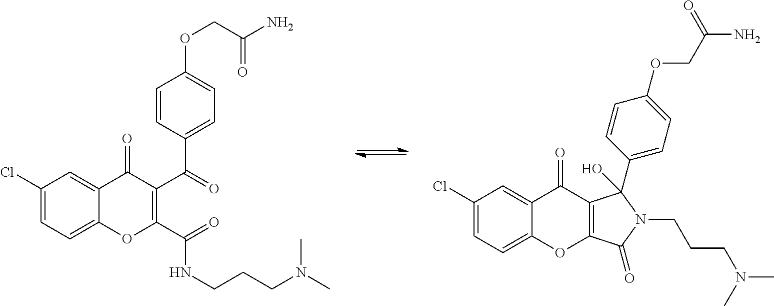 [SMILES: CN(C)CCCNC(=O)c1oc2ccc(Cl)cc2c(=O)c1C(=O)c1ccc(OCC(N)=O)cc1 ; CN(C)CCCN1C(=O)c2oc3ccc(Cl)cc3c(=O)c2C1(O)c1ccc(OCC(N)=O)cc1]

The preparation of 3-(4-(2-amino-2-oxoethoxy)benzoyl)-6-chloro-N-(3-(dimethylamino)propyl)-4-oxo-4H-chromene-2-carboxamide was conducted according to the chemical reaction scheme as shown above. ¹H NMR (400 MHz, CD₂Cl₂) δ 8.11 (d, J=2.4 Hz, 1H), 7.73-7.62 (m, 2H), 7.47 (d, J=8.4 Hz, 2H), 6.94 (d, J=8.5 Hz, 2H), 6.49 (s, 1H), 5.58 (s, 1H), 4.48 (s, 2H), 3.68 (dt, J=14.6, 4.7 Hz, 1H), 2.95-2.73 (m, 1H), 2.62-2.55 (m, 2H), 2.39-2.27 (m, 1H), 2.24 (s, 6H), 1.75-1.73 (n, 1H). 3:1 ratio. Major to minor. Only major NMR shown. LCMS RT (Method 1)=3.463 min, m/z 486.2 [M+H⁺]; HRMS (ESI) m/z calcd for C₂₄H₂₅ClN₃O₆⁺ [M+H⁺] 486.1432, found 486.1247.

### Example 33: Preparation of 3-Benzoyl-6-chloro-N-(3-(dimethylamino)propyl)-4-oxo-4H-chromene-2-carboxamide NCGC00589436-01

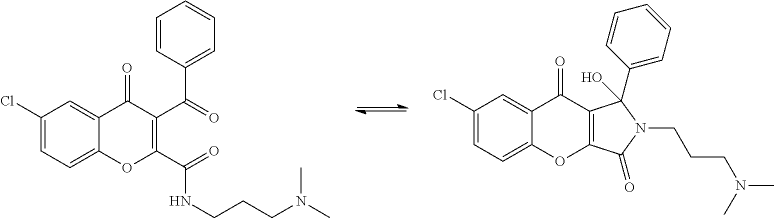 [SMILES: CN(C)CCCNC(=O)c1oc2ccc(Cl)cc2c(=O)c1C(=O)c1ccccc1 ; CN(C)CCCN1C(=O)c2oc3ccc(Cl)cc3c(=O)c2C1(O)c1ccccc1]

The preparation of 3-benzoyl-6-chloro-N-(3-(dimethylamino)propyl)-4-oxo-4H-chromene-2-carboxamide was conducted according to the chemical reaction scheme as shown above. ¹H NMR (400 MHz, CD₂Cl₂) δ 8.11 (d, J=2.4 Hz, 1H), 7.73-7.64 (m, 2H), 7.54-7.48 (m, 2H), 7.42-7.31 (m, 3H), 3.68 (dt, J=14.5, 4.6 Hz, 1H), 2.84 (ddd, J=14.2, 10.4, 3.1 Hz, 1H), 2.60-2.43 (m, 2H), 2.38-2.25 (m, 1H), 2.22 (s, 6H), 1.75-1.62 (m, 1H). 10:1 ratio. Major to minor. Only major NMR shown. LCMS RT (Method 1)=3.976 min, m/z 413.1 [M+H⁺]; HRMS (ESI) m/z calcd for C₂₂H₂₂ClN₂O₄⁺ [M+H⁺] 413.1268, found 413.1115.

### Example 34: Preparation of 6-chloro-3-(3,4-dichlorobenzoyl)-N-(3-(dimethylamino)propyl)-4-oxo-4H-chromene-2-carboxamide NCGC00589472-01

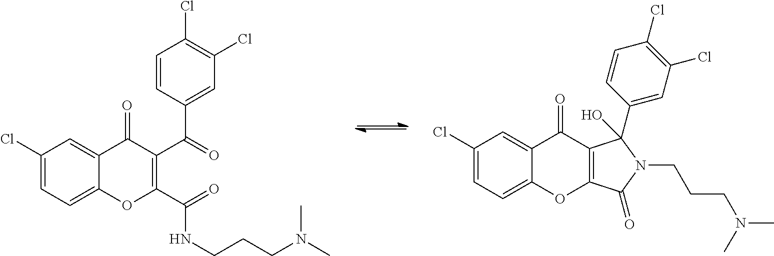 [SMILES: CN(C)CCCNC(=O)c1oc2ccc(Cl)cc2c(=O)c1C(=O)c1ccc(Cl)c(Cl)c1 ; CN(C)CCCN1C(=O)c2oc3ccc(Cl)cc3c(=O)c2C1(O)c1ccc(Cl)c(Cl)c1]

The preparation of 6-chloro-3-(3,4-dichlorobenzoyl)-N-(3-(dimethylamino)propyl)-4-oxo-4H-chromene-2-carboxamide was conducted according to the chemical reaction scheme as shown above. ¹H NMR (400 MHz, CD₂Cl₂) δ 8.10 (d, J=2.5 Hz, 1H), 7.75-7.62 (m, 31H), 7.45 (d, J=8.5 Hz, 1H), 7.34 (dd, J=8.5, 2.2 Hz, 1H), 3.72 (ddd, J=14.6, 5.7, 3.5 Hz, 1H), 2.84 (ddd, J=14.0, 10.1, 3.0 Hz, 1H), 2.75-2.52 (m, 21H), 2.38-2.25 (m, 1H), 2.27 (s, 6H), 1.82-1.72 (m, 1H). 20:1 ratio. Major to minor. Only major NMR shown; LCMS RT (Method 1)=4.601 min, m/z 481.1 [M+H⁺]; HRMS (ESI) n z calcd for C₂₂H₂₀Cl₃N₂O₄⁺ [M+H⁺] 481.0489, found 481.0309.

### Example 35: Preparation of 6-Chloro-N-(3-(dimethylamino)propyl)-3-(4-ethoxy-3-methoxybenz-yl)-4-oxo-4H-chromene-2-carboxamide NCGC00589435-01

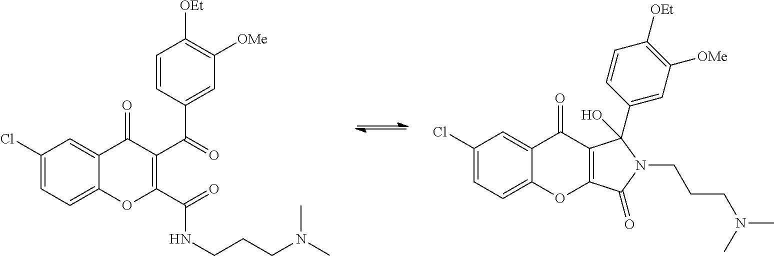 [SMILES: CCOc1ccc(C(=O)c2c(C(=O)NCCCN(C)C)oc3ccc(Cl)cc3c2=O)cc1OC ; CCOc1ccc(C2(O)c3c(oc4ccc(Cl)cc4c3=O)C(=O)N2CCCN(C)C)cc1OC]

The preparation of 6-chloro-N-(3-(dimethylamino)propyl)-3-(4-ethoxy-3-methoxybenzoyl)-4-oxo-4H-chromene-2-carboxamide was conducted according to the chemical reaction scheme as shown above. ¹H NMR (400 MHz, CD₂Cl₂) δ 8.12 (d, J=2.5 Hz, 1H), 7.73-7.63 (m, 2H), 7.08 (s, 1H), 6.96 (dd, J=7.8, 1.9 Hz, 1H), 6.83 (d, J=8.4 Hz, 1H), 4.04 (q, J=7.0 Hz, 2H), 3.84 (s, 3H), 3.68 (dt, J=14.4, 4.5 Hz, 1H), 2.88 (ddd, J=14.2, 10.4, 3.2 Hz, 1H), 2.60-2.48 (m, 2H), 2.37-2.25 (m, 1H), 2.21 (s, 6H), 1.75-1.65 (m, 1H), 1.41 (t, J=7.0 Hz, 3H). 2:1 ratio. Major to minor. Only major NMR shown; LCMS RT (Method 1)=4.159 min, m/z 487.1 [M+H⁺]; HRMS (ESI) m/z calcd for C₂₅H₂₈ClN₂O₆⁺ [M+H⁺] 487.1636, found 487.1545.

### Example 36: Preparation of 6-Chloro-3-(3,4-dimethoxybenzoyl)-N-(3-(dimethylamino)propyl)-N-methyl-4-oxo-4H-chromene-2-carboxamide NCGC00538279-09

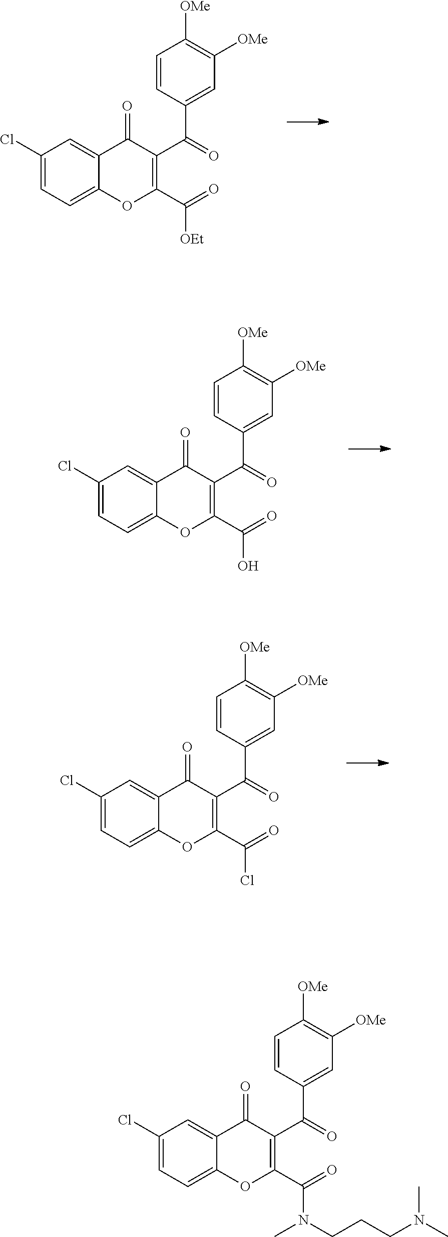 [SMILES: CCOC(=O)c1oc2ccc(Cl)cc2c(=O)c1C(=O)c1ccc(OC)c(OC)c1 ; COc1ccc(C(=O)c2c(C(=O)O)oc3ccc(Cl)cc3c2=O)cc1OC ; COc1ccc(C(=O)c2c(C(=O)Cl)oc3ccc(Cl)cc3c2=O)cc1OC ; COc1ccc(C(=O)c2c(C(=O)N(C)CCCN(C)C)oc3ccc(Cl)cc3c2=O)cc1OC]

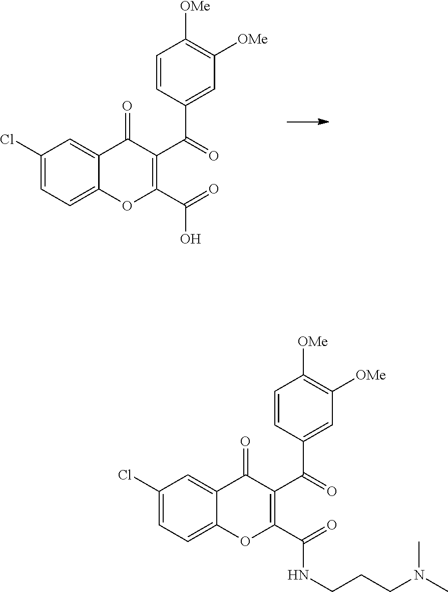 [SMILES: COc1ccc(C(=O)c2c(C(=O)O)oc3ccc(Cl)cc3c2=O)cc1OC ; COc1ccc(C(=O)c2c(C(=O)NCCCN(C)C)oc3ccc(Cl)cc3c2=O)cc1OC]

The preparation of 6-chloro-3-(3,4-dimethoxybenzoyl)-N-(3-(dimethylamino)propyl)-N-methyl-4-oxo-4H-chromene-2-carboxamide was conducted according to the chemical reaction scheme as shown above.

### Example 37: Preparation of 6-Chloro-3-(3,4-dimethoxybenzoyl)-4-oxo-4H-chromene-2-carboxylic acid

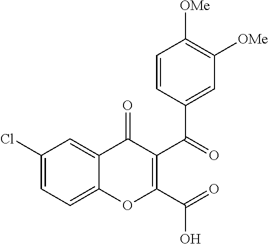 [SMILES: COc1ccc(C(=O)c2c(C(=O)O)oc3ccc(Cl)cc3c2=O)cc1OC]

To a solution of ethyl 6-chloro-3-(3,4-dimethoxybenzoyl)-4-oxo-4H-chromene-2-carboxylate (20 mg, 0.048 mmol) in MeOH (3 mL) was added 1N NaOH (245 mL, 0.245 mmol). The reaction mixture was stirred for 1 hr at room temperature under N₂. The reaction as quenched with 1N HCl (aq) and extracted with EtOAc. The combined organic layers were dried with MgSO₄, filtered and rotovaped. The crude product was purified by column chromatography (hexanes/EtOAc) to yield the desired product as a white solid (7.5 mg, 40%). ¹H NMR (400 MI-z, DMSO-d₆) δ 8.00-7.93 (m, 21H), 7.88 (d, J=9.7 Hz, 1H), 7.52 (dd, J=8.3, 2.0 Hz, 1H), 7.47 (d, J=2.0 Hz, 1H), 6.97 (d, J=8.4 Hz, 1H), 3.83 (s, 3H), 3.81 (s, 3H). LCMS RT (Method 2)=2.780 min, m/z 389.0 [M+H⁺].

### Example 38: Preparation of 6-chloro-3-(3,4-dimethoxybenzoyl)-N-(3-(dimethylamino)propyl)-N-methyl-4-oxo-4H-chromene-2-carboxamide NCGC00841413-02

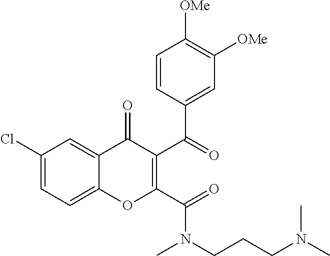 [SMILES: COc1ccc(C(=O)c2c(C(=O)N(C)CCCN(C)C)oc3ccc(Cl)cc3c2=O)cc1OC]

To a solution of 6-chloro-3-(3,4-dimethoxybenzoyl)-4-oxo-4H-chromene-2-carboxylic acid (16 mg, 0.041 mmol) in DCM (3 mL) was added 1 drop of DMF then oxalyl chloride (10.8 mL, 0.123 mmol). Reaction mixture was stirred at room temperature under N₂. After 1 hr, the reaction mixture was rotovaped and dried under high vacuum. To this residue was added DCM (3 mL) then N¹,N¹,N³-trimethylpropane-1,3-diamine (12.2 mL, 0.083 mmol). Reaction was stirred at room temperature overnight under N₂. Reaction was rotovaped and crude product was purified by column chromatography (DCM/MeOH) to yield the desired product as a brown oil (5.2 mg, 21%). ¹H NMR (400 m/z, CD₂Cl₂) δ 8.17-8.16 (m, 1H), 7.78-7.75 (m, 1H), 7.63-7.59 (m, 1H), 7.47-7.40 (m, 2H), 6.92-6.83 (m, 1H), 3.97-3.87 (m, 6H), 3.59-3.51 (m, 2H), 3.13 (s, 3H), 2.99-2.96 (m, 2H), 2.84 (s, 1H, minor), 2.80 (s, 5H, major), 2.11-2.05 (m, 2H); LCMS RT (Method 1)=4.364 min, n z 487.1 [M+H⁺]; FIRMS (ESI) i/z calcd for C₂₅H₂₈ClN₂O₆⁺[M+H⁺] 487.1636, found 487.1571.

### Example 39: Preparation of 6-Chloro-N-(3-(dimethylamino)propyl)-3-(4-methoxybenzoyl)-4-oxo-4H-chromene-2-carboxamide NCGC00842995-01

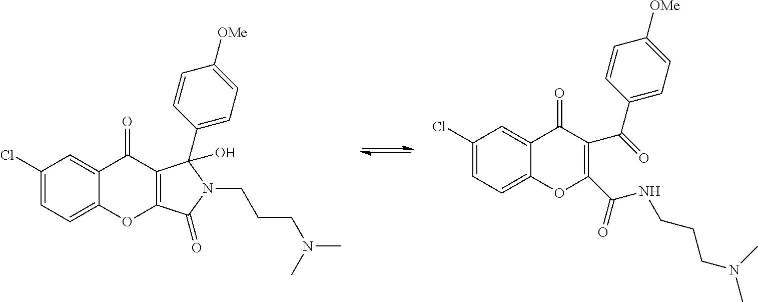 [SMILES: COc1ccc(C2(O)c3c(oc4ccc(Cl)cc4c3=O)C(=O)N2CCCN(C)C)cc1 ; COc1ccc(C(=O)c2c(C(=O)NCCCN(C)C)oc3ccc(Cl)cc3c2=O)cc1]

7-Chloro-2-(3-(dimethylamino) propyl)-1-(4-methoxyphenyl)-1,2-dihydrochromeno[2,3-c] pyrrole-3,9-dione (101.4 ng, 0.238 mmol) was dissolved in DMSO (10 ml) with heating. The clear DMSO solution was stirred with air bubbling at 70° C. for 12 hours. The DMSO was evaporated. The crude product was purified by HPLC (H₂O/MeCN). Yield=20.82%. ¹H NMR (400 MHz, CD₂Cl₂) δ 8.11 (d, J=2.4 Hz, 1H), 7.71-7.62 (m, 21H), 7.44-7.38 (m, 2H), 6.91-6.85 (m, 2H), 3.80 (d, J=0.9 Hz, 3H), 3.67 (dt, J=14.7, 4.7 Hz, 1H), 2.90-2.83 (m, 1H), 2.57 (s, 2H), 2.35-2.29 (m, 1H), 2.25 (s, 6H), 1.76 (s, 1H). 2:1 ratio. Major to minor. Only major NMR shown. LCMS RT (Method 1)=4.055 min, m/z=443.1 [M+H⁺]; HRMS (ESI) m/z calcd for C₂₃H₂₄ClN₂O₅⁺ [M+H⁺] 443.1374, found 443.1242.

### Example 40: Preparation of 6-chloro-3-(2,3-dihydrobenzo[b][1,4]dioxine-6-carbonyl)-N-(3-(dimethylamino)propyl)-4-oxo-4H-chromene-2-carboxamide NCGC00841421-01

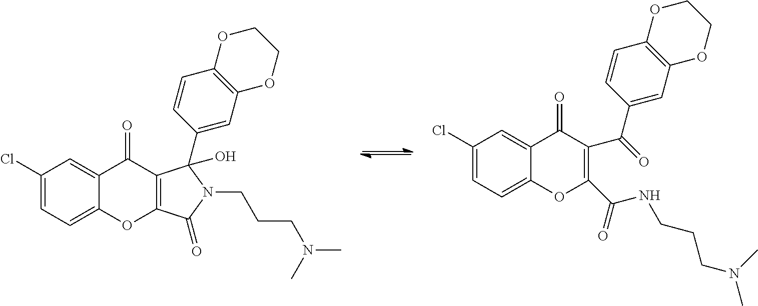 [SMILES: CN(C)CCCN1C(=O)c2oc3ccc(Cl)cc3c(=O)c2C1(O)c1ccc2c(c1)OCCO2 ; CN(C)CCCNC(=O)c1oc2ccc(Cl)cc2c(=O)c1C(=O)c1ccc2c(c1)OCCO2]

7-Chloro-1-(2,3-dihydrobenzo[b][1,4]dioxin-6-yl)-2-(3-(dimethylamino)propyl)-1,2-dihydrochromeno[2,3-c]pyrrole-3,9-dione (180.7 mg, 0.397 mmol) was dissolved in DMSO. The DMSO solution was stirred with air-bubbling for 12 hr. The crude product was purified by HPLC (H₂O/MeCN). Yield 18.33%. ¹H NMR (400 MHz, CD₂Cl₂) δ 8.12 (d, J=2.5 Hz, 1H), 7.72-7.62 (m, 2H), 7.03 (d, J=2.2 Hz, 1H), 6.95-6.89 (m, 1H), 6.82 (d, J=8.5 Hz, 1H), 4.24 (s, 4H), 3.71-3.60 (m, 1H), 2.91-2.84 (m, 1H), 2.60 (m, 2H), 2.29 (s, 1H), 2.25 (s, 6H), 1.76 (d, J=10.0 Hz, 1H). 3:1 ratio. Major to minor. Only major NMR shown. LCMS RT (Method 1)=3.970 min, m/z=471.1 [M+H⁺]; HRMS (ESI) m/z calcd for C₂₄H₂₄ClN₂O₆⁺[M+H⁺] 471.1323, found 471.1169.

### Example 41: Preparation of 6-Chloro-3-(3,5-dimethoxybenzoyl)-N-(3-(dimethylamino)propyl)-4-oxo-4H-chromene-2-carboxamide NCGC00842994-01

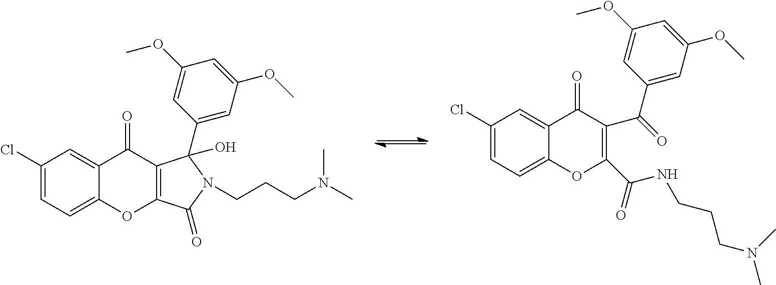 [SMILES: COc1cc(OC)cc(C2(O)c3c(oc4ccc(Cl)cc4c3=O)C(=O)N2CCCN(C)C)c1 ; COc1cc(OC)cc(C(=O)c2c(C(=O)NCCCN(C)C)oc3ccc(Cl)cc3c2=O)c1]

7-Chloro-1-(3,5-dimethoxyphenyl)-2-(3-(dimethylamino) propyl)-1,2-dihydrochromeno[2,3-c] pyrrole-3,9-dione (330 ng, 0.722 mmol) was dissolved in DMSO (10 ml). The solution was stirred overnight with air bubbling. The crude product was purified by HPLC (H₂O/MeCN). Yield=6.70%. ¹H NMR (400 MHz, CD₂Cl₂) δ 8.13 (d, J=2.5 Hz, 1H), 7.74-7.63 (m, 21H), 6.65 (d, J=2.3 Hz, 2H), 6.42 (t, J=2.3 Hz, 1H), 3.77 (s, 6H), 3.68 (ddd, J=14.7, 5.7, 3.7 Hz, 1H), 2.88 (ddd, J=14.0, 10.3, 3.2 Hz, 1H), 2.57 (dd, J=9.0, 3.9 Hz, 2H), 2.38-2.26 (m, 1H), 2.23 (s, 6H), 1.72 (d, J=15.4 Hz, 1H). LCMS RT (Method 1)=4.058 min, m/z=473.14 [M+H⁺]; HRMS (ESI) m/z calcd for C₂₄H₂₆ClN₂O₆⁺ 473.1479, found 473.1407.

### Example 42: Preparation of 6-Chloro-3-(3,4-difluorobenzoyl)-N-(3-(dimethylamino)propyl)-4-oxo-4H-chromene-2-carboxamide NCGC0842509-01

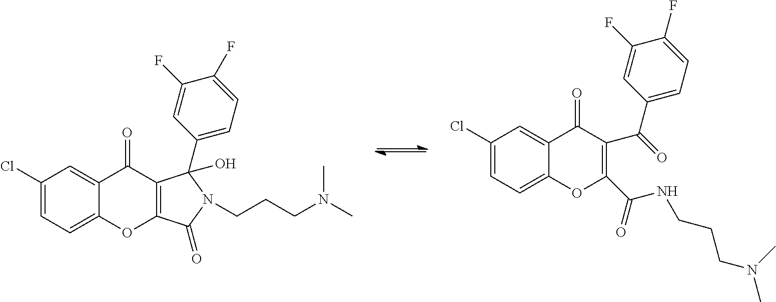 [SMILES: CN(C)CCCN1C(=O)c2oc3ccc(Cl)cc3c(=O)c2C1(O)c1ccc(F)c(F)c1 ; CN(C)CCCNC(=O)c1oc2ccc(Cl)cc2c(=O)c1C(=O)c1ccc(F)c(F)c1]

7-Chloro-1-(3,4-difluorophenyl)-2-(3-(dimethylamino) propyl)-1,2-dihydrochromeno[2,3-c] pyrrole-3,9-dione (190 mg, 0.439 mmol) was dissolved in 10 mL DMSO with heating. The DMSO solution was stirred with air-bubbling until the starting compound was consumed completely. The crude product was purified by HPLC (H₂O/MeCN). Yield=29.9%. ¹H NMR (400 MHz, CD₂Cl₂) δ 8.12 (d, J=2.5 Hz, 1H), 7.74-7.63 (m, 2H), 7.47 (ddd, J=11.6, 7.6, 2.1 Hz, 1H), 7.22-7.10 (m, 2H), 3.71 (ddd, J=14.6, 5.6, 3.6 Hz, 1H), 2.83 (ddd, J=14.2, 10.5, 3.1 Hz, 1H), 2.65-2.46 (m, 2H), 2.35 (dddd, J=15.8, 9.4, 6.7, 3.7 Hz, 1H), 2.22 (s, 6H), 1.77-1.66 (m, 1H). LCMS RT (Method 1)=4.172 min, m/z=449.10 [M+H⁺]; HRMS (ESI) m/z calcd for C₂₂H₂₀ClF₂NO₄⁺[M+H⁺] 449.1080, found 449.0957.

### Example 43: Preparation of 3-(3-Bromo-4-methoxybenzoyl)-6-chloro-N-(3-(dimethylamino)propyl)-4-oxo-4H-chromene-2-carboxamide NCGC00842510-01

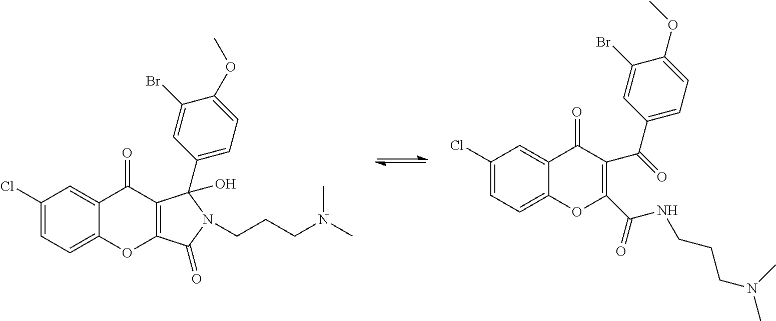 [SMILES: COc1ccc(C2(O)c3c(oc4ccc(Cl)cc4c3=O)C(=O)N2CCCN(C)C)cc1Br ; COc1ccc(C(=O)c2c(C(=O)NCCCN(C)C)oc3ccc(Cl)cc3c2=O)cc1Br]

1-(3-Bromo-4-methoxyphenyl)-7-chloro-2-(3-(dimethylamino)propyl)-1,2-dihydrochromeno[2,3-c] pyrrole-3,9-dione (106.1 mg, 0.210 mmol) was dissolved in DMSO (10 ml) with heating. The solution was stirred at 70° C. for 12 hr with air bubbling. The crude product was purified by HPLC (H₂O/MeCN). Yield=16.90%. ¹H NMR (400 MHz, CD₂Cl₂) δ 8.12 (d, J=2.5 Hz, 1H), 7.73-7.64 (m, 31), 7.46 (dd, J=8.7, 2.4 Hz, 1H), 6.92 (d, J=8.1 Hz, 1H), 3.88 (d, J=0.8 Hz, 3H), 3.70 (dt, J=14.0, 4.5 Hz, 1H), 2.86 (ddd, J=13.7, 10.5, 2.9 Hz, 1H), 2.63-2.55 (m, 2H), 2.38-2.29 (m, 1H), 2.23 (s, 6H), 1.72 (d, J=15.4 Hz, 1H). 5:1 ratio. Major to minor. Only major NMR shown. LCMS RT (Method 1)=4.248 min, m z=521.10 [M+H⁺]; HRMS (ESI) m/z calcd for C₂₃H₂₃BrClN₂O₅⁺ [M+H⁺] 523.0458, found 523.0346.

### Example 44: Preparation of 6-chloro-N-(3-(dimethylamino)propyl)-3-(3-fluoro-4,5-dimethoxybenzoyl)-4-oxo-4H-chromene-2-carboxamide NCGC00842513-01

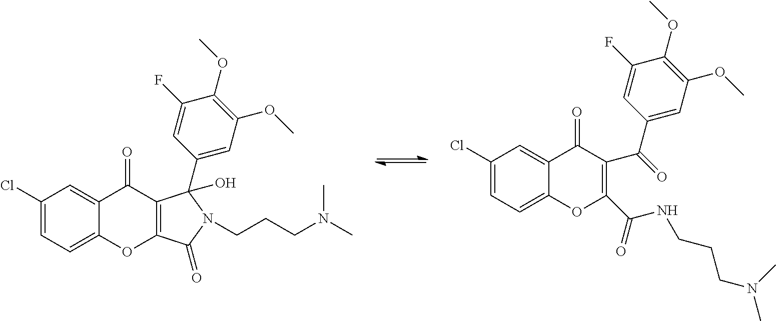 [SMILES: COc1cc(C2(O)c3c(oc4ccc(Cl)cc4c3=O)C(=O)N2CCCN(C)C)cc(F)c1OC ; COc1cc(C(=O)c2c(C(=O)NCCCN(C)C)oc3ccc(Cl)cc3c2=O)cc(F)c1OC]

7-Chloro-2-(3-(dimethylamino)propyl)-1-(3-fluoro-4,5-dimethoxyphenyl)-1,2-dihydrochromeno[2,3-c]pyrrole-3,9-dione (308 mg, 0.649 mmol) was dissolved in DMSO (10 ml). The DMSO solution was stirred overnight at 70° C. with air-bubbling. The crude product was purified by HPLC (H₂O/MeCN). Yield=15.11%. ¹H NMR (400 Hz, CD₂Cl₂) δ 8.13 (dd, J=2.5, 0.5 Hz, 1H), 7.74-7.64 (m, 2H), 6.94 (t, J=1.9 Hz, 1H), 6.82 (dd, J=11.4, 2.1 Hz, 1H), 3.89-3.85 (m, 6H), 3.70 (ddd, J=14.4, 5.4, 3.6 Hz, 1H), 2.86 (ddd, J=14.6, 10.5, 3.2 Hz, 1H), 2.63-2.50 (m, 21H), 2.39-2.27 (m, 1H), 2.22 (s, 61H), 1.77-1.68 (m, 1H). LCMS RT (Method 1)=4.060 min, m/z=491.1 [1M+H⁺]; HRMS (ESI) m/z calcd for C₂₄H₂₅ClFN₂O₆⁺ [M+H⁺] 491.1380, found 491.1256.

### Example 45: Preparation of 6-Chloro-N-(3-(dimethylamino)propyl)-3-(3-hydroxy-4-methoxybenzoyl)-4-oxo-4H-chromene-2-carboxamide NCGC00689952-01

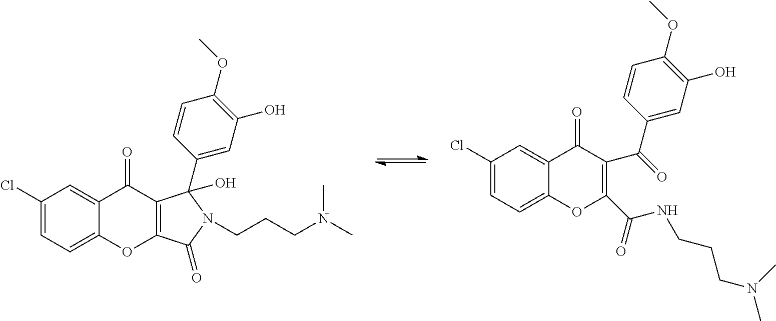 [SMILES: COc1ccc(C2(O)c3c(oc4ccc(Cl)cc4c3=O)C(=O)N2CCCN(C)C)cc1O ; COc1ccc(C(=O)c2c(C(=O)NCCCN(C)C)oc3ccc(Cl)cc3c2=O)cc1O]

7-Chloro-2-(3-(dimethylamino)propyl)-1-(3-hydroxy-4-methoxyphenyl)-1,2-dihydrochromeno[2,3-c]pyrrole-3,9-dione (36 mg, 0.081 mmol) was dissolved in DMSO (10 ml). The solution was stirred at room temperature with air bubbling for 2 days. The crude product was purified by HPLC (H₂O/MeCN). Yield=19.57%. ¹H NMR (400 Hz, CD₂Cl₂) δ 8.11 (d, J=2.5 Hz, 1H), 7.72-7.62 (m, 2H), 7.08 (dd, J=8.4, 2.3 Hz, 1H), 6.94 (d, J=2.3 Hz, 1H), 6.87 (d, J=8.4 Hz, 1H), 3.88 (s, 3H), 3.65 (dt, J=14.5, 4.6 Hz, 1H), 2.85 (ddd, J=14.3, 10.6, 3.3 Hz, 1H), 2.57-2.46 (m, 2H), 2.36-2.27 (m, 1H), 2.20 (s, 6H), 1.69 (ddd, J=12.3, 6.1, 3.1 Hz, 1H). 3:1 ratio. Major to minor. Only major NMR shown. LCMS RT (Method 1)=3.433 min, m/z=459.1 [M+H⁺]; HRMS (ESI) m/z calcd for C₂₃H₂₄ClN₂O₆⁺ [M+H⁺] 459.1323, found 459.1169.

### Example 46: Preparation of 8-Chloro-3-(3,4-dimethoxybenzoyl)-N-(3-(dimethylamino)propyl)-4-oxo-4H-chromene-2-carboxamide NCGC00842986-01

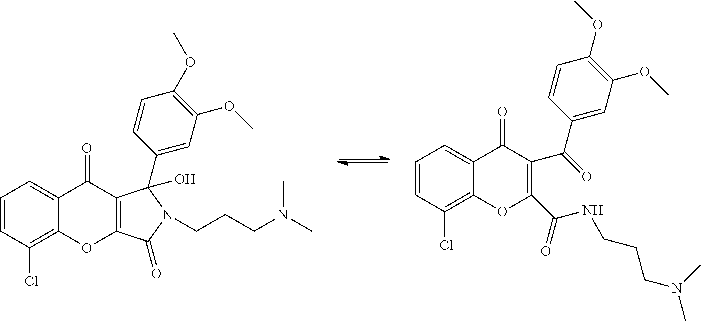 [SMILES: COc1ccc(C2(O)c3c(oc4c(Cl)cccc4c3=O)C(=O)N2CCCN(C)C)cc1OC ; COc1ccc(C(=O)c2c(C(=O)NCCCN(C)C)oc3c(Cl)cccc3c2=O)cc1OC]

5-Chloro-1-(3,4-dimethoxyphenyl)-2-(3-(dimethylamino)propyl)-1,2-dihydrochromeno[2,3-c]pyrrole-3,9-dione (201.3 mg, 0.441 mmol) was dissolved in DMSO (10 ml). The DMSO solution was stirred for 12 hours at 70° C. with air bubbling. The crude product was purified by HPLC (H₂O/MeCN). Yield=11.33%. ¹H NMR (400 MHz, CD₂Cl₂) δ 8.07 (dt, J=8.1, 1.6 Hz, 1H), 7.82 (dt, J=7.8, 1.6 Hz, 1H), 7.40 (td, J=7.9, 1.4 Hz, 1H), 7.08 (d, J=1.9 Hz, 1H), 7.03-6.98 (m, 1H), 6.85 (dt, J=8.5, 1.6 Hz, 1H), 3.83 (dd, J=4.2, 1.4 Hz, 6H), 3.75-3.68 (m, 1H), 2.93 (t, J=11.5 Hz, 1H), 2.68 (s, 2H), 2.36 (m, 1H), 2.33 (s, 6H), 1.87 (s, 1H). 3:2 ratio. Major to minor. Only major NMR shown. LCMS RT (Method 1)=3.728 min, m/z=473.2 [M+H⁺]; HRMS (ESI) m/z calcd for C₂₄H₂₅ClN₂O₆⁺ [M+H⁺] 473.1479, found 473.1379.

### Example 47: Preparation of 3-(Benzo[d][1,3]dioxole-5-carbonyl)-6-chloro-N-(3-(dimethylamino)propyl)-4-oxo-4H-chromene-2-carboxamide NCGC00842518-01

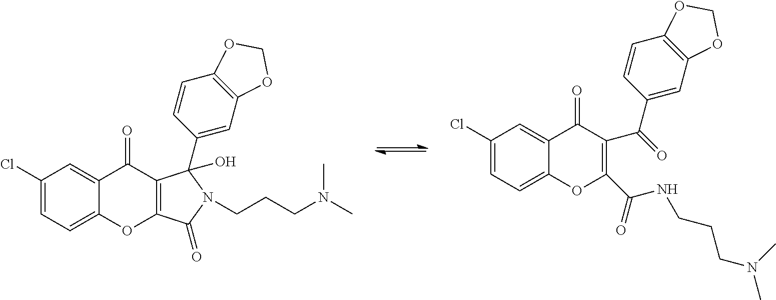 [SMILES: CN(C)CCCN1C(=O)c2oc3ccc(Cl)cc3c(=O)c2C1(O)c1ccc2c(c1)OCO2 ; CN(C)CCCNC(=O)c1oc2ccc(Cl)cc2c(=O)c1C(=O)c1ccc2c(c1)OCO2]

1-(Benzo[d][1,3]dioxol-5-yl)-7-chloro-2-(3-(dimethylamino)propyl)-1,2-dihydrochromeno[2,3-c]pyrrole-3,9-dione (259 mg, 0.587 mmol) was dissolved in DMSO (10 ml). The DMSO solution was stirred overnight at 70° C. The crude product was purified by HPLC (H₂O/MeCN). Yield=8.16%. ¹H NMR (400 MHz, CD₂Cl₂) δ 8.12 (d, J=2.5 Hz, 1H), 7.72-7.64 (m, 2H), 7.39 (d, J=6.7 Hz, 1H), 6.98 (d, J=1.9 Hz, 1H), 6.79 (d, J=8.1 Hz, 1H), 5.98-5.95 (m, 2H), 3.67 (ddd, J=14.3, 5.5, 3.6 Hz, 1H), 2.91-2.82 (m, 1H), 2.61-2.51 (m, 2H), 2.36-2.26 (m, 1H), 2.22 (s, 6H), 1.78-1.68 (m, 1H). 3:1 ratio. Major to minor. Only major NMR shown. LCMS RT (Method 1)=3.910 min, m/z=457.2 [M+H⁺]; HRMS (ESI) m/z calcd for C₂₃H₂₂ClN₂O₆⁺ [M+H⁺] 457.1166, found 457.1037.

### Example 48: Preparation of 3-(3,4-Dimethoxybenzoyl)-N-(3-(dimethyliminio)propyl)-6-fluoro-4-oxo-4H-chromene-2-carboxamide NCGC00842991-01

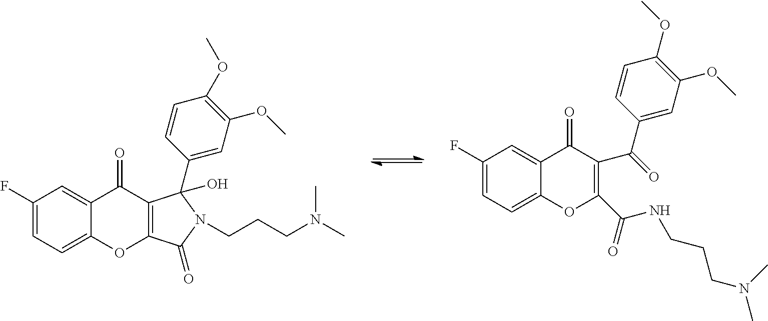 [SMILES: COc1ccc(C2(O)c3c(oc4ccc(F)cc4c3=O)C(=O)N2CCCN(C)C)cc1OC ; COc1ccc(C(=O)c2c(C(=O)NCCCN(C)C)oc3ccc(F)cc3c2=O)cc1OC]

1-(3,4-Dimethoxyphenyl)-2-(3-(dimethylamino) propyl)-7-fluoro-1,2-dihydrochromeno[2,3-c] pyrrole-3,9-dione (299 mg, 0.679 mmol) was dissolved in DMSO (10 ml). The DMSO solution was stirred overnight at 70° C. with air bubbling. The crude product was purified by HPLC (H₂O/MeCN). Yield=3.68%. ¹H NMR (400 MHz, CD₂Cl₂) δ 7.80 (dd, J=8.4, 3.3 Hz, 1H), 771 (dd, J=9.3, 4.2 Hz, 1H), 7.48 (ddd, J=8.9, 7.3, 3.0 Hz, 1H), 7.10 (d, J=2.2 Hz, 1H), 6.99 (dd, J=8.6, 2.1 Hz, 1H), 6.84 (d, J=8.4 Hz, 1H), 3.83 (d, J=5.7 Hz, 6H), 3.69 (dt, J=14.3, 4.6 Hz, 1H), 2.93 (d, J=12.1 Hz, 1H), 2.64 (m, 2H), 2.32 (m, 1H), 2.29 (s, 6H), 1.79 (s, 1H). 1:1 ratio. Major to minor. Only major NMR shown. LCMS RT (Method 1)=3.621 min, m/z=457.10 [M+H⁺]; HRMS (ESI) m/z calcd for C₂₄H₂₆FN₂O₆⁺[M+H⁺] 457.1775, found 457.1688.

### Example 49: Preparation of 6-(tert-Butyl)-3-(3,4-dimethoxybenzoyl)-N-(3-(dimethylamino)propyl)-4-oxo-4H-chromene-2-carboxamide NCGC00842988-01

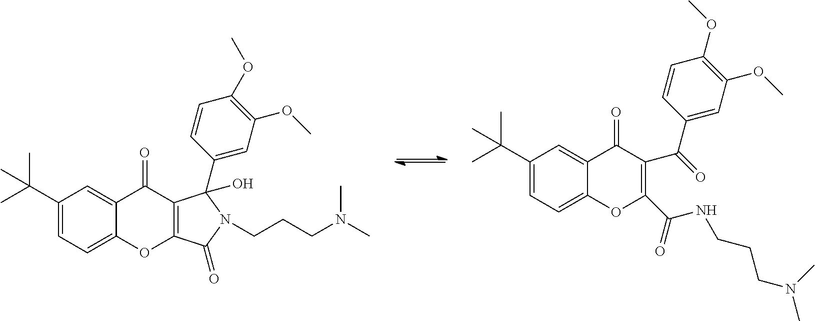 [SMILES: COc1ccc(C2(O)c3c(oc4ccc(C(C)(C)C)cc4c3=O)C(=O)N2CCCN(C)C)cc1OC ; COc1ccc(C(=O)c2c(C(=O)NCCCN(C)C)oc3ccc(C(C)(C)C)cc3c2=O)cc1OC]

7-(tert-Butyl)-1-(3,4-dimethoxyphenyl)-2-(3-(dimethylamino) propyl)-1,2-dihydrochromeno[2,3-c] pyrrole-3,9-dione (175.8 mg, 0.367 mmol) was dissolved in DMSO (10 ml). The solution was stirred at RT for 12 hours with air-bubbling. The crude product was purified by HPLC (H₂O/MeCN). Yield=10.79%. ¹H NMR (400 MHz, CD₂Cl₂) δ 8.17-8.13 (m, 1H), 7.62-7.54 (m, 2H), 7.13-7.08 (m, 1H), 6.98 (dt, J=8.4, 1.9 Hz, 1H), 6.83 (dt, J=8.5, 1.6 Hz, 1H), 3.83 (dd, J=8.1, 1.6 Hz, 6H), 3.69 (dt, J=14.9, 4.5 Hz, 1H), 2.90 (t, J=11.2 Hz, 1H), 2.53 (m, 21H), 2.33 (s, 1H), 2.23 (s, 61H), 1.71 (s, 1H), 1.37 (d, J=1.7 Hz, 9H). 1:1 ratio. Major to minor. Only major NMR shown. LCMS RT (Method 1)=4.332 min, m/z=495.2 [M+H⁺]; HRMS (ESI) m/z calcd for C₂₈H₃₅N₂O₆⁺ [M+H⁺] 495.2495, found 495.2406.

### Example 50: Preparation of 6-bromo-3-(3,4-dimethoxybenzoyl)-N-(3-(dimethylamino)propyl)-4-oxo-4H-chromene-2-carboxamide NCGC00842989-01

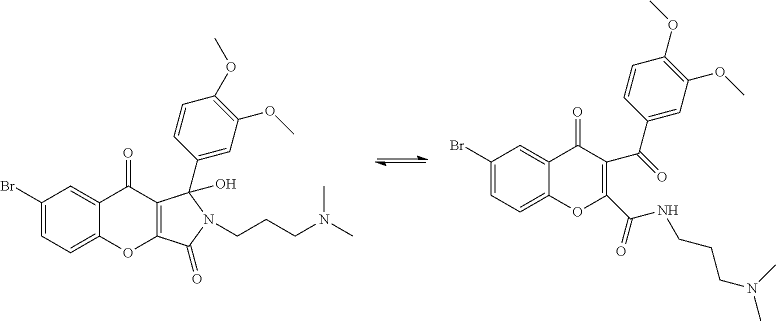 [SMILES: COc1ccc(C2(O)c3c(oc4ccc(Br)cc4c3=O)C(=O)N2CCCN(C)C)cc1OC ; COc1ccc(C(=O)c2c(C(=O)NCCCN(C)C)oc3ccc(Br)cc3c2=O)cc1OC]

7-Bromo-1-(3,4-dimethoxyphenyl)-2-(3-(dimethylamino)propyl)-1,2-dihydrochromeno[2,3-c]pyrrole-3,9-dione (265.4 mg, 0.529 mmol) was dissolved in DMSO (10 ml). The solution was stirred at 50° C. overnight with air-bubbling. The crude product was purified by HPLC (H₂O/MeCN). Yield=2.85%. ¹H NMR (400 Mz, CD₂Cl₂) δ 8.27 (d, J=2.5 Hz, 1H), 7.83 (dd, J=8.9, 2.5 Hz, 1H), 7.08 (d, J=2.3 Hz, 1H), 6.98 (dd, J=8.4, 2.2 Hz, 1H), 6.84 (d, J=8.3 Hz, 2H), 3.83 (d, J=4.7 Hz, 6H), 3.68 (dt, J=14.7, 4.2 Hz, 1H), 2.94-2.83 (m, 1H), 2.58 (s, 2H), 2.32 (s, 1H), 2.25 (s, 6H), 1.74 (s, 1H). 3:2 ratio. Major to minor. Only major NMR shown. LCMS RT (Method 1)==3.933 min, m/z=517.1 [M+H⁺]; HRMS (ESI) m/z calcd for C₂₄H₂₆BrN₂O₆⁺[M+H⁺] 517.0974, found 519.0835.

### Example 51: Preparation of 3-(3,4-Dimethoxybenzoyl)-N-(3-dimethylamino)propyl)-4-oxo-4H-chromene-2-carboxamide NCGC00842992-01

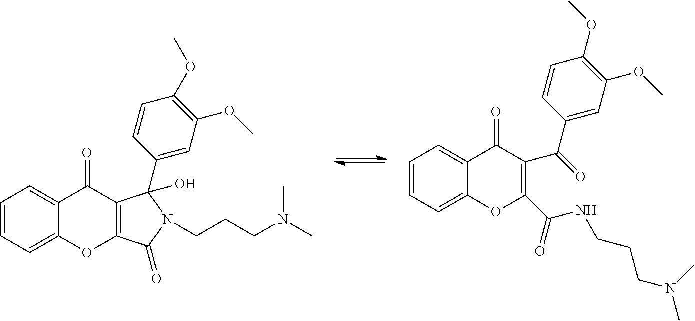 [SMILES: COc1ccc(C2(O)c3c(oc4ccccc4c3=O)C(=O)N2CCCN(C)C)cc1OC ; COc1ccc(C(=O)c2c(C(=O)NCCCN(C)C)oc3ccccc3c2=O)cc1OC]

1-(3,4-Dimethoxyphenyl)-2-(3-(dimethylamino) propyl)-1,2-dihydrochromeno[2,3-c] pyrrole-3,9-dione (305 mg, 0.722 mmol) was dissolved in DMSO (10 ml) with heating. Then the solution was stirred for 16 hr with air-bubbling at 70° C. The crude product was purified by HPLC (H₂O/MeCN). Yield=15.86%. ¹H NMR (400 MHz, CD₂C₂) δ 8.16 (dd, J=8.0, 1.7 Hz, 1H), 7.75 (ddd, J=8.6, 7.0, 1.8 Hz, TH), 7.68 (d, J=7.5 Hz, 1H), 7.49-7.42 (m, 1H), 7.10 (d, J=2.2 Hz, 1H), 6.99 (dd, J=8.4, 2.2 Hz, 1H), 6.84 (d, J=8.4 Hz, 1H), 3.83 (d, J=5.7 Hz, 6H), 3.73-3.63 (m, 1H), 2.89 (ddd, J=13.8, 10.1, 3.1 Hz, 1H), 2.58 (dd, J=8.8, 4.3 Hz, 2H), 2.37-2.28 (m, 1H), 2.24 (s, 6H), 1.73 (d, J=15.1 Hz, 1H). 3:2 ratio. Major to minor. Only major NMR shown. LCMS RT (Method 1) 3.406 min, m/z=439.2 [M+H⁺]; HRMS (ESI) m/z calcd for C₂₄H₂₇N₂O₆⁺ [M+H⁺] 439.1869, found 439.1769.

### Example 52: Preparation of 3-(3,4-dimethoxybenzoyl)-N-(3-(dimethylamino)propyl)-6-ethyl-4-oxo-4H-chromene-2-carboxamide NCGC00842993-01

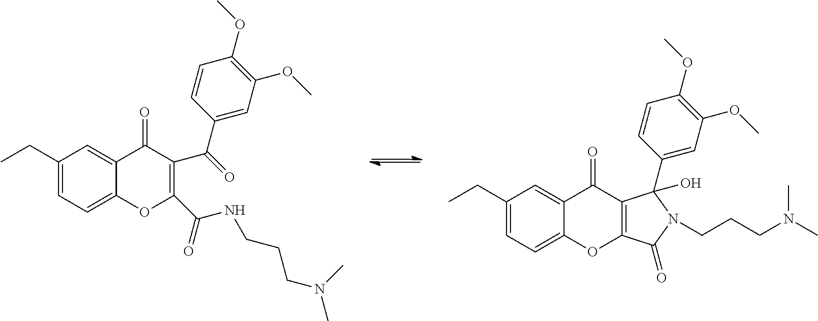 [SMILES: CCc1ccc2oc(C(=O)NCCCN(C)C)c(C(=O)c3ccc(OC)c(OC)c3)c(=O)c2c1 ; CCc1ccc2oc3c(c(=O)c2c1)C(O)(c1ccc(OC)c(OC)c1)N(CCCN(C)C)C3=O]

1-(3,4-dimethoxyphenyl)-2-(3-(dimethylamino) propyl)-7-ethyl-1,2-dihydrochromeno[2,3-c] pyrrole-3,9-dione (125 mg, 0.277 mmol) was dissolved in DMSO (10 ml) and stirred with air bubbling for 12 hours. The product was purified by HPLC (H₂O/MeCN) Yield=18.16%. ¹H NMR (400 MHz, CD₂Cl₂) δ 7.98-7.95 (m, 1H), 7.61-7.57 (m, 2H), 7.10 (d, J=2.2 Hz, 1H), 6.98 (dd, J=8.4, 2.2 Hz, 1H), 6.83 (d, J=8.4 Hz, 1H), 3.82 (d, J=6.2 Hz, 6H), 3.72-3.63 (m, 1H), 2.89 (ddd, J=13.5, 10.2, 3.1 Hz, 1H), 2.78 (dd, J=15.6, 7.8 Hz, 2H), 2.60-2.52 (m, 2H), 2.36-2.27 (m, 1H), 2.22 (s, 6H), 1.72 (d, J=14.6 Hz, 1H), 1.28 (dt, J=16.1, 7.6 Hz, 3H). 3:2 ratio. Major to minor. Only major NMR shown. LCMS RT (Method 1)=3.980 mi, m/z=467.2 [M+H⁺]; HRMS (ESI) m/z calcd for C₂₆H₃₁N₂O₆⁺ [M+H⁺] 467.2182, found 467.2078.

### Example 53: Preparation of 5-chloro-3-(3,4-dimethoxybenzoyl)-N-(3-(dimethylamino)propyl)-4-oxo-4H-chromene-2-carboxamide NCGC00842996-01

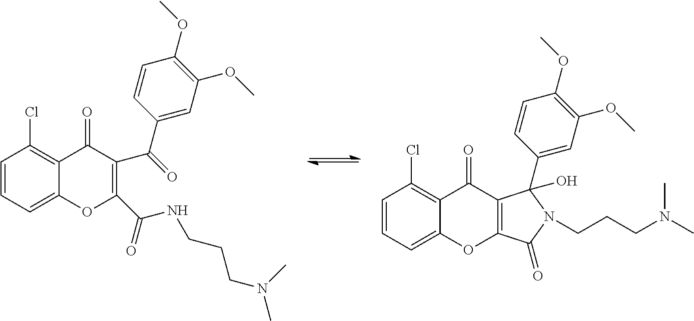 [SMILES: COc1ccc(C(=O)c2c(C(=O)NCCCN(C)C)oc3cccc(Cl)c3c2=O)cc1OC ; COc1ccc(C2(O)c3c(oc4cccc(Cl)c4c3=O)C(=O)N2CCCN(C)C)cc1OC]

8-Chloro-1-(3,4-dimethoxyphenyl)-2-(3-(dimethylamino)propyl)-1,2-dihydrochromeno[2,3-c]pyrrole-3,9-dione (308 mg, 0.674 mmol) was dissolved in DMSO (10 ml). The solution was stirred overnight with air-bubbling. The crude product was purified by HPLC (H₂O/MeCN). Yield=1.456%. ¹H NMR (400 MHz, CD₂Cl₂) δ 7.66 (d, J=6.7 Hz, 1H), 7.62-7.56 (m, 2H), 7.06 (d, J=2.3 Hz, 1H), 7.04-6.98 (m, 1H), 6.85 (d, J=8.5 Hz, 2H), 3.83 (d, J=3.3 Hz, 6H), 3.66 (dt, J=14.4, 4.7 Hz, 1H), 2.92-2.82 (m, 1H), 2.55 (s, 2H), 2.35-2.27 (m, 1H), 2.24 (s, 6H), 1.71 (s, 1H). 1:1 ratio. Major to minor. Only major NMR shown. LCMS RT (Method 1)=3.683 min, m/z=473.1 [M+H⁺]; HRMS (ESI) m/z calcd for C₂₄H₂₆ClN₂O₆⁺ [M+H⁺] 473.1479, found 473.1398.

### Example 54: Preparation of 3-(3,4-dimethoxybenzoyl)-N-(3-(dimethylamino)propyl)-4-oxo-6-(trifluoromethyl)-4H-chromene-2-carboxamide NCGC00842997-02

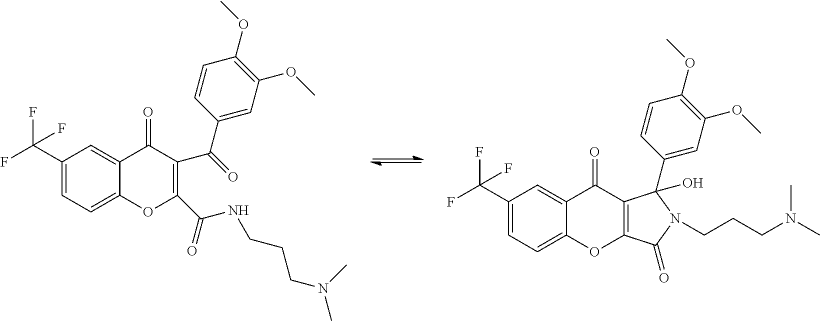 [SMILES: COc1ccc(C(=O)c2c(C(=O)NCCCN(C)C)oc3ccc(C(F)(F)F)cc3c2=O)cc1OC ; COc1ccc(C2(O)c3c(oc4ccc(C(F)(F)F)cc4c3=O)C(=O)N2CCCN(C)C)cc1OC]

1-(3,4-Dimethoxyphenyl)-2-(3-(dimethylamino) propyl)-7-(trifluoromethyl)-1,2-dihydrochromeno[2,3-c] pyrrole-3,9-dione (219 mg, 0.447 mmol) was dissolved in DMSO (10 ml) with air bubbling for 12 hours. The crude product was purified by HPLC (H₂O/MeCN). Yield=11.36%. ¹H NMR (400 MHz, CD₂Cl₂) δ 8.46 (d, J=2.3 Hz, 1H), 7.97 (dd, J=8.8, 2.4 Hz, 1H), 7.83 (d, J=8.8 Hz, 1H), 7.10 (d, J=2.2 Hz, 1H), 6.99 (dd, J=8.4, 2.2 Hz, 1H), 6.84 (d, J=8.4 Hz, 1H), 3.83 (d, J=7.1 Hz, 6H), 3.71 (ddd, J=14.5, 7.5, 3.7 Hz, 1H), 2.88 (ddd, J=14.3, 10.7, 3.3 Hz, 1H), 2.56 (td, J=9.1, 4.5 Hz, 2H), 2.38-2.31 (m, 1H), 2.20 (s, 611), 1.72-1.67 (m, 1H). 3:1 ratio. Major to minor. Only major NMR shown. LCMS RT (Method 1)=4.039 min, m/z=507.1 [M+H⁺]; HRMS (ESI) m/z calcd for C₂₅H₂₆F₃N₂O₆⁺ [M+H⁺] 507.1743, found 507.1604.

### Example 55: Preparation of 6-Chloro-N-(3-(dimethylamino)propyl)-3-(5-methoxynicotinoyl)-4-oxo-4H-chromene-2-carboxamide NCGC00842486-02

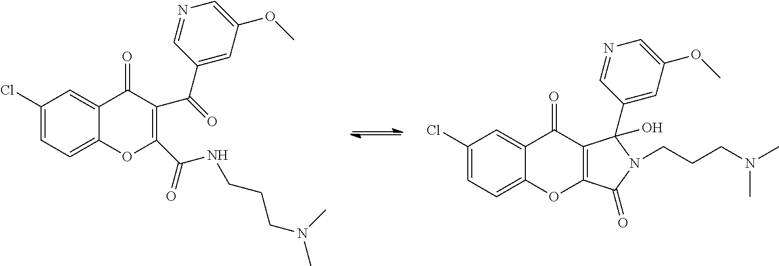 [SMILES: COc1cncc(C(=O)c2c(C(=O)NCCCN(C)C)oc3ccc(Cl)cc3c2=O)c1 ; COc1cncc(C2(O)c3c(oc4ccc(Cl)cc4c3=O)C(=O)N2CCCN(C)C)c1]

7-Chloro-2-(3-(dimethylamino) propyl)-1-(5-m-ethoxypyridin-3-yl)-1,2-dihydrochromeno[2,3-c] pyrrole-3,9-dione (338 mg, 0.790 mmol) was dissolved in DMSO (10 ml). The DMSO solution was stirred overnight at 70° C. The crude product was purified by HPLC (1120/MeCN). Yield=5.36%. ¹H NMR (400 MHz, CD₂Cl₂) δ 8.23 (d, J=3.0 Hz, 1H), 8.18 (d, J=1.7 Hz, 1H), 8.11 (d, J=2.6 Hz, 1H), 7.73-7.62 (m, 2H), 7.55-7.51 (m, 1H), 3.89 (s, 3H), 3.76 (dt, J=14.6, 4.5 Hz, 1H), 2.90 (ddd, J=14.3, 10.2, 3.3 Hz, 1H), 2.68-2.50 (m, 2H), 2.34 (td, J=9.6, 5.2 Hz, 1H), 2.25 (s, 6H), 1.80-1.71 (m, 1H). LCMS RT (Method 1) 3.266 min, m/z=444.1 [M+H⁺]; HRMS (ESI) m/z calcd for C₂₂H₂₃ClN₃O₆⁺ [M+H⁺] 444.1326, found 444.1193.

### Example 56: Preparation of 6-Chloro-3-(5,6-di methoxynicotinoyl)-N-(3-(dimethylamino)propyl)-4-oxo-4H-chromene-2-carboxamide NCGC00842483-02

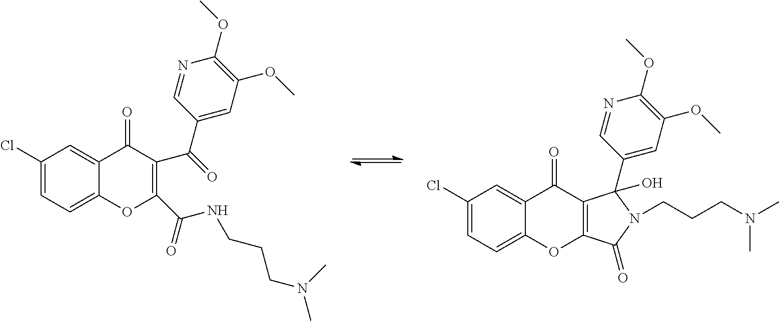 [SMILES: COc1cc(C(=O)c2c(C(=O)NCCCN(C)C)oc3ccc(Cl)cc3c2=O)cnc1OC ; COc1cc(C2(O)c3c(oc4ccc(Cl)cc4c3=O)C(=O)N2CCCN(C)C)cnc1OC]

7-Chloro-1-(5,6-dimethoxypyridin-3-yl)-2-(3-(dimethylamino)propyl)-1,2-dihydrochromeno[2,3-c]pyrrole-3,9-dione (306 mg, 0.668 mmol) was dissolved in DMSO (10 ml). The DMSO solution was stirred overnight at 70° C. with air bubbling. The crude product was purified by HPLC (H₂O/MeCN). Yield=11.34%. ¹H NMR (400 MHz, CD₂Cl₂) δ 8.99 (s, 1H), 8.12 (d, J=2.5 Hz, 1H), 8.10 (1, J=2.2 Hz, 1H), 8.04 (d, J=2.0 Hz, 1H), 7.76 (dd, J=8.8, 2.4 Hz, 2H), 4.00 (s, 3H), 3.84 (s, 3H), 3.51-3.46 (m, 2H), 3.08 (t, J=6.8 Hz, 2H), 2.77 (s, 6H), 2.09 (q, J=6.2 Hz, 2H). 1:1 ratio. Major to minor. Only major NMR shown. LCMS RT (Method 1)=5.292 min, m/z=474.1 [M+H⁺]; HRMS (ESI) n/z calcd for C₂₃H₂₅ClN₃O₆⁺ [M+H⁺] 474.1432, found 474.1314.

### Example 57: Preparation of 6-Chloro-3-(3-cyano-4-methoxybenzoyl)-N-(3-(dimethylamino)propyl)-4-oxo-4H-chromene-2-carboxamide NCGC00842484-02

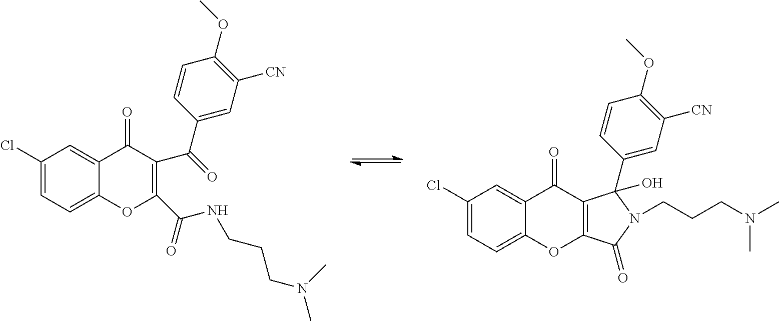 [SMILES: COc1ccc(C(=O)c2c(C(=O)NCCCN(C)C)oc3ccc(Cl)cc3c2=O)cc1C#N ; COc1ccc(C2(O)c3c(oc4ccc(Cl)cc4c3=O)C(=O)N2CCCN(C)C)cc1C#N]

5-(7-Chloro-2-(3-(dimethylamino) propyl)-3,9-dioxo-1,2,3,9-tetrahydrochromeno[2,3-c] pyrrol-1-yl)-2-methoxybenzonitrile (280 mg, 0.620 mmol)] was dissolved in DMSO (10 ml). The DMSO solution was stirred for 16 hours with air-bubbling. Yield=1.483%. ¹H NMR (400 MHz, CD₂Cl₂) δ 8.10 (d, J=2.5 Hz, 1H), 7.76-7.60 (m, 4H), 7.00 (d, J=8.9 Hz, 1H), 3.94 (s, 3H), 3.71 (ddd, J=14.5, 5.7, 3.8 Hz, 1H), 2.82 (ddd, J=14.3, 10.3, 3.3 Hz, 1H), 2.57-2.47 (m, 2H), 2.38-2.28 (m, 1H), 2.24 (s, 6H), 1.73 (d, J=15.4 Hz, 1H). LCMS RT (Method 1)=4.026 min, m/z=468.1 [M+H⁺]; HRMS (ESI) m/z calcd for C₂₄H₂₃ClN₃O₅⁺ [M+H⁺] 468.1326, found 468.1072.

### Example 58: Preparation of 7-Chloro-3-(3,4-dimethoxybenzoyl)-N-(3-(dimethylamino)propyl)-4-oxo-4H-chromene-2-carboxamide NCGC00842990-02

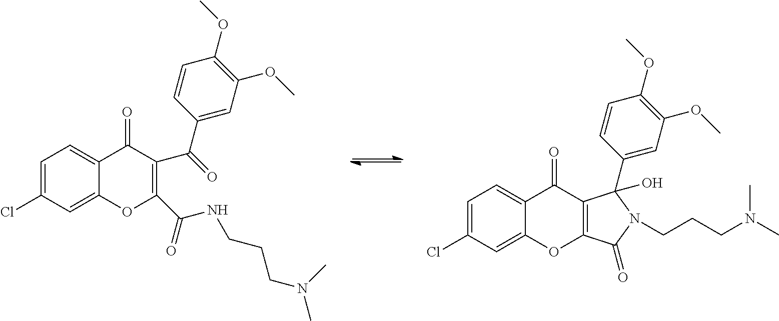 [SMILES: COc1ccc(C(=O)c2c(C(=O)NCCCN(C)C)oc3cc(Cl)ccc3c2=O)cc1OC ; COc1ccc(C2(O)c3c(oc4cc(Cl)ccc4c3=O)C(=O)N2CCCN(C)C)cc1OC]

6-Chloro-1-(3,4-dimethoxyphenyl)-2-(3-(dimethylamino) propyl)-1,2-dihydrochromeno[2,3-c] pyrrole-3,9-dione (220 mg, 0.481 mmol) was dissolved in DMSO with heating. Then the solution was stirred for 16 hours with air-bubbling at 70° C. The crude product was purified by HPLC (H₂O/MeCN). Yield=7.38%. ¹H NMR (400 MHz, CD₂Cl₂) a 8.10 (d, J=8.6 Hz, 1H), 7.70 (d, J=1.7 Hz, 1H), 7.43 (dd, J=8.6, 1.8 Hz, 1H), 7.08 (d, J=1.9 Hz, 1H), 6.99 (dd, J=8.4, 2.0 Hz, 1H), 6.84 (d, J=8.4 Hz, 1H), 3.83 (d, J=5.5 Hz, 6H), 3.68 (dt, J=14.7, 4.7 Hz, 1H), 2.87 (ddd, J=14.5, 10.5, 3.6 Hz, 1H), 2.60-2.54 (m, 2H), 2.31 (tt, J=7.6, 4.2 Hz, 1H), 2.19 (s, 6H), 1.71-1.64 (m, 1H). 2:1 ratio. Major to minor. Only major NMR shown. LCMS RT (Method 1)=3.751 min, m/z=473.1 [M+H⁺]; HRMS (ESI) m/z calcd for C₂₄H₂₆ClN₂O₆⁺ [M+H⁺] 473.1479, found 473.1078.

### Example 59: Preparation of 6,7-Dichloro-3-(3,4-dimethoxybenzoyl)-N-(3-(dimethylamino)propyl)-4-oxo-4H-chromene-2-carboxamide NCGC00842987-02

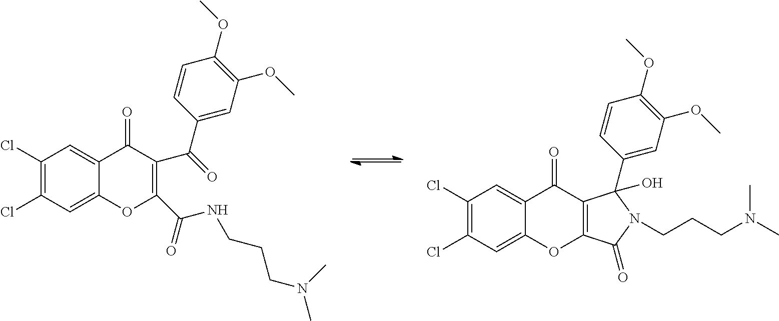 [SMILES: COc1ccc(C(=O)c2c(C(=O)NCCCN(C)C)oc3cc(Cl)c(Cl)cc3c2=O)cc1OC ; COc1ccc(C2(O)c3c(oc4cc(Cl)c(Cl)cc4c3=O)C(=O)N2CCCN(C)C)cc1OC]

6,7-Dichloro-1-(3,4-dimethoxyphenyl)-2-(3-(dimethylamino) propyl)-1,2-dihydrochromeno[2,3-c] pyrrole-3,9-dione (172.5 mg, 0.351 mmol) was dissolved in DMSO with heating. Then the solution was stirred for 16 hr with air-bubbling at 70° C. The crude product was purified by HPLC (H₂O/MeCN). Yield=23.47%. ¹H NMR (400 MHz, CD₂Cl₂) δ 8.23 (s, 1H), 7.84 (s, 1H), 7.08 (d, J=1.9 Hz, 1H), 6.98 (dd, J=8.5, 2.0 Hz, 1H), 6.84 (d, J=8.4 Hz, 1H), 3.83 (d, J=4.9 Hz, 6H), 3.68 (dt, J=14.3, 4.6 Hz, 1H), 2.93-2.82 (m, 1H), 2.56 (q, J=5.9 Hz, 2H), 2.30 (d, J=10.0 Hz, 1H), 2.23 (s, 6H), 1.73 (d, J=15.6 Hz, 1H). 2:1 ratio. Major to minor. Only major NMR shown. LCMS RT (Method 1)=4.159 min, m/z=507.1 [M+H⁺]; HRMS (ESI) m/z calcd for C₂₄H₂₅Cl₂N₂O₆⁺ [M+H⁺] 507.1090, found 507.0685.

### Example 60: Preparation of 6-Chloro-3-(3,4-dihydroxybenzoyl)-N-(3-(dimethylamino)propyl)-4-oxo-4H-chromene-2-carboxamide NCGC00846211-01

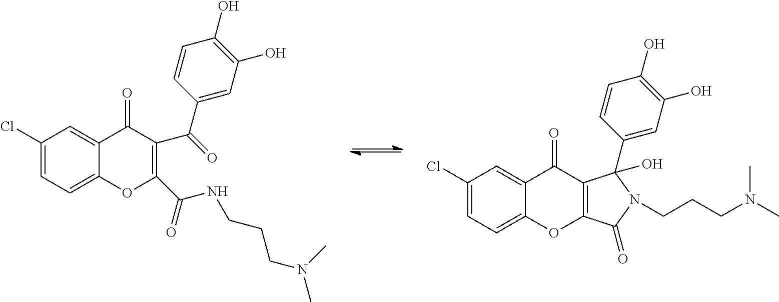 [SMILES: CN(C)CCCNC(=O)c1oc2ccc(Cl)cc2c(=O)c1C(=O)c1ccc(O)c(O)c1 ; CN(C)CCCN1C(=O)c2oc3ccc(Cl)cc3c(=O)c2C1(O)c1ccc(O)c(O)c1]

7-Chloro-1-(3,4-dihydroxyphenyl)-2-(3-(dimethylamino)propyl)-1,2-dihydrochromeno[2,3-c]pyrrole-3,9-dione (141 mg. 0.329 mmol) was dissolved in DMSO (10 ml). The solution was stirred for 12 hours with air bubbling. The crude product was purified by normal phase column (20˜50% MeOH in EA). Yield=6.91%. ¹H NMR (400 MHz, Acetic acid-d₄) δ 8.16 (t, J=2.4 Hz, 1H), 7.82 (ddd, J=11.8, 8.9, 2.7 Hz, 2H), 7.50 (d, J=2.2 Hz, 1H), 7.38 (dd, J=8.4, 2.1 Hz, 1H), 6.91 (dd, J=8.3, 2.3 Hz, 1H), 3.48 (q, J=6.1 Hz, 2H), 3.12 (q, J=6.7 Hz, 2H), 2.81 (s, 6H), 2.75 (d, J=9.5 Hz, 2H). 2:1 ratio. Major to minor. Only major NMR shown. LCMS RT (Method 1)=3.406 min, m/z=445.1 [M+H⁺]; HRMS (ESI) m/z calcd for C₂₂H₂₂ClN₂O₆⁺[M+H⁺] 445.1166, found 445.0875.

### Example 61: Preparation of 6-Chloro-3-(3,4-dimethoxybenzoyl)-N-(3-(dimethylamino)propyl)-4-oxo-4H-chromene-2-carboxamide

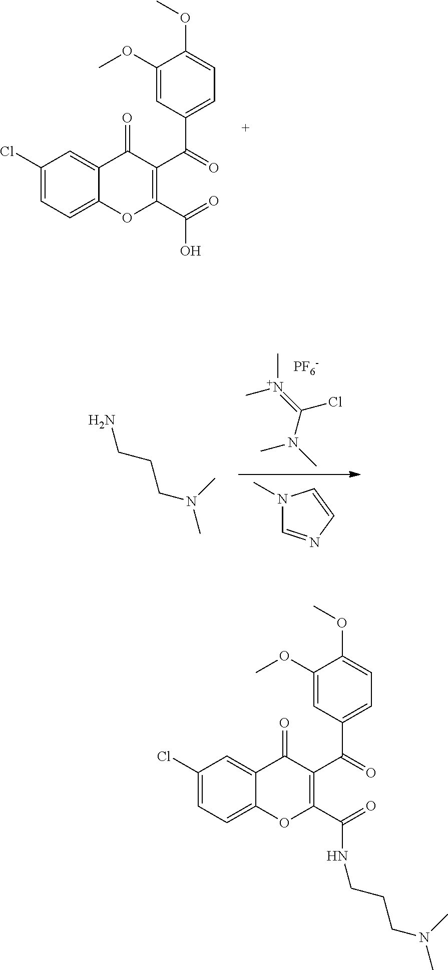

To the solution of 6-chloro-3-(3,4-dimethoxybenzoyl)-4-oxo-4H-chromene-2-carboxylic acid (10 mg, 0.026 mmol) and N¹,N¹-dimethylpropane-1,3-diamine (4.21 μl, 0.033 mmol) in acetonitrile (2 ml) was added 1-methyl-1H-imidazole (7.18 μl, 0.090 mmol), followed by N-(chloro(dimethylamino)methylene)-N-methylmethananinium hexafluorophosphate(V) (8.30 mg, 0.030 mmol). The reaction mixture was stirred at room temperature overnight. The acetonitrile solution was dried up and the residue was dissolved in ethylacetate, followed by addition of 3 mL of water. The separated organic phase was dried with sodium sulfate to obtain the crude product.

### Example 62: Preparation of 6-Chloro-3-(3,4-dimethoxybenzoyl)-4-oxo-N-(pyrindin-5-ylmethyl)-4H-chromene-2-carboxamide NCGC00846210-01

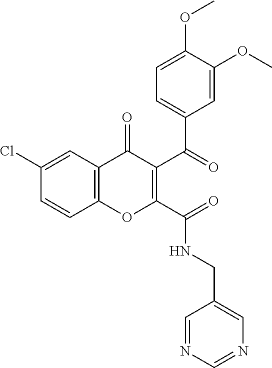 [SMILES: COc1ccc(C(=O)c2c(C(=O)NCc3cncnc3)oc3ccc(Cl)cc3c2=O)cc1OC]

To the solution of pyrimidin-5-ylmethanamine (10 mg, 0.092 mmol) and 6-chloro-3-(3,4-dimethoxybenzol)-4-oxo-4H-chromene-2-carboxylic acid (27.4 mg, 0.070 mmol) in acetonitrile was added 1-methyl-1H-imidazole (19.67 μl, 0.247 mmol), followed by N-(chloro(dimethylamino)methylene)-N-methylmethanaminium hexafluorophosphate(V) (22.74 mg, 0.081 mmol). The reaction mixture was stirred overnight at room temperature. The crude product was purified by silica column (50-100% EA in Hex). Yield=16.20%. ¹H NMR (400 MHz, Acetic acid-d₄) δ 9.11 (s, 1H), 8.77 (s, 2H), 7.71 (d, J=2.4 Hz, 1H), 7.51 (dd, J=8.8, 2.5 Hz, 1H), 7.23 (d, J=9.0 Hz, 1H), 7.02 (d, J=8.7 Hz, 1H), 6.87 (s, 1H), 6.75 (d, J=8.1 Hz, 1H), 4.86-4.50 (m, 2H), 3.81-3.62 (m, 6H). LCMS RT (Method 1)=4.238 min, m/z=480.1 [M+H⁺]; HRMS (ESI) m/z calcd for C₂₄H₁₉ClN₃O₆[M+H⁺] 480.0962, found 480.0645.

### Example 63: Preparation of 6-Chloro-3-(3,4-dimethoxybenzoyl)-4-oxo-N-(pyridin-4-ylmethyl)-4H-chromene-2-carboxamide NCGC00846208-01

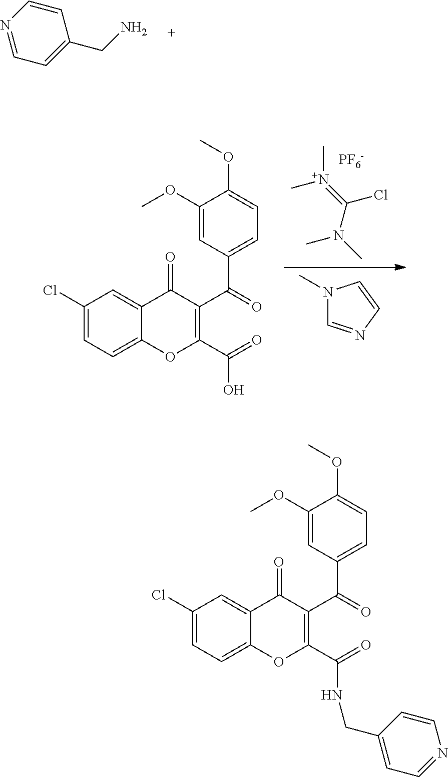

4-(Aminomethyl)pyridine (3.41 μl, 0.033 mmol) and 6-chloro-3-(3,4-dimethoxybenzoyl)-4-oxo-4H-chromene-2-carboxylic acid (10 mg, 0.026 mmol) were dissolved in acetonitrile (3 ml). To the solution was added 1-methyl-1H-imidazole (7.18 μl, 0.090 mmol), followed by the addition of N-(chloro(dimethylamino)methylene)-N-methylmethananinium hexafluorophosphate(V) (8.30 mg, 0.030 mmol). The reaction mixture was stirred overnight at room temperature. The crude product was purified by normal phase column (50-100% EA in Hex). Yield=44.6%. ¹H NMR (400 MHz, CD₂Cl₂) δ 8.52 (d, J=5.6 Hz, 2H), 8.14 (d, J=2.7 Hz, 1H), 7.77 (ddd, J=9.1, 2.7, 1.3 Hz, 11), 7.60-7.54 (m, 2H), 7.31 (dt, J=8.4, 1.7 Hz, 1H), 7.22 (d, J=5.8 Hz, 2H), 6.83 (t, =8.6 Hz, 1H), 4.57 (d, J=6.2 Hz, 2H), 3.93-3.88 (m, 6H). 3:1 ratio. Major to minor. Only major NMR shown. LCMS RT (Method 1)=3.895 min, m/z=479.1 [M+H⁺]; HRMS (ESI) m/z calcd for C₂₅H₂₀ClN₂O₆⁺ [M+H⁺] 479.1010, found 479.0705.

### Example 64: Preparation of 6-Chloro-3-(3,4-dimethoxybenzoyl)-N-(3-(methyl(phenyl)amino)propyl)-4-oxo-4H-chromene-2-carboxamide NCGC00846233-01

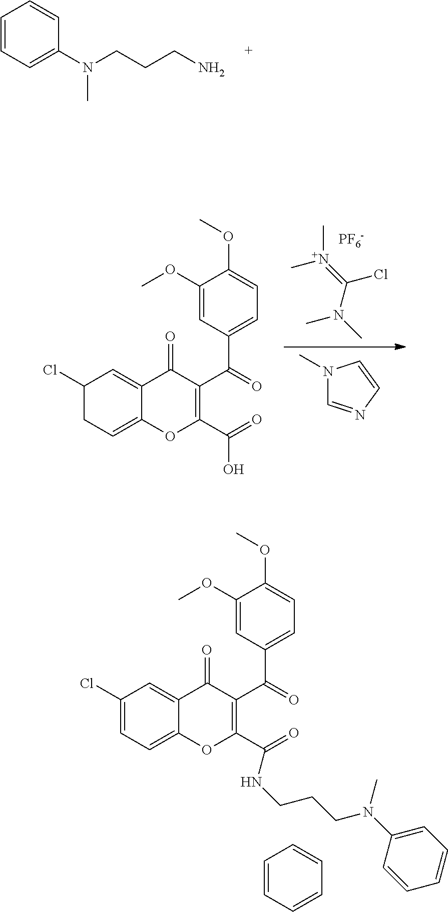

To the solution of N¹-methyl-N¹-phenylpropane-1,3-diamine (5.46 μl, 0.033 mmol) and 6-chloro-3-(3,4-dimethoxybenzoyl)-4-oxo-6,7-dihydro-4H-chromene-2-carboxylic acid (10 mg, 0.026 mmol) in MeCN(3 mL) was added 1-methyl-1H-imidazole (7.14 μl, 0.090 mmol) and N-(chloro(dimethylamino)methylene)-N-methylmethanaminium hexafluorophosphate(V) (8.26 mg, 0.029 mmol) sequentially. The reaction mixture was stirred overnight at room temperature. The crude product was purified by normal phase column (20-50% EA in Hex). Yield=40.7%. ¹H NMR (400 MHz, CD₂Cl₂) δ 8.12 (t, J=2.6 Hz, 1H), 7.77-7.65 (m, 2H), 7.57 (t, J=2.1 Hz, 1H), 7.30-7.21 (m, 3H), 6.87-6.76 (m, 4H), 3.90 (dd, J=9.4, 2.3 Hz, 6H), 3.51-3.44 (m, 2H), 3.41 (td, J=6.7, 2.2 Hz, 2H), 2.91 (d, J=2.3 Hz, 3H), 1.91 (td, J=6.8, 2.3 Hz, 2H). 5:1 ratio. Major to minor. Only major NMR shown. LCMS RT (Method 1)=4.934 and 5.134 min, m/z=535.1 [M+H⁺]; HRMS (ESI) m/z calcd for C₂₉H₂₈ClN₂O₆⁺ [M+H⁺] 535.1636, found 535.1654.

### Example 65: Preparation of 6-chloro-3-(3,4-dimethoxybenzoyl)-4-oxo-N-(pyridin-3-ylmethyl)-4H-chromene-2-carboxamide NCGC00846491-01

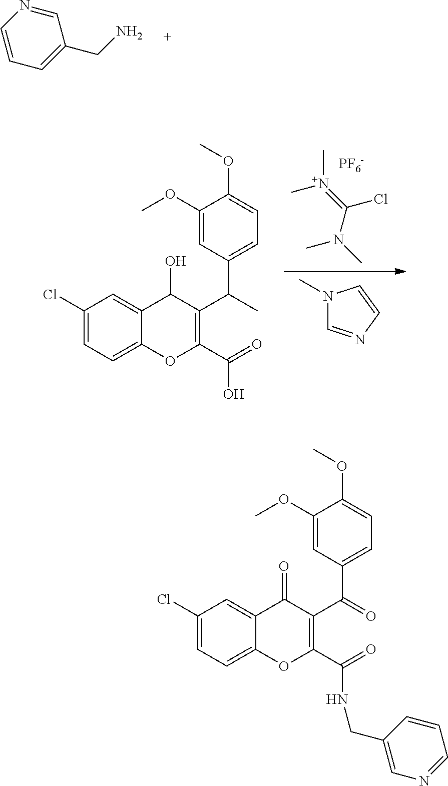

To the solution of 6-chloro-3-(1-(3,4-dimethoxyphenyl)ethyl)-4-hydroxy-4H-chromene-2-carboxylic acid (10 mg, 0.026 mmol) in MeCN (3 ml) was added pyridin-3-ylmethanamine (3.39 μl, 0.033 mmol), 1-methyl-1H-imidazole (7.14 μl, 0.090 mmol), and N-(chloro(dimethylamino)methylene)-N-methylmethanaminium hexafluorophosphate(V) (8.26 mg, 0.029 mmol) sequentially. The reaction mixture was stirred overnight at room temperature. The crude product was purified by normal phase column (50-100% EA in Hex). Yield=55.5%. ¹H NMR (400 MHz, CD₂Cl₂) δ 8.55-8.51 (m, 1H), 8.49 (dd, J=5.3, 1.3 Hz, 1H), 8.13 (d, J=2.5 Hz, 1H), 7.75 (dd, J=9.0, 2.6 Hz, 1H), 7.68-7.64 (m, 1H), 7.58 (d, J=2.1 Hz, 1H), 7.55 (d, J=9.0 Hz, 1H), 7.31 (dd, J=8.3, 2.1 Hz, 1H), 7.28 (dd, J=7.9, 4.7 Hz, 1H), 6.85 (dd, J=8.5, 1.1 Hz, 1H), 4.56 (d, J=6.1 Hz, 2H), 3.91 (d, J=4.6 Hz, 6H). 2:1 ratio. Major to minor. Only major NMR shown. LCMS RT (Method 1)=2.756 min, m/z=479.1 [M+H⁺]; HRMS (ESI) m/z calcd for C₂₅H₂₀ClN₂O₆⁺[M+H⁺] 479.1010, found 479.1004.

### Example 66: Preparation of 6-Chloro-3-(3,4-dimethoxybenzoyl)-N-((1-methyl-1H-imidazol-5-yl)methyl)-4-oxo-4H-chromene-2-carboxamide NCGC00846488-01

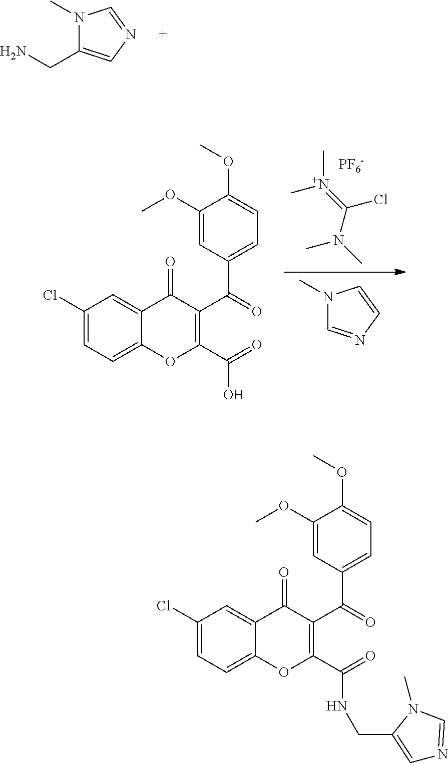

To the solution of 6-chloro-3-(3,4-dimethoxybenzoyl)-4-oxo-4H-chromene-2-carboxylic acid (10 mg, 0.026 mmol) in MeCN (3 ml) was added (1-methyl-1H-imidazol-5-yl)methanamine (3.10 μl, 0.033 mmol), 1-methyl-1H-imidazole (7.18 μl, 0.090 mmol), and N-(chloro(dimethylamino)methylene)-N-methylmethanaminium hexafluorophosphate(V) (8.30 mg, 0.030 mmol) sequentially. The reaction mixture was stirred overnight at room temperature. The crude product was purified by normal phase column (50 to 100% EA in Hex and 0 to 30% MeOH in EA). Yield=20.98%. ¹H NMR (400 MHz, CD₂Cl₂) a 8.14 (d, J=3.8 Hz, 1H), 7.74 (dd, J=9.2, 3.5 Hz, 1H), 7.63-7.53 (m, 3H), 7.31 (dt, J=8.3, 1.6 Hz, 1H), 6.92-6.82 (m, 2H), 4.54 (d, J=5.7 Hz, 2H), 3.91 (dd, J=6.5, 1.7 Hz, 6H), 3.51 (s, 3H). 2:1 ratio. Major to minor. Only major NMR shown. LCMS RT (Method 1)=3.898 min, m/z=482.1 [M+H⁺]; HRMS (ESI) m/z calcd for C₂₄H₂₁ClN₃O₆⁺ [M+H⁺] 482.1119, found 482.1127.

### Example 67: Preparation of 6-Chloro-3-(3,4-dimethoxybenzoyl)-N((1-methyl-1H-imidazol-4-yl)methyl)-4-oxo-4H-chromene-2-carboxamide NCGC00846490-01

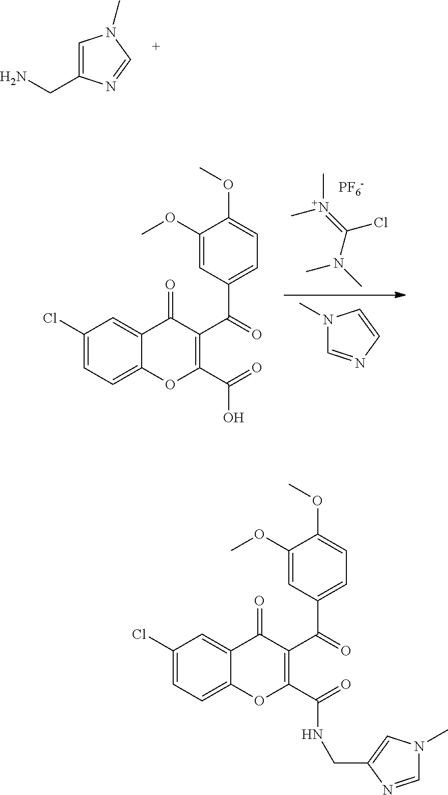

To the solution of 6-chloro-3-(3,4-dimethoxybenzoyl)-4-oxo-4H-chromene-2-carboxylic acid (10 mg, 0.026 mmol) in MeCN (3 ml) was added (1-methyl-1H-imidazol-4-yl)methanamine (3.20 μl, 0.033 mmol), 1-methyl-TH-imidazole (7.18 μl, 0.090 mmol), and N-(chloro(dimethylamino)methylene)-N-methylmethanaminium hexafluorophosphate(V) (8.30 mg, 0.030 mmol) sequentially. The reaction mixture was stirred overnight at room temperature. The crude product was purified by normal phase column (50 to 100% EA in Hex and 0 to 20% MeOH in EA). Yield=23.40%. ¹H NMR (400 MHz, CD₂Cl₂) δ 8.13 (dd, J=2.6, 1.4 Hz, 1H), 7.79-7.66 (m, 2H), 7.64 (dd, J=8.9, 1.2 Hz, 1H), 7.48 (s, 1H), 7.40 (s, 1H), 6.94-6.88 (m, 2H), 4.42 (d, J=6.1 Hz, 2H), 3.85-3.79 (m, 6H), 3.69 (d, J=1.3 Hz, 3H). 1:1 ratio. Major to minor. Only major NMR shown. LCMS RT (Method 1)=3.863 min, m/z=482.1 [M+H⁺]; HRMS (ESI) n/z calcd for C₂₄H₂₁ClN₃O₆⁺[M+H⁺] 482.1119, found 482.1122.

### Example 68: Preparation of 6-Chloro-3-(3,4-dimethoxybenzoyl)-4-oxo-N-(pyridazin-4-ylmethyl)-4H-chromene-2-carboxamide NCGC00846489-01

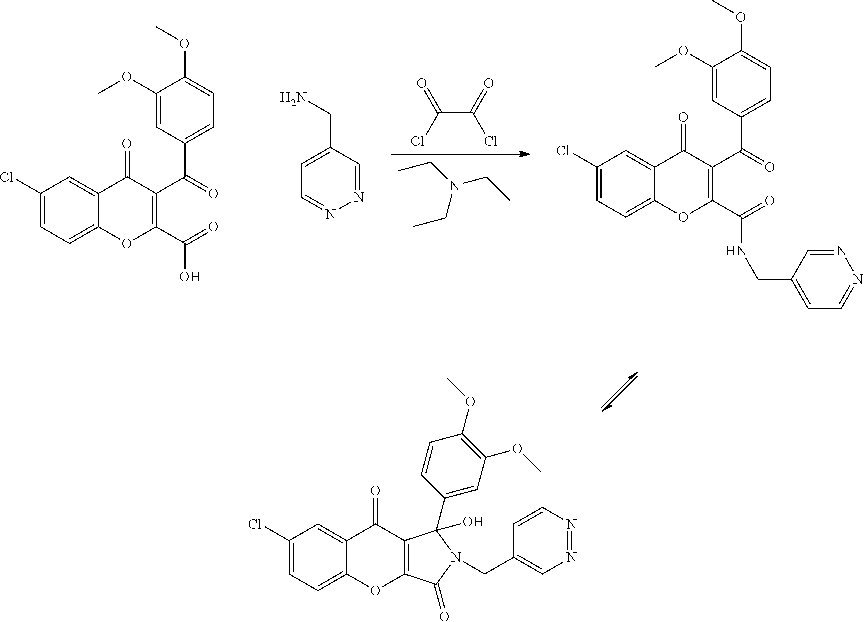

To the solution of 6-chloro-3-(3,4-dimethoxybenzoyl)-4-oxo-4H-chromene-2-carboxylic acid (20 mg, 0.051 mmol) in DCM (4 mL) under ice-bath was added oxalyl dichloride (9.79 mg, 0.077 mmol) and two drops of DMF with stirring for one hour, then continued stirring for three hours at room temperature. The DCM in the reaction mixture was removed by flushing with nitrogen gas. The residue was dissolved in DCM (2 ml). To the solution was added triethylamine (35.9 μl, 0.257 mmol), pyridazin-4-ylmethanamine (7.30 mg, 0.067 mmol) in DMF (2.0 ml). The reaction mixture was stirred overnight. The crude product was purified by normal phase column (50 to 100% EA in Hex and 0 to 20% MeOH in EA). Yield=8.1%. ¹H NMR (400 MHz, CD₂Cl₂) δ 9.09 (d, J=5.4 Hz, 2H), 8.18-8.14 (i, 1H), 7.78 (dd, J=8.9, 2.6 Hz, 1H), 7.72 (dd, J=3.7, 1.6 Hz, 1H), 7.59 (s, 1H), 7.40-7.32 (m, 2H), 6.87 (d, J=9.2 Hz, 1H), 4.60 (d, J=6.4 Hz, 2H), 3.91 (dd, J=3.6, 1.4 Hz, 6H). 3:2 ratio. Major to minor. Only major NMR shown. LCMS RT (Method 1)=2.604 min, m/z=480.1 [M+H⁺]; HRMIS (ESI) m/z calcd for C₂₄H₁₉ClN₃O₆⁺[M+H⁺ ] 480.0962, found 480.0972.

### Example 69: N8279 was a Potent Agonist of GHSR1a-Mediated Gαq Signaling

Initial SAR screening suggested that the N8279 precursors N1965 and N6164 may exhibit Gα_{q/11} bias (FIG. 5B and Table 1). To confirm this with the active congener, N8279 (FIGS. 1A, 1B, 5C, and Table 2), concentration-response (C/R) analyses were performed in cells stably expressing the GHSR1a and an iCa²⁺ reporter. The results showed that N8279 was nearly an order of magnitude (8.9-fold) more potent than the endogenous ligand ghrelin and was a full agonist (FIG. 1F). N8279 was 3.4- and 5.3-fold less potent than the high affinity, unbiased small molecule agonists L585 and MK-0677, respectively (FIG. 1F). Ghrelin was relatively weak at stimulating iCa²⁺ compared to its GHSR1a binding affinity (FIGS. 1E and 1F). Conversely, the iCa²⁺ EC₅₀ of N8279 was 41-fold more potent than its GHSR1a binding IC₅₀ (FIGS. 1E and 1F), suggesting possible allosteric activity. Only ghrelin and N8279 each had a Hill slope (h)>1, suggesting that >2 molecules or GHSR1a binding sites were required for these ligands to elicit full efficacy in this cell system (FIG. 1F).

The iCa²⁺ evoked by EC₅₀ N8279 (FIG. 7B), ghrelin (FIG. 7C), or L585 (FIG. 7D), was competitively inhibited by the GHSR1a antagonists YIL781 and JMV2959, supporting GHSR1a-dependent effects. For each agonist, YIL781 was the more potent inhibitor (FIGS. 7B-7D). To determine if N8279 elicited GHSR1a-mediated iCa²⁺ through Gα_{q/11} specifically, iCa²⁺ was tested in Gα_{q/11} knockout (KO) and wild-type (WT) cells. Resulting data confirmed that 10 μM ghrelin-, L585-, and N8279-induced iCa²⁺ was abolished (FIG. 7E).

The effect of N8279 on ghrelin-induced iCa²⁺ signaling was next evaluated to test for ago-allosteric activity. Parenthetically, ago-allosteric agonists interacted with topographically-distinct receptor sites (allosteric) from the endogenous ligand (orthosteric), elicit agonist behavior on their own, and cooperatively act as a positive (PAM), negative (NAM), or silent (SAM) modulator of orthosteric ligand affinity, potency, and/or efficacy. N8279 displayed intrinsic GHSR1a agonism on its own (FIG. 1G, left dashes). At 10 μM, N8279 produced an ˜3-fold increase in ghrelin's potency (FIG. 1G). N8279 additively increased˜EC₂₀ ghrelin (100 nM) efficacy in a concentration-dependent manner (FIG. 1G, right dashes/upward arrow) and marginally increased the ghrelin Eₘₐₓ (FIG. 1G, upward arrow), supporting weak ago-PAM activity. To assess reciprocal cooperativity, iCa²⁺ was tested upon concomitant treatment of EC25 MK-0677 or EC₅₀ ghrelin and a N8279 C/R. In the presence of these orthosteric agonists, N8279 was equi-potent relative to N8279 alone; however, the h of N8279 alone (h>1) was reduced to unity (h=1) (FIG. 7F). These findings indicated that N8279 evoked a complete and potent signaling response despite simultaneous occupancy of the GHSR1a orthosteric binding pocket.

To model how N8279 could co-occupy the monomeric GHSR1a with ghrelin, molecular docking was employed with an NMR-based homology model of the ghrelin-bound GHSR1a. Concomitant N8279 docking to ghrelin (1-17)-bound GHSR1a suggested that the propylamine moiety of N8279 could form a strong, ionic bond with a negatively charged ECL2 (Asp191) (FIG. 7G). In this pose, N8279 was found to bind the GHSR1a atop ghrelin, enabling ghrelin's N-terminus to insert into the deep orthosteric binding pocket and interact with Glu124 in TMIII, the proposed agonist-induced activation mechanism of GHSR1a. Thus, N8279 may exhibit state-dependent, allosteric GHSR1a binding by anchoring to ECL2.

### Example 70. N8279 Biased GHSR1a Towards Gαq Coupling Over Other Gα Subunits

To evaluate the effect of N8279 on Gα proximal to the GHSR1a, parallel NanoBiT- and bioluminescence resonance energy transfer (BRET, TRUPATH)-based heterotrimeric G protein dissociation approaches were employed. In both assays, N8279 was a full agonist for Gα_{q} activation with a potency comparable to that of iCa²⁺ (FIGS. 1F, 1H, 7H). In NanoBiT-Gα_{q} assays, N8279 and ghrelin potencies were statistically equivalent (FIG. 7H). In BRET-Gα_{q} assays, N8279 was 6.1- and 1.7-fold less potent than ghrelin and L585, respectively (FIG. 1H). In contrast to iCa²⁺ assays (FIG. 1F), the h<1 for each agonist. Notably, ghrelin was ˜30-50-fold more potent at activating Gα_{q} proximally than eliciting downstream iCa²⁺ (FIGS. 1F, 1H, 7H). These findings suggested that in these cell systems, low ghrelin concentrations were sufficient to dissociate the heterotrimeric Gα_{q} complex from the GHSR1a, whereas high ghrelin concentrations were required to fully engage downstream signaling. Collectively, these results demonstrated that N8279 was a potent agonist of Gαq signaling at the GHSR1a.

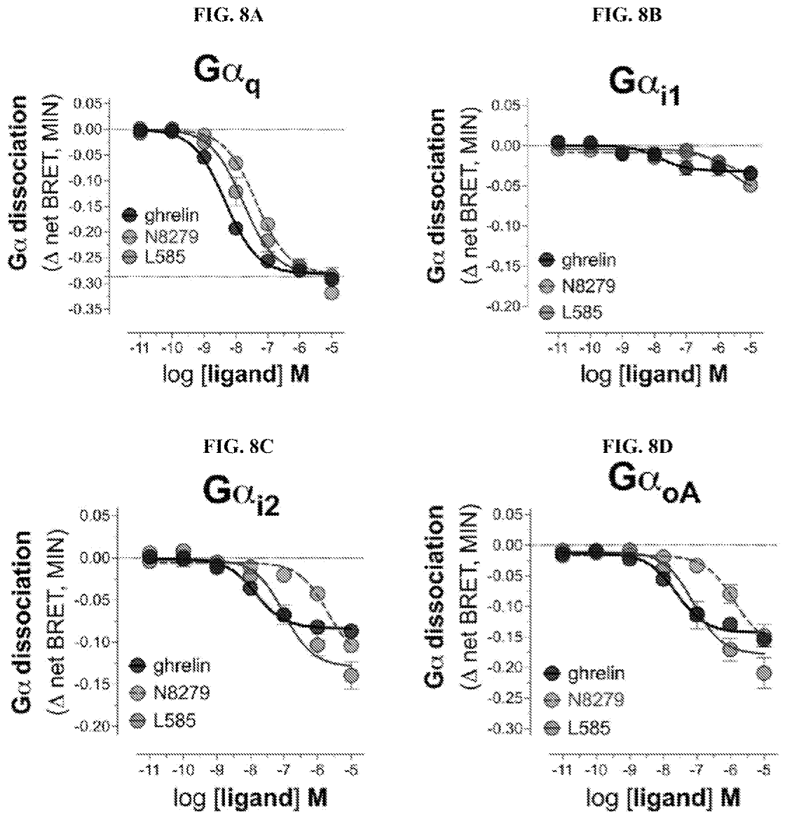

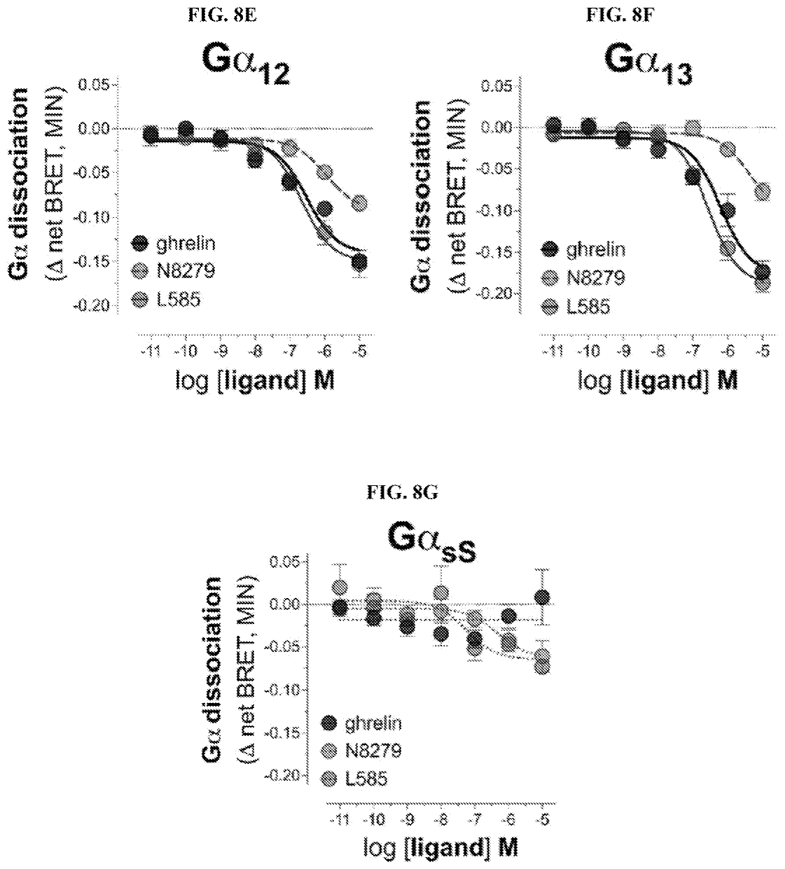

In an independent set of experiments, N8279 signaling was evaluated through other G proteins to compare to Gα_{q}. Gα subunits that are both expressed highly in midbrain DAergic neurons and reported to exhibit GHSR1a coupling, including Gα_{sS} (Gαₛ), Gαᵢ₁, Gαᵢ₂, Gα_{oA}, Gα₁₂, and Gα₁₃ were selectively tested. Relative to Gα_{q}, ghrelin potency was statistically equivalent for each Gα_{i/o} and was reduced moderately for Gα₁₂ and Gα₁₃ (FIGS. 1I, 1J, 8A-8G). Ghrelin did not activate Gαₛ. L585 displayed a similar profile, except that it had reduced potency at Gαᵢ₁ and only a statistical trend for reduced potency at Gα₁₂. In contrast, N8279 potency was significantly reduced to concentrations>1 μM for every Gα (FIGS. 1I, 1J, 8A-8G); thereby, supporting bias towards Gα_{q} coupling. Although N8279 and L585 showed statistically equi-potent activation of Gαₛ, their max efficacies (10 μM) at Gαₛ were markedly reduced (FIGS. 1K, 8A-8G). Moreover, N8279 max efficacy was significantly reduced at Gα_{12/13} compared to ghrelin and L585, as well as at Gαᵢ₂ and Gα_{oA} compared to L585 (FIGS. 1K, 8A-8G). For each ligand, max efficacy was reduced at every Gα relative to their respective effect on Gαq (FIGS. 1K, 8A-8G).

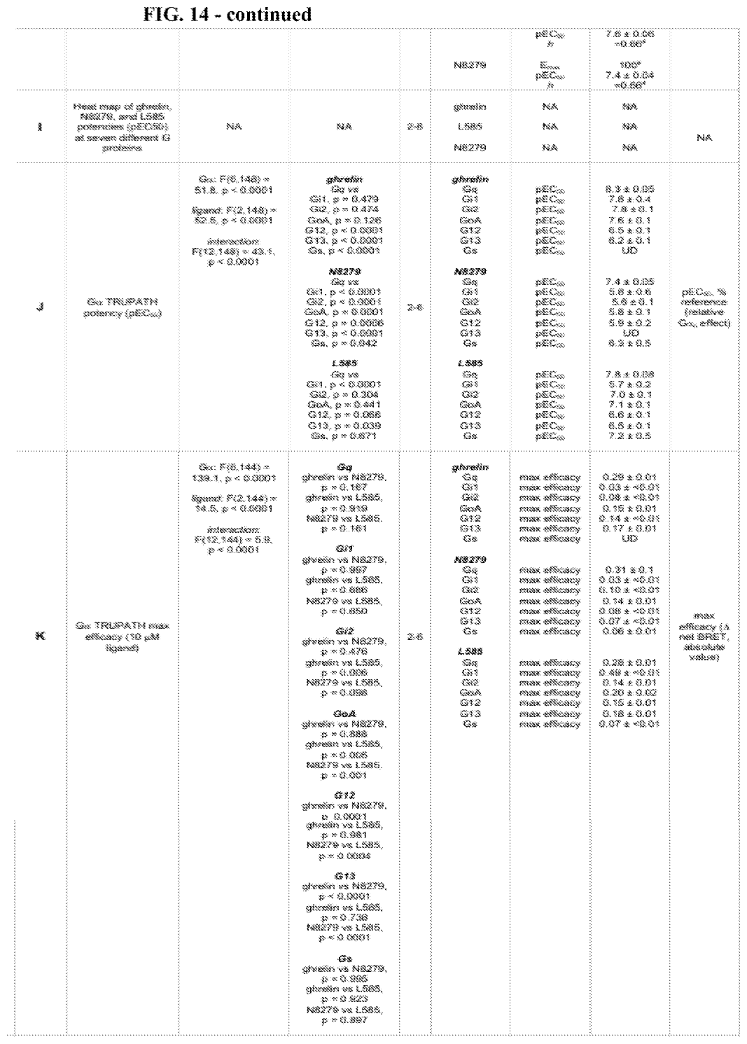

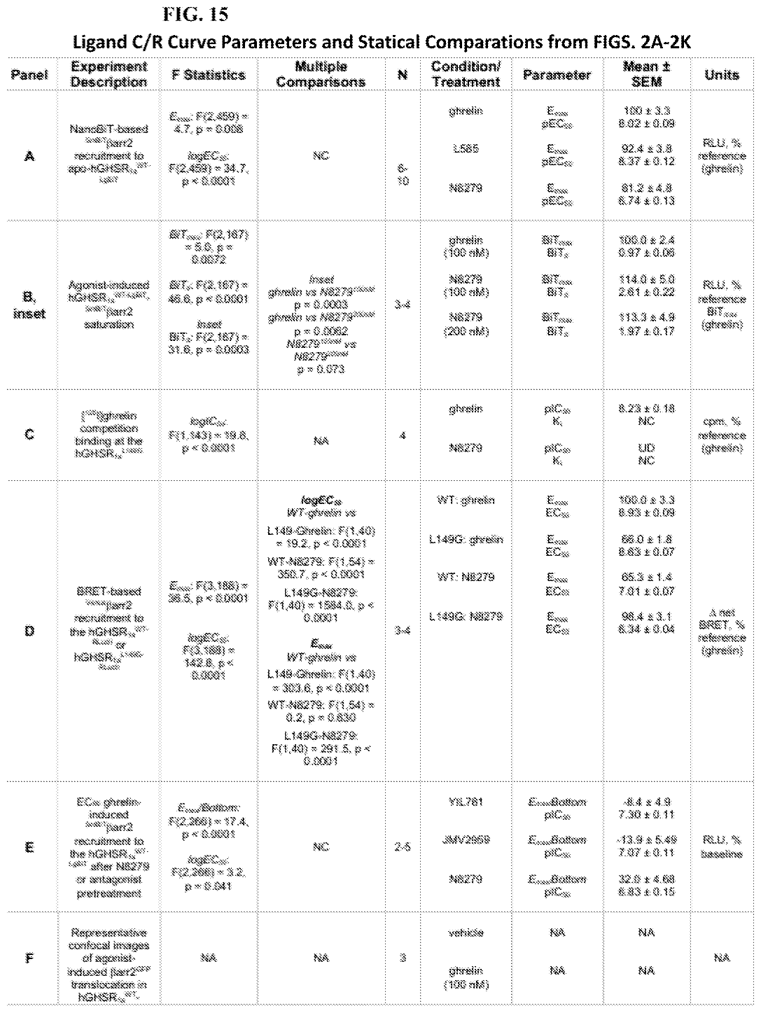

Together, the data in FIGS. 1A-1K support that N8279 was a potent GHSR1a agonist with bias towards Gα_{q}. All pharmacological results (IC₅₀, Kᵢ, log EC₅₀, Eₘₐₓ, h±SEM) and statistical comparisons for FIGS. 1A-1K are shown in FIG. 14.

### Example 71. N8279 Recruited β-Arr2 to GHSR1a in a Weaker Manner than Ghrelin

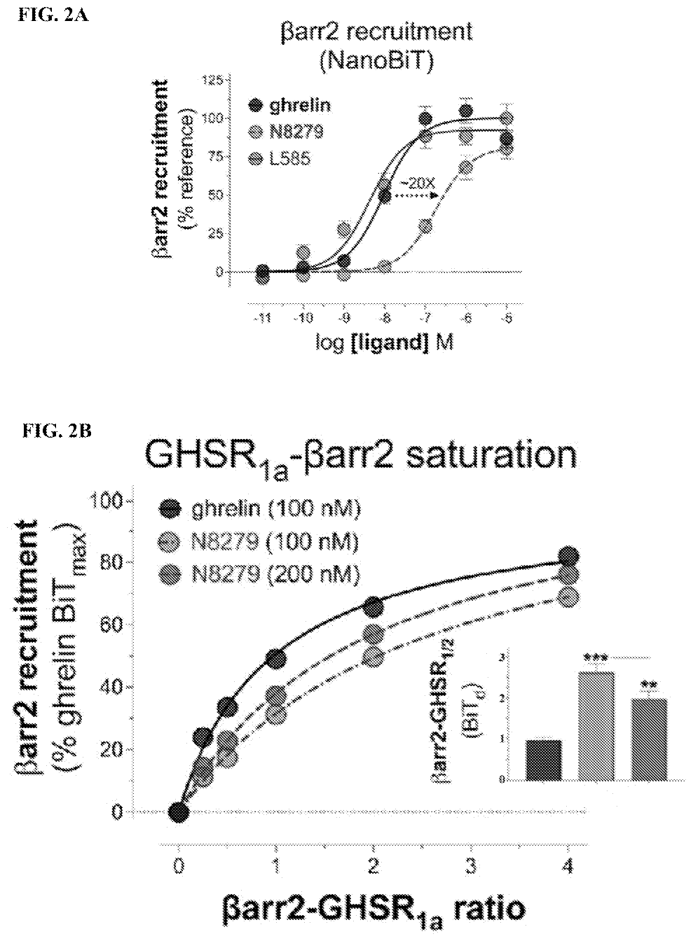

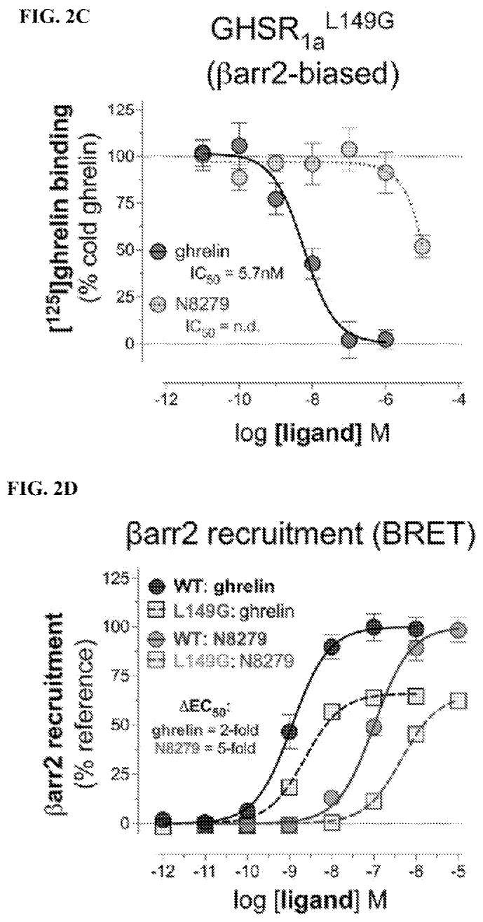

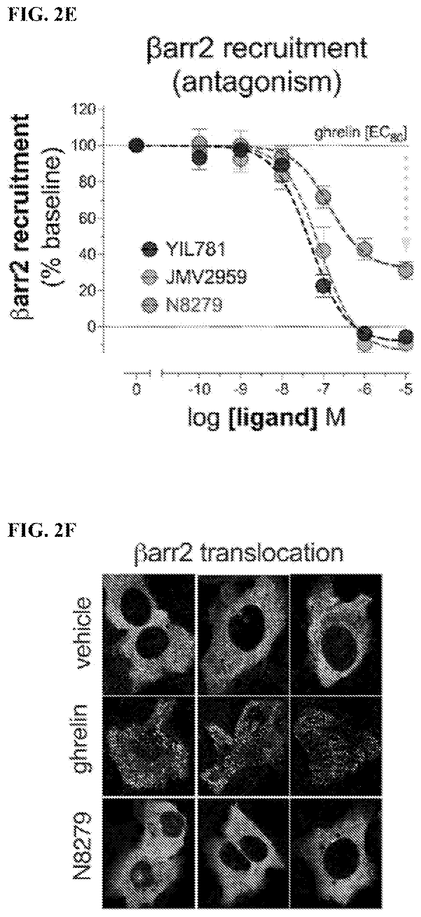

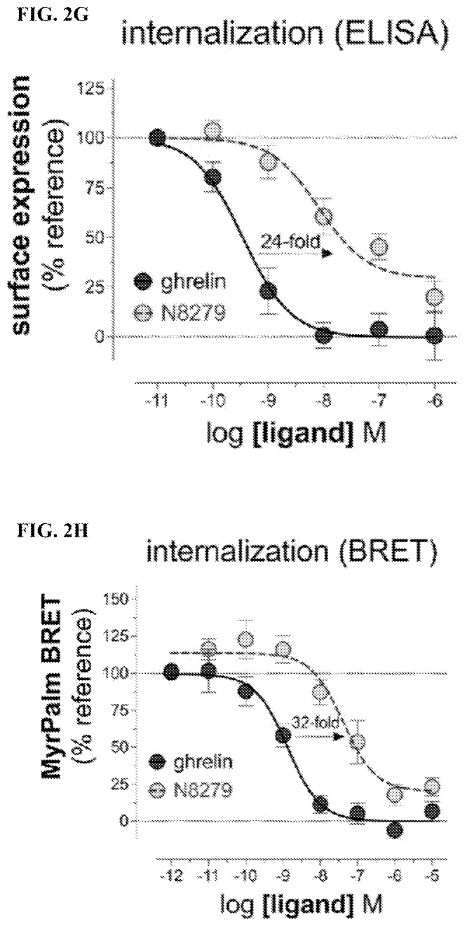

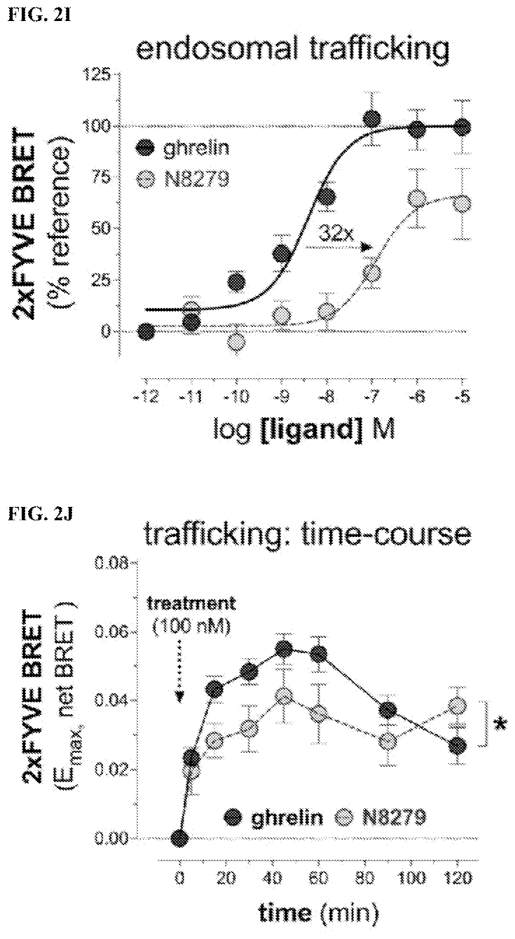

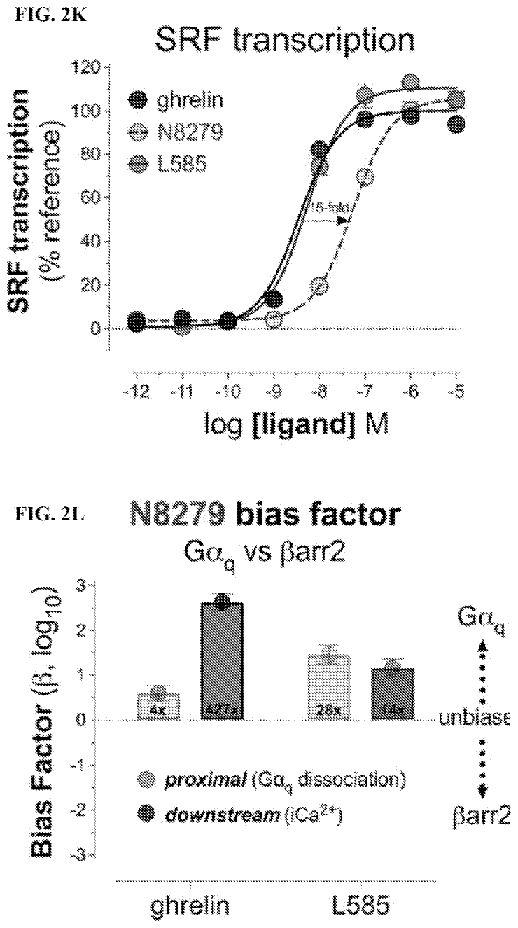

Having established that N8279 was a potent activator Gαq signaling, its effect on GHSR1a-mediated β-arr recruitment was next assessed using a NanoBiT-based approach. In brief, cells expressing a fixed ratio of GHSR1a^{L}g^{BiT} and ^{smBiT}β-arr2 were treated with ghrelin, L585, or N8279. Here, N8279 was ˜20-fold less potent than ghrelin and it approached full agonism (FIG. 2A). Conversely, L585 recruited β-arr2 with moderately higher potency and equivalent efficacy to ghrelin (FIG. 2A) and comparatively, N8279 was ˜43-fold less potent than L585 in this assay. Thus, N8279 was a weaker agonist of GHSR1a-β-arr2 recruitment than ghrelin and L585, supporting that it exhibited functional selectivity towards Gαq over β-arr coupling (FIGS. 1A-1K).

Next, cells expressing a variable ratio of GHSR1a^{L}g^{BiT} and ^{SmBiT}β-arr2 were treated with ghrelin (100 nM) or ˜EC₈₀ N8279 for Gα signaling (100-200 nM, FIGS. 1F, 1H, 7H). These analyses revealed that that the relative affinity (BiTd) of β-arr2 for the GHSR1a was 22.5-fold weaker in N8279-treated cells than in ghrelin-treated cells (FIG. 2B, inset). Furthermore, in competitive binding studies with the ICL2 mutant GHSR1a^{L149G} (FIG. 2C), a 0-arr2-biased receptor, the ability of N8279 to displace [¹²⁵I]ghrelin was diminished and shifted rightward by an order of magnitude (IC₅₀>10 μM) relative to GHSR1a^{WT} (FIG. 1E). In contrast, the IC₅₀ of unlabeled ghrelin for the GHSR1a^{L149G} was reduced by only ˜2-fold compared to the GHSR1a^{WT} (FIGS. 1E, 2C). A follow-up, BRET-based GHSR1a^{L149G-RLucl_Venus}β-arr2 recruitment assay showed distinct properties between ghrelin and N8279 at the β-arr-biased GHSR1a^{L149G}. While the GHSR1a^{L149G} reduced the Eₘₐₓ of ghrelin and N8279 to similar extents (˜35%), the N8279 potency was reduced by ˜5-fold, whereas the ghrelin potency was reduced by only ˜2-fold relative to the GHSR1a^{WT} (FIG. 2D). Inter-assay comparisons revealed that N8279 was ˜100 fold less potent than ghrelin in BRET-based GHSR1a^{WT}-β-arr2 recruitment assays (FIG. 2D), but only ˜20-fold less potent in NanoBiT-based measurements (FIG. 2A). These findings together suggested that N8279 stabilized GHSR1a conformations that disfavor GHSR1a-β-arr2 coupling relative to ghrelin, and reciprocally, that GHSR1a conformations preferentially supporting β-arr2 coupling (GHSR1a^{L149G}) diminished N8279-GHSR1a interaction(s).

Next, it was assessed if N8279 could behave functionally as a β-arr2 antagonist in the presence ghrelin. Cells expressing GHSR1a^{L}g^{BiT} and ^{smBiT}β-arr2 were pretreated with increasing concentrations of the antagonists YIL781 or JMV2959, or N8279, followed by EC₈₀ ghrelin. N8279 inhibited ghrelin-induced β-arr2 recruitment significantly, but incompletely, in a concentration-dependent manner and was 1.7 and 2.9-fold less potent than JMV2959 and YIL781 (FIG. 2E). These data suggested that N8279 stabilized GHSR1a conformation(s) that weaken β-arr2 coupling in both apo-(FIGS. 2A, 2B, 2D) and ghrelin-bound (FIG. 2E) receptor states.

While the GHSR1a^{L149G} reduced Eₘₐₓ similarly for both ghrelin and N8279 relative to GHSR1aWT (˜35%), N8279-induced β-arr2 recruitment potency to the GHSR1a^{L149G} was reduced by ˜5-fold, whereas ghrelin potency was decreased by merely 2-fold (FIG. 2D). Relative to ghrelin, N8279 potency reduction at the WT GHSR1a in BRET-based β-arr2 recruitment assays were greater in magnitude than in NanoBiT-biased GHSR1a^{WT}-β-arr2 recruitment assays (FIG. 2A)—i.e., ˜100-fold vs 20-fold, respectively. This distinction may be due to assay-specific differences in GHSR1a-β-arr2 expression ratios (NanoBiT=1:1 vs BRET=1:15) and/or biosensor interaction kinetics/measurement durations (NanoBiT=5 minutes vs BRET=60 minutes). Together, these findings suggested that, relative to ghrelin, N8279 binding stabilized GHSR1a conformations that attenuated GHSR1a-β-arr2 coupling and reciprocally, that GHSR1a conformations preferentially supporting β-arr2 coupling (GHSR1a^{L149G}) diminished N8279-GHSR1a interaction(s).

To further characterize N8279 modulation of β-arr2 recruitment to the ghrelin-bound GHSR1a, cells expressing GHSR1a^{L}g^{BiT} and ^{SmBiT}β-arr2 were pretreated with increasing concentrations of YIL781, JMV2959, or N8279 followed by EC₈₀ ghrelin. N8279 inhibited ghrelin-induced β-arr2 recruitment to the GHSR1a in a concentration-dependent manner, albeit incompletely and with a potency 1.7-2.9-fold less than JMV2959 and YIL781, respectively (FIG. 2E). Thus, N8279 pretreatment partially antagonized β-arr2 recruitment to the ghrelin-bound GHSR1a, suggesting that it stabilized GHSR1a conformation(s) that do not favor β-arr2 coupling in both apo- (FIGS. 2A-C) and ghrelin-bound (FIG. 2E) receptor states.

### Example 72. N8279 Reduced β-Arr-Dependent Cellular Responses Relative to Ghrelin

Qualitative microscopy of U2OS cells expressing the GHSR1a and β-arr2^{GFP} showed minimal response to 100 nM N8279 and displayed diffusely distributed cytosolic β-arr2^{GFP} Similar to vehicle-treated cells (FIG. 2F). Conversely, 100 nM ghrelin-treated cells exhibited marked accumulation of cytosolic puncta, indicative of robust β-arr2^{GFP} translocation and GHSR1a endocytosis/trafficking (FIG. 1F). Though ghrelin produced a robust response, the relatively weak response produced by N8279 in these experiments may reflect differences in engagement with early (e.g., GPCR kinases) or late molecular mediators of receptor endocytosis and/or endosomal trafficking. Thus, ligand-induced GHSR1a endocytosis was evaluated using three independent methods. First, in a quantitative, cell surface enzyme-linked immunosorbent assay (ELISA) approach, both ghrelin and N8279 stimulated GHSR1a internalization in a concentration-dependent manner (FIG. 2G). However, N8279 internalization potency was reduced by 24-fold and efficacy was reduced modestly (˜30%) relative to ghrelin (FIG. 2G). Second, a bystander BRET (bBRET)-based plasma membrane sensor, MyrPalm^{Venus38}, was employed and it was found that N8279-induced GHSR1a internalization (over 60 minutes) potency was reduced by 32-fold and efficacy was attenuated by ˜20% relative to ghrelin (FIG. 2H). Last, the bBRET-based sensor for early endosomes, 2×FYVEVenus, showed that N8279-induced GHSR1a endosomal transit was 32-fold less potent and less efficacious (˜35%) than ghrelin over 60-minute post-treatment (FIG. 2I). Temporal analyses revealed that N8279-stimulated GHSR1a endosomal trafficking occurred on a time-course similar to ghrelin, albeit with reduced efficacy across the entire 120-minute measurement (FIG. 2J).

β-arr2 is required for GHSR1a-mediated RhoA GTPase/ROCK signaling, leading to transcriptional activation and cytoskeletal rearrangement by induction of actin polymerization. To test if N8279 affected these processes, the RhoA-dependent transcriptional reporter, SRF-RE, was utilized. Here, N8279 was a full agonist with mildly increased maximal efficacy relative to ghrelin (FIG. 2K). However, N8279 potency was reduced ˜10-15-fold relative to L585 and ghrelin, respectively. The enhanced efficacy of N8279 in these assays compared to FIGS. 2G-2J could be a time-dependent effect, in part, due to the 6-hour treatment duration and/or the partial contribution(s) of Gα_{12/13} or MAPK/ERK signaling to SRF transcription.

To quantitatively assess N8279 bias between the Gα_{q} and β-arr2 (FIG. 2A) pathways, the intrinsic relative activities (RAi) model was used. Relative to ghrelin, N8279 had a proximal (Gα_{q} dissociation, FIG. 1H) bias factor (β) of 0.59 (˜4-fold) and a downstream (iCa²⁺, FIG. 1F) bias factor of 2.63 (˜427-fold) relative to β-arr2 recruitment (NanoBiT, FIG. 2A) (FIG. 2L, left). Relative to L585, N8279 had a proximal bias factor of 1.45 (˜28-fold) and a downstream bias factor of 1.15 (˜14-fold) (FIG. 2L, right). Notably, assay-standardized bias calculations (i.e., Gαq-β-arr2 BRET vs Gαq-β-arr2 NanoBiT) using ghrelin as a reference ligand showed that N8279 had a proximal bias factor of 1.16 (˜14-fold) and 1.36 (˜23-fold) when employing paired BRET-Gαq/BRET-β-arr2 or NanoBiT-Gαq/NanoBiT-β-arr2 assays, respectively (FIG. 9). Ultimately, these analyses together supported that N8279 was a G protein-biased agonist of both proximal and downstream GHSR1a-Gαq signaling.

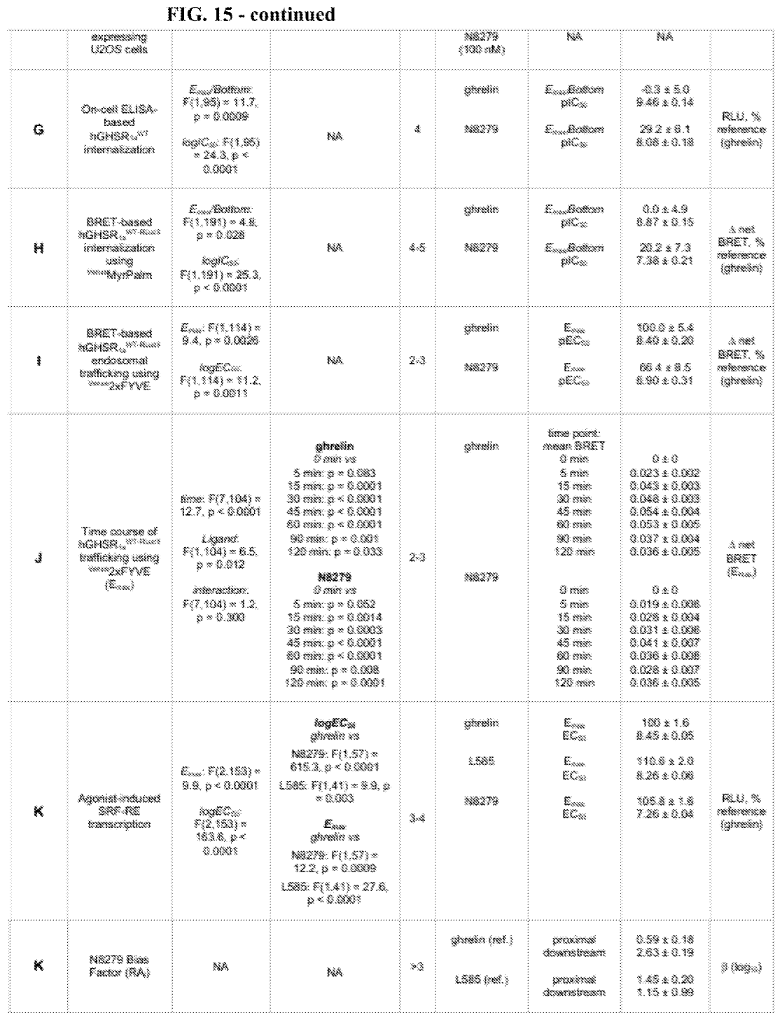

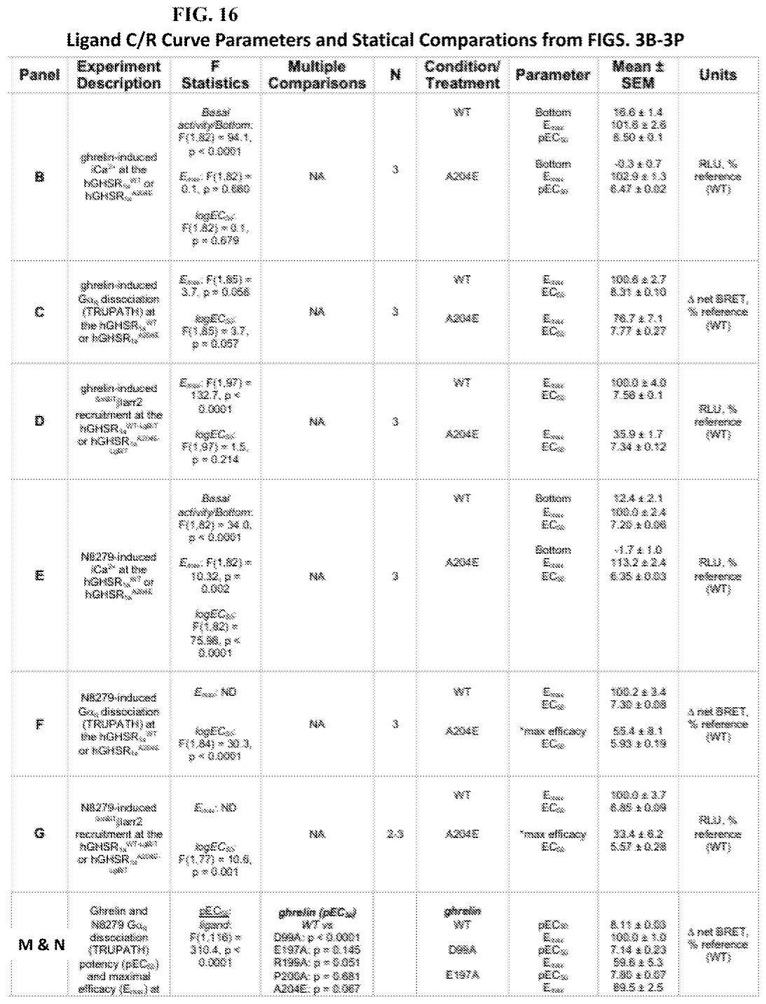

Collectively, the data in FIGS. 2A-2J supported that N8279 was a weak agonist of GHSR1a-mediated, β-arr-dependent signaling relative to ghrelin and thus, N8279 G protein-biased GHSR1a agonist. All pharmacological results and statistical comparisons for FIGS. 2A-2J are shown in FIG. 15.

### Example 73. GHSR1a Mutagenesis and Molecular Docking Suggested an ECD-Dependent, Extended Binding Mode of N8279

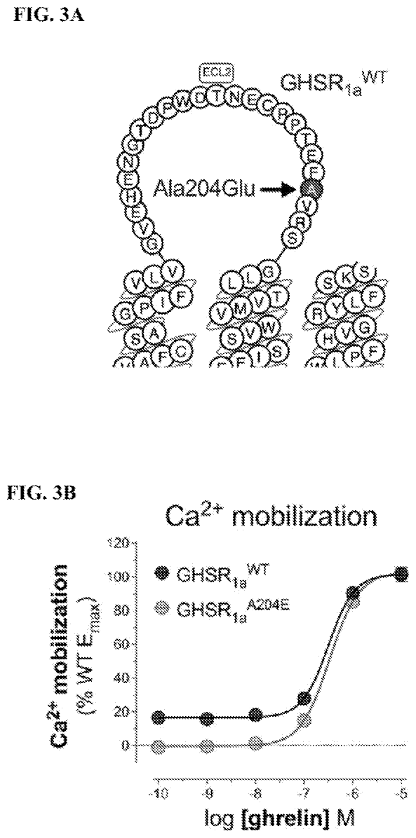

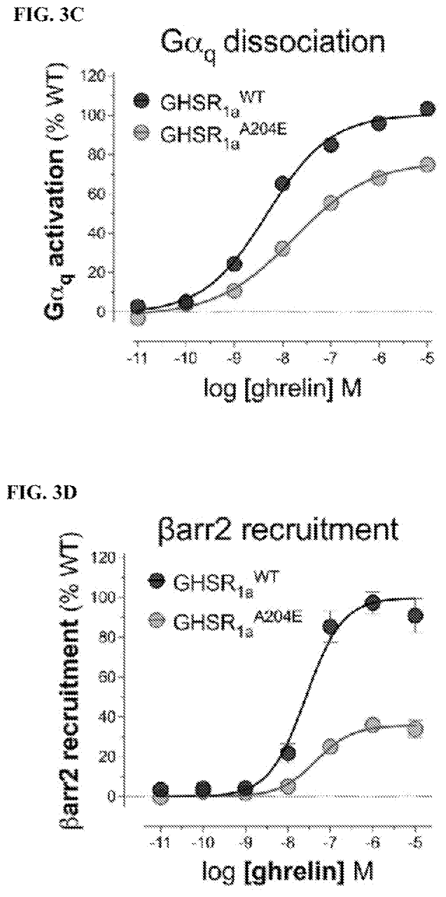

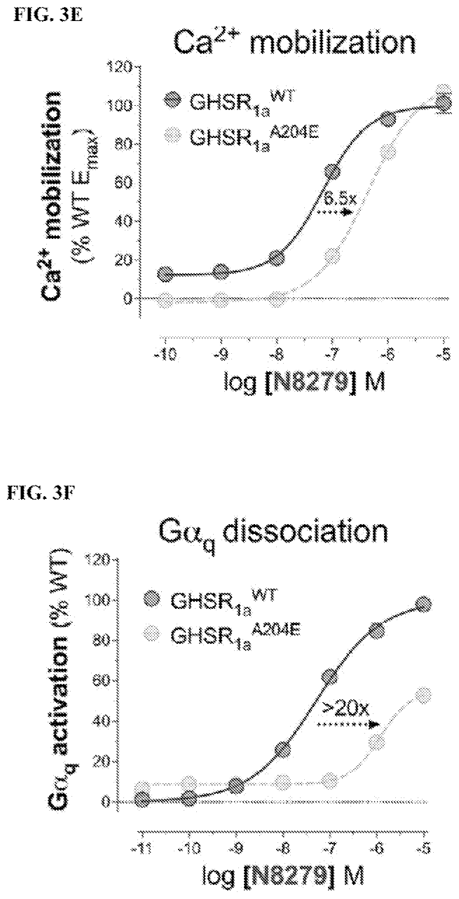

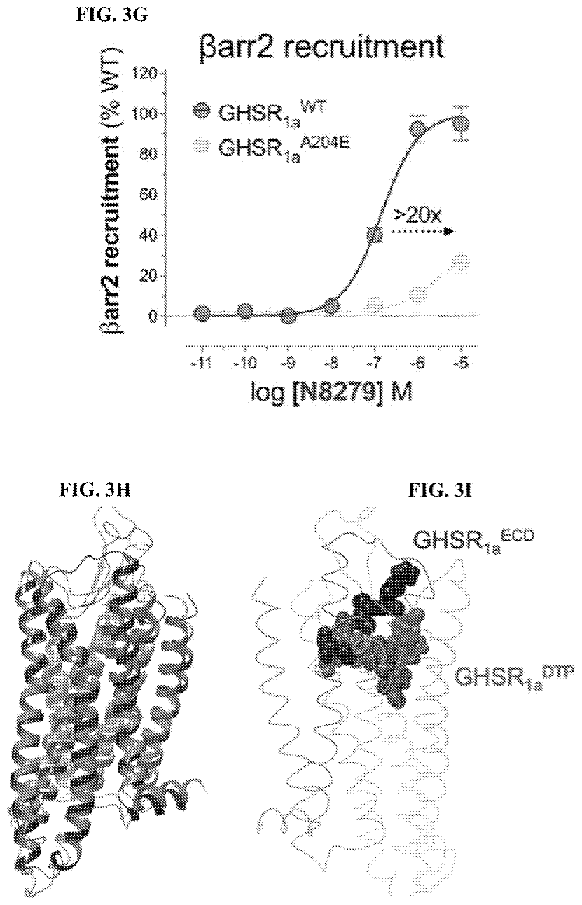

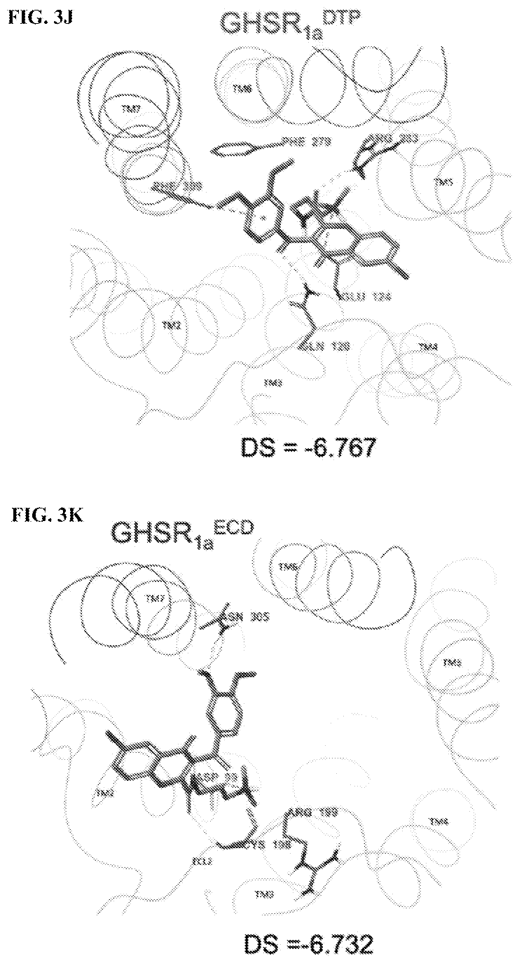

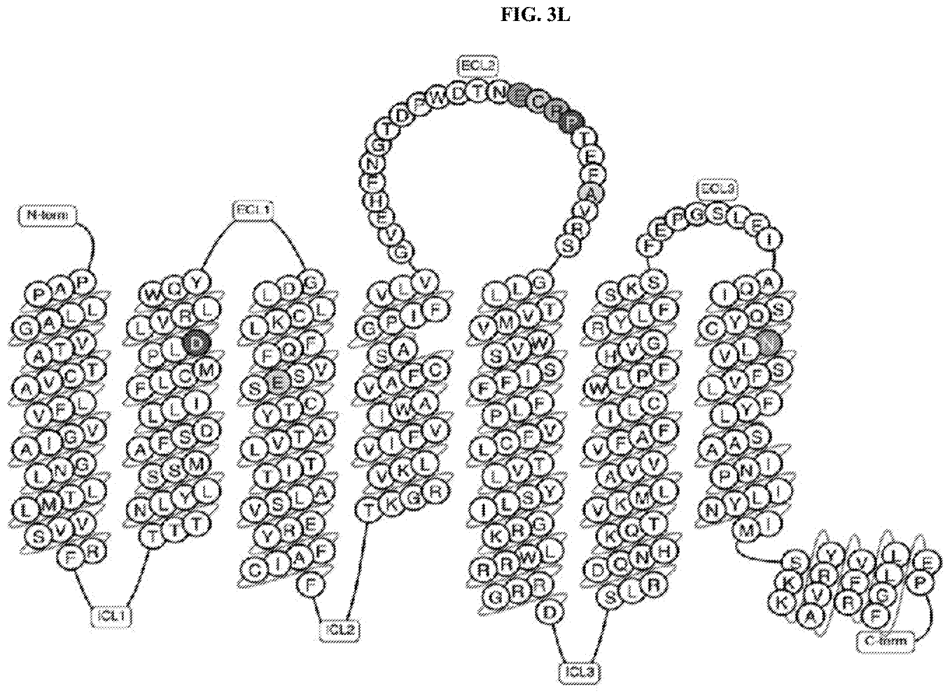

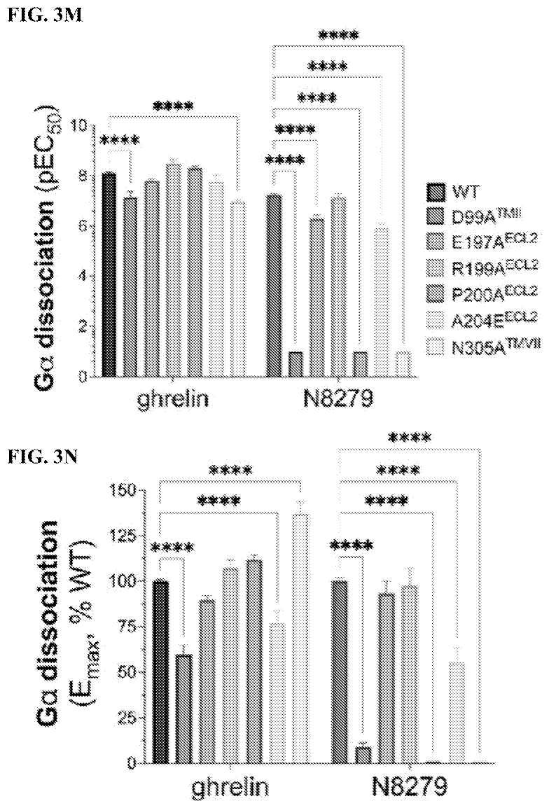

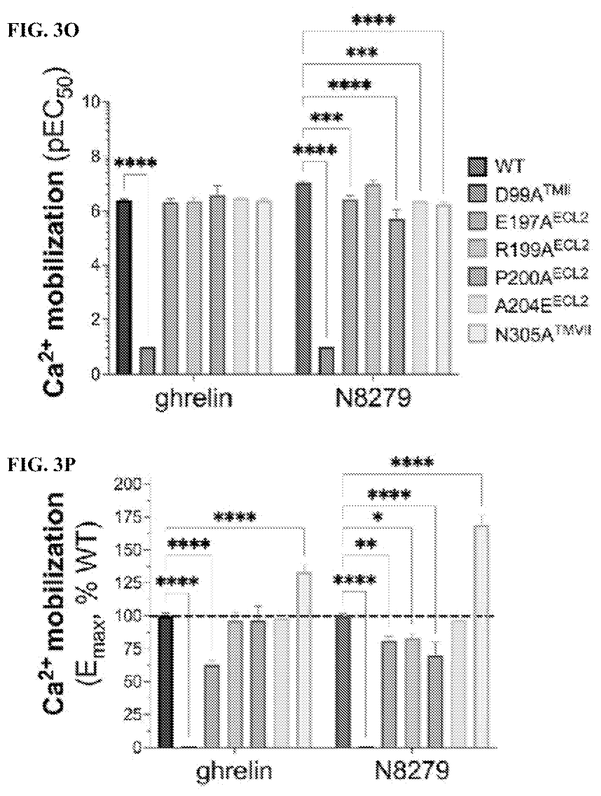

Next it was evaluated whether determinants outside the orthosteric binding pocket are required for N8279 signaling by first using a naturally-occurring variant, GHSR1a^{A204E} (FIG. 3A). Substitution of glutamic acid at this ECL2 site abolished constitutive activity and caused short stature in humans. However, the GHSR1a^{A204E} mutation did not appreciably affect ghrelin binding or ghrelin-induced Gαq signaling supporting that it lies outside the orthosteric binding pocket.

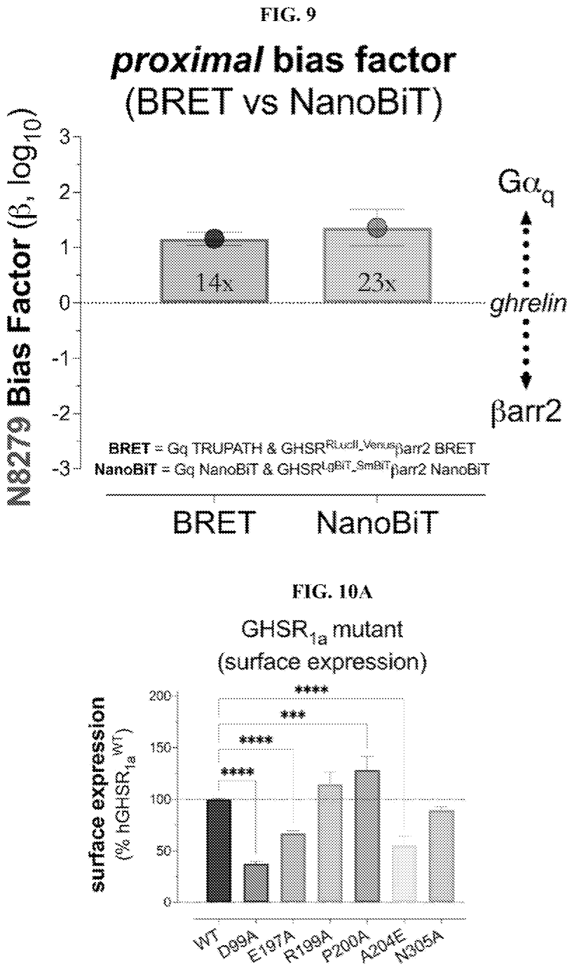

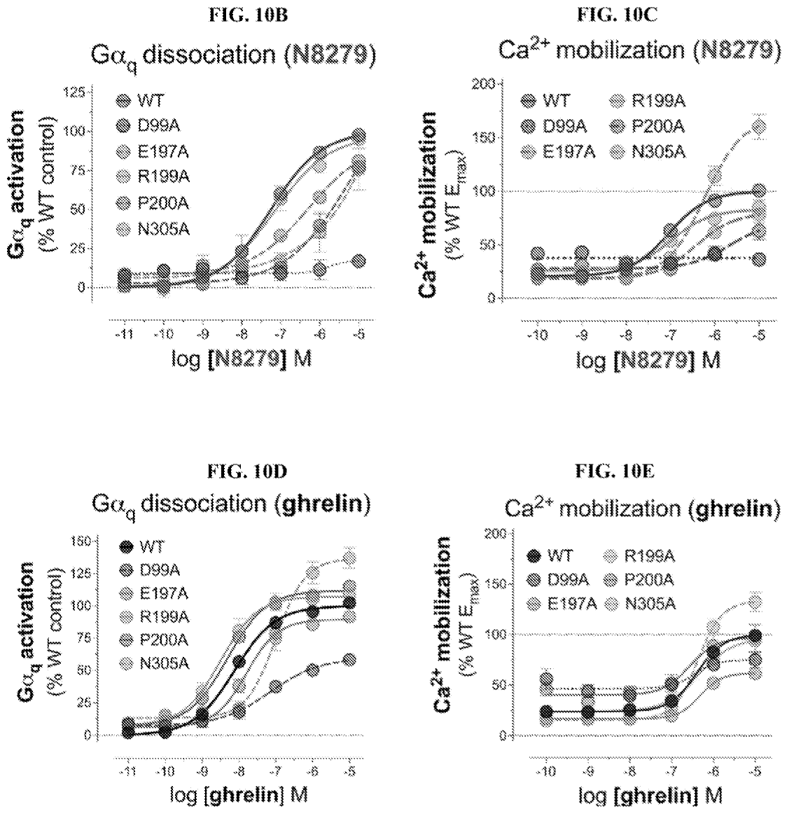

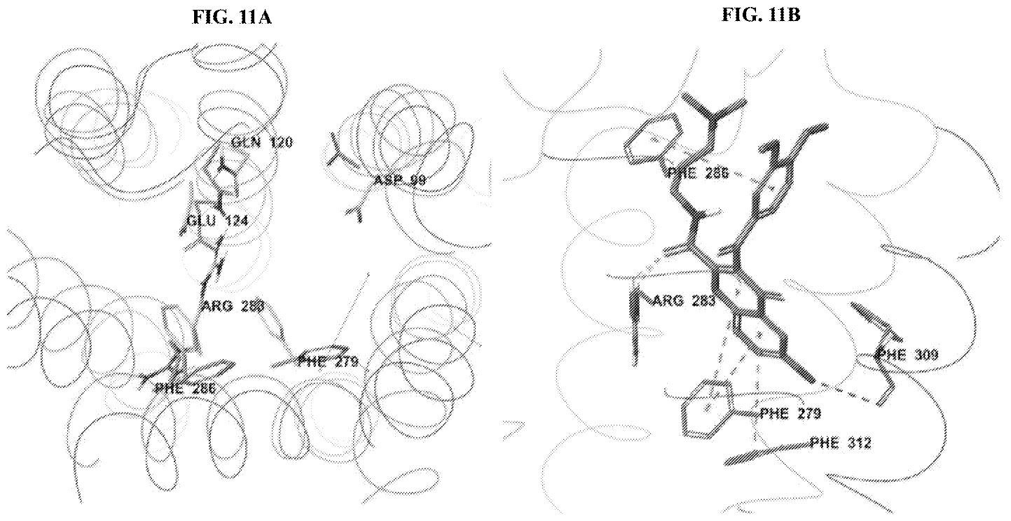

GHSR1a^{A204E} showed no basal iCa²⁺ activity (FIGS. 3B-3C), surface expression was reduced by ˜50% (FIG. 10A), it had minimal-to-no effect on ghrelin-stimulated Gα_{q} dissociation and iCa²⁺ (FIGS. 3B-3C), and ghrelin-stimulated D-arr2 recruitment efficacy, but not potency, was reduced (FIG. 3D). In contrast, N8279-induced iCa²⁺ potency was reduced by 6.5-fold while full agonism was retained (FIG. 3E). Furthermore, N8279-induced Gα_{q} dissociation signaling was reduced dramatically such that the C/R curve did not saturate, supporting that the N8279 potency is blunted by >20-fold and the maximal efficacy decreased by ˜45% (FIG. 3F). The effect magnitude discrepancy between measurements of iCa²⁺ and Gα dissociation likely reflected signal amplification differences between the assays. N8279-induced β-arr2 recruitment potency at the GHSR1a^{A204E} was similarly diminished (>20-fold) and did not reach saturation, with a maximal efficacy comparable to ghrelin (˜35%, FIG. 3G). Thus, relative to ghrelin, N8279 signaling required distinct ECL2 sites and/or extracellular domain (ECD)-dependent conformational states.

Next, the NMR-based homology model of the ghrelin-bound GHSR1a (FIG. 7G) was used to simulate N8279-GHSR1a binding. The antagonist-bound GHSR1a crystal structure (FIGS. 3H, 11A-11B) better modeled the inactive GHSR1a conformation. Docking N8279 with ghrelin removed disclosed two potential binding modes for N8279 within the apo-GHSR1a (FIG. 3I). Both modes displayed strong ionic interactions between N8279's propylamine moiety and specific acidic (negatively charged) GHSR1a residues. In one mode (FIG. 3J, red), N8279's terminal tertiary amine group forms a salt bridge with the conserved TMIII residue Glu124, located within the deep transmembrane pocket (GHSR1a^{DTP}). In the second mode (FIG. 3K, blue), N8279's propylamine moiety formed a salt bridge with Asp99 toward the top of TMII, enabling an extended binding mode into the ECD (GHSR1a^{ECD}) or extracellular vestibule, including the extracellular end of TMVII and ECL2. Notably, the superficial residue Asp99 was too distant from Glu124 (12.7 Å) for N8279 to interact with both sites simultaneously (FIG. 3I). Thus, both docking models suggested that N8279 binds the GHSR1a via ‘ionic locks’ with spatially distinct anchor residues. In the GHSR1a^{ECD}, N8279's methoxy-aromatic and amide moieties form hydrogen bonds with, or adjacent to, potential allosteric sites, including Asn305 on TMVII and Glu197, Arg199, or Pro200 in ECL2 (FIG. 3K). Here, N8279's amide group formed a H bond with Cys198, a highly conserved GPCR residue that constrains ECL2 flexibility (FIG. 3K). Notably, N8279 had comparable docking scores within both potential binding pockets: GHSR1aECD (˜6.732) and GHSR1aDTP (−6.767) (FIGS. 3J, 3K).

To test the model, point mutations were made to predict N8279-GHSR1a interaction sites or residue clusters (FIG. 3L). Given the evidence for critical ECL2-dependent contributions to N8279 signaling (FIGS. 3E-3G), as well as the absolute requirement of the GHSR1aDTP anchor residue, Glu124, for GHSR1a function/activation the GHSR1a^{ECD_}N8279 docking model was prioritized for mutagenesis. Alanine substitution to the putative anchor residue Asp99 (FIG. 3K, blue) abolished N8279 signaling (FIGS. 3M-3P, 10B, 10C). However, the surface expression of this was reduced markedly (FIG. 10A). Mutations were not made to Cys198ECL2 (FIG. 3K, light blue) because it precluded GPCR stability and ligand binding. Instead, alanine substitutions were made to three adjacent, putative allosteric and/or structurally integral ECL2 residues: Glu197, Arg199, and Pro200 (FIG. 3K, blue). Asn305 was located at the extracellular end of TMVII of GHSR1a (FIG. 3K, blue) and thus, was considered within the ECD.

Relative to the WT receptor, the surface expression of the GHSR1a^{E197A} was reduced moderately, the GHSR1a^{R199A} was comparable, and the GHSR1a^{P200A} was increased mildly (FIG. 10A). N8279 potency at the GHSR1a^{E197A} was diminished by ˜3-10-fold in iCa²⁺ and Gα_{q} dissociation, respectively; whereas, the N8279 Eₘₐₓ was reduced in iCa², but not Gα_{q} dissociation assays (FIGS. 3M-3P, 10B, 10C). The GHSR1a^{R199A} mutation did not affect N8279 potency in either assay, but its Eₘₐₓ, was reduced mildly in iCa²⁺ assays (FIGS. 3M-3P, 10B, 10C). N8279 potency and Eₘₐₓ were reduced dramatically at the GHSR1a^{P200A} in both Gα_{q} dissociation and iCa²⁺ assays (FIGS. 3M-3P, 10B, 10C). Grouped analysis of the N8279 potency and Eₘₐₓ at the GHSR1a^{A204E} relative to other mutants supported markedly decreased N8279-induced Gα_{q} signaling at this ECL2 residue, despite its location being outside of the putative GHSR1a^{ECD} binding pocket (FIGS. 3M-3P, 3E-3F, 3A, and 3K). Alanine substitution to Asn305 did not affect surface expression relative to the WT receptor (FIG. 10A). Although, N8279-Gα_{q} dissociation was reduced dramatically (FIGS. 3M-3N, 10B) and N8279-iCa²⁺ potency was reduced moderately (6.5-fold). Surprisingly, the N8279 Eₘₐₓ was elevated at GHSR1a^{N305A} in both assays (FIGS. 3O-3P, 10C). Any observed differences between N8279-induced Gα_{q} dissociation or iCa²⁺ at these mutants could be due to distinctions in assay kinetics or signal amplification, GPCR/transducer expression ratios, and/or the involvement of other G proteins (e.g., Gα₁₁, Gα_{i/o}, Gβγ) in the iCa²⁺ response. Together, these mutagenesis findings supported that N8279 signaling required receptor sites within and/or conformational states determined by the GHSR1a^{ECD}, especially in ECL2.

For comparison, ghrelin-stimulated Gα_{q} dissociation and iCa²⁺ was evaluated for each GHSR1a^{ECD} mutant. Ghrelin signaling efficacy was reduced markedly in both GU& dissociation and iCa²⁺ assays respectively (FIGS. 3M-3P, 10D, 10E). However, ghrelin potency at the GHSR1a^{D99A} was similar to the GHSR1aWT in Gα_{q} dissociation assays (FIGS. 3M-3N, 10D). Mutations to Glu197, Arg199, or Pro200 did not significantly affect ghrelin-stimulated Gα_{q} dissociation or iCa²⁺ potency (FIGS. 3O-3P, 10E). Ghrelin's Eₘₐₓ was unaffected at the GHSR1a^{R199A} and GHSR1a^{P200A} but it was reduced moderately at the GHSR1a^{E197A} consistent with its reduced expression (FIG. 10A). Furthermore, grouped analyses (derived from FIGS. 3B-3C) supported that ghrelin potency was unaffected at the GHSR1a^{A204E} mutant and its Eₘₐₓ was decreased only in Gα_{q} dissociation assays, consistent with reduced expression of this mutant (FIGS. 3M-3P, 10A). Ghrelin-Gag dissociation potency was reduced at the GHSR1a^{N305A}, albeit to a much lesser extent than N8279 (FIGS. 3M-3N, 10B, 10D). Nonetheless, ghrelin-iCa²⁺ potency was not significantly affected by the N305A mutation (FIGS. 3M-3N, 10D). The ghrelin Eₘₐₓ was increased in both assays (FIGS. 3M-3P, 10D-10E), similar to that seen for N8279-induced iCa²⁺ (FIGS. 3O-3P, 10C). Collectively, these findings demonstrated that Glu197, Pro200, Ala204, and, in part, Asn305 were critical and specific GHSR1aECD sites for N8279 relative to ghrelin.

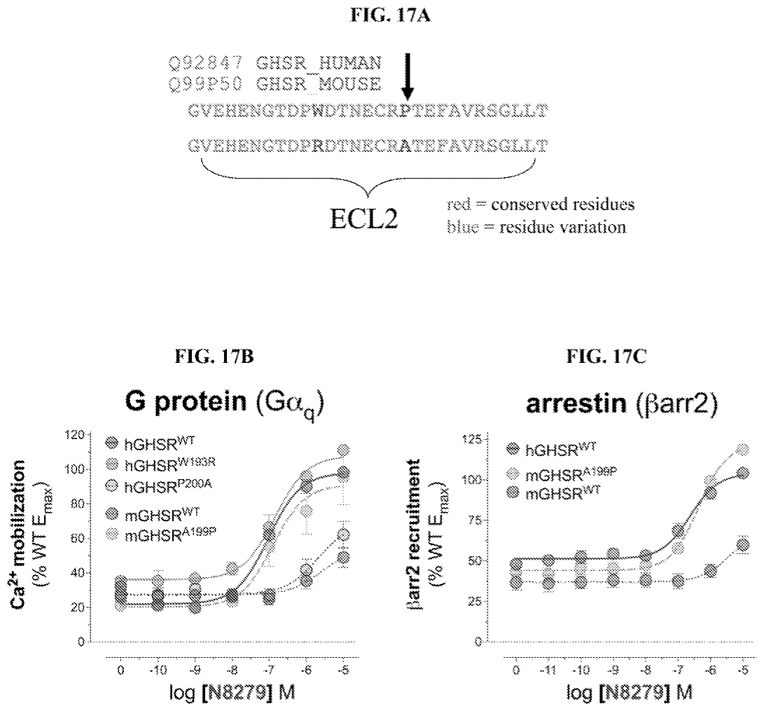

To assess if N8279 signaling required receptor sites within and/or conformational states are conserved from the mouse receptor to the human receptor, the amino acid sequences for variations within the ECL2 were first determined (FIG. 17A). Mutations were made to human GHSR1a receptor (hGHSR) for W193R and P200A and to mouse GHSR1a receptor (mGHSR) for A119P. N8279-stimulated Gα_{q} dissociation (FIG. 17B) and β-arr2 recruitment (FIG. 17C) were evaluated for each hGHSR and mGHSR mutants in comparison to endogenous (WT) receptors. FIGS. 17B-17C show that A199P mutation to mGHSR (‘humanizing’) rescues N8279 signaling.

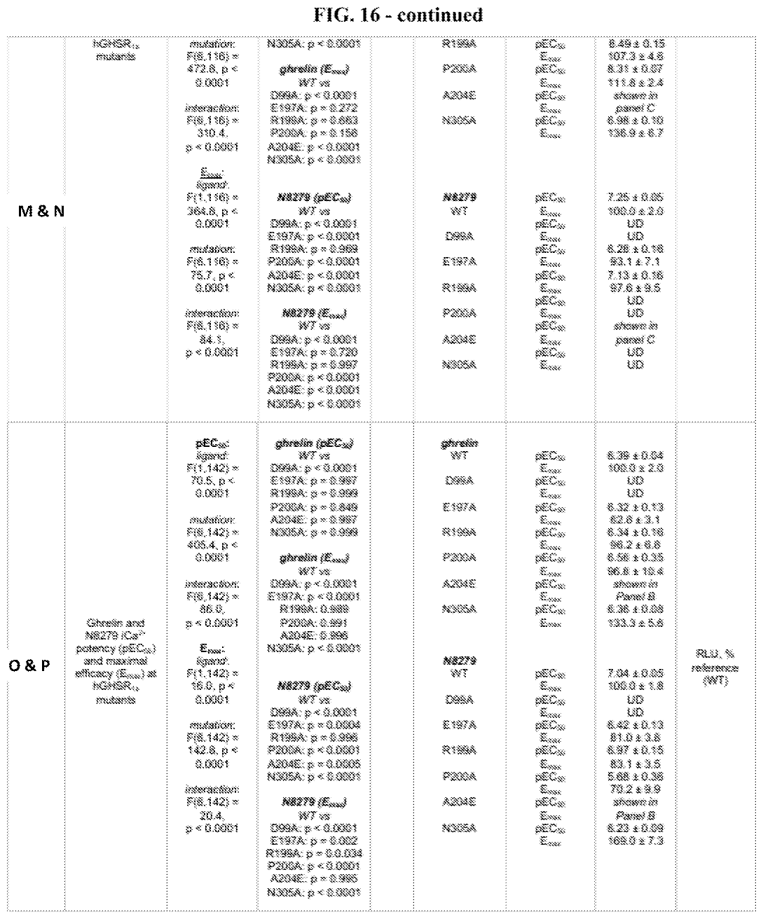

Collectively, the data in FIGS. 3A-3P support that N8279 signaling required molecular determinants within the GHSR1aECD and in particularly, ECL2. All pharmacological results and statistical comparisons for FIGS. 3A-3P are shown in FIG. 16.

### Example 74. N8279 was Brain Penetrant and Attenuated DA-Driven Behavior

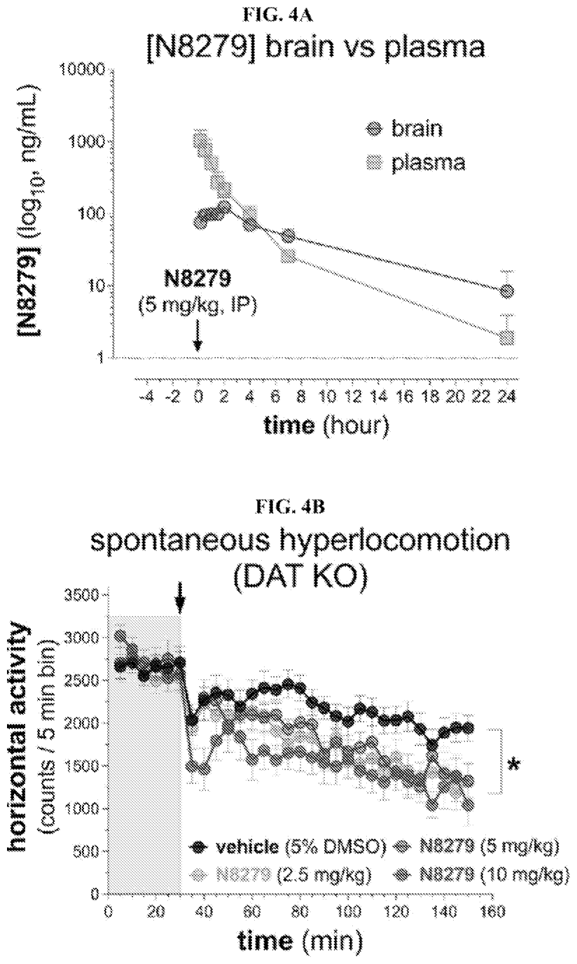

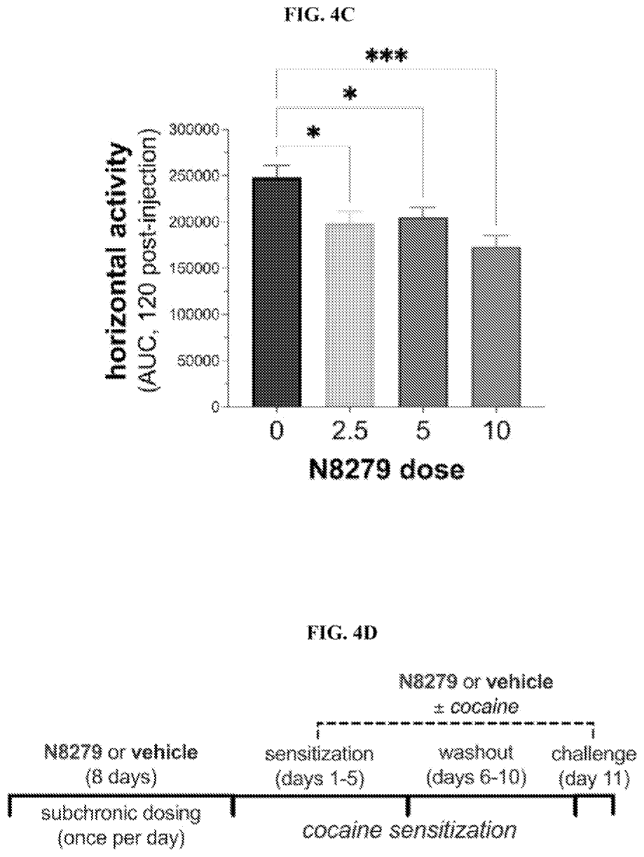

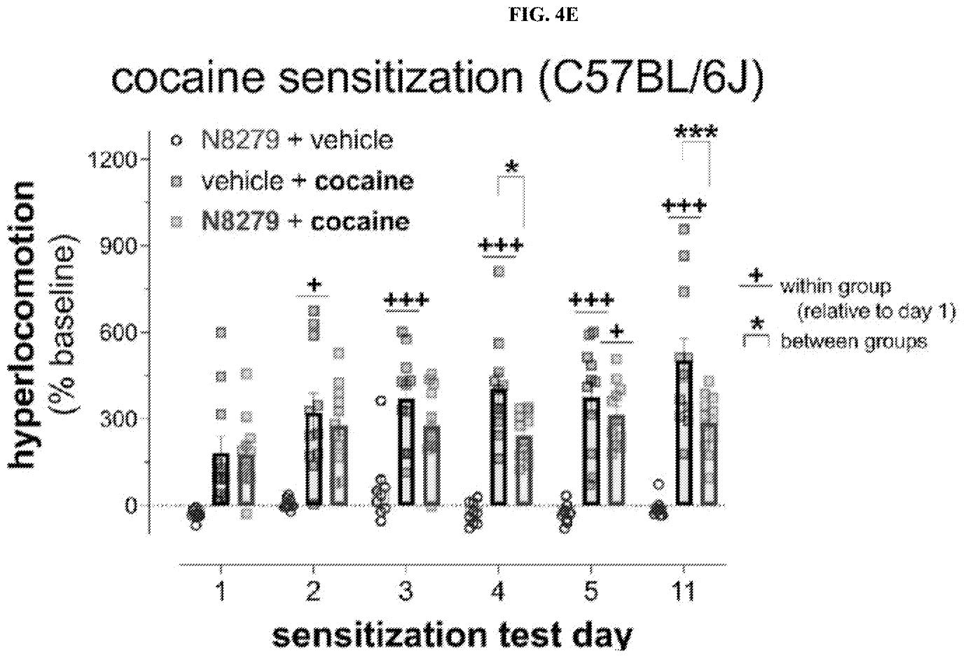

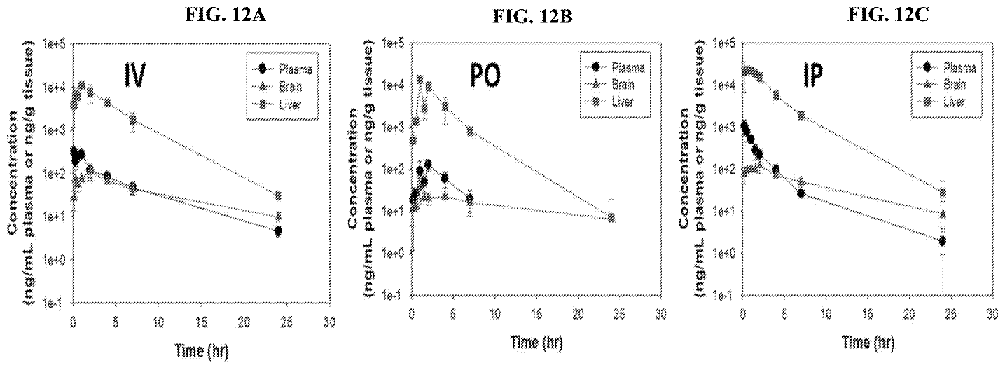

Pharmacokinetic studies with oral gavage (PO; 5 mg/kg), intraperitoneal (IP; 5 mg/kg), and intravenous (IV; 1 mg/kg) administration in C57BL/6 mice revealed a PO bioavailability of 7% and IP bioavailability of 27% (FIGS. 12A-12C, Table 3). Significantly, IP administration of N8279 (5 mg/kg) delivered pharmacologically-relevant levels (˜200 nM) in brain within 15 minutes, reaching peak concentrations (Cmax) of 259 nM at 2 hours, followed by a slow decline and elimination by 24 hours (FIGS. 4A, 12A-12C and Table 3). In brain, N8279 had a half-life (t1/2) of 6.6-11 hrs after IP and PO administration, maintaining levels above its Gαq/iCa²⁺ EC50 (˜35 nM, FIGS. 1F-1H) for an extended period (>7 hours) with a brain:plasma ratio for N8279 (IP) in the range of 0.6-0.9:1 (FIGS. 4A, 12A-12C and Table 3). In summary, N8279 achieved rapid, sustained, and pharmacologically-relevant concentrations in mouse brain following systemic administration.

TABLE 3

| | | N8279 | | | | | | | | |
|---|---|---|---|---|---|---|---|---|---|---|
| | | Sample | | | | | | | | |
| | | Plasma | Brain | Liver | Plasma | Brain | Liver | Plasma | Brain | Liver |
| | | Route | | | | | | | | |
| Parameter | Units | IV | | | PO | | | IP | | |
| Dose | mg/kg | | 1 | | | 5 | | | 5 | |
| Cₘₐₓ | ng/mL | 310 | 109 | 11000 | 126 | 37.7 | 13200 | 1030 | 123 | 22300 |
| Tₘₐₓ | hr | 0.083* | 2 | 1 | 2 | 1 | 1 | 0.167 | 2 | 0.5 |
| AUC₀₋ₜ | ng · hr/mL | 1270 | 933 | 57100 | 418 | 333 | 36200 | 1750 | 1050 | 86700 |
| AUC_{0-inf} | ng · hr/mL | 1300 | 1040 | 52200 | 470 | 436 | 36200 | 1760 | 1130 | 86800 |
| AUCₑₓₜᵣₐₚ | % | 2.4 | 10 | 0.2 | 11 | 24 | 0.1 | 0.6 | 7.1 | 0.1 |
| t_{1/2} | hr | 4.9 | 7.5 | 2.8 | 1.9 | 11 | 2.4 | 3.8 | 6.6 | 2.7 |
| CLₚ** | mL/min/kg | 13 | | | | | | | | |
| V_{dss}** | L/kg | 3.9 | | | | | | | | |
| Bioavailability | % | | | | 7.2 | | | 27 | | |
| (F)*** | | | | | | | | | | |
| AUC ratio | | | 0.8 | 40 | | 0.9 | 77 | | 0.6 | 49 |
| (tissue/plasma) | | | | | | | | | | |

*First sampling time point after IV adminstration
**CLₚ: Plasma clearance
***Bioavailablity was calculated by the AUC ratio between PO and IV administration or IP and IV administration

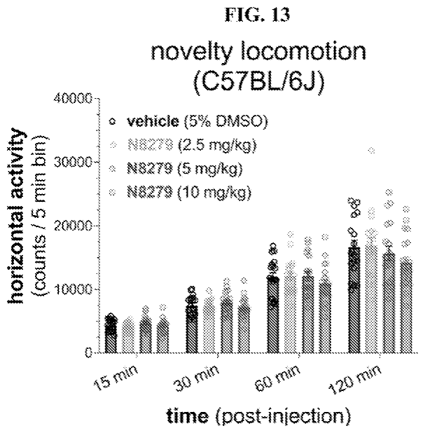

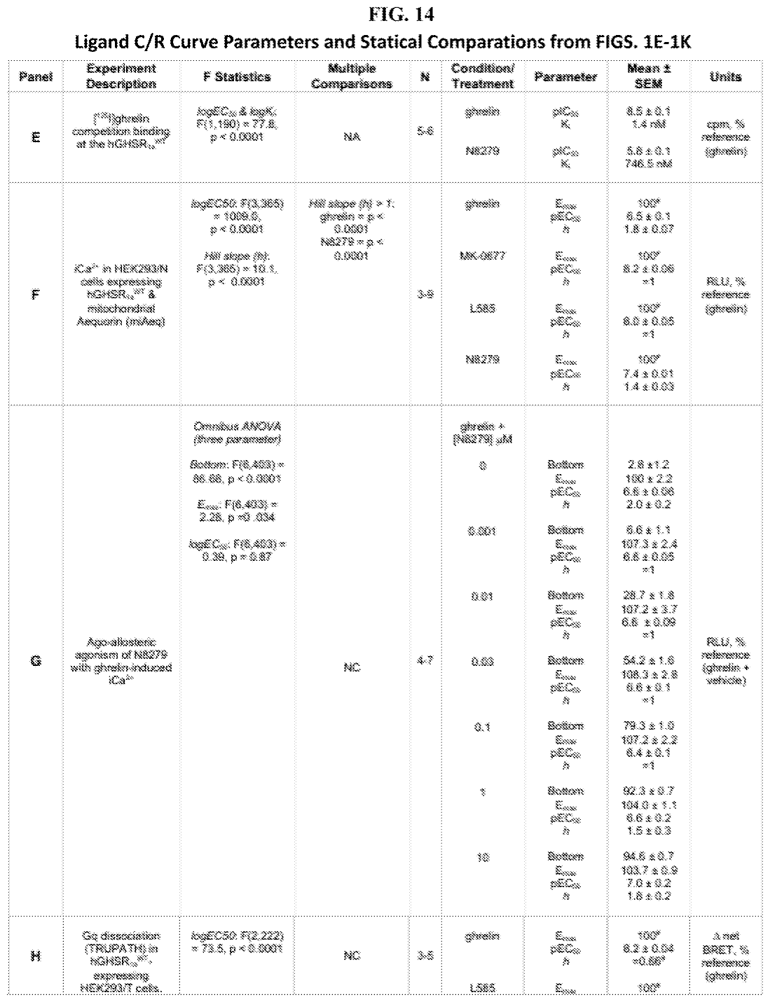

To evaluate the effect of N8279 on DAergic-modulated behavior in vivo, DAT knockout (KO) mice, which have constitutively elevated extracellular DA levels and consequently, spontaneous hyperactivity in a novel, open-field were used. After a 30 minute acclimation period, male and female DAT KO mice were administered vehicle or pharmacologically-relevant, brain penetrant doses of N8279 (FIG. 4A; 2.5, 5, or 10 mg/kg, IP), then were returned to the open-field and locomotion measured for 120 minutes. Each dose of N8279 reduced overall hyperlocomotion in DAT KO mice relative to vehicle controls (FIGS. 4B-4C). Parallel control experiments with inbred male and female C57BL/6J mice indicated that N8279 did not affect novelty-induced open-field locomotion (FIG. 13).

Next, cocaine-induced behavioral sensitization was assessed in male and female C57BL/6J mice following subchronic (8-day) administration of the vehicle or N8279 (5 mg/kg, IP) in the home-cage. Subsequently, mice were given the same treatments as described above, followed with an injection (IP) of vehicle or cocaine (20 mg/kg) in the open-field once per day for 5 days (FIG. 4D). A 5-day hiatus (washout) was imposed and behavioral sensitization was assessed the next day by giving (IP) vehicle or cocaine (challenge). The cumulative results showed that post-injection locomotor activities were low in the N8279+vehicle group and were significantly reduced from the cocaine-treated mice across all days (FIG. 4E). By comparison, motor activities were stimulated acutely (day 1) to similar extents in the vehicle+cocaine and N8279+cocaine mice (FIG. 4E). Locomotion was increased from day 1 though each day to day 5 in the vehicle-+cocaine group, whereas significantly enhanced activity was observed on day 5 in the N8279+cocaine mice. Moreover, at challenge (day 11), locomotor activity was augmented relative to day 1 in the vehicle+cocaine mice, whereby no change in activity was evident in the N8279+cocaine mice. Thus, N8279 both delayed the appearance of sensitization across days and abrogated the expression of sensitization following washout (challenge, day 11). Collectively, these results indicated that a pharmacologically-relevant and brain penetrant levels of N8279 ameliorated aberrant DA-mediated behavior in two models of persistently disrupted DA homeostasis.

### Methods Used in Examples 1 and 69-74

The following exemplary methods were performed in Examples 1 and 69-74 as described herein:

Chemicals and Compounds. Full-length, human acyl-ghrelin (Cat. no. 1463), L-692,585 (Cat. no. 2261), YIL781 hydrochloride (Cat. no. 3959), and MK-0677 (Cat No. 5272) were purchased from Tocris Biosciences (Bristol, United Kingdom). JMV2959 was purchased from Sigma Aldrich (Cat no. 345888). [¹²⁵I]ghrelin was purchased from Perkin-Elmer (Waltham, MA; Cat no. NEX3880101UC). Ghrelin peptide was maintained as a 1 mM stock in 50% glycerol, whereas all other small molecule ligands (including N8279) were maintained as 10 mM stocks in dimethyl sulfoxide (DMSO).

Plasmids. The 3×HA-hGHSR1a^{WT} plasmid was originally purchased from the cDNA Resource Center and consisted of an N-terminally 3×HA-tagged human GHSR1a^{WT} coding sequence cloned into a pcDNA3.1+ backbone. This construct was used to generate 3×HA-hGHSR1a^{A204E} by QuikChange site-directed mutagenesis (Agilent Technologies, Santa Clara, CA). The expression vector for the bioluminescent, mitochondria-targeted apo-aequorin Ca²⁺ sensor (miAeq ¹) was a gift from Dr. Stanley Thayer (University of Minnesota). To generate a C-terminally-tagged 3×HA-hGHSR1a^{WT} plasmid with LgBiT, the coding sequence for 3×HA-hGHSR1a^{WT} was inserted 5′ into a vector containing the coding sequence for the LgBiT NanoLuc fragment (pBiT1.2-C). To generate a complementary N-terminally-tagged β-arrestin2^{WT} plasmid, β-arrestin2^{WT} was inserted 3′ of the coding sequence for the SmBiT NanoLuc fragment (pBiT2.2-N). Both LgBiT and SmBiT vector backbones were purchased from Promega. GαqLgBiT, ^{smBi}T-J31, untagged Gα2, and RIC8A were generous gifts from Dr. Asuka Inoue. The hGHSR1a^{WT-RLuclI} and 2×-FYVE^{mVenus} plasmids used for bBRET analyses were made by the Caron lab. The RhoA-dependent serum response element plasmid SRF-RE-Luciferase, cloned into the pGL4.34[luc2P/SRF-RE/Hygro] vector backbone, was purchased from Promega (Cat. no. E1350). pcDNA3.1+ was used as empty vector in all experiments and all constructs were validated by sequencing.

Cell Culture & Transfections. U2OS, HEK293/T, and HEK293/N cells were obtained from the American Type Culture Collection (ATCC, Manassas, VA) and cultured in Dulbecco's modified Eagle's medium (DMEM) supplemented with 10% (v/v) of Fetal Bovine Serum (FBS) and 1× antibiotic-antimycotic solution (100 IU-1 penicillin, 100 μg/mL streptomycin and 250 ng/mL amphotericin B; MilliporeSigma). HEK293/S Gαq/11 KO and its parental WT line were a generous gift from Dr. Asuka Inoue (Tohoku University, Miyagi, Japan). U2OS cells stably expressing 3×HA-hGHSR1a^{WT} and green fluorescent protein (GFP) tagged-β-arrestin2 and HEK293/N cells stably expressing 3×HA-hGHSR1a^{WT} and the luminescent Ca²⁺ sensor mitochondrial-Aequorin (miAeq) were made by the Caron lab. All cell lines were grown in a humidified incubator at 37° C. (5% CO2). All transient transfections were performed using a standard calcium phosphate method.

High-Throughput and Directed Library Compound Screening. Quantitative high-throughput screening (qHTS). qHTS was performed at the National Center for Advancing Translational Sciences (NCATS) against 47,000 compounds from the NCATS Sytravon library and Pharmacological collection (NPC) using the PathHunter U2OS GHSR1a β-arr1 cells and the β-arr Assay kit (DiscoverX, Fremont, CA), which measures recruitment of β-arr to the GHSR1a. Prior to the screening, the assay was miniaturized to a 1536-well format and optimized in terms of signal-to-background window (S/B), Z factor, and potency of ghrelin control. The initial assay validation was performed with Library of Pharmacologically Active Compounds (LOPAC¹²⁸⁰, Sigma-Aldrich) to confirm plate-to-plate reproducibility of parameters, hit rate identification, etc. For qHTS, two doses of the compounds—11 μM and 57 μM—were used to measure GHSR1a activation using a fully automated robotic screening system (Kalypsys, San Diego, CA). Briefly, 1.2×10³ cells were seeded with MultiDrop Combi dispenser (Thermo Scientific, Logan, UT) into white solid-bottom tissue culture-treated 1536-well plates (Aurora Microplates, Whitefish, MT) in 3 μL of AssayComplete Cell Plating 5 Reagent (DiscoverX) and cultured overnight. Next, 1 μl/well of 1 μM Ghrelin diluted in HBSS+10 mM HEPES (HH buffer) was added to one column of the plate, while all other wells were dispensed with 1 μL/well of HH buffer for matching the assay conditions. Subsequently, the libraries' compounds in DMSO solution were transferred from the source plates to the assay plates at 23 mL/well. The plates were incubated for 90 minutes followed by addition of 1.5 μL/well PathHunter Detection Reagent prepared according to the manufacturer's instructions. After 60 minutes of incubation at ambient temperature, the luminescent signal was measured on ViewLux uHTS Microplate Imager (Perkin Elmer, Waltham, MA) with 20 seconds exposure. Quality of the screening was evaluated based on the median characteristics of Z factor and S/B which were 0.53 and ˜4.5-fold, respectively. Hit detection window parameters were calculated based on results obtained from the vehicle control (ECO) and ghrelin as the positive control (EC100=250 nM final concentration) conditions. A cut-off of >60% activation by either the lower or higher compound's dose was used to select primary hits. A follow-up PathHunter U2OS GHSR1a β-arr1 assay was performed on 145 selected primary compounds. They were re-tested at 7 doses in the range [57 μM-3.7 nM] applying the same protocol as for qHTS. Thirty-six hits were selected based on curve response class (CRC) 1,2,3 for further validation.

X-Ray Diffraction. Single crystal X-ray diffraction studies were conducted on a Bruker Kappa Photon 11 CPAD diffractometer equipped with Cu Kₐ radiation (λ=1.54178 Å). Crystals of the subject compound were grown by dissolving approximately 1 mg of sample in 350 μL of 90/10 Dichloroethane/Methanol solution, which was then vapor diffused with Pentane over several days. A 0.267×0.243×0.228 mm piece of a colorless block was mounted on a Cryoloop with Paratone oil. Data were collected in a nitrogen gas stream at 285K using Φ and ω scans. The crystal-to-detector distance was 40 mm using variable exposure time (20 s-90 s) depending on Θ with a scan width of 2.0°. Data collection was 99.5% complete to 59.009° in Θ (0.90 Å). A total of 58959 reflections were collected covering the indices, −17<=h<=17, −16<=k<=16, −24<=1<=24. 6947 reflections were found to be symmetry independent, with a Rᵢₙₜ of 0.0596. Indexing and unit cell refinement indicated a primitive, monoclinic lattice. The space group was found to be P21/c. The data were integrated using the Bruker SAINT software program and scaled using the SADABS software program. Solution by direct methods (SHELXT) produced a complete phasing model for refinement. All nonhydrogen atoms were refined anisotropically by full-matrix least-squares (SHELXL-2014). All carbon bonded hydrogen atoms were placed using a riding model. Their positions were constrained relative to their parent atom using the appropriate HFIX command in SHELXL-2014. All other hydrogen atoms (H-bonding) were located in the difference map. Their relative positions were restrained using DFIX commands and their thermals freely refined. Crystallographic data are summarized in Table 2.

GHSR1a Radioligand Binding Assays. Saturation: [^{I125}]ghrelin (Perkin-Elmer, Waltham, MA) saturation binding experiments were performed as described. Briefly, hGHSR1aWT (1 μg) was transiently transfected into HEK293/T cells cultured on 1 cm dishes. After 48 hours, cells were collected, resuspended in hypotonic membrane lysis buffer (50 mM Tris-HCl, pH 7.4), then centrifuged at 21,000×G for 20 minutes to obtain crude membrane pellets. Membranes were resuspended in assay buffer (50 mM Tris-HCl, 5 mM EDTA, 5 mM MgCl2, 1% BSA), protein content was measured by BCA protein assay, and diluted to 2 μg/mL. Ten μL of GHSR1a-expressing membrane (20 μg total protein) was then incubated with increasing concentrations of cold ghrelin (0.8-10 nM) and [^{I125}]ghrelin with its specific activity diluted 1:100. Non-specific binding was defined in parallel reactions containing 10 μM YIL781. Binding reactions proceeded for 1 hour on ice and were terminated by rapid filtration over 0.3% PEI-soaked GF/B filters with a 96-well Brandel harvester, followed by four washes with ice-cold wash buffer (Tris HCl, pH 7.4).

Competition: Equilibrium [^{I125}]ghrelin competition binding assays were performed by incubating 2-20 μg of hGHSR1a^{WT} (1 μg transfected, 10 cm dish) or hGHSR1a^{L149G} (0.5 μg transfected, 10 cm dish)-expressing HEK293 cell membranes with a fixed concentration of [^{I125}]ghrelin (3.2 nM) and increasing concentrations of cold acyl-ghrelin (1-28), N8279, N6164, or N1956. hGHSR1a^{L149G} was transfected at half the total amount of cDNA relative to hGHSR1a^{WT} because it exhibited enhanced cell surface expression. The data for each ligand was normalized to its respective vehicle condition, representing 100% binding. Equilibrium binding reactions proceeded for 1 hour and were terminated by rapid filtration over 0.3% PEI-soaked GF/B filters using a 96-well Brandel harvester, followed by four washes with ice-cold wash buffer.

Gα_{q}-Dependent Intracellular Ca²⁺ Mobilization. HEK293/N cells stably expressing 3×HA-hGHSR1a^{WT} and the mitochondrial-Aequorin (miAeq) Ca²⁺ sensor were grown in standard DMEM supplemented with 10% FBS and antibiotic-antimycotic, as well as selection media (G418 and Puromycin). Cells were split into and seeded into 10 cm dishes without antibiotic selection and grown to confluency overnight. The next morning, cells were serum starved in 10 mL of clear opti-MEM (supplemented with L-glutamine and HEPES; Gibco, Waltham, MA) for 3-4 hours in a 37° C. incubator with 5% CO2. Then, 25 μL of coelenterazine H (NanoLight Technology, Pinetop, AZ) was added to each 10 mL dish and incubated for another 1-2 hours. For antagonist assays, test compounds were either added directly to the media in 10 cm dishes, or cells were collected by gentle washing/trituration and plated onto white, clear-bottomed 96-well plates for 30 minutes—1 hour. Once the coelenterazine H (or antagonist) incubation was complete, cells were harvested from the 10 cm dish by gentle washing/trituration and collected in a 15 mL conical tube. Cell suspensions were then immediately subjected to assay by injecting cells onto a white, clear-bottomed 96-well plate containing 2× test compounds. Luminescence was read without delay and for 10 seconds per well. To control for cell number variability, cells were lysed in 2× lysis buffer (100 mM CaCl₂+0.2% Triton-X) immediately following the assay. The max ligand-induced response (Ca²⁺ mobilization: net-miAeq) over 10 seconds was normalized by dividing the ligand-induced luminescence counts (L-miAeq) by the ligand-induced luminescence counts (L-MiAeq) plus the luminescence measured upon cell lysis (Lysis-miAeq). Relative, lysis-normalized data were then normalized to the appropriate control/reference condition.

For experiments with WT and Gt KO HEK293/S cells, 2.5 pig of hGHSR1a^{WT}, 10 μg of miAEQ, and 2.5 μg of pcDNA3.1 was transiently transfected into 10 cm dishes and incubated overnight. For experiments evaluating hGHSR1a^{A204E}-mediated Ca²⁺ mobilization, HEK293/T (10 cm dishes) cells were transiently transfected with hGHSR1a^{WT} (2.5 μg) or hGHSR1a^{A204E} (5 μg), 10 μg of miAEQ, and 2.5 or 0 μg of pcDNA3.1, then incubated overnight. hGHSR1a^{A204E} was expressed at 2×hGHSR1a^{WT} because it exhibited ˜50% the cell surface expression of the WT receptor (see FIG. 9). The next morning, transfection media was exchanged with fresh DMEM (supplemented with 10% FBS and antibiotic-antimycotic). Twenty-four hours later, experiments proceeded in accordance with the procedures described above. To control for any possible kinetic differences in genetically modified cell lines or GHSR1a variants, the average ligand-induced response over 10 seconds was calculated, then normalized by dividing the L-miAeq by the L-miAeq plus the Lysis-miAeq and normalized to the appropriate control/reference condition.

β-arr2^{GFP} Translocation. Compound Screening: β-arr2 translocation was assessed using U2OS cells stably expressing the human hGHSR1a-vasopressin receptor 2 tail chimera (hCHSR1a^{V2T}) and green fluorescent protein (GFP)-tagged β-arr2. Replacement of the C terminal sequence of GHSR1a to the C terminal tail of a class B GPCR, vasopressin receptor 2 leads to the formation of stable GHSR1a/β-arrs complexes in endocytic vesicles. On day 1, stable cells were split into MGB101-1-2-LG glass-bottom 384-well plates (MatriCal, Spokane, WA) using a Multidrop 384 liquid dispenser (Thermo Scientific, Hudson, Nil). Each well contained 30 μL aliquots of 8,000 cells in Minimum Eagle's medium (MEM) containing 10% fetal bovine serum (FBS) and 100 U/mL penicillin/streptomycin (Life Technologies, Grand Island, NY). The plates were incubated overnight at 37° C. in 5% CO2, and on the following day, media was changed to 30 LL clear MEM without serum. Compounds at 50 μM in 5% DMSO from the Molecular Libraries Small Molecule Repository (MLSMR) collection were added to each well using a MicroLab StarLET liquid handler (Hamilton Robotics, Reno, NV) and diluted 10-fold to 5 μM final concentration. The plates were returned to the incubator for 40 min, and the cells were fixed by adding 30 μL of 2% paraformaldehyde-phosphate buffered saline (PBS) to each well. Plates were stored at 4° C. until analysis on an ImageXpress Ultra (Molecular Devices, Sunnyvale, CA) at 488 nm. Images were analyzed using a wavelet algorithm to measure formation of fluorescence aggregates. Image results were also confirmed visually.

Evaluation of Ghrelin- and N8279-Induced β-arr2^{GFP} Translocation. U2OS cells stably expressing the human GHSR1a^{WT} and β-arr2^{GFP} were seeded onto 35 mm MatTek (Ashland, MA, USA) glass coverslip dishes in opti-MEM supplemented with 2% FBS and antibiotic-antimycotic and grown overnight in a humified incubator (5% CO2, 37° C.). The next morning, media was aspirated and replaced with fresh, serum-free opti-MEM (supplemented with antibiotic-antimycotic). Four hours later, cells were treated with vehicle, ghrelin (100 nM), or N8279 (100 nM), placed back in a humified incubator (5% CO2, 37° C.) and incubated for 45 minutes. Next, treatment was terminated by aspiration of media, a PBS wash, and fixation with 4% paraformaldehyde (20 minutes, room temperature (RT)). Fixed cells were either stored at 4° C. or immediately imaged for β-arr2^{GFP} translocation with a Zeiss LSM 510 meta confocal microscopy.

NanoBiT-based Gaq Dissociation & β-arr2 Recruitment Assays. Gαq dissociation/activation: GαqLgBiT and ^{smBiT-}β1 were transiently co-transfected with human 3×HA-GHSR1a^{WT}, untagged Gγ2, and RIC8A into monolayers of HEK293S-Gαq knockout cells. 3×HA-GHSR1a^{WT} and GαqLgBiT cDNA was transfected at a 1:1 ratio. The next morning, media (DMEM supplemented with 10% FBS and antibiotic/antimycotic) was exchanged and after 4-6 hours, cells were plated on white 96-well, clear-bottomed assay plates at a density of 45,000 cells/well in opti-MEM supplemented with 2% FBS and antibiotic/antimycotic. The next morning, opti-MEM was removed and cells were incubated with 80 μL HBSS supplemented with 20 mM HEPES (pH 7.4) for 4 hours. Cells were then treated with 10 μL of the luminescent substrate, coelenterazine H (5 μM final), for 15 minutes at room temperature prior to the addition of 10 μL of test compound. Two minutes ligand application, a stable luminescence signal was established and measured by a Mithras 940 plate reader over 20 minutes. The average response was utilized for generating C/R curves in FIG. 7H.

β-arr2 recruitment: In agonist and antagonist assays of NanoBiT-based β-arr2 recruitment, hGHSR1a^{WT-L}g^{BiT} and ^{SmBiT}-β^{WT} were transiently co-transfected at a 1:1 ratio (250 μg each) into monolayers of HEK293/T cells on 6-well plates along with 2 μg of pcDNA3.1. For assays employing the Ala204Glu variant, hGHSR1a^{A204E-L}g^{BiT} was transfected at 2×hGHSR1a^{WT-L}g^{BiT}, as performed for iCa²⁺ assays with hGHSR1a^{A204E} described above. For GHSR1a-β-arr2 saturation assays, a fixed amount of hGHSR1a^{WT-L}g^{BiT} cDNA (250 μg) was transfected with 0, 62.5 (1:0.25), 125 (1:0.5), 250 (1:1), 500 (1:2), or 1000 (1:4) μg of ^{SmBiT}β-arr^{WT}. The morning after transfection, media was exchanged and after 4-6 hours, cells were then plated on white 96-well, clear-bottomed assay plates at a density of 45,000 cells/well in opti-MEM supplemented with 2% FBS and antibiotic/antimycotic. The following morning, opti-MEM was removed and cells were incubated with 70-80 μL HBSS supplemented with 20 mM HEPES (pH 7.4) for 4 hours. Cells were then treated with 10 μL of coelenterazine H (5 μM final) for 15-20 minutes at room temperature. Cells were then stimulated with 10 μL of test compounds and 2 minutes later, luminescence was measured over 5 minutes, which encompassed the peak β-arr2 recruitment signal. For antagonist assays, cells were pretreated with the indicated ligand for 5 minutes prior to stimulation with EC₈₀ ghrelin (40 nM).

BRET-based Gα_{q} Dissociation & β-arr2 Recruitment Assays. TRUPATH Gα dissociation: Gα_{q/i1/i2/OA/12/13/sS}^{RLuc8} and Gγ8^{GFP2} or Gγ9^{GFP2} were transiently co-transfected with human 3×HA-GHSR1a^{WT} and untagged Gβ₃ at a 1:1:1:1 ratio (100 μg each) into monolayers of HEK293/T cells along with 1.6 μg pcDNA3.1. The next morning, media (DMEM supplemented with 10% FBS and antibiotic/antimycotic) was exchanged and after 4-6 hours, cells were plated on white 96-well, clear-bottomed assay plates at a density of 40,000-50,000 cells/well in opti-MEM supplemented with 2% FBS and antibiotic/antimycotic. The next morning, opti-MEM was removed and cells were incubated with 80 μL HBSS supplemented with 20 mM HEPES (pH 7.4) for 4 hours. Cells were then treated with 10 μL of the luminescent substrate, coelenterazine 400a (5 μM final), for 5 minutes at room temperature prior to the addition of 10 μL of test compound. Five minutes after ligand application, luminescence was measured by a Mithras 940 plate reader over 30 minutes and the maximal response was utilized for generating all C/R curves.

β-arr2 recruitment: 3×HA-hGHSR1^{WT-RLucII} and ^{mVenus}-βarr2^{WT} were transiently co-transfected at a 1:15 ratio (100 ng:1.5 μg) into monolayers of HEK293/T cells on 6-well plates along with 0.9 μg of pcDNA3.1. Due to increased surface expression of GHSR1aL149G, 50 ng of 3×HA-hGHSR1a^{L149G-}RLucII was transfected with 750 ng of ^{mVenus}-β-arr2^{WT} (1:15 ratio). The morning after transfection, media was exchanged and after 4-6 hours, cells were then plated on white 96-well, clear-bottomed assay plates at a density of 45,000 cells/well in opti-MEM supplemented with 2% FBS and antibiotic/antimycotic. The following morning, opti-MEM was removed and cells were incubated with 70-80 μL HBSS supplemented with 20 mM HEPES (pH 7.4) for 4 hours. Cells were then treated with 10 μL of coelenterazine H (5 μM final) for 5-10 minutes at room temperature. Cells were then stimulated with 10 μL of test compounds and 2 minutes later, luminescence was measured over 60 minutes and the maximal response was utilized for generating C/R curves in FIG. 2D.

Chemiluminescent Fixed-Cell ELISA. 3×HA-hGHSR1a^{WT} or pcDNA3.1 was transiently transfected by the calcium phosphate method into monolayers of HEK293/T cells on 6-well plates. The next morning, media (DMEM supplemented with 10% FBS and antibiotic/antimycotic) was exchanged. Later that afternoon, cells were plated on white 96-well, clear-bottomed assay plates at a density of 40,000 cells/well in opti-MEM (supplemented with 2% FBS and antibiotic/antimycotic). The next morning, media was removed and cells were serum starved with 90 μL of opti-MEM (containing no serum) for 2-3 hours at 37° C. Cells were then stimulated with 10× test compounds (diluted in serum-free opti-MEM) for 45 minutes at 37° C. in a humidified incubator. After 45 minutes, cells were fixed with 4% paraformaldehyde for 15 minutes (RT). Fixed cells were then washed three times with PBS (pH 7.4, Gibco) and blocked with fish gelatin in TBS (Rockland, Gilbertsville, PA) for 45 minutes. Cells were then incubated with a rabbit, horseradish peroxidase (HRP)-conjugated anti-HA antibody (1:2, 500; Novus Biologicals, Littleton, CO) for 1 hour (RT). After three washes with PBS, cells were treated with 50 μL of SuperSignal West Pico PLUS Chemiluminescent Substrate (Thermo Scientific, Waltham, MA) and luminescence was measured by a Mithras 940 plate reader over ˜12 minutes. The average luminescence was used and the data for each ligand were normalized its corresponding vehicle treatment conditions.

Bystander BRET (bBRET). hGHSR1a^{WT-RLucII} and plasma membrane marker, MyrPalm^{mVenus}, or the early endosomal marker, 2×-FYVE^{mVenus}, was transiently co-transfected in HEK293/T cells at a ratio of 1:5 or 1:12.5, respectively. The next morning, media (DMEM supplemented with 10% FBS and antibiotic/antimycotic) was exchanged. Later that afternoon, cells were plated on white 96-well, clear-bottomed assay plates at a density of 45,000 cells/well in opti-MEM (supplemented with 2% FBS and antibiotic/antimycotic). The next morning, opti-MEM was removed, and cells were incubated with 70-80 μL HBSS+20 mM HEPES for 4 hours at 37° C. Cells were treated with 10 μL of coelenterazine H (5 μM final) for 5-15 minutes at 37° C., then stimulated with 10 μL of 10× test compound for 5 minutes at 37° C. Plates were then read by a Mithras 940 plate reader every 5 minutes over 1 hour (MyrPalm^{mVenus}) or 2 hours (2×FYVE^{mVenus}) to determine a BRET ratios. To generate CR curves, the average net BRET over 60 minutes post-treatment was used.

SRF-RE Transcriptional Activity. hGHSR1a^{WT} and SRF-RE (Promega) were transiently co-transfected at a 1:40 ratio by the calcium phosphate method into monolayers of HEK293/T cells on 6-well plates. The next morning, standard media was exchanged and later that afternoon, cells were plated on white 96-well, clear-bottomed assay plates at a density of 45,000 cells/well in opti-MEM supplemented with 2% FBS and antibiotic/antimycotic. The next morning, media was removed and cells were serum starved with 90 μL of opti-MEM (containing no serum) for 2 hours at 37° C. Cells were then stimulated with 10× test compounds (diluted in serum-free opti-MEM) for 5 hours at 37° C. in a humidified incubator. Cells were then lysed in 20 μL of 1× passive lysis buffer (Promega) for 10 minutes on a shaker (RT). To measure luminescence, 40 μL of luciferin (in HBSS supplemented with 20 mM HEPES) was injected into in each well and immediately read by a Mithras 940 plate reader for 10 seconds.

Molecular Docking. Molecular docking studies were performed using the Glide and Maestro user interface (Release 2019-4, Schrodinger LLC, New York, NY). The model structure of ghrelin-bound GHSR1a and the X-ray crystal structure of antagonist-bound GHSR1a were used to represent the active and inactive state of the GHSR1a, respectively. The Protein Preparation Wizard function was used to assign bond orders, add hydrogen atoms, and remove water molecules that did not participate in interactions. The GHSR1a models were subjected to energy minimization using the OPLS3 force field. A receptor grid box of 30×30×30 Å³ with a default inner box (10×10×10 Å³) was centered on the ligand binding pocket. The ligand structures were generated and prepared using the LigPrep function with the OPLS3 force field. Flexible ligand docking was performed using the “standard precision” Glide algorithm, and after the post-docking minimization the pose with the best docking score was evaluated.

Pharmacokinetic Analysis. Male C57BL/6 mice were used for pharmacokinetic studies and were purchased from Charles River Laboratories. DAT KO mice were backcrossed for >10 generations onto a C57BL/6J (Jackson Laboratory, Bar Harbor, ME) genetic background. Mice were bred and maintained on a standard 12:12 hour light:dark cycle, socially housed, and supplied with standard laboratory chow and water ad libitum, except during testing. Male C57BL/6 mice (n=3/time-point) were administered N8279 at 1 mg/kg IV, 5 mg/kg PO and 5 mg/kg IP. Plasma, brain and liver samples were collected over 24 hours. N8279 concentrations in plasma, brain and liver homogenates were determined by LC-MS/MS. The mean concentration from 3 animals at each time-point was used in the pharmacokinetic (PK) analysis. PK parameters were calculated with Phoenix WinNonlin Software (Ver. 8.0, Certara).

Novelty-induced locomotor activity in DAT KO and inbred C57BL/6J mice. Open-field locomotor activity in mice was performed similar to that described in Barak et al., ACS Chem Biol. (2016)11(7):1880-90, the disclosure of which is incorporated herein in its entirety. In brief, Age- and sex-matched, littermate DAT KO and inbred C57BL/6J mice between 2-6 months of age were used to measure locomotor activity in an Omnitech Digiscan activity monitor (20×20 cm2; Accuscan Instruments, Columbus, OH). Locomotor activity was measured at 5 minutes intervals. To evaluate the effects of compounds on locomotor behavior, the mice were placed in an activity monitor for a 20-minute habituation period, and afterwards injected with drug or vehicle, returned to the monitor, and locomotor activity was recorded over a period of 90 minutes.

Cocaine Sensitization. Male and female C57BL6/J mice (Jackson Labs, Bar Harbor, ME) were administered (IP) vehicle or N8279 (5 mg/kg) subchronically for 8 consecutive days (once/day) (FIG. 4D). Subsequently, mice were placed into the open-field (Accuscan Instruments, Columbus, OH) for 30 min, they were removed, injected (IP) with vehicle or N8279, returned to the open field for 30 min, then the mice were given (IP) vehicle or cocaine (20 mg/kg) and returned to the open-field for 120 minutes (FIG. 4D). This procedure was repeated once/day for 5 consecutive days. A drug-free hiatus in the home-cage was imposed for 5 days (washout), and then on day 11 the mice were challenged (IP) with vehicle or cocaine (20 mg/kg) to test for behavioral sensitization. Since the injections of vehicle and N8279 occurred 30 minutes prior to cocaine administration, this time-period was taken as baseline activity. Since this baseline locomotor activity declined across days in the N8279 group, the results are presented as percent change from baseline activities.

Statistics. All data are presented as the mean±SEM derived from multiple independent experiments or animals. For binding and signaling assays, >2 technical replicates were included in each experiment. These data were plotted and analyzed in GraphPad Prism version 9.0 with a statistical significance threshold defined as p<0.05. Non-linear regression parameters and best-fit models for all C/R data were determined statistically by an extra sum-of-squares F-test. The behavioral data were analyzed by repeated measures ANOVA followed by the appropriate post-hoc multiple comparisons test.

One skilled in the art will readily appreciate that the present disclosure is well adapted to carry out the objects and obtain the ends and advantages mentioned, as well as those inherent therein. The present disclosure described herein are presently representative of preferred embodiments, are exemplary, and are not intended as limitations on the scope of the present disclosure. Changes therein and other uses will occur to those skilled in the art which are encompassed within the spirit of the present disclosure as defined by the scope of the claims.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise.

The present disclosure shall control in the event there are any disparities between any definitions and/or description found in the cited references.

1. A compound of Formula (I), analogs, isomers, pharmaceutically acceptable salts, and prodrugs thereof or a pharmaceutically acceptable salt thereof: 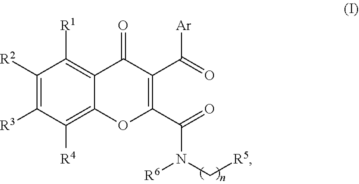
wherein R¹, R², R³, and R⁴ are independently selected from a group consisting of H, NO₂, CN, CHO, F, Cl, Br, I, CF₃, unsubstituted C₁-C₆ alkyl, substituted alkyl, COR¹², CO₂H, CO₂R¹³, CONH₂, CONHR¹⁴, and CONR¹⁵R¹⁶;
R⁵ is selected from a group consisting of OH, OR¹⁷, NH₂, NHR¹⁸ NR¹⁹R²⁰ NHCOR²¹, NHCO₂R²² and heterocyclic;
R⁶ is H or an unsubstituted C₁-C₆ alkyl,
Ar is 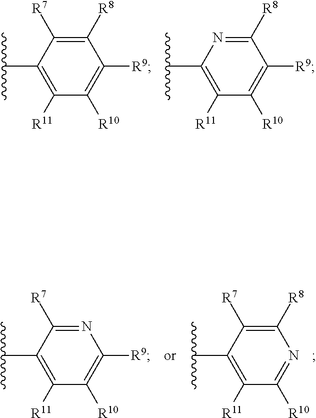
R⁷, R⁸, R⁹, R¹⁰, and R¹¹ are independently H, F, Cl, Br, I, CF₃, CN, OH, OR²³, NH₂, NHR²⁴ NR²⁵R²⁶, NHCOR², NHCO₂R²⁸, unsubstituted C₁-C₆ alkyl, substituted C₁-C₆ alkyl, unsubstituted C₂-C₆ cycloalkyl, substituted C₂-C₆ cycloalkyl, unsubstituted C₁-C₆ alkenyl, substituted C₁-C₆ alkenyl, unsubstituted C₁-C₆ alkynyl, substituted C₁-C₆ alkynyl, unsubstituted phenyl, substituted phenyl, R⁷ and R⁸ may be taken together to form an unsubstituted C₁-C₆ alkyl, or a substituted C₁-C₆ alkyl, R⁸ and R⁹ may be taken together to form an unsubstituted C₁-C₆ alkyl, or a substituted C₁-C₆ alkyl, R⁹ and R¹⁰ may be taken together to form an unsubstituted C₁-C₆ alkyl, or a substituted C₁-C₆ alkyl, R¹⁰ and R¹¹ may be taken together to form an unsubstituted C₁-C₆ alkyl, or a substituted C₁-C₆ alkyl;
R¹², R¹³, R¹⁴, R¹⁵, R¹⁶, R¹⁷, R¹⁸, R¹⁹, R²⁰R²¹, R²², R²³, R²⁴, R²⁵, R²⁶, R²⁷ and R²⁸ are independently selected from a group consisting of H, unsubstituted C₁-C₆ alkyl, substituted C₁-C₆ alkyl, unsubstituted phenyl, substituted phenyl, heterocyclic, R¹⁵ and R¹⁶ may be taken together to form an unsubstituted C₁-C₆ alkyl or a substituted C₁-C₆ alkyl, and R¹⁹ and R²⁰ may be taken together to form an unsubstituted C₁-C₆ alkyl or a substituted C₁-C₆ alkyl; and
n is an integer from 1 to 6;
wherein an unsubstituted alkyl, cycloalkyl, or phenyl group comprises all carbon atoms, and a substituted alkyl, cycloalkyl, or aryl group comprises at least one nitrogen atom, oxygen atom, halogen atom, or a combination thereof for at least one carbon atom.

2. The compound of claim 1, wherein R¹, R², R³, and R⁴ are independently selected from a group consisting of H, N02, CN, CHO, F, Cl, Br, I, CF₃, unsubstituted C₁-C₆ alkyl, substituted C₁-C₆ alkyl, COR¹², CO₂H, CO₂R¹³, CONH₂, CONHR¹⁴, and CONR¹⁵R¹⁶;
R⁵ is selected from a group consisting of OH, OR¹⁷, NH₂, NHR¹⁸, NR¹⁹R²⁰, NHCOR²¹, and NHCO₂R²²;
R⁶ is H or an unsubstituted C₁-C₆ alkyl;
Ar is 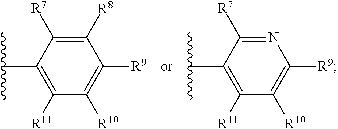
R⁷, R⁸, R⁹, R¹⁰, and R¹¹ are independently H, F, Cl, Br, I, CF₃, CN, OH, OR²³, unsubstituted C₁-C₄ alkyl, substituted C₁-C₄ alkyl, unsubstituted phenyl, or substituted phenyl, R⁷ and R⁸ may be taken together to form an unsubstituted C₁-C₄ alkyl, or a substituted C₁-C₄ alkyl, R⁸ and R⁹ may be taken together to form an unsubstituted C₁-C₄ alkyl, or a substituted C₁-C₄ alkyl, R⁹ and R¹⁰ may be taken together to form an unsubstituted C₁-C₄ alkyl, or a substituted C₁-C₄ alkyl, R¹⁰ and R¹¹ may be taken together to form an unsubstituted C₁-C₄ alkyl, or a substituted C₁-C₄ alkyl;
R¹², R¹³, R¹⁴, R¹⁵, R¹⁶, R¹⁷, R¹⁸, R¹⁹, R²⁰, R²¹, R²², and R²³, are independently selected from a group consisting of H, C₁-C₄ alkyl, unsubstituted phenyl, substituted phenyl, heterocycle, and R¹⁹ and R²⁰ may be taken together to form an unsubstituted C₁-C₆ alkyl or a substituted C₁-C₆ alkyl; and
n is an integer from 1 to 4;
wherein an unsubstituted alkyl, cycloalkyl, or phenyl group comprises all carbon atoms, and a substituted alkyl, cycloalkyl, or aryl group comprises at least one nitrogen atom, oxygen atom, halogen atom, or a combination thereof for at least one carbon atom.

3. The compound of claim 2, wherein R¹, R², R³, and R⁴ are independently selected from a group consisting of H, F, Cl, Br, I, CF₃, CH₃, CH₂CH₃, CH₂CH₂CH₃, CH(CH₃)₂, and C(CH₃)₃;
R⁵ is selected from a group consisting of NHCH₃, N(CH₃)₂, NHCH₂CH₃, N(CH₂CH₃)₂, NHCH₂CH₂CH₃, N(CH₂CH₂CH₃)₂, NHCH(CH₃)₂, N(CH(CH₃)₂)₂, NCH₃Ph, NCH₂CH₃Ph, NCH₃CH(CH₃)₂, NH(C(CH₃)₃, pyridine, pyrazole, pyridazine, and pyrimidine;
R is H or CH₃;
Ar is 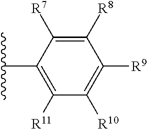
R⁷, R⁸, R⁹, R¹⁰, and R¹¹ are independently selected from a group consisting of H, F, Cl, Br, I, CF₃, CN, OH, OCH₃, OCH₂CH₃, OPh, CH₃, CH and n is an integer from 1 to 4.

4. The compound of claim 3, wherein R¹, R³, R⁴, and R⁶ are H; R² is Cl; R⁵ is N(CH₃)₂;
Ar is 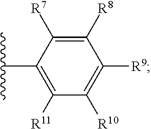
R⁷, R⁸, and R¹¹ are H; R⁸ and R⁹ are OCH₃; and n=3 as shown in the compound comprising Formula (III):

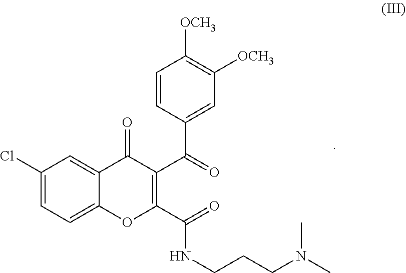 [SMILES: COc1ccc(C(=O)c2c(C(=O)NCCCN(C)C)oc3ccc(Cl)cc3c2=O)cc1OC]

5.-8. (canceled)

9. The compound according to claim 1, wherein the compound comprises agonistic activity toward growth hormone secretagogue receptor 1a (GHSR1a).

10. The compound according to claim 1, wherein the compound comprises a biased agonist of GHSR1a.

11. The compound according to claim 10, wherein the compound comprises a G protein-bias.

12. The compound according to claim 1, wherein the compound comprises a G protein-bias to one or more G alpha subunits.

13. The compound according to claim 12, wherein the one or more G alpha subunits comprises Gαₛ, Gα_{olf}, Gαᵢ₁, Gαᵢ₂, Gαᵢ₃, Gα_{oA}, Gα_{oB}, Gα_{z}, Gα_{q}, Gα₁₁, Gα₁₄, Gα₁₅, Gα₁₂, Gα₁₃, or any combination thereof.

14. The compound according to claim 1, wherein the compound comprises a G protein-bias to Gα_{q}.

15. The compound according to claim 1, wherein the compound does not compete with ghrelin peptide for binding to the GHSR1a.

16. A pharmaceutical composition comprising the compound according to claim 1 and at least one pharmaceutically acceptable excipient.

17. A method for treating a subject having a health condition, comprising administering to the subject having a health condition, the compound according to claim 1 or the pharmaceutical composition according to claim 16.

18. The method according to claim 17, wherein the subject having a health condition comprises an imbalance in brain dopamine homeostasis.

19. The method according to claim 18, wherein the imbalance in brain dopamine homeostasis comprises Parkinson's disease, attention deficit hyperactivity disorder, Tourette syndrome, schizophrenia, bipolar disorder, Alzheimer's Disease, addiction, eating disorder, or any combination thereof.

20. The method according to claim 18, wherein the imbalance in brain dopamine homeostasis comprises a neurological disorder associated with involuntary motor movements.

21. The method according to claim 20, wherein the neurological disorder associated with involuntary motor movements is Parkinson's disease.

22. The method according to claim 18, wherein the imbalance in brain dopamine homeostasis comprises an addition to a drug, alcohol, food, or any combination thereof.

23. The method according to claim 18, wherein the imbalance in brain dopamine homeostasis comprises an eating disorder.

24. The method according to claim 17, wherein the compound according to claim 1 or the pharmaceutical composition according to claim 16 is administered to the subject systemically, topically, subcutaneously, or by direct administration into a brain tissue.

25. The method according to claim 24, wherein the compound according to claim 1 or the pharmaceutical composition according to claim 16 is administered to the subject orally.

26. The method according to claim 25, wherein the compound according to claim 1 or the pharmaceutical composition according to claim 16 is administered to the subject orally at a concentration ranging from about 0.5 mg/kg to about 50 mg/kg.

27. The method according to claim 26, wherein the compound according to claim 1 or the pharmaceutical composition according to claim 16 is administered to the subject orally, wherein the compound according to claim 1 comprises an oral bioavailability of 1% to 15%.

28. The method according to claim 24, wherein the compound according to claim 1 or the pharmaceutical composition according to claim 16 is administered to the subject intraperitoneally.

29. The method according to claim 28, wherein the compound according to claim 1 or the pharmaceutical composition according to claim 16 is administered to the subject intraperitoneally at a concentration ranging from about 0.5 mg/kg to about 50 mg/kg.

30. The method according to claim 29, wherein the compound according to claim 1 or the pharmaceutical composition according to claim 16 is administered to the subject intraperitoneally, wherein the compound according to claim 1 comprises an IP bioavailability of 10% to 35%.

31. A kit comprising the compound according to claim 1 or the pharmaceutical composition according to claim 16, and at least one container.

32. The kit according to claim 31, wherein the kit is of use to treat a health condition in a subject having or suspected of having an imbalance in brain dopamine homeostasis.
